SEQUENCE LISTING (28 sequences)

SEQ ID NO: 1 - DNA, 365 bp, Artificial Sequence
  source: /note=“Description of Artificial Sequence Synthetic polynucleotide”
aggtgcagctggtggagtctgggggaaacttggaacagcctggggggtcccttagactctcctgtacagcctctggattcacctttagcagatctgccatgaactgggtccgccgggctccagggaaggggctggagtgggtctcaggaattagtggtagtggtggtcgaacatactacgcagactccgtgaagggccggttcaccatctccagagacaattccaagaatacgctatatctgcaaatgaacagcctgagcgccgaggacacggccgcatattactgtgcgaaagattcgtatactaccagttggtacggaggtatggacgtctggggccacgggaccacggtcaccgtctcctca

SEQ ID NO: 2 - protein, 121 aa, Artificial Sequence
  source: /note=“Description of Artificial Sequence Synthetic polypeptide”
VQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 3 - DNA, 23 bp, Artificial Sequence
  source: /note=“Description of Artificial Sequence Synthetic oligonucleotide”
gattcacctttagcagatctgcc

SEQ ID NO: 4 - protein, 7 aa, Artificial Sequence
  source: /note=“Description of Artificial Sequence Synthetic peptide”
FTFSRSA

SEQ ID NO: 5 - DNA, 23 bp, Artificial Sequence
  source: /note=“Description of Artificial Sequence Synthetic oligonucleotide”
ttagtggtagtggtggtcgaaca

SEQ ID NO: 6 - protein, 7 aa, Artificial Sequence
  source: /note=“Description of Artificial Sequence Synthetic peptide”
SGSGGRT

SEQ ID NO: 7 - DNA, 44 bp, Artificial Sequence
  source: /note=“Description of Artificial Sequence Synthetic oligonucleotide”
cgaaagattcgtatactaccagttggtacggaggtatggacgtc

SEQ ID NO: 8 - protein, 14 aa, Artificial Sequence
  source: /note=“Description of Artificial Sequence Synthetic peptide”
KDSYTTSWYGGMDV

SEQ ID NO: 9 - DNA, 323 bp, Artificial Sequence
  source: /note=“Description of Artificial Sequence Synthetic polynucleotide”
acatccagatgacccagtctccatcttccgtgtctgcatctgtaggagacagagtcaccatcacttgtcgggcgagtcagggtattttcagctggttagcctggtatcagcagaaaccaggaaaagcccctaagctcctgatctatgctgcttccagtttacaaagtggggtcccatcaagattcagcggcagtggatctgggacagatttcactctcaccatcagcagcctgcagcctgaggattttgcaatttactattgtcaacaggctaacagtgtcccgatcaccttcggccaagggacacgactggagattaaacga

SEQ ID NO: 10 - protein, 107 aa, Artificial Sequence
  source: /note=“Description of Artificial Sequence Synthetic polypeptide”
IQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKR

SEQ ID NO: 11 - DNA, 17 bp, Artificial Sequence
  source: /note=“Description of Artificial Sequence Synthetic oligonucleotide”
agggtattttcagctgg

SEQ ID NO: 12 - protein, 5 aa, Artificial Sequence
  source: /note=“Description of Artificial Sequence Synthetic peptide”
GIFSW

SEQ ID NO: 13 - DNA, 8 bp, Artificial Sequence
  source: /note=“Description of Artificial Sequence Synthetic oligonucleotide”
ctgcttcc

SEQ ID NO: 14 - protein, 2 aa, Artificial Sequence
  source: /note=“Description of Artificial Sequence Synthetic peptide”
AS

SEQ ID NO: 15 - DNA, 26 bp, Artificial Sequence
  source: /note=“Description of Artificial Sequence Synthetic oligonucleotide”
aacaggctaacagtgtcccgatcacc

SEQ ID NO: 16 - protein, 8 aa, Artificial Sequence
  source: /note=“Description of Artificial Sequence Synthetic peptide”
QANSVPIT

SEQ ID NO: 17 - DNA, 1349 bp, Artificial Sequence
  source: /note=“Description of Artificial Sequence Synthetic polynucleotide”
aggtgcagctggtggagtctgggggaaacttggaacagcctggggggtcccttagactctcctgtacagcctctggattcacctttagcagatctgccatgaactgggtccgccgggctccagggaaggggctggagtgggtctcaggaattagtggtagtggtggtcgaacatactacgcagactccgtgaagggccggttcaccatctccagagacaattccaagaatacgctatatctgcaaatgaacagcctgagcgccgaggacacggccgcatattactgtgcgaaagattcgtatactaccagttggtacggaggtatggacgtctggggccacgggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 448 aa, Artificial Sequence
  source: /note=“Description of Artificial Sequence Synthetic polypeptide”
VQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 644 bp, Artificial Sequence
  source: /note=“Description of Artificial Sequence Synthetic polynucleotide”
acatccagatgacccagtctccatcttccgtgtctgcatctgtaggagacagagtcaccatcacttgtcgggcgagtcagggtattttcagctggttagcctggtatcagcagaaaccaggaaaagcccctaagctcctgatctatgctgcttccagtttacaaagtggggtcccatcaagattcagcggcagtggatctgggacagatttcactctcaccatcagcagcctgcagcctgaggattttgcaatttactattgtcaacaggctaacagtgtcccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 213 aa, Artificial Sequence
  source: /note=“Description of Artificial Sequence Synthetic polypeptide”
IQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, Artificial Sequence
  source: /note=“Description of Artificial Sequence Synthetic peptide”
GFTFRDYA

SEQ ID NO: 22 - protein, 8 aa, Artificial Sequence
  source: /note=“Description of Artificial Sequence Synthetic peptide”
ISGSGGNT

SEQ ID NO: 23 - protein, 16 aa, Artificial Sequence
  source: /note=“Description of Artificial Sequence Synthetic peptide”
AKDRLSITIRPRYYGL

SEQ ID NO: 24 - protein, 11 aa, Artificial Sequence
  source: /note=“Description of Artificial Sequence Synthetic peptide”
QSLLYSIGYNY

SEQ ID NO: 25 - protein, 3 aa, Artificial Sequence
  source: /note=“Description of Artificial Sequence Synthetic peptide”
LGS

SEQ ID NO: 26 - protein, 9 aa, Artificial Sequence
  source: /note=“Description of Artificial Sequence Synthetic peptide”
MQALQTPYT

SEQ ID NO: 27 - protein, 124 aa, Artificial Sequence
  source: /note=“Description of Artificial Sequence Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 28 - protein, 112 aa, Artificial Sequence [sequence omitted]

(19) United States
(12) Patent Application Publication
(10) Pub. No.: US 2021/0000949 A1
(43) Pub. Date: Jan. 7, 2021

(54) METHODS FOR TREATING OR PREVENTING ASTHMA BY ADMINISTERING AN IL-33 ANTAGONIST

(71) Applicants: SANOFI BIOTECHNOLOGY, Paris (FR); REGENERON PHARMACEUTICALS, INC., Tarrytown, NY (US)

(72) Inventors: Helene Goulaouic, Bridgewater, NJ (US); Andreas Jessel, Bridgewater, NJ (US); Raolat Abdulai, Bridgewater, NJ (US); Ariel Teper, Bridgewater, NJ (US); Marcella Ruddy, Wellesley, MA (US); Nikhil Amin, Chappaqua, NY (US); Sivan Harel, Tarrytown, NY (US); Weronika Szafran, Newburgh, NY (US); John Huber, Oradell, NJ (US); Joanna Pols, Morristown, NJ (US)

(21) Appl. No.: 16/863,800

(22) Filed: Apr. 30, 2020

Related U.S. Application Data
(60) Provisional application No. 62/898,900, filed on Sep. 11, 2019.
(60) Provisional application No. 62/848,248, filed on May 15, 2019.
(60) Provisional application No. 62/841,481, filed on May 1, 2019.

(51) Int. Cl. A61K 39/395; C07K 16/24; C07K 16/28; A61K 31/56; A61K 31/58; A61K 31/138; A61P 11/06

(52) U.S. Cl. CPC A61K 39/3955; C07K 16/244; C07K 16/2866; A61K 31/56; A61K 31/58; A61K 2039/507; A61P 11/06; C07K 2317/51; C07K 2317/515; C07K 2317/76; A61K 31/138

(57) ABSTRACT

The invention provides methods for treating or preventing asthma and associated conditions in a patient. The methods featured in the invention comprise administering to a subject in need thereof a therapeutic composition comprising an interleukin-33 (IL-33) antagonist, such as an anti-IL-33 antibody. The methods featured in the invention further comprise administering to a subject in need thereof a first therapeutic composition comprising an interleukin-33 (IL-33) antagonist, such as an anti-IL-33 antibody, and a second therapeutic composition comprising an interleukin-4 receptor (IL-4R) antagonist, such as an anti-IL-4R antibody.

### RELATED APPLICATIONS

This application claims priority to U.S. Provisional Patent Application Serial Nos. 62/841,481, filed May 1, 2019, 62/848,248, filed May 15, 2019, and 62/898,900, filed Sep. 11, 2019. The entire disclosure of each of these applications is hereby incorporated herein by reference.

### FIELD OF THE INVENTION

The invention relates to the treatment and/or prevention of asthma and related conditions. More specifically, the invention relates to the administration of an interleukin-33 (IL-33) antagonist to treat or prevent asthma in a patient in need thereof. The invention also relates to the administration of an IL-33 antagonist and an interleukin-4 receptor (IL-4R) antagonist to treat or prevent asthma in a patient in need thereof.

### BACKGROUND

Asthma is a chronic inflammatory disease of the airways characterized by airway hyper responsiveness, acute and chronic bronchoconstriction, airway edema, and mucus plugging. The inflammation component of asthma is thought to involve many cell types, including mast cells, eosinophils, T lymphocytes, neutrophils, and epithelial cells, and their biological products. Patients with asthma most often present with symptoms of wheezing, shortness of breath, cough, and chest tightness. For most asthma patients, a regimen of controller therapy and bronchodilator therapy provides adequate long-term control. Inhaled corticosteroids (ICS) are considered the “gold standard” in controlling asthma symptoms, and inhaled beta2-agonists are the most effective bronchodilators currently available. Studies have shown that combination therapy of an ICS with an inhaled long-acting beta2-agonist (LABA) provides better asthma control than high doses of ICS alone. Consequently, combination therapy has been the recommended treatment for subjects who are not controlled on low doses of ICS alone.

Nonetheless, it is estimated that 5% to 10% of the population with asthma has symptomatic disease despite maximum recommended treatment with combinations of anti-inflammatory and bronchodilator drugs. Furthermore, this severe asthma population accounts for up to 50% of the total health cost through hospital admissions, use of emergency services, and unscheduled physician visits. There is an unmet need for a new therapy in this severe asthma population as many of these patients are poorly responsive to ICS due to a number of cellular and molecular mechanisms. In addition, the long term adverse effects of systemic and inhaled corticosteroids on bone metabolism, adrenal function, and growth in children lead to attempts to minimize the amount of corticosteroid usage. Although a large portion of asthma patients are managed reasonably well with current treatments, patients with severe uncontrolled asthma have few therapeutic treatment options that can adequately control the disease. The consequence of unresponsiveness to therapy or lack of compliance with therapy is loss of asthma control and ultimately asthma exacerbation.

An estimated 45% of patients with severe asthma require systemic glucocorticoids to control their disease, and to prevent life-threatening exacerbations associated with increased risk of permanent damage to lung tissue, progressive fixed airway obstruction, and accelerated decline in lung function. However, systemic glucocorticoids act non-selectively and are associated with significant multi-organ toxicities and broad immunosuppression. There is a need for safer and more effective targeted therapies that prevent exacerbations and lung function impairment, improve asthma symptoms and control, and reduce or obviate the need for oral glucocorticoids.

Approximately 20% of patients with asthma have uncontrolled, moderate-to-severe disease with recurrent exacerbations and persistent symptoms despite maximized standard-of-care controller therapy. This population is at an increased risk of morbidity (especially exacerbations) and accounts for significant healthcare resources. These patients have substantially reduced lung function, despite maximum treatment, and are destined to inexorably further lose lung function. No currently approved treatments have been shown to slow this inexorable decline in these patients, or to consistently and meaningfully increase lung function.

Accordingly, a need exists in the art for novel targeted therapies for the treatment and/or prevention of asthma.

### BRIEF SUMMARY OF THE INVENTION

According to one aspect, a method for treating asthma in a subject in need thereof comprising administering to the subject an initial dose of about 300 mg of an antibody or antigen-binding fragment thereof that specifically binds interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16, and one or more maintenance doses of about 300 mg of the antibody or antigen-binding fragment thereof is provided.

In certain exemplary embodiments, loss of asthma control (LOAC) is reduced in the subject.

In certain exemplary embodiments, one or more asthma-associated parameter(s) are improved in the subject.

In certain exemplary embodiments, the asthma-associated parameter is selected from the group consisting of forced expiratory volume in 1 second (FEV1), peak expiratory flow (PEF), forced vital capacity (FVC), forced expiratory flow (FEF) 25%-75%, frequency or dosage of a long-acting β2 adrenergic agonist (LABA), frequency or dosage of an inhaled corticosteroid, and frequency or dosage of a systemic steroid.

In certain exemplary embodiments, pre-bronchodilator FEV1 is improved.

In certain exemplary embodiments, the subject has a blood eosinophil count of: greater than or equal to about 300 cells per μl; of about 150 to 299 cells/μL; or of about <150 cells/μL.

In certain exemplary embodiments, the subject has a blood eosinophil count of greater than or equal to about 300 cells per μl.

In certain exemplary embodiments, blood eosinophil levels are reduced.

In certain exemplary embodiments, the subject has high blood periostin levels of greater than or equal to about 60 ng/ml, greater than or equal to about 65 ng/ml, greater than or equal to about 70 ng/ml, greater than or equal to about 75 ng/ml, greater than or equal to about 80 ng/ml, or greater than or equal to about 74.4 ng/mL, or the subject has high blood periostin levels of less than about 80 ng/mL, less than about 75 ng/mL, less than about 70 ng/mL, less than about 65 ng/mL, or less than about 60 ng/mL, or less than about 74.4 ng/mL. In certain exemplary embodiments, the subject has high blood periostin levels of greater than or equal to about 60 ng/ml, greater than or equal to about 65 ng/ml, greater than or equal to about 70 ng/ml, greater than or equal to about 75 ng/ml, or greater than or equal to about 80 ng/ml. In certain exemplary embodiments, the subject has high blood periostin levels of greater than or equal to about 74.4 ng/mL.

In certain exemplary embodiments, one or both of asthma control questionnaire 5-question version (ACQ-5) score and asthma quality of life questionnaire with standardized activities (AQLQ) score are improved.

In certain exemplary embodiments, emotional function score of the AQLQ is improved.

In certain exemplary embodiments, the frequency or the dosage of the long-acting β2 adrenergic agonist (LABA) is reduced, the frequency or the dosage of the inhaled corticosteroid is reduced, or the frequency or the dosage of the systemic steroid is reduced.

In certain exemplary embodiments, the asthma is moderate-to-severe asthma that is not well-controlled on a background therapy.

In certain exemplary embodiments, the background therapy comprises an inhaled corticosteroid (ICS) and a long-acting β2 adrenergic agonist (LABA).

In certain exemplary embodiments, the background therapy comprises moderate-to-high dose ICS/LABA.

In certain exemplary embodiments, the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10.

In certain exemplary embodiments, the antibody comprises SAR440340.

In certain exemplary embodiments, the antibody or antigen-binding fragment thereof is administered every other week.

In certain exemplary embodiments, the antibody or antigen-binding fragment thereof is administered subcutaneously.

In certain exemplary embodiments, the antibody or antigen-binding fragment thereof is administered as two injections.

In certain exemplary embodiments, the antibody or antigen-binding fragment thereof is administered subcutaneously using an autoinjector, a needle and syringe, or a pen delivery device.

According to another aspect, a method for treating asthma in a subject in need thereof comprising administering to the subject an initial dose of about 300 mg of a first antibody or antigen-binding fragment thereof that specifically binds interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16, one or more maintenance doses of about 300 mg of the first antibody or antigen-binding fragment thereof, an initial dose of about 300 mg of a second antibody or antigen-binding fragment thereof that specifically binds interleukin-4 receptor (IL-4R) and comprises three heavy chain complementary determining region (HCDR) sequences comprising SEQ ID NOs: 21, 22 and 23, and three light chain complementary determining region (LCDR) sequences comprising SEQ ID NOs: 24, 25 and 26, and one or more maintenance doses of about 300 mg of the second antibody or antigen-binding fragment thereof is provided.

In certain exemplary embodiments, LOAC is reduced in the subject.

In certain exemplary embodiments, one or more asthma-associated parameter(s) are improved in the subject.

In certain exemplary embodiments, the asthma-associated parameter is selected from the group consisting of forced expiratory volume in 1 second (FEV1), peak expiratory flow (PEF), forced vital capacity (FVC), forced expiratory flow (FEF) 25%-75%, frequency or dosage of a long-acting (32 adrenergic agonist (LABA), frequency or dosage of an inhaled corticosteroid, and frequency or dosage of a systemic steroid.

In certain exemplary embodiments, post-bronchodilator FEV1 is improved.

In certain exemplary embodiments, the subject has a blood eosinophil count of: greater than or equal to about 300 cells per μl; of about 150 to 299 cells/μL; or of about <150 cells/μL.

In certain exemplary embodiments, the subject has high blood periostin levels of greater than or equal to about 60 ng/ml, greater than or equal to about 65 ng/ml, greater than or equal to about 70 ng/ml, greater than or equal to about 75 ng/ml, greater than or equal to about 80 ng/ml, or greater than or equal to about 74.4 ng/mL, or the subject has high blood periostin levels of less than about 80 ng/mL, less than about 75 ng/mL, less than about 70 ng/mL, less than about 65 ng/mL, less than about 60 ng/mL, or less than about 74.4 ng/mL. In certain exemplary embodiments, the subject has high blood periostin levels of greater than or equal to about 60 ng/ml, greater than or equal to about 65 ng/ml, greater than or equal to about 70 ng/ml, greater than or equal to about 75 ng/ml, greater than or equal to about 80 ng/ml, or greater than or equal to about 74.4 ng/mL.

In certain exemplary embodiments, the asthma is moderate-to-severe asthma that is not well-controlled on a background therapy.

In certain exemplary embodiments, the first antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10.

In certain exemplary embodiments, the first antibody comprises SAR440340.

In certain exemplary embodiments, the second antibody or antigen-binding fragment thereof comprises a heavy chain variable region (HCVR) comprising the amino acid sequence of SEQ ID NO: 27 and a light chain variable region (LCVR) comprising the amino acid sequence of SEQ ID NO: 28.

In certain exemplary embodiments, the second antibody comprises dupilumab.

In certain exemplary embodiments, the first antibody or antigen-binding fragment thereof and the second antibody or antigen-binding fragment thereof are each administered every other week.

In certain exemplary embodiments, the first antibody or antigen-binding fragment thereof and the second antibody or antigen-binding fragment thereof are each administered subcutaneously.

In certain exemplary embodiments, the second antibody or antigen-binding fragment thereof is administered to the subject before, after, or concurrent with the first antibody or antigen-binding fragment thereof.

In certain exemplary embodiments, the first antibody or antigen-binding fragment thereof is administered as two injections and the second antibody or antigen-binding fragment thereof is administered as one injection.

In certain exemplary embodiments, the first antibody or antigen-binding fragment thereof and the second antibody or antigen-binding fragment thereof are each administered subcutaneously using an autoinjector, a needle and syringe, or a pen delivery device.

In certain exemplary embodiments, at least one additional therapeutic agent is administered to the subject.

In certain exemplary embodiments, the at least one additional therapeutic agent comprises one or both of an ICS and a LABA.

In certain exemplary embodiments, the ICS is fluticasone or budesonide.

In certain exemplary embodiments, the LABA is salmeterol or formoterol.

In certain exemplary embodiments, the ICS and LABA are both administered, the ICS is fluticasone and the LABA is salmeterol.

In another aspect, a method for treating moderate-to-severe asthma in a subject in need thereof comprising administering to the subject an initial dose of about 300 mg of SAR440340, and one or more maintenance doses of about 300 mg of SAR440340, wherein SAR440340 is administered subcutaneously every other week, is provided.

In another aspect, a method for treating moderate-to-severe asthma in a subject in need thereof comprising administering to the subject an initial dose of about 300 mg of SAR440340, one or more maintenance doses of about 300 mg of SAR440340, an initial dose of about 300 mg of dupilumab, and one or more maintenance doses of about 300 mg of dupilumab, wherein SAR440340 and dupilumab are administered subcutaneously every other week, is provided.

In another aspect, a method for reducing an asthma patient's dependence or one or both of an inhaled corticosteroid (ICS) and a long-acting β2 adrenergic agonist (LABA) for the treatment of one or more asthma exacerbations comprising administering to a subject who has moderate-to-severe asthma that is partially controlled or uncontrolled with a background asthma therapy comprising an ICS, a LABA, or a combination thereof, a defined dose of an antibody or antigen-binding fragment thereof that specifically binds to interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16, at a defined frequency for an initial treatment period while maintaining the subject's background asthma therapy for the initial treatment period, and gradually reducing or eliminating the dosage of the ICS, the LABA or the combination thereof administered to the subject over the course of a subsequent treatment period while continuing to administer the antibody or antigen-binding fragment thereof to the subject at the defined frequency and dose used during the initial treatment period, is provided.

In certain exemplary embodiments, the ICS is fluticasone, budesonide, or mometasone, and the LABA is salmeterol or formoterol.

In certain exemplary embodiments, an ICS/LABA combination is selected from the group consisting of fluticasone/salmeterol, budesonide/formoterol, and mometasone/formoterol.

In certain exemplary embodiments, the dosage of one or both of the LABA and the ICS are eliminated at the end of the initial treatment period.

In certain exemplary embodiments, the dosage of one or both of the LABA and the ICS are gradually reduced or eliminated over the course of 2 to 8 weeks.

In certain exemplary embodiments, the method further comprises administering to the subject a second antibody or antigen-binding fragment thereof that specifically binds interleukin-4 receptor (IL-4R) and comprises three HCDR sequences comprising SEQ ID NOs: 21, 22 and 23, and three LCDR sequences comprising SEQ ID NOs: 24, 25 and 26.

In another aspect, a method for treating asthma in a subject in need thereof comprising administering to the subject a dose of about 300 mg of an antibody or antigen-binding fragment thereof that specifically binds interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16, is provided.

In certain exemplary embodiments, loss of asthma control (LOAC) is reduced in the subject.

In certain exemplary embodiments, one or more asthma-associated parameter(s) are improved in the subject. In certain exemplary embodiments, the asthma-associated parameter is selected from the group consisting of forced expiratory volume in 1 second (FEV1), peak expiratory flow (PEF), forced vital capacity (FVC), forced expiratory flow (FEF) 25%-75%, frequency or dosage of a long-acting β2 adrenergic agonist (LABA), frequency or dosage of an inhaled corticosteroid, and frequency or dosage of a systemic steroid.

In certain exemplary embodiments, pre-bronchodilator FEV1 is improved.

In certain exemplary embodiments, the subject has a blood eosinophil count of: greater than or equal to about 300 cells per μl; of about 150 to 299 cells/μL; or of about <150 cells/μL. In certain exemplary embodiments, the subject has a blood eosinophil count of greater than or equal to about 300 cells per μl. In certain exemplary embodiments, blood eosinophil levels are reduced.

In certain exemplary embodiments, the subject has high blood periostin levels or low blood periostin levels. In certain exemplary embodiments, the subject has high blood periostin levels of about ≥74.4 ng/mL.

In certain exemplary embodiments, one or both of asthma control questionnaire 5-question version (ACQ-5) score and asthma quality of life questionnaire with standardized activities (AQLQ) score are improved. In certain exemplary embodiments, emotional function score of the AQLQ is improved.

In certain exemplary embodiments, the frequency or the dosage of the long-acting β2 adrenergic agonist (LABA) is reduced, the frequency or the dosage of the inhaled corticosteroid is reduced, or the frequency or the dosage of the systemic steroid is reduced.

In certain exemplary embodiments, the asthma is moderate-to-severe asthma that is not well-controlled on a background therapy.

In certain exemplary embodiments, the background therapy comprises an inhaled corticosteroid (ICS) and a long-acting β2 adrenergic agonist (LABA).

In certain exemplary embodiments, the background therapy comprises moderate-to-high dose ICS/LABA.

In certain exemplary embodiments, the antibody or antigen-binding fragment thereof is administered subcutaneously using an autoinjector, a needle and syringe, or a pen delivery device.

In certain exemplary embodiments, the antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the antibody comprises SAR440340.

In another aspect, a method for treating asthma in a subject in need thereof comprising administering to the subject a dose of about 300 mg of a first antibody or antigen-binding fragment thereof that specifically binds interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16, and a dose of about 300 mg of a second antibody or antigen-binding fragment thereof that specifically binds interleukin-4 receptor (IL-4R) and comprises three heavy chain complementary determining region (HCDR) sequences comprising SEQ ID NOs: 21, 22 and 23, and three light chain complementary determining region (LCDR) sequences comprising SEQ ID NOs: 24, 25 and 26, is provided.

In certain exemplary embodiments, loss of asthma control (LOAC) is reduced in the subject.

In certain exemplary embodiments, one or more asthma-associated parameter(s) are improved in the subject. In certain exemplary embodiments, the asthma-associated parameter is selected from the group consisting of forced expiratory volume in 1 second (FEV1), peak expiratory flow (PEF), forced vital capacity (FVC), forced expiratory flow (FEF) 25%-75%, frequency or dosage of a long-acting β2 adrenergic agonist (LABA), frequency or dosage of an inhaled corticosteroid, and frequency or dosage of a systemic steroid.

In certain exemplary embodiments, pre-bronchodilator FEV1 is improved.

In certain exemplary embodiments, the subject has a blood eosinophil count of: greater than or equal to about 300 cells per μl; of about 150 to 299 cells/μL; or of about <150 cells/μL. In certain exemplary embodiments, the subject has a blood eosinophil count of greater than or equal to about 300 cells per μl. In certain exemplary embodiments, blood eosinophil levels are reduced.

In certain exemplary embodiments, the subject has high blood periostin levels or low blood periostin levels. In certain exemplary embodiments, the subject has high blood periostin levels of about ≥74.4 ng/mL.

In certain exemplary embodiments, one or both of asthma control questionnaire 5-question version (ACQ-5) score and asthma quality of life questionnaire with standardized activities (AQLQ) score are improved. In certain exemplary embodiments, emotional function score of the AQLQ is improved.

In certain exemplary embodiments, the frequency or the dosage of the long-acting β2 adrenergic agonist (LABA) is reduced, the frequency or the dosage of the inhaled corticosteroid is reduced, or the frequency or the dosage of the systemic steroid is reduced.

In certain exemplary embodiments, the asthma is moderate-to-severe asthma that is not well-controlled on a background therapy.

In certain exemplary embodiments, the background therapy comprises an inhaled corticosteroid (ICS) and a long-acting β2 adrenergic agonist (LABA).

In certain exemplary embodiments, the background therapy comprises moderate-to-high dose ICS/LABA.

In certain exemplary embodiments, the antibody or antigen-binding fragment thereof is administered subcutaneously using an autoinjector, a needle and syringe, or a pen delivery device.

In certain exemplary embodiments, the first antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10. In certain exemplary embodiments, the first antibody comprises SAR440340.

In certain exemplary embodiments, the second antibody or antigen-binding fragment thereof comprises a heavy chain variable region (HCVR) comprising the amino acid sequence of SEQ ID NO: 27 and a light chain variable region (LCVR) comprising the amino acid sequence of SEQ ID NO: 28. In certain exemplary embodiments, the second antibody comprises dupilumab.

Other embodiments will become apparent from a review of the ensuing detailed description, drawings, tables and accompanying claims.

### BRIEF DESCRIPTION OF THE FIGURES

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 depicts a study flow chart of the 12-week proof of concept (PoC) study that is designed to assess the efficacy, safety, and tolerability of SAR440340, and the co-administration of SAR440340 and dupilumab, in patients with moderate-to-severe asthma who are not well controlled on inhaled ICS/LABA therapy.

FIG. 2 depicts a table corresponding to the flow chart of FIG. 1. Abbreviations: ACQ-5, asthma control questionnaire-5; AQLQ(S), asthma quality of life questionnaire; β-hCG, beta human chorionic gonadotropin; D, day; EOT, end of treatment; FEF, forced expiratory flow; FeNO, fraction of exhaled nitric oxide; FEV1, forced expiratory volume in 1 second; ICS, inhaled corticosteroid; IgE, Immunoglobulin E; IMP, investigational medicinal product; IL33, interleukin-33; IVRS/IWRS, interactive voice/web response system; LABA, long-acting β2 adrenergic agonist; LOAC, loss of asthma control; BD, bronchodilator; PARC, pulmonary and activation-regulated chemokine; PEF, peak expiratory flow; PGx, pharmacogenomics; PK, pharmacokinetic; RNA, ribonucleic acid; RQLQ, rhinoconjunctivitis quality of life questionnaire; SAE, serious adverse event; sST2, soluble IL33 receptor; V, visit; W, week. ^{a}The study visits occur on the planned dates (relative to the first injection), as scheduled. The visit schedule should be adhered to within ±3 days for the screening period and randomized IMP treatment period, and ±5 days for the visits during the post IMP treatment safety follow-up period. ^{b}After 5 weeks of ICS/LABA withdrawal phase, patients with high dose ICS (fluticasone) background at visit 2/baseline will be on IMP treatment without background therapy for 3 weeks. ^{c}After 4 weeks of ICS/LABA withdrawal phase, patients with medium dose fluticasone background at visit 2/baseline will be on IMP treatment without background therapy for 4 weeks. ^{d}End of IMP treatment (EOT) visit: Patients who discontinue prematurely from the study (i.e., early treatment discontinuation (ETD)), prior to completing the 12-week IMP treatment (e.g., due to a LOAC event or due to other reasons), will be evaluated as soon as possible at the individual patients' EOT Visit, using procedures as planned for the EOT Visit at week 12 (Visit 14). At their EOT visit, all patients will resume their prescreening ICS/LABA background therapy and enter the 20-week post-IMP treatment period (V15 to V17). If a patient's asthma cannot be adequately controlled by the prescreening ICS/LABA therapy, additional controller therapies may be prescribed based on the Investigator's clinical judgment. ^{e}The post-IMP treatment period will start at week 12 for patients who complete the IMP treatment period, and may start earlier than week 12 for patients who meet the criteria for a LOAC or discontinue IMP treatment early (due to other reasons) prior to completing the 12-week IMP treatment. ^{f}Visit 16 visit can be either an on-site visit or a phone call. ^{g}Patients with reversibility of at least 12% and 200 mL in FEV1 after administration of 2 to 4 puffs (200-400 mcg) of albuterol/salbutamol or levalbuterol/levosalbutamol during screening or documented history of a reversibility test that meets this criteria within 12 months prior to visit 1 or documented positive response to methacholine challenge (a decrease in FEV by 20% [PC20] of <8 mg/mL) within 12 months prior to visit 1/screening is considered acceptable to meet this inclusion criterion. If the subject does not meet the qualifying criteria for reversibility at visit 1/screening, up to 2 additional attempts during the screening period, each on a different day prior to visit 2/baseline, may be performed. When reversibility assessment is repeated during the screening period, the prebronchodilator FEV1 should again meet the inclusion criterion (I 03) of >40% of predicted normal. Patients should be monitored by site personnel for at least 30 minutes after administration of all IMP injections. Monitoring period may be extended as per country specific requirements. ⁱElectronic diary/PEF meter is a handheld device used for daily recording of salbutamol/albuterol or levosalbutamol/levalbuterol use, asthma controller drug use, asthma symptom score numerical rating scale (NRS), nocturnal awakenings due to asthma symptoms and AM and PM PEF, and recording of patient's answers to the ACQ-5, AQLQ(S), and RQLQ questionnaires during the scheduled visits. This handheld device is dispensed at visit 1 (including instructions for use) and recorded information is downloaded from this device on the other indicated days. If not already done so, patient will return electronic devices to the site at EOS. Electronic devices will be returned to the sponsor at EOS as the latest. ^{j}After evaluation for LOAC events, all patients that do not meet the criteria for LOAC at week 4/V6, will have the LABA (salmeterol) withdrawn from their background therapy and will be switched from fluticasone/salmeterol combination therapy to clinically comparable ICS dose of fluticasone monotherapy. ^{k}After evaluation for LOAC events, all patients that do not meet the criteria for LOAC at V8, V9, V10 (and V11), will have ICS (fluticasone) withdrawn by 3 or 4 steps of dose reduction depending on their medium or high dose ICS (fluticasone) background treatment at visit 2/baseline, respectively. ^{l}Complete physical examinations will include skin, nasal cavities, eyes, ears, respiratory, cardiovascular, gastrointestinal, neurological, lymphatic, and musculoskeletal systems. ^{m}Vital signs, including systolic and diastolic blood pressure (mmHg), pulse rate (beats per minute), and respiratory rate (breaths per minute) will be measured at all visits detailed in the flowchart. Height (cm) will be measured at screening (visit 1) only. Body weight (kg) will be measured at visit 1/screening, visit 2/baseline and visit 14/EOT. ⁿHematology will include hemoglobin, hematocrit, platelet count, total white blood cell count, differential count, and total red blood cell count. Serum chemistry will include creatinine, blood urea nitrogen, glucose, uric acid, total cholesterol, total protein, albumin, total bilirubin, alanine aminotransferase, aspartate aminotransferase, alkaline phosphatase, electrolytes (sodium, potassium, chloride), bicarbonate, and creatine phosphokinase. Urinalysis will include specific gravity, pH, glucose, ketones, blood, protein, nitrate, leukocyte esterase, urobilinogen, and bilirubin. If any parameter on the dipstick is abnormal, a urine sample should be sent to the central laboratory for quantitative measurement. If positive for protein and/or red blood cells, microscopic analysis will be performed by the central laboratory. Clinical laboratory testing at screening visit 1 will include hepatitis screen covering hepatitis B surface antigen (HBs Ag), hepatitis B surface antibody (HBs Ab), hepatitis B core antibody (HBc Ab), hepatitis C virus antibodies (HCV Ab), human immunodeficiency virus (HIV) screen (Anti-HIV-1 and HIV-2 antibodies) and anti-nuclear antibody (ANA). In case of results showing HBs Ag (negative), and HBc Ab (positive), an HBV DNA testing may be performed prior to randomization to rule out a false positivity if the Investigator believes the patient is a false positive, or to clarify the serological status if the investigator finds it unclear to interpret in absence of known HBV infection. In case of results showing HCV Ab (positive), an HCV RNA testing may be performed to rule out a false positivity, if the Investigator believes the patient is a false positive. Note: Anti-ds DNA antibody will be tested if ANA is positive (≥1:160 titer). The blood sample for serum chemistry must be taken with the patient in fasting state which means no intake of any food or drink except for water for at least 8 hours (if the visit can only be done at a different time of the day and the patient is not fasting, then he/she should be advised to eat light food and the site should document that serum chemistry was not obtained under fasting conditions). Quantiferon gold should be collected for all patients at the screening visit 1. If the result is confirmed positive, the patient should be referred to an infectious disease specialist. Please refer to the central laboratory manual for additional details. ^{p}Only for women of childbearing potential: serum pregnancy test at screening/v1 and urine pregnancy tests at V2, V6, V10, V14/EOT, and V17/EOS. A negative result must be obtained at V1 and at V2 prior to randomization. In case of positive urine test the study treatment will be withheld and a serum pregnancy test to confirm the pregnancy should be performed as soon as possible. Pregnancy will lead to definitive treatment discontinuation in all cases. ^{q}If ADA assessment at week 12 (or the first post-treatment time point analyzed) is positive, additional measurements may be performed from PK samples collected at week 4. ^{r}Hematology sample will be drawn for eosinophils and neutrophils (with other critical values reported per lab manual). ^{s}Exhaled nitric oxide assessment will be conducted prior to spirometry and following a fast of at least 1 hour. ^{t}Spirometry (pre-BD FEV1, post-BD FEV1, and PEF, FVC, FEF) should be performed not earlier than 6 hours after last dose of albuterol or levalbuterol (if any) and withholding the last dose of LABA for at least 12 hours, and prior to administration of investigational product. The postbronchodilator spirometry may be repeated several times within 30 minutes after administration of bronchodilator. ^{u}The ACQ-5, AQLQ(S), and RQLQ are to be completed on the patient's handheld device during clinic visits.

FIG. 3 summarizes efficacy results. SAR440340 demonstrated significant efficacy across multiple endpoints.

FIG. 4 graphically depicts loss of asthma control (LOAC) in the intent to treat (ITT) population. A significant reduction in the proportion of patients with LOAC events in both the SAR440340 and dupilumab arms.

FIG. 5 depicts the distribution of reasons for LOAC in the ITT population. The most frequent reason for LOAC was a failure to meet the peak expiratory flow (PEF) criteria.

FIG. 6 graphically depicts time to LOAC results in the ITT population. There was a significant effect for both SAR440340 and dupilumab in time to LOAC.

FIG. 7A-FIG. 7C graphically depict LOAC by subgroup: (FIG. 7A) eosinophilic subgroup; (FIG. 7B) FeNO subgroup; (FIG. 7C) periostin subgroup. SAR440340 demonstrated balanced reduction in LOAC across eosinophil and FeNO levels. SAR440340 demonstrated greater efficacy in the high periostin subpopulation.

FIG. 8A-FIG. 8D depict forest plots of incidence of LOAC by baseline blood eosinophil count for SAR440340 (FIG. 8A) and dupilumab (FIG. 8B), as well as LOAC by baseline FeNO and periostin for SAR440340 (FIG. 8C) and dupilumab (FIG. 8D).

FIG. 9A-FIG. 9B depict pre-bronchodilator (pre-BD) FEV1 change in baseline as LS mean (SE) (FIG. 9A) and as a percent change from baseline (FIG. 9B).

FIG. 10 depicts pre-BD FEV1 mean change over time in an ITT population. Rapid onset and sustained effect in improvement of FEV1 were observed for both SAR440340 and dupilumab.

FIG. 11 depicts mean change from baseline in pre-BD FEV1 (L) over time in an ITT population with baseline Eos of less than 0.3×10⁹/L. No significant effect was observed over placebo in the low Eos population

FIG. 12 depicts change from baseline in pre-BD FEV1 (L) over time in an ITT population with baseline Eos greater than or equal to 0.3×10⁹/L. SAR440340 has rapid onset and sustained effect on FEV1 over 12 weeks.

FIG. 13 depicts Pre-BD FEV1 mean change from baseline by Eos subgroup.

FIG. 14 depicts change from baseline in pre-bronchodilator FEV1 (L) over time in an ITT population with baseline FeNO<25 ppb. There was no significant effect of FEV1 in the low FeNO population.

FIG. 15 depicts change from baseline in pre-bronchodilator FEV1 (L) over time in an ITT population with baseline FeNO>=25 ppb. SAR440340 had rapid onset and sustained effect on FEV1 over a 12-week period.

FIG. 16 depicts pre-BD FEV1 mean change from baseline by FeNO subgroup. SAR440340 demonstrated a significant effect on FEV1.

FIG. 17 depicts pre-BD FEV1 mean change from baseline by periostin subgroup. SAR440340 demonstrated a significant effect on FEV1 in the periostin high group.

FIG. 18 depicts a forest plot of change from baseline in pre-bronchodilator FEV1 (L) at week 12 by baseline blood eosinophil count in an ITT population. A high placebo effect was observed in the 150 to less than 300 group, which may have led to a negative effect on treatment groups.

FIG. 19 depicts a forest plot of change from baseline in pre-BD FEV1 (L) at week 12 by baseline FeNO and periostin subgroups in a modified ITT (mITT) population.

FIG. 20 depicts post-BD FEV1 absolute change from baseline, LS Mean. SAR440340 had no significant effect on post-BD FEV1.

FIG. 21 depicts post-BD FEV1 mean change over time in an ITT population. The combination of SAR440340 and dupilumab as well as and dupilumab alone both had a rapid effect on post-BD FEV1, however, the effect was sustained only in the dupilumab arm.

FIG. 22 depicts change from baseline in ACQ-5 in an ITT population. SAR440340 demonstrated significant improvement in ACQ-5 by week 12.

FIG. 23 depicts change from baseline in ACQ-5 over time in a mITT population. SAR440340 effect on ACQ-5 was rapid and sustained over 12 weeks.

FIG. 24 depicts AQLQ in an ITT population. SAR440340 demonstrated significant improvement in AQLQ over 12 weeks.

FIG. 25 depicts AQLQ, change from baseline in AQLQ(S) overall score over time in an ITT population. SAR440340 had a rapid and sustained effect on AQLQ over the 12-week period.

FIG. 26 depicts AQLQ, change from baseline in AQLQ(S) emotional function score over time in an ITT population. SAR440340 demonstrated significant improvement in the AQLQ-emotional function score.

FIG. 27 depicts mean blood eosinophil count (10⁹/L) over time in an ITT population.

FIG. 28 depicts mean and median change from baseline of blood eosinophil count over time in an ITT population. SAR440340 consistently lowered eosinophils over the 12-week period.

FIG. 29 depicts mean FeNO (ppb) over time in an ITT population.

FIG. 30 depicts mean and median change from baseline in FeNO (ppb) over time in an ITT population. SAR440340 demonstrated a modest effect on FeNO during the 12-week period.

FIG. 31 depicts mean periostin (ng/mL) over time in an ITT population.

FIG. 32 depicts mean and median change from baseline in periostin (ng/mL) over time in an ITT population. SAR440340 demonstrated a modest effect on periostin levels over the 12 weeks.

FIG. 33 depicts mean eotaxin-3 (pg/mL) over time in an ITT population.

FIG. 34 depicts mean and median change from baseline in eotaxin-3 (pg/mL) over time in an ITT population. SAR440340 had no clear effect on eotaxin-3.

FIG. 35 depicts mean PARC (pg/mL) over time in an ITT population.

FIG. 36 depicts mean and median change from baseline in PARC (pg/mL) over time in an ITT population. SAR440340 had no clear effect on PARC.

FIG. 37 depicts mean total IgE (IU/mL) over time in an ITT population.

FIG. 38 depicts mean and median change from baseline in total IgE (IU/mL) over time in an ITT population. SAR440340 had no clear effect on IgE.

FIG. 39 depicts mean total IL33 (pg/mL) over time.

FIG. 40 depicts mean and median change from baseline in total IL33 (pg/mL) over time in a safety population. As expected, SAR440340 increased IL-33 levels.

FIG. 41 depicts mean sST2 (pg/mL) over time in an ITT population.

FIG. 42 depicts mean and median change from baseline in sST2 (pg/mL) over time in an ITT population. SAR440340 had no clear effect on sST2 levels.

FIG. 43 depicts mean calcitonin (pg/mL) over time in an ITT population.

FIG. 44 depicts mean change from baseline in calcitonin (pg/mL) over time in an ITT population. SAR440340 had no effect on calcitonin levels.

FIG. 45 depicts mean blood neutrophil count over time in an ITT population.

FIG. 46 depicts mean and median change from baseline in blood neutrophil count over time in an ITT population. SAR440340 demonstrated a modest effect on blood neutrophils.

FIG. 47 depicts serum concentration of SAR440340 (ng/mL) over time in a PK population. A concentration above 17 mg/L was reached at week 2. A lower concentration of SAR440340 was obtained in the SAR440340 and dupilumab combination arm at week 12.

FIG. 48 depicts mean change in blood Eos per blood Eos strata. There was a more pronounced blood Eos decrease in high blood Eos subgroup.

FIG. 49 depicts median change blood Eos per blood Eos strata. There was a more pronounced blood Eos decrease in the high blood Eos subgroup.

FIG. 50 depicts the mean change in blood Eos per FeNO strata. There was a more pronounced blood Eos decrease in high FeNO patients.

FIG. 51 depicts the median change in blood Eos per FeNO strata. There was a more pronounced blood Eos decrease in high FeNO patients.

FIG. 52 depicts the mean change in neutrophils per Eos strata. There was no influence of blood Eos level on neutrophils decrease (trend).

FIG. 53 depicts the median change in neutrophils per Eos strata. There was no decrease in neutrophils.

FIG. 54 depicts the mean change in blood neutrophils per FeNO strata. There was no influence by FeNO level on neutrophils decrease (trend).

FIG. 55 depicts the median change in blood neutrophils per FeNO strata. There was no decrease in neutrophils.

FIG. 56 depicts the mean change FeNO per blood Eos strata. There was a slight decrease of FeNO in high blood Eos patients.

FIG. 57 depicts the median change FeNO per blood Eos strata. There was a slight decrease of FeNO in high blood Eos patients.

FIG. 58 depicts the mean change FeNO per FeNO strata. There was a slight decrease of FeNO in high FeNO patients.

FIG. 59 depicts the median change FeNO per FeNO strata. There was no significant decrease of FeNO, even in high FeNO patients.

FIG. 60 depicts the mean change PARC per blood Eos strata. There was a PARC decrease in high blood Eos patients only.

FIG. 61 depicts the median change PARC per blood Eos strata. There was no significant PARC decrease in high blood Eos patients.

FIG. 62 depicts the mean change PARC per FeNO strata. There was a decrease in PARC in high FeNO patients only.

FIG. 63 depicts the median change PARC per FeNO strata. There was no significant PARC decrease.

FIG. 64 depicts the mean change eotaxin-3 per blood Eos strata. There was an increase of eotaxin-3 in low blood Eos patients, and no change in high blood Eos patients.

FIG. 65 depicts the median change eotaxin-3 per blood Eos strata. There was no significant change in eotaxin-3.

FIG. 66 depicts the mean change eotaxin-3 per FeNO strata. There was a slight increase of eotaxin-3 in high FeNO patients.

FIG. 67 depicts the median change eotaxin-3 per FeNO strata. There was no significant change in eotaxin-3.

FIG. 68 depicts the mean change IgE per blood Eos strata. There was a greater decrease of IgE in high blood Eos patients.

FIG. 69 depicts the median change in IgE per blood Eos strata. There was no significant decrease of IgE.

FIG. 70 depicts the mean change in IgE per FeNO strata. There was a greater decrease of IgE in high FeNO patients.

FIG. 71 depicts the median change in IgE per FeNO strata. There was no significant decrease of IgE.

FIG. 72 depicts the mean change in periostin per blood Eos strata. A similar decrease of periostin was observed across blood Eos strata.

FIG. 73 depicts the median change in periostin per blood Eos strata.

FIG. 74 depicts the mean change in periostin per FeNO strata. There was a periostin decrease only in low FeNO patents.

FIG. 75 depicts the median change in periostin per FeNO strata.

FIG. 76 depicts a forest plot of incidence of LOAC by baseline ICS dose level in a mITT population.

FIG. 77 depicts pre-BD FEV1 mean change from baseline by ICS subgroup.

FIG. 78 depicts LOAC by FeNO subgroup.

FIG. 79 depicts LOAC by ICS subgroup.

FIG. 80 depicts LOAC by periostin subgroup.

FIG. 81 depicts post-BD FEV1 percent change from baseline (%) in an ITT population.

FIG. 82 depicts post-BD FEV1 absolute change from baseline by Eos subgroup.

FIG. 83 depicts post-BD FEV1 absolute change from baseline by FeNO subgroup.

FIG. 84 depicts post-BD FEV1 absolute change from baseline by FeNO subgroup.

FIG. 85 depicts post-BD FEV1 absolute change from baseline by periostin subgroup.

FIG. 86 depicts a forest plot of change from baseline in post-bronchodilator FEV1 (L) at week 12 by baseline blood eosinophil count in an ITT population.

FIG. 87 depicts Forest plot of change from baseline in post-bronchodilator FEV1 (L) at week 12 by baseline blood eosinophil count in an ITT population.

FIG. 88 depicts Forest plot of change from baseline in post-bronchodilator FEV1 (L) at week 12 by baseline ICS dose level in a mITT population.

FIG. 89 depicts LOAC by ICS subgroup.

FIG. 90 depicts AQLQ in an ITT population.

FIG. 91 depicts Bayesian analyses.

FIG. 92 depicts asthma proof of concept key inclusion and exclusion criteria. Patients were enrolled across a broad baseline eosinophil level.

FIG. 93 outlines patient disposition. *Loss of asthma control (LOAC) was a criterion for discontinuation. **Two patients were discontinued due to adverse effects (AS) before end of trial (EOT). †One patient in the dupilumab group died due to ethyl alcohol poisoning in the post-treatment follow-up period (information received after database lock).

FIG. 94 shows baseline demographics, which were generally balanced across the 3 treatment arms and placebo.

FIG. 95 shows baseline disease characteristics, which were generally balanced across treatment arms.

FIG. 96A-96C graphically depict baseline Eos levels (FIG. 96A) and baseline FeNO levels (FIG. 96B), which were evenly distributed across active treatment arms. (FIG. 96C) shows baseline distribution by ICS. Most patients (65.9%) were on high dose ICS.

FIG. 97 depicts pre-BD FEV1 mean change over time to week 32 in an ITT population. SAR440340 demonstrated persistent efficacy weeks after discontinuation.

FIG. 98 shows distributions of ACQ-5 and AQLQ responders by subgroup, at week 4 and week 12.

FIG. 99 depicts change from baseline in AM and PM PEF over time in an ITT population. SAR440340 demonstrated no improvement in AM or PM PEF.

FIG. 100 depicts change from baseline in forced vital capacity (FVC) and forced expiratory flow at 25-75% (FEF25-75) over time in an ITT population. SAR440340 demonstrated no improvement in FVC or FEF25-75.

FIG. 101 depicts change from baseline in AM and PM asthma symptom score over time in an ITT population. SAR440340 demonstrated no improvement in AM or PM asthma symptom score.

FIG. 102 depicts change from baseline in number of nocturnal awakenings and reliever use over time in an ITT population. SAR440340 demonstrated no reduction in nocturnal awakenings or reliever use.

FIG. 103 depicts change from baseline in RQLQ(S) overall score over time in an ITT population with comorbid allergic rhinitis. SAR440340 demonstrated no reduction in RQLQ score.

FIG. 104 depicts serum concentration of SAR440340 (ng/mL) over time. A concentration above 17 mg/L (deduced from house dust mite (HDM) mouse model) was reached at week 2. Lower concentrations of SAR440340 were observed in the dupilumab combination arm at week 12.

FIG. 105 depicts total IL-33 (pg/mL) over time, which were consistent with Phase 1 studies.

FIG. 106 depicts type 2 pharmacodynamic (PD) biomarkers results (median change). SAR440340 induced a decrease of blood eosinophils, and had a modest effect on other type 2 biomarkers.

FIG. 107 depicts median change in blood Eos levels to week 32. SAR440340 continued to keep blood Eos levels lowered after discontinuation.

FIG. 108 depicts spaghetti plots of change from baseline in blood Eos levels in an ITT population. Despite a few outliers, SAR440340 demonstrated consistent reduction in blood Eos levels.

FIG. 109 depicts the median change of blood neutrophil, sST2 and calcitonin biomarkers. SAR440340 induced a modest decrease in blood neutrophils.

FIG. 110 depicts serum concentrations of SAR440340 over time by anti-drug antibody (ADA) status in the SAR440340 monotherapy and combination therapy arms. No patients had a positive ADA to SAR440340.

FIG. 111 depicts a spaghetti plot of serum concentration of SAR440340 over time by peak post-baseline titer category against SAR440340 in an ADA population. No patients had a positive ADA to SAR440340.

FIG. 112 depicts a spaghetti plot of serum concentration of dupilumab over time by peak post-baseline titer category against dupilumab. Dupilumab ADA occurred as expected in the monotherapy arm. There was a numerically greater ADA-positive rate in the combination arm.

FIG. 113 depicts a spaghetti plot of serum concentration of dupilumab over time by peak post-baseline titer category against dupilumab in an ADA population. Dupilumab ADA-positive patients has a trend of lower exposure (overlap of ADA positive and ADA negative in PK exposure).

FIG. 114 depicts forest plots of change from baseline in pre-BD FEV1 (L) at week 12 by demographics subgroups.

FIG. 115 depicts forest plots of change from baseline in pre-BD FEV1 (L) at week 12 by disease characteristics subgroups.

FIG. 116 depicts forest plots of change from baseline in post-BD FEV1 (L) at week 12 by demographics subgroups.

FIG. 117 depicts forest plots of change from baseline in post-BD FEV1 (L) at week 12 by disease characteristics subgroups.

FIG. 118 depicts blood Eos kinetics up to week 32 (mean change).

FIG. 119 depicts mean change from baseline in blood Eos count (10⁹/L) across visits in patients by baseline blood Eos (cutoff at less than 0.5 or greater than or equal to 0.5). SAR440340 reduced blood Eos, though the effect was greater ion the less than 0.5 group.

FIG. 120 depicts the median change in blood Eos per FeNO strata. There was a more pronounced blood Eos decrease in high FeNO patients.

FIG. 121 depicts the median change in FeNO per blood Eos strata. There was a slight decrease of FeNO in high blood Eos patients.

FIG. 122 depicts the median change in periostin per blood Eos strata. There was a decrease in periostin in high blood Eos patients.

FIG. 123 depicts a flow diagram of the multiple ascending dose study of the safety, tolerability, pharmacokinetics and pharmacodynamic effects of subcutaneously administered SAR440340 in adult patients with moderate asthma.

FIG. 124 depicts percent change from baseline in eosinophil levels. Eosinophil levels were reduced by approximately 35% from baseline at day 29, and the effect was sustained until day 197.

FIG. 125 depicts mean eosinophil levels over time.

FIG. 126 depicts mean eosinophil levels over time excluding an outlier for the 75 mg dose.

FIG. 127 depicts percent change from baseline in eosinophil levels. 75 mg and 150 mg doses were not significantly different.

FIG. 128 depicts percent change from baseline in eosinophil levels in a 75 mg and 150 mg pooled population. Eosinophil levels were reduced approximately 35% from baseline at day 29, and the effect was sustained until day 197.

FIG. 129 depicts individual eosinophil level profiles consistent with group means.

FIG. 130 depicts mean FeNO levels over time. The baseline levels of the 75 mg cohort were higher than the 150 mg cohort and the placebo cohort.

FIG. 131 depicts percent change in FeNO levels over time. There was a modest effect on FeNO levels in the 150 mg cohort.

FIG. 132 depicts percent change in FeNO levels over time for a pooled population of 75 mg and 150 mg. There was an approximately 10% decrease in the SAR440340 cohorts.

FIG. 133 depicts percent change from baseline in FeNO levels over time for a pooled population of 75 mg and 150 mg.

FIG. 134 depicts individual change in FeNO levels consistent with group means. An outlier in the 75 mg cohort drove up mean levels.

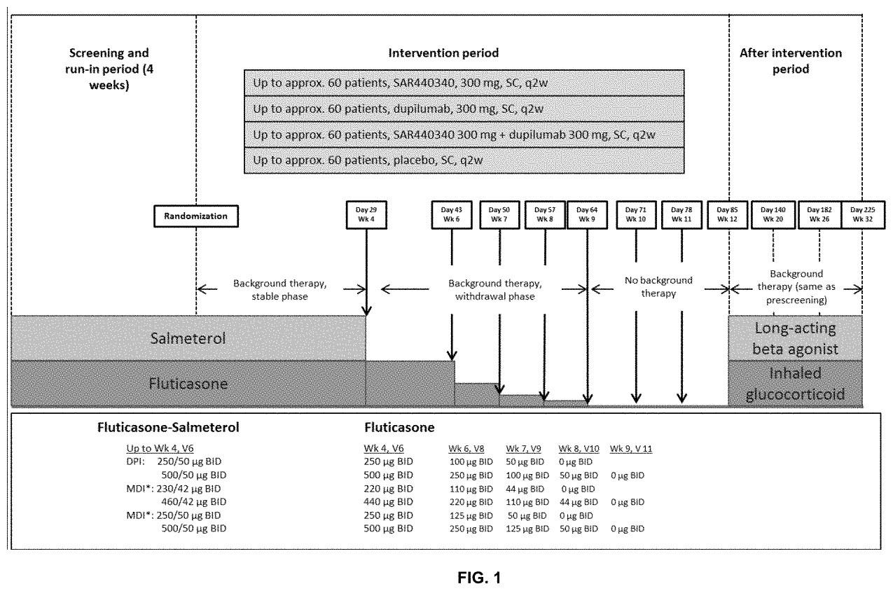

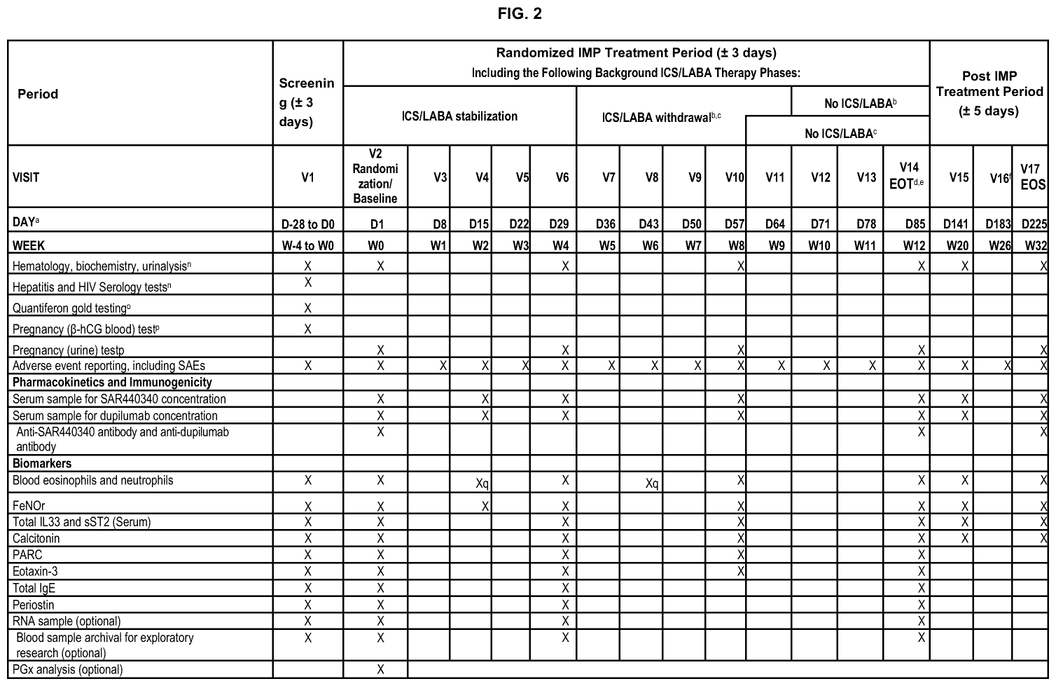

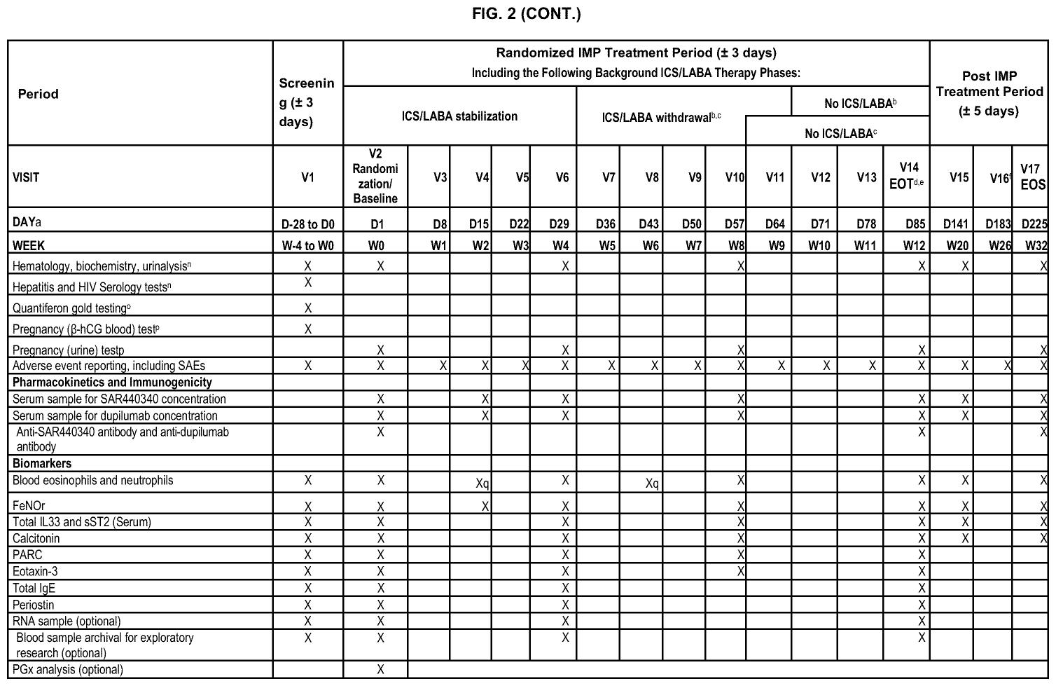

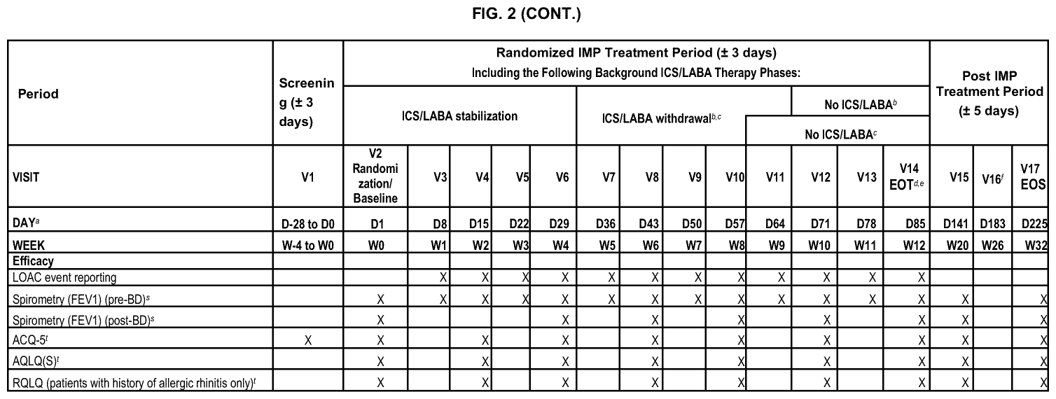

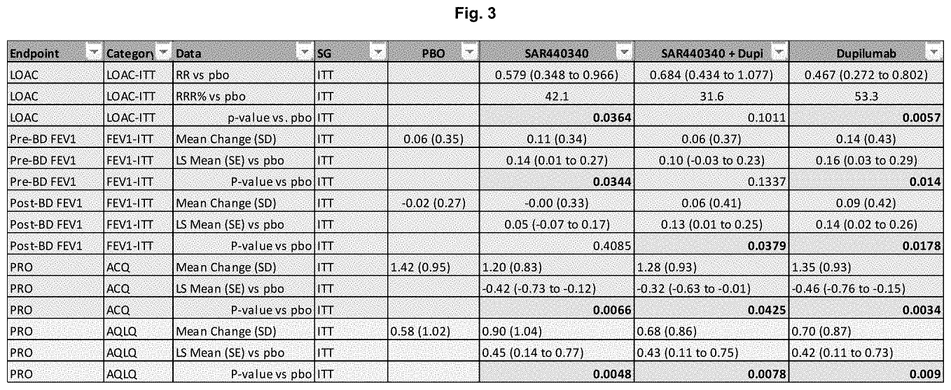

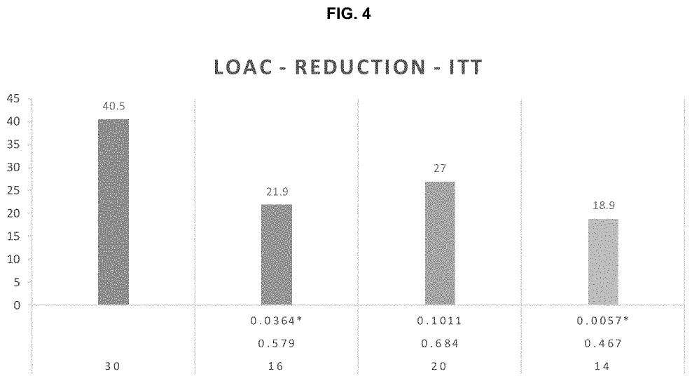

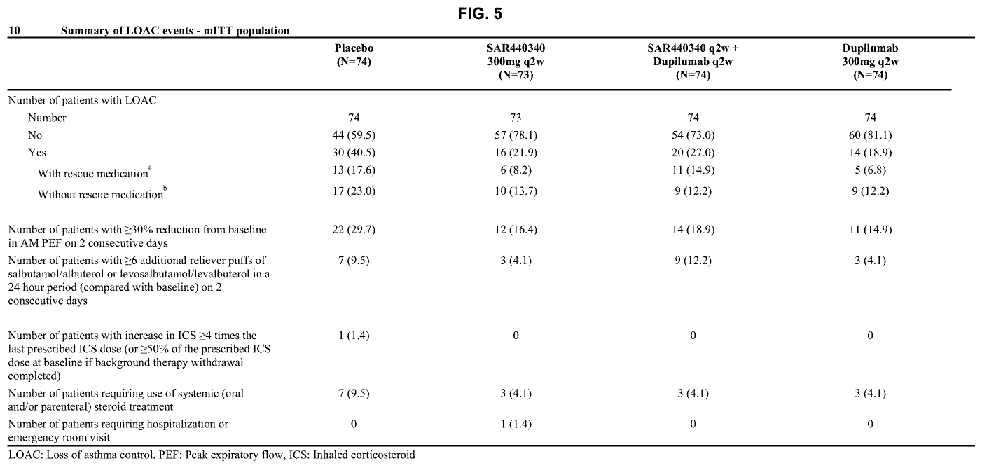

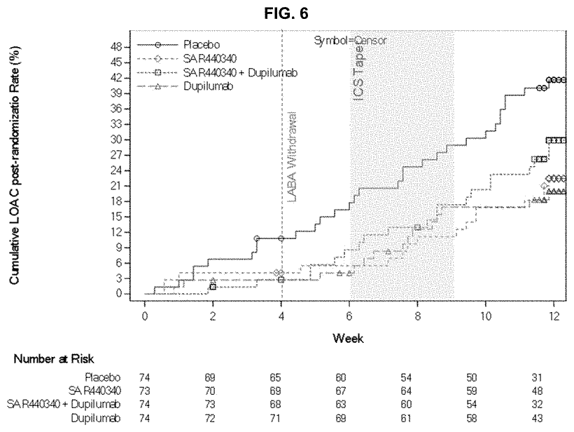

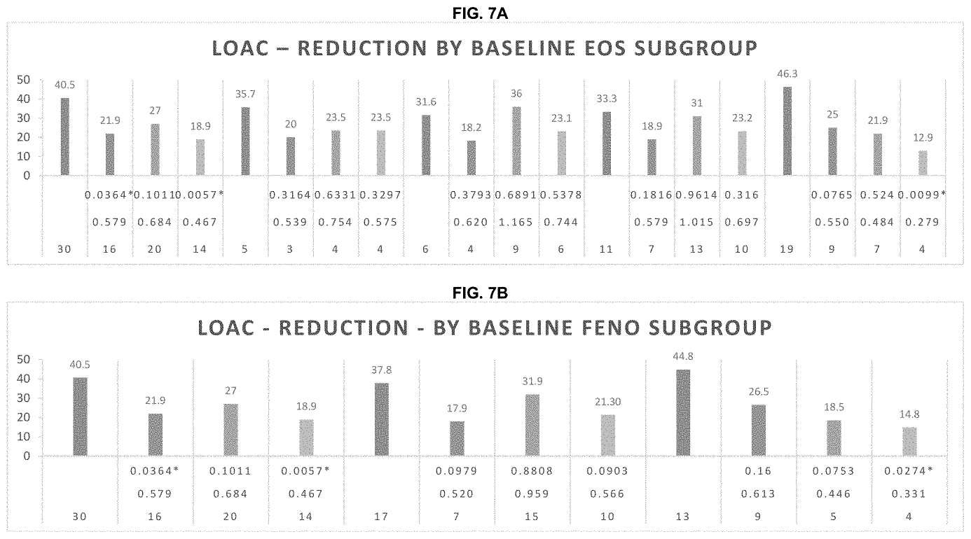

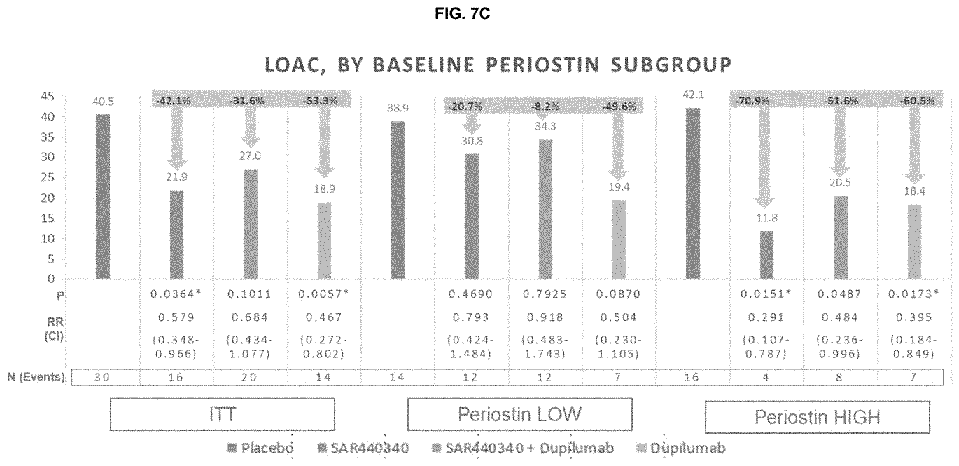

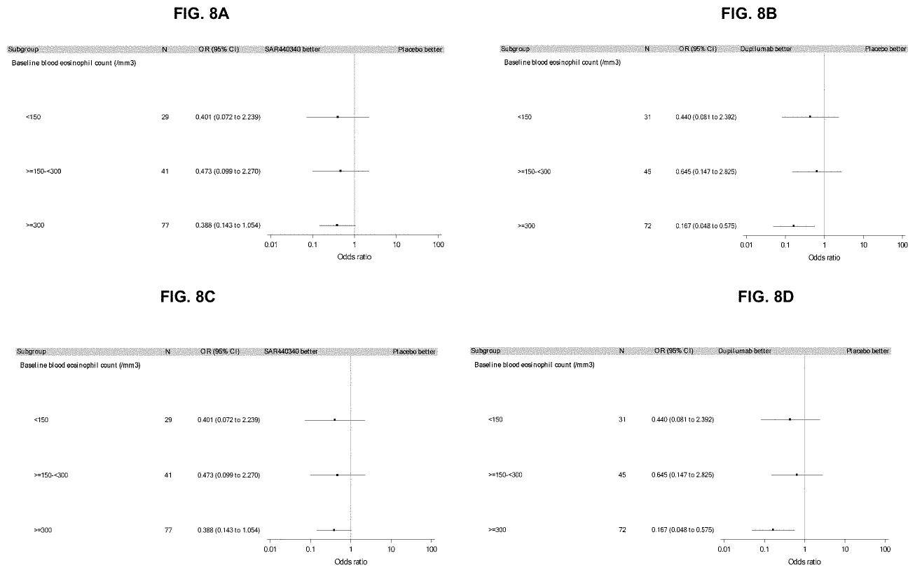

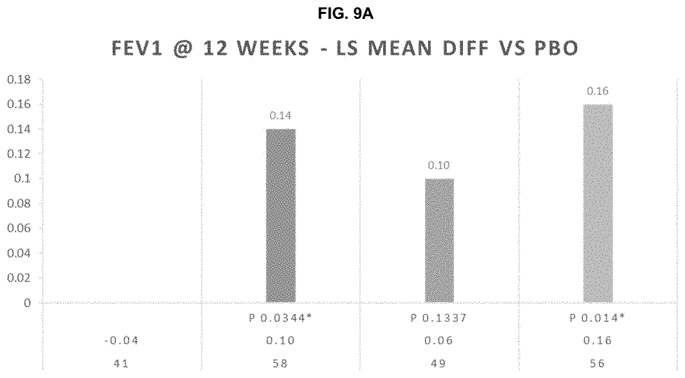

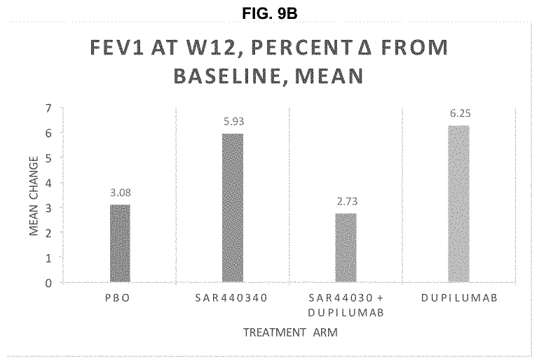

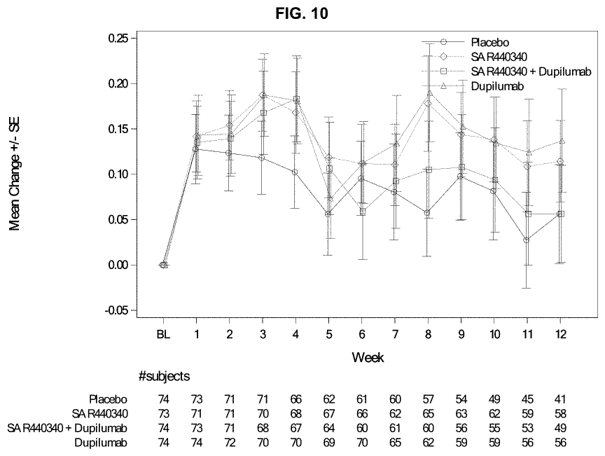

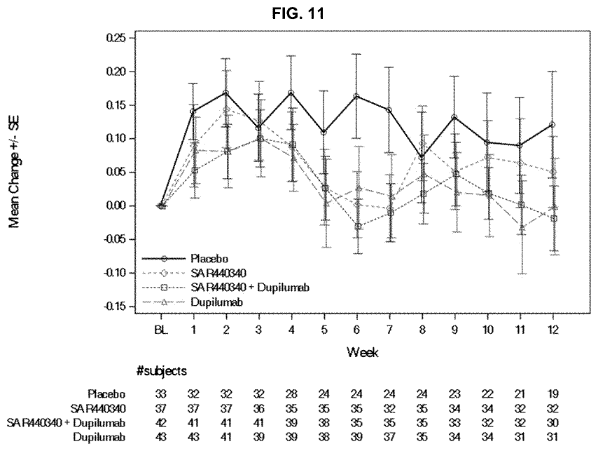

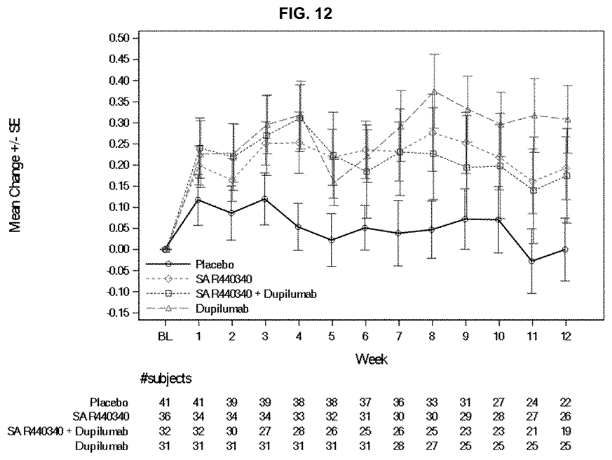

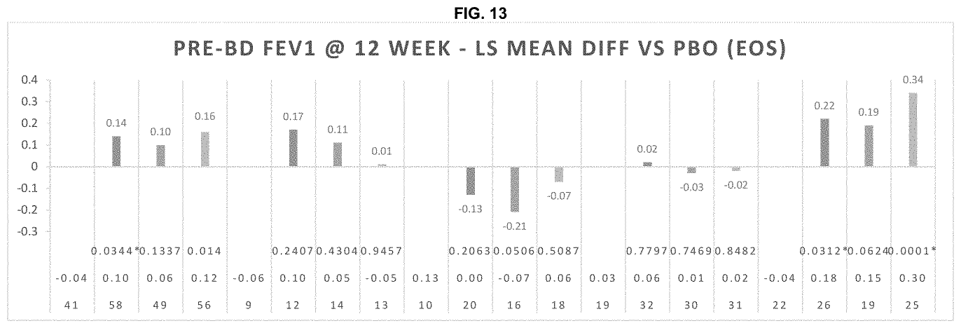

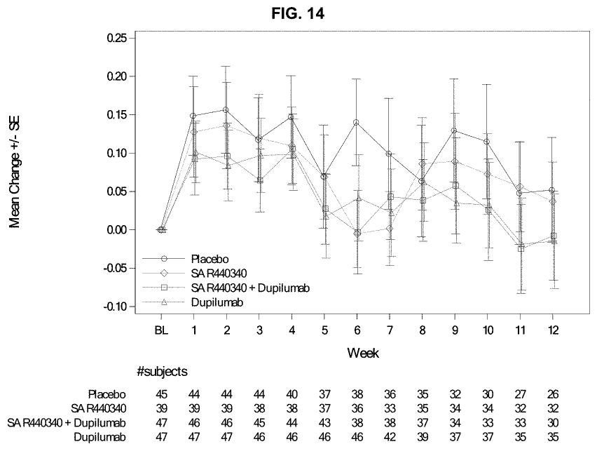

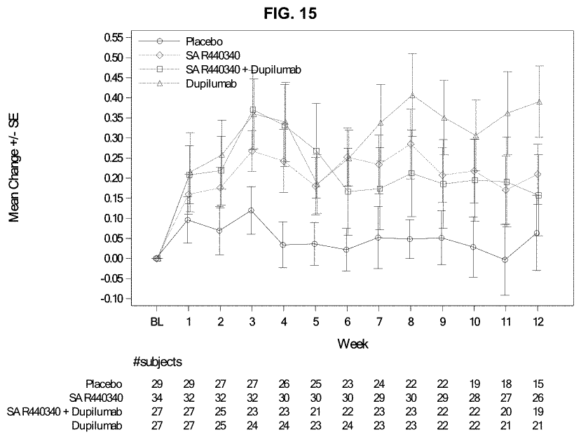

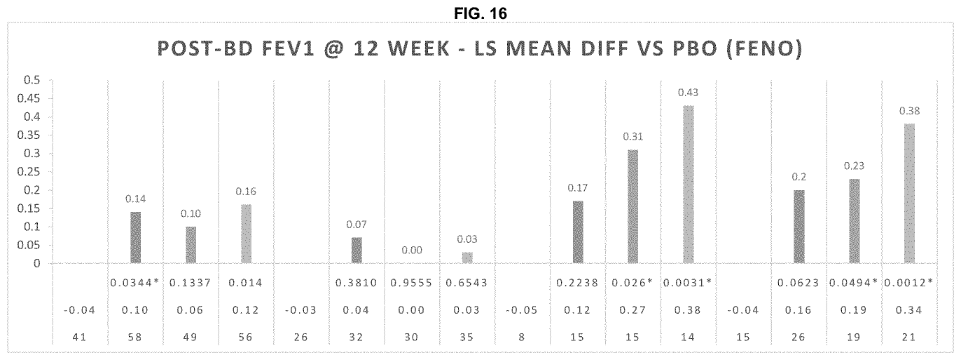

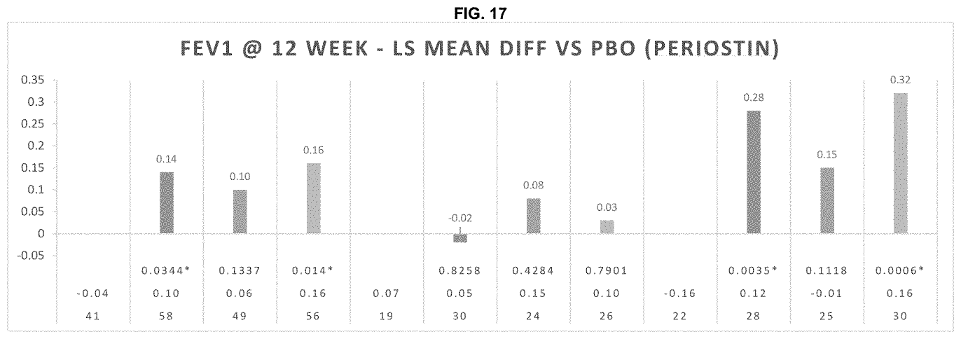

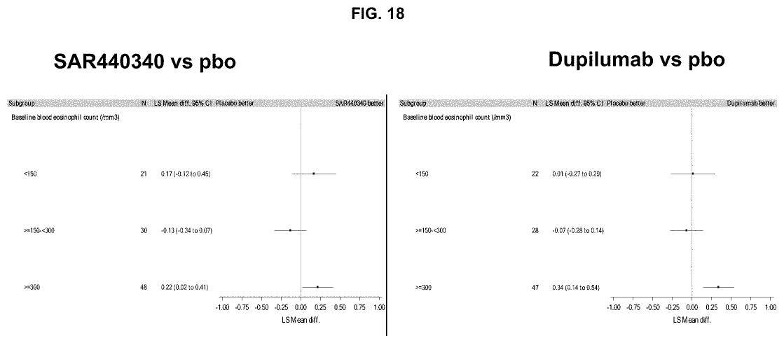

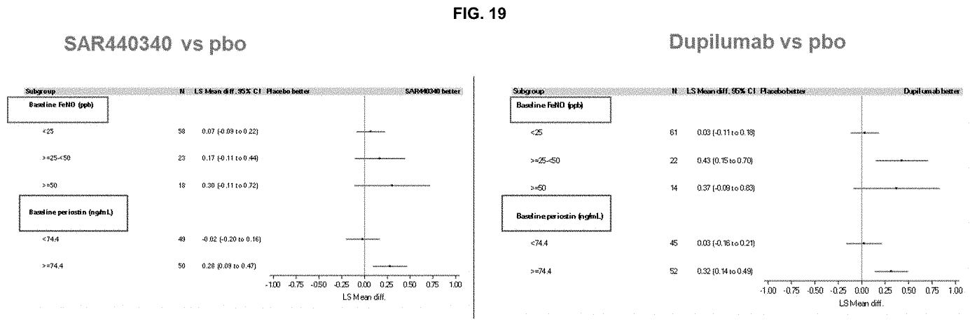

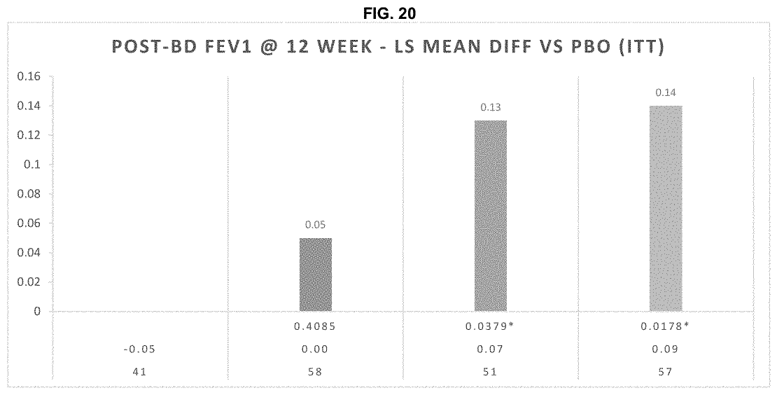

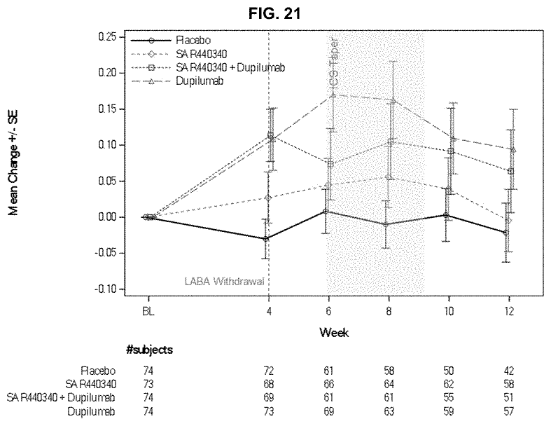

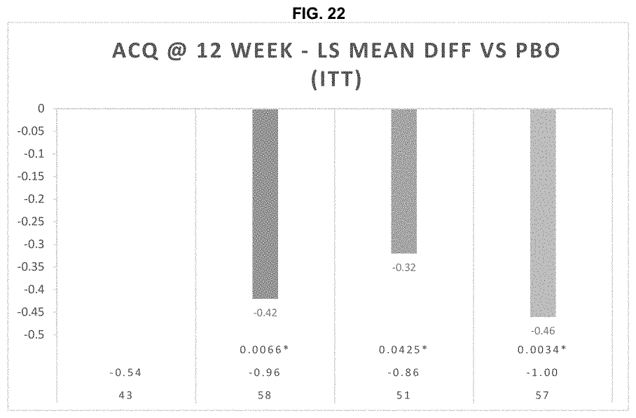

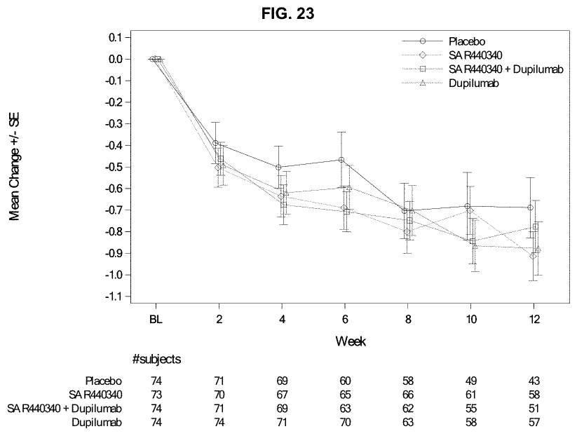

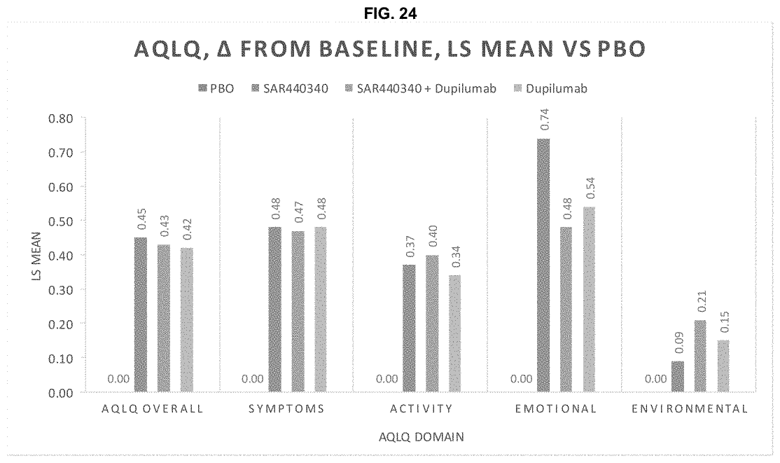

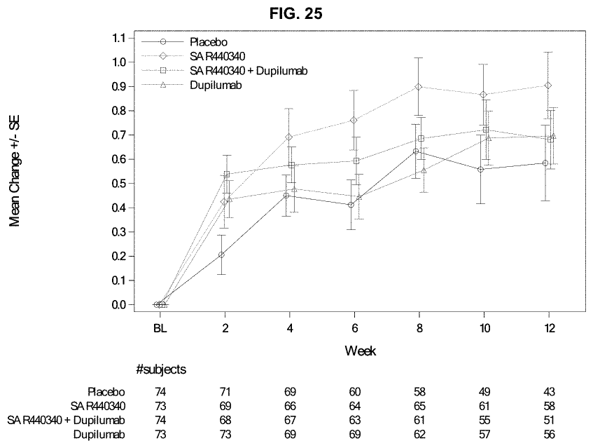

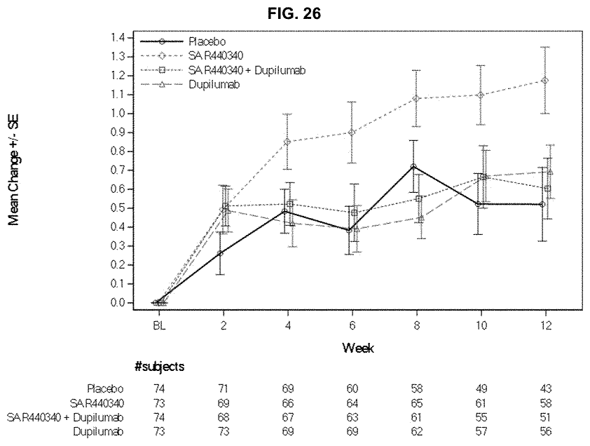

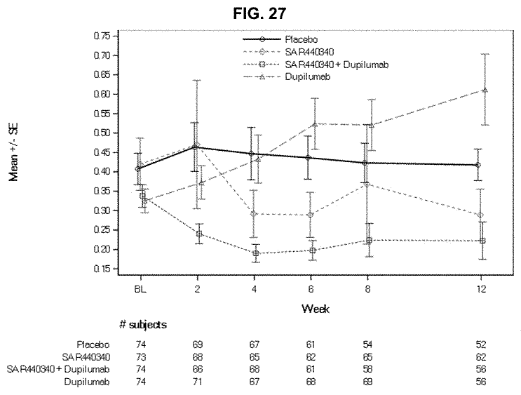

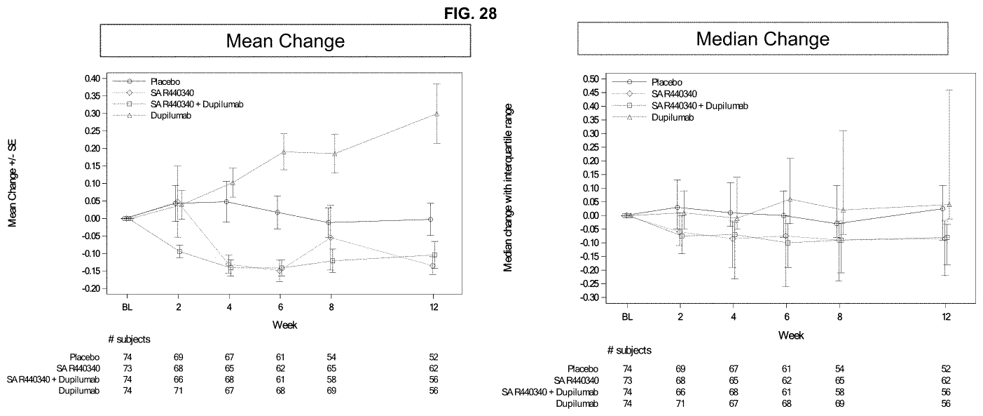

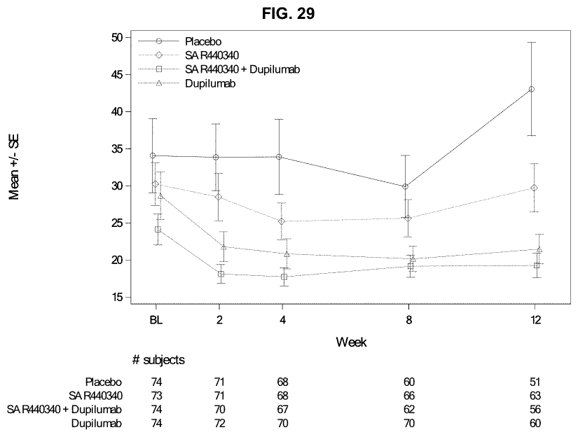

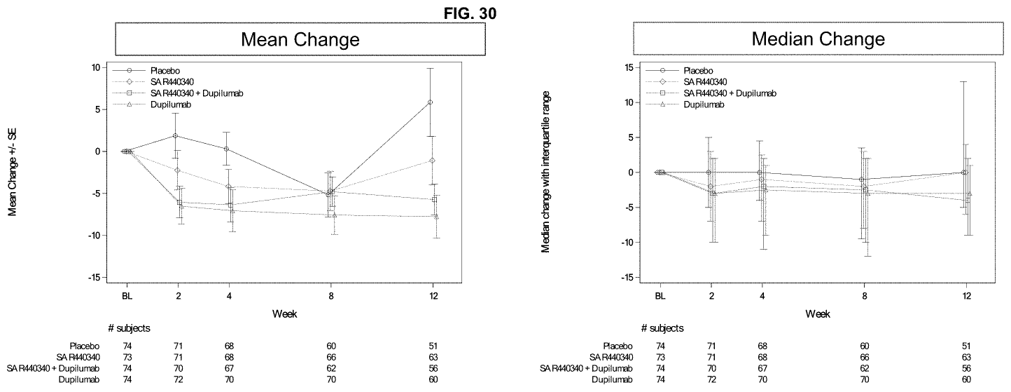

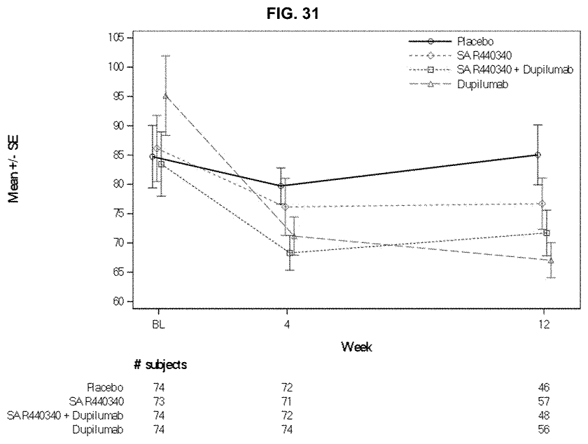

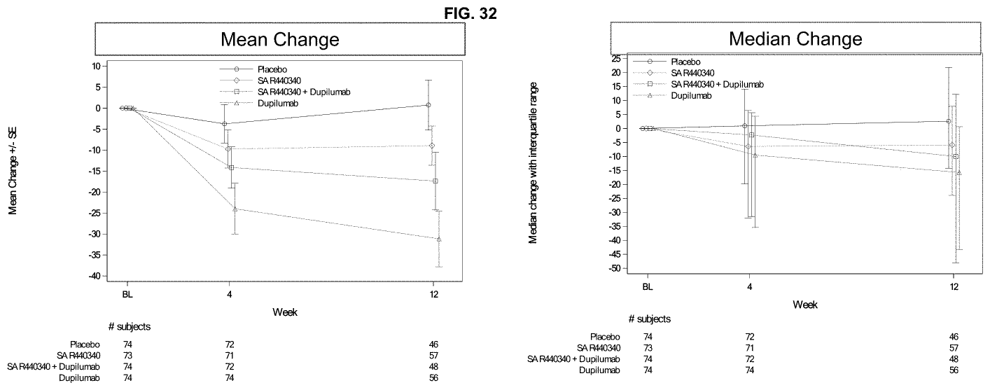

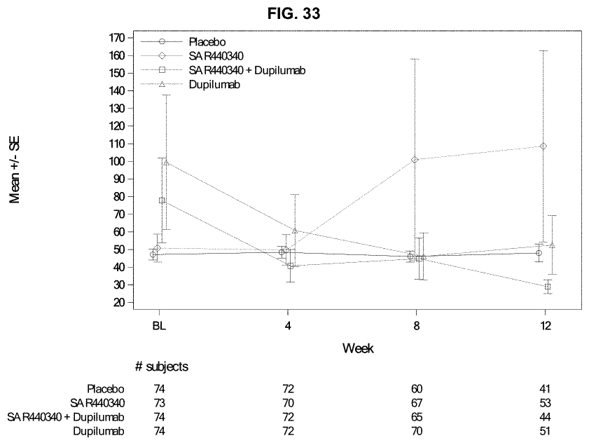

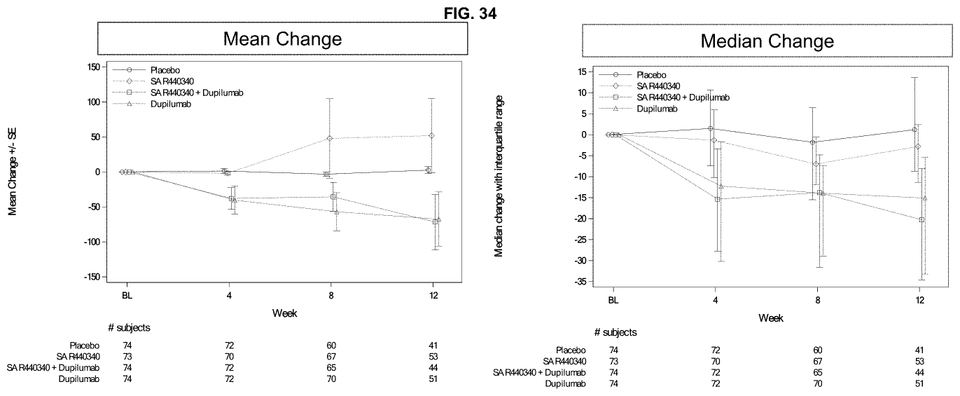

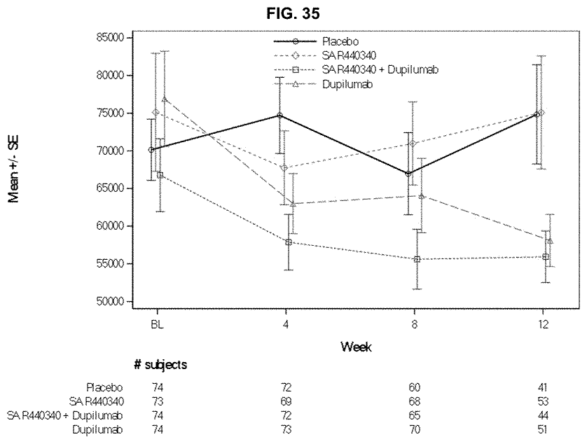

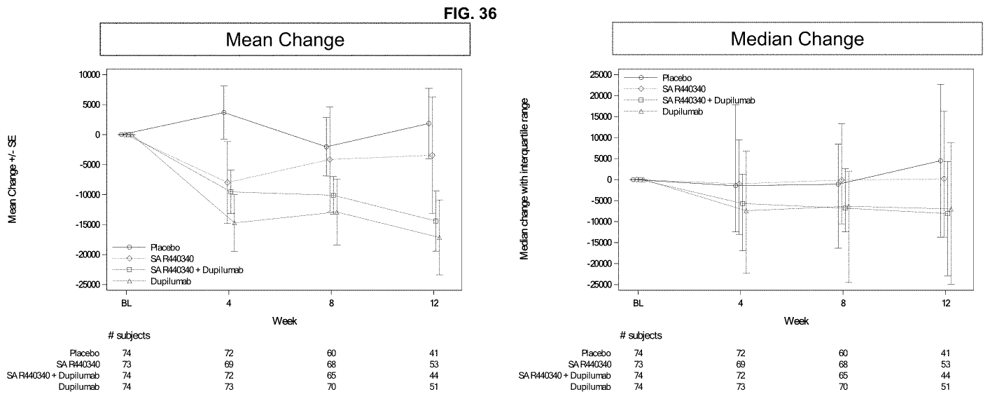

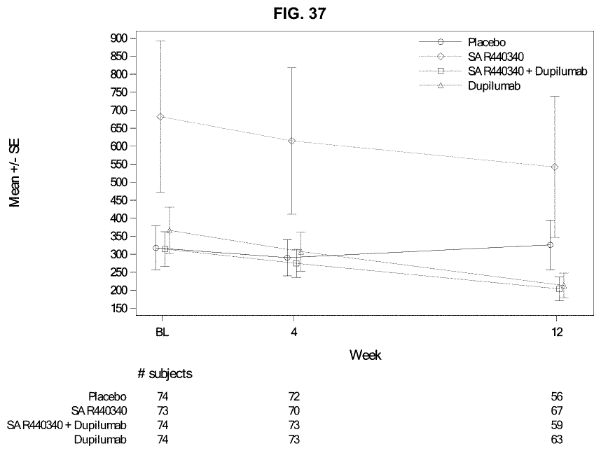

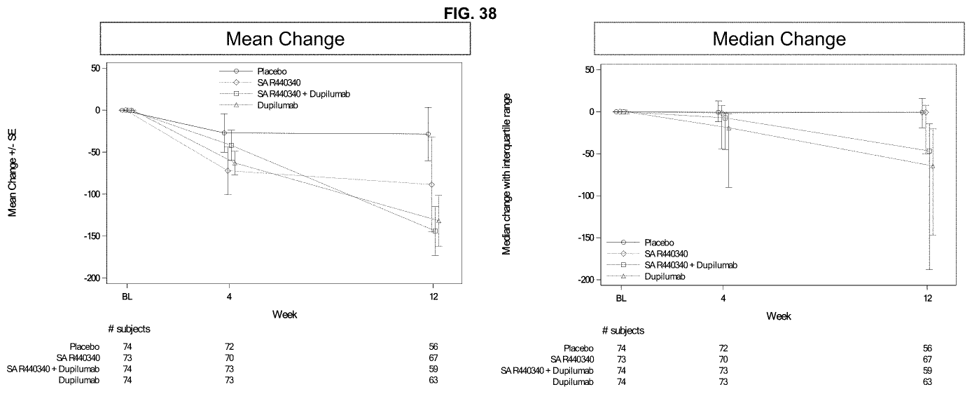

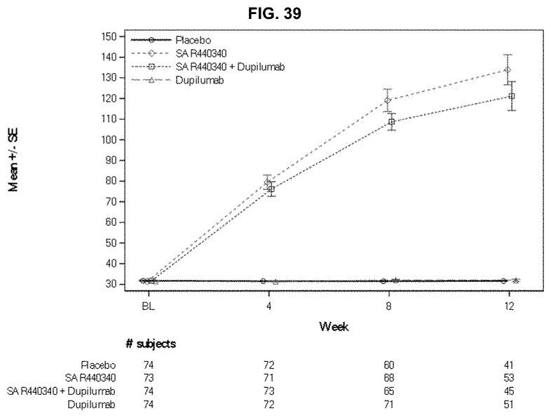

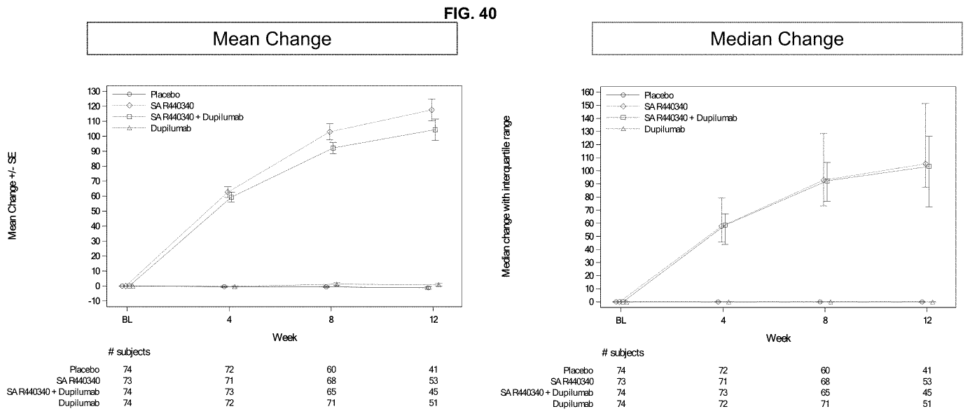

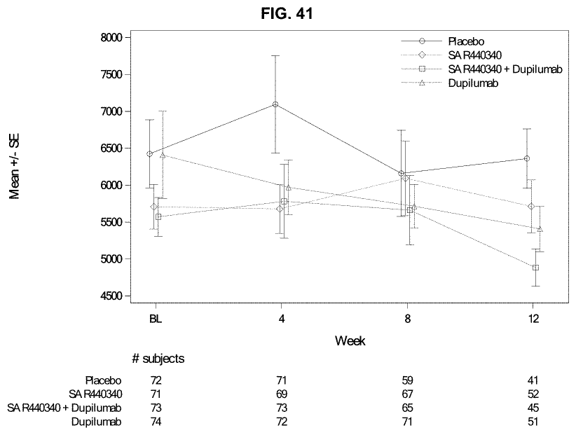

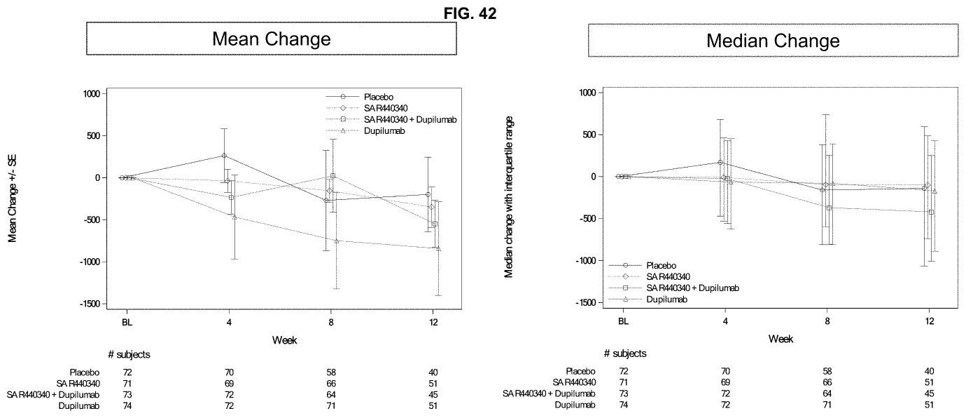

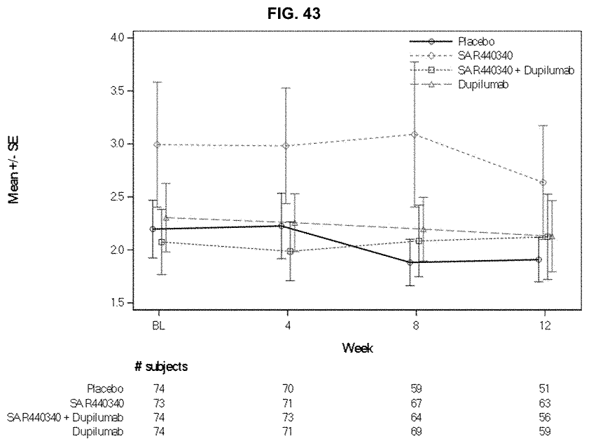

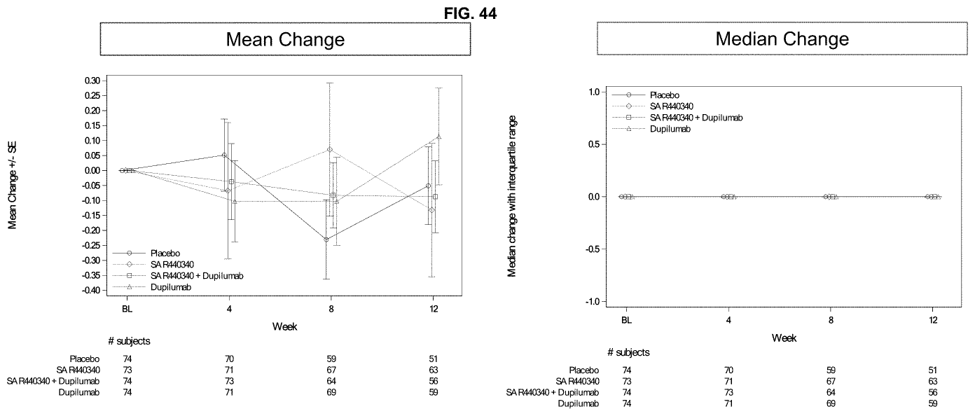

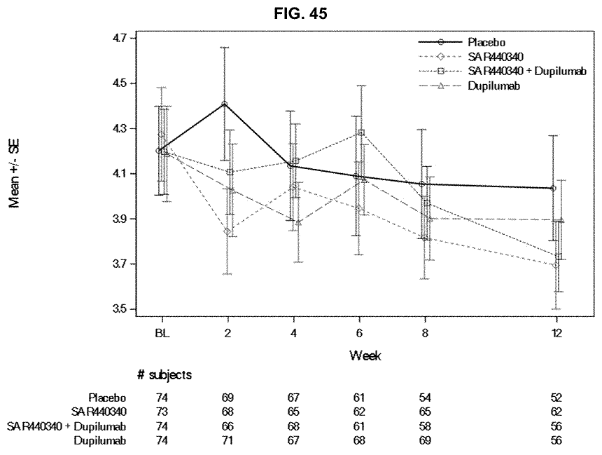

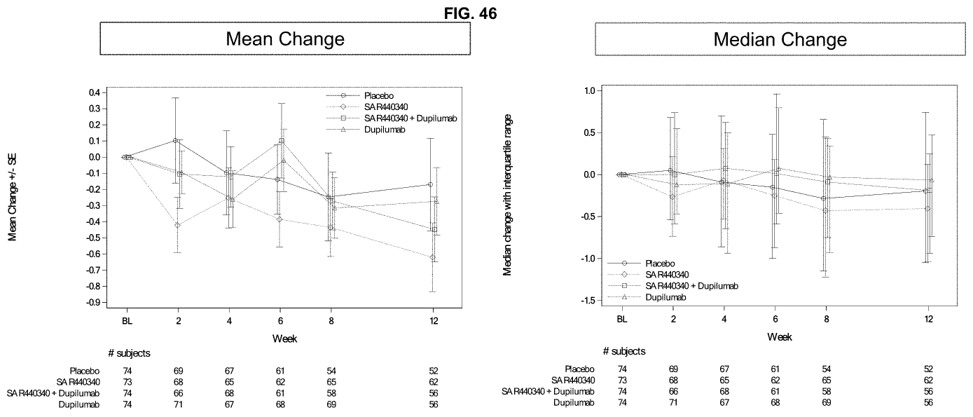

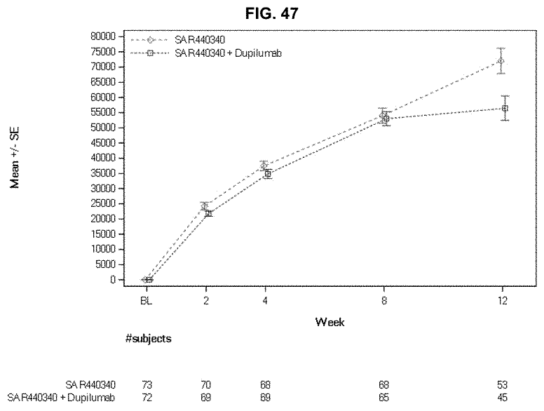

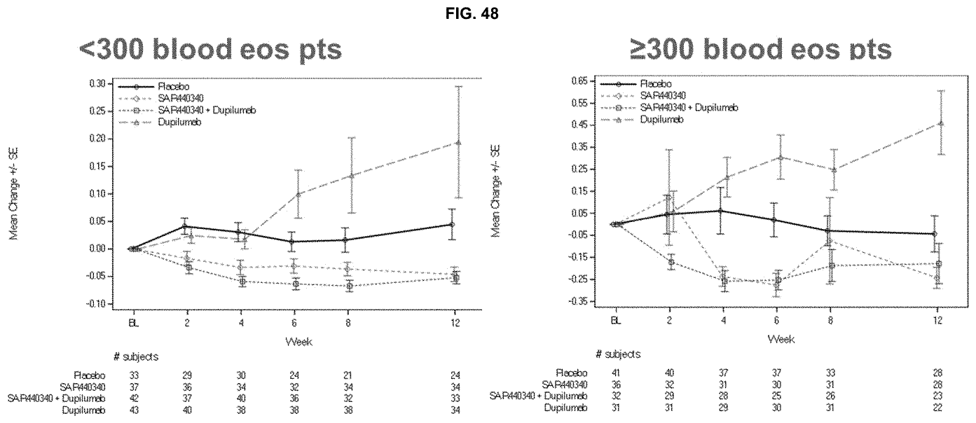

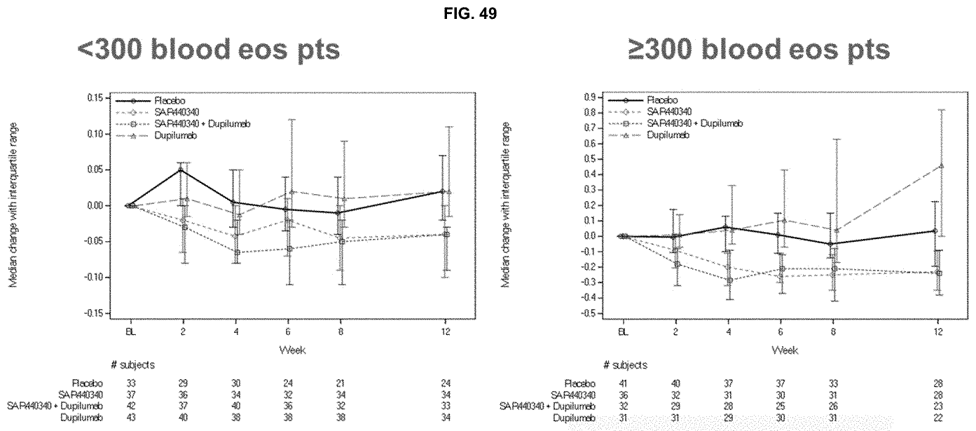

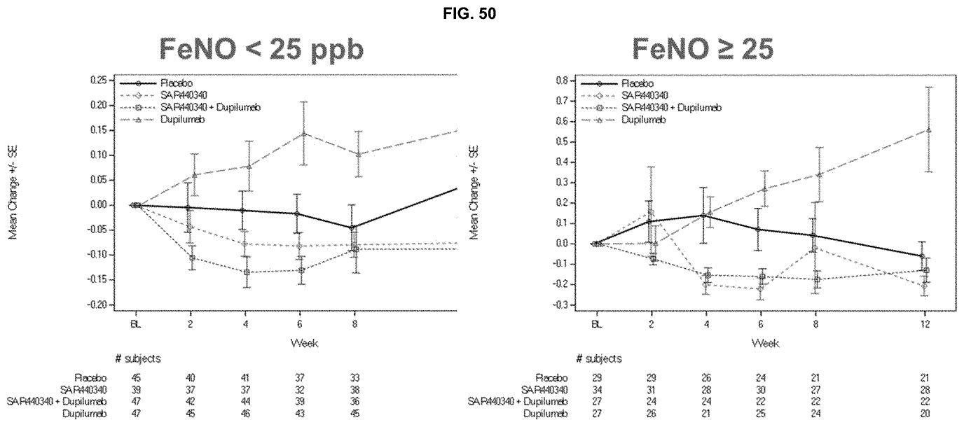

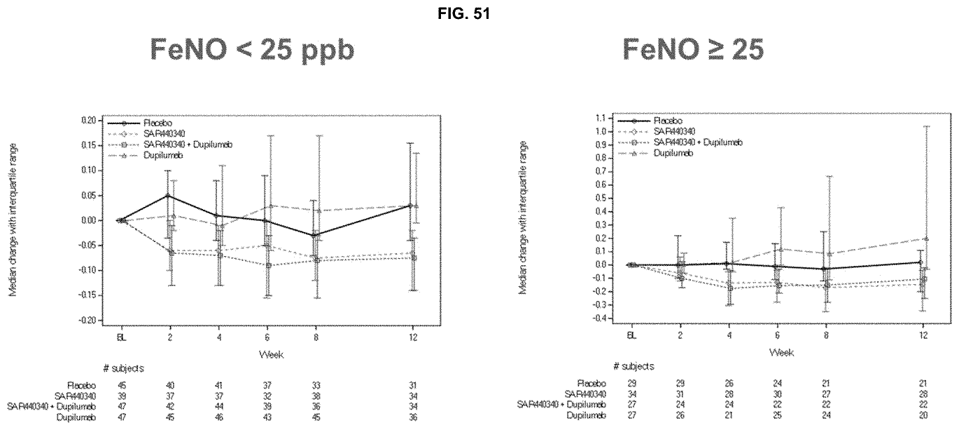

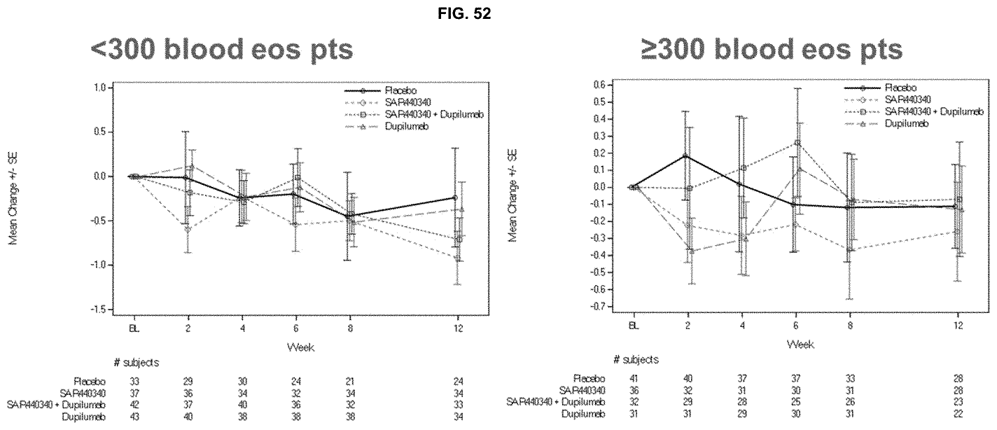

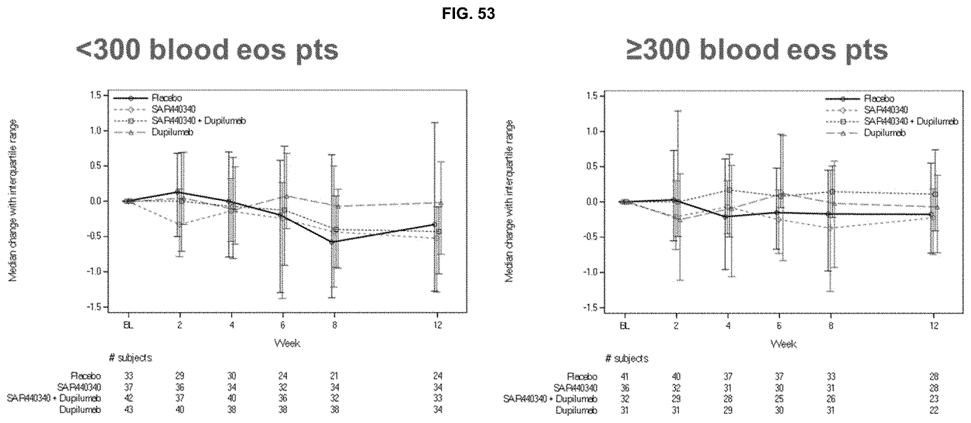

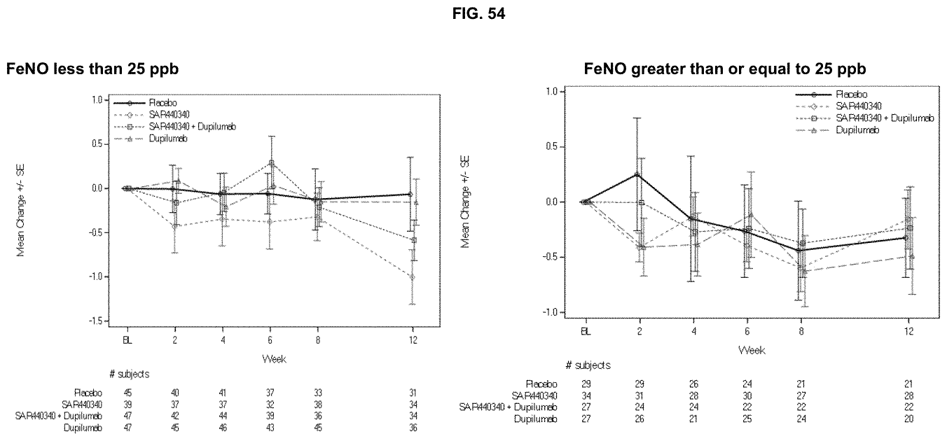

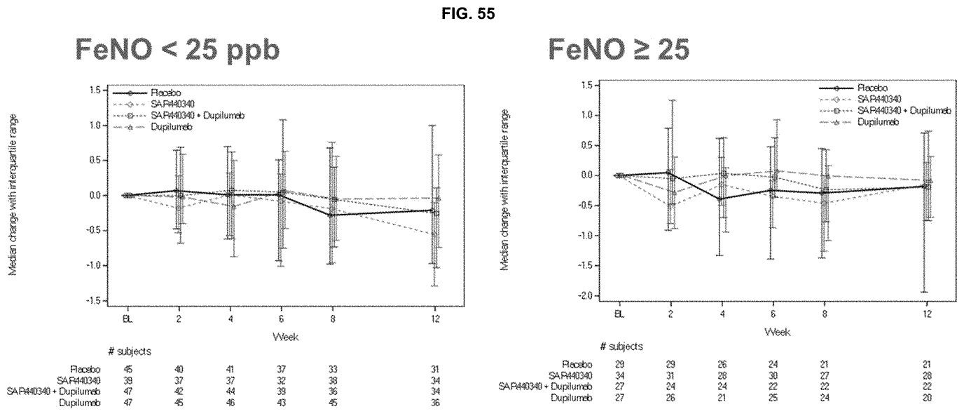

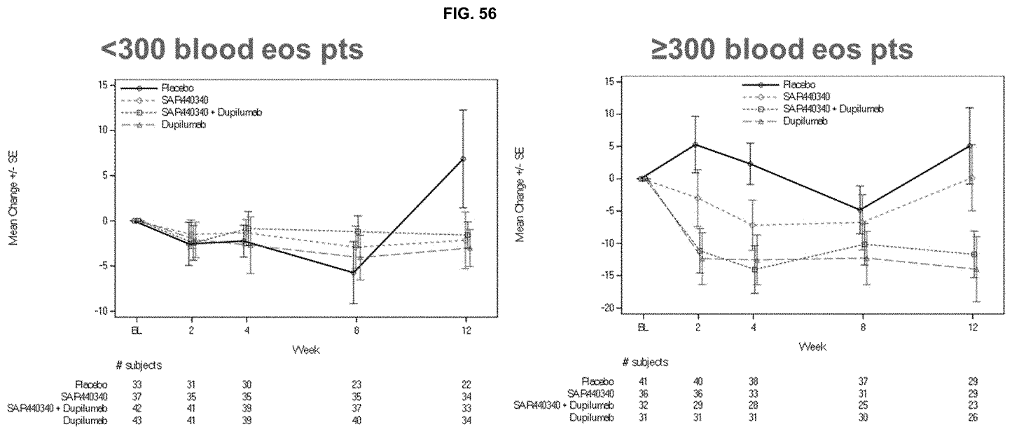

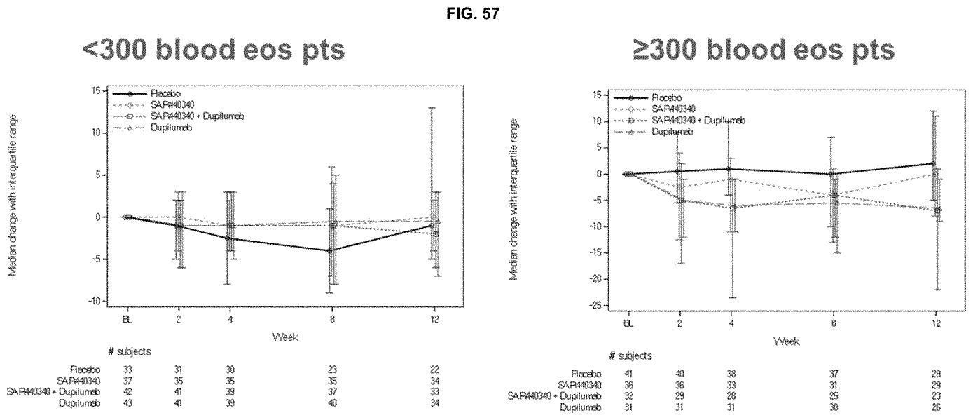

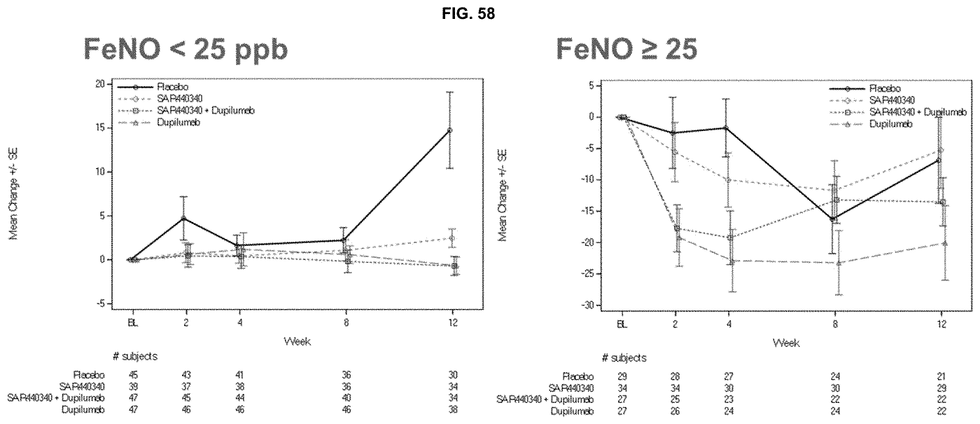

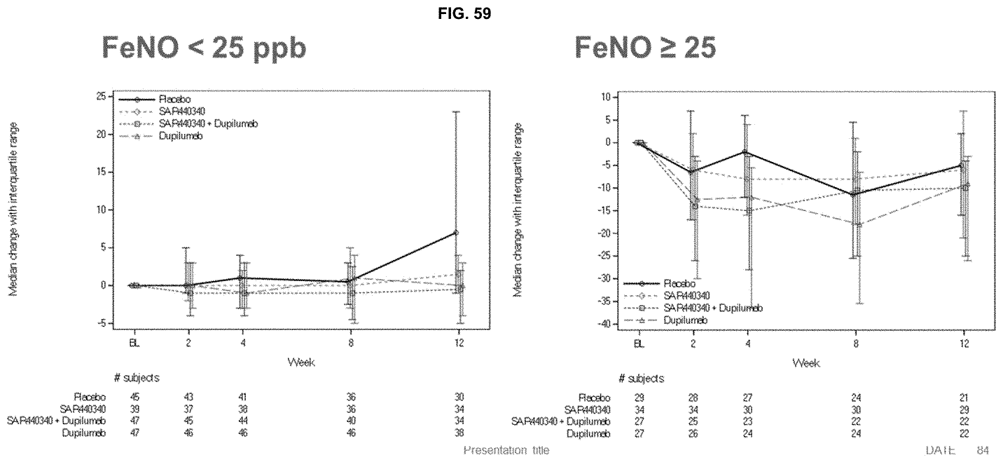

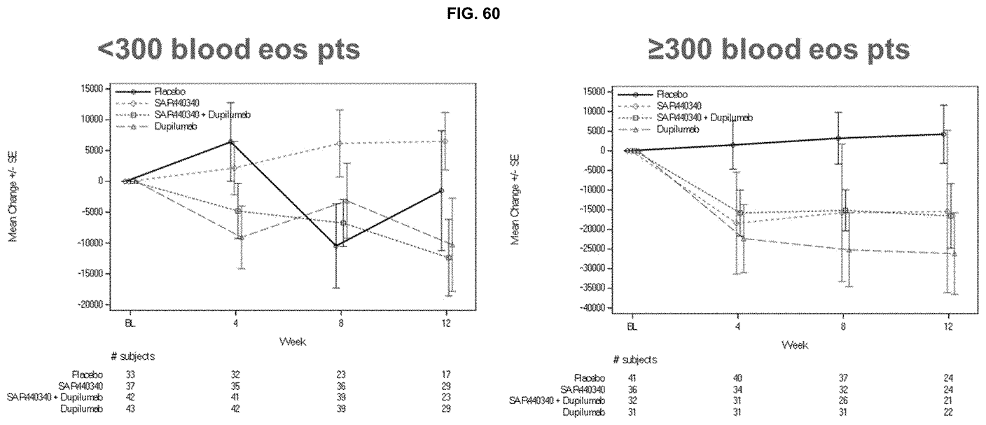

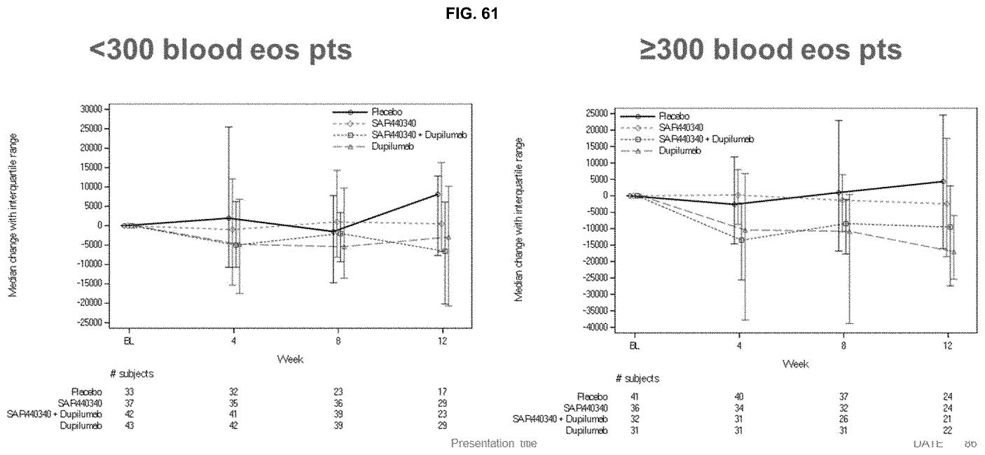

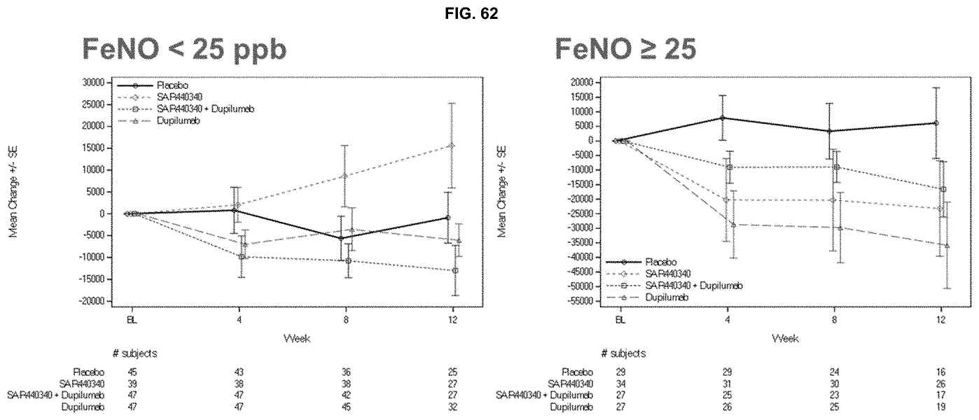

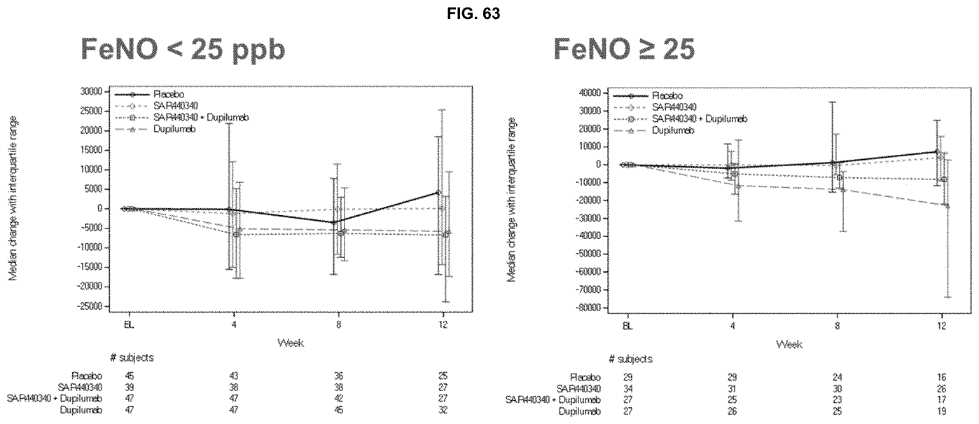

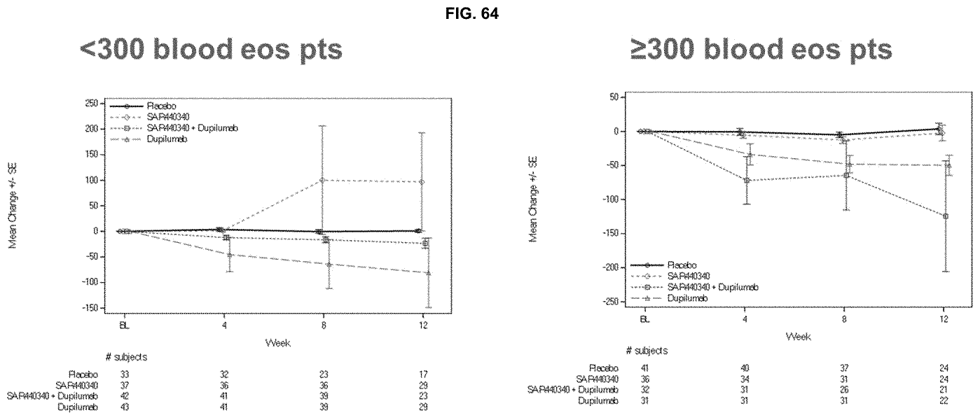

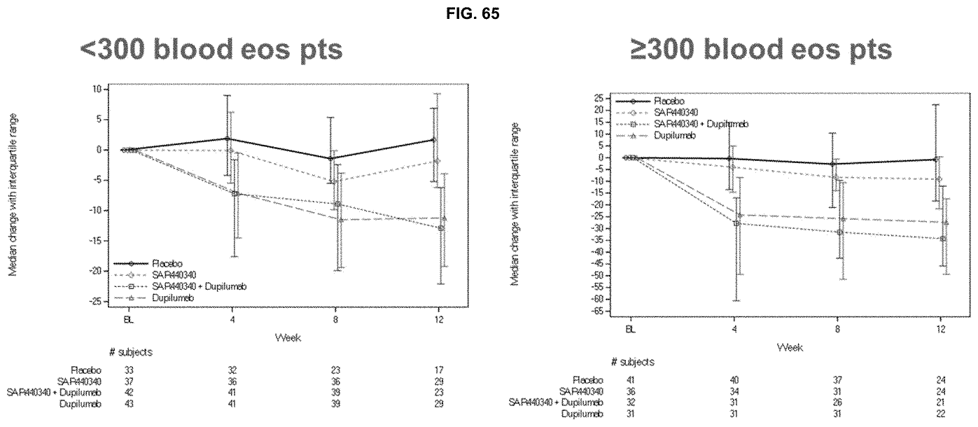

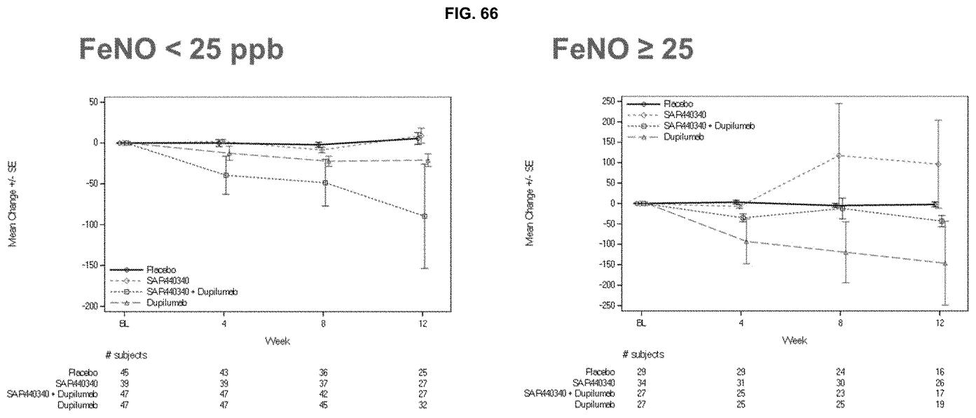

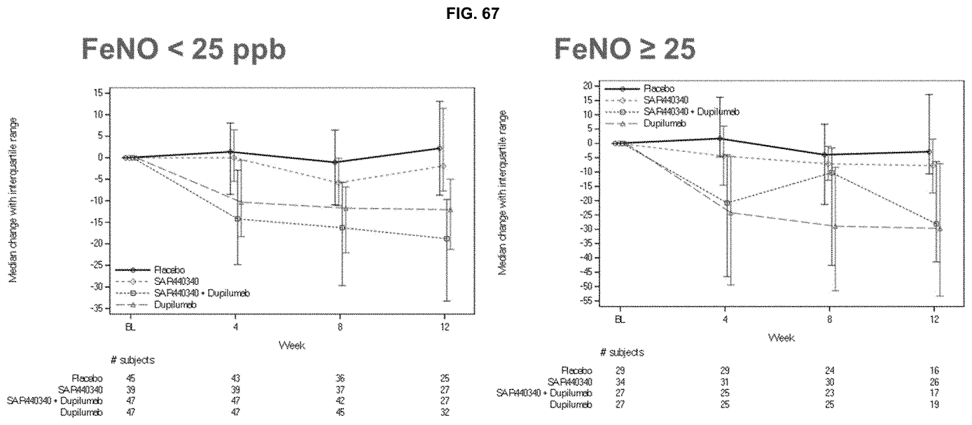

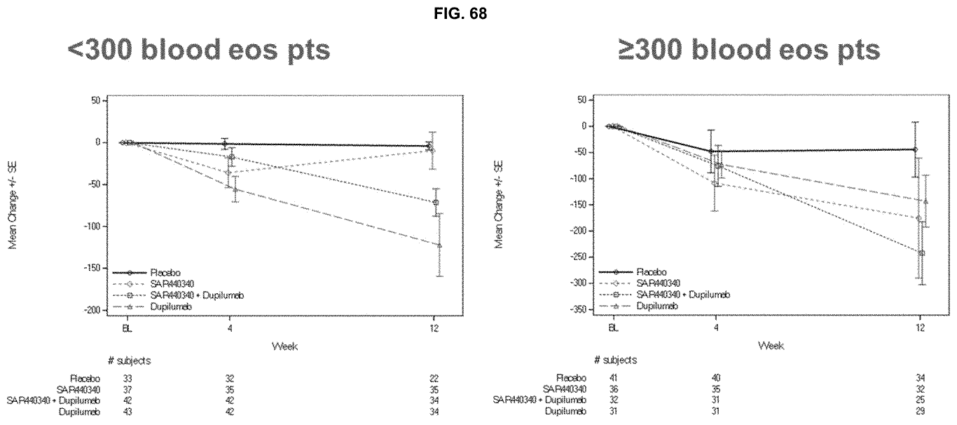

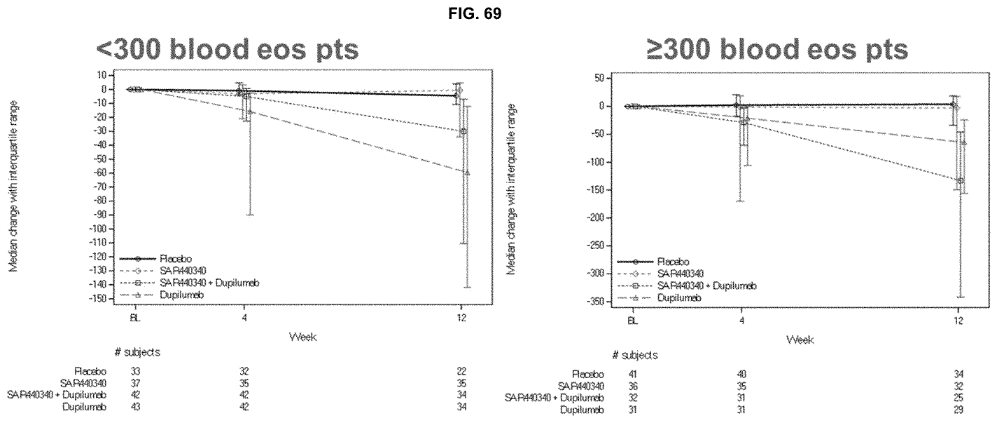

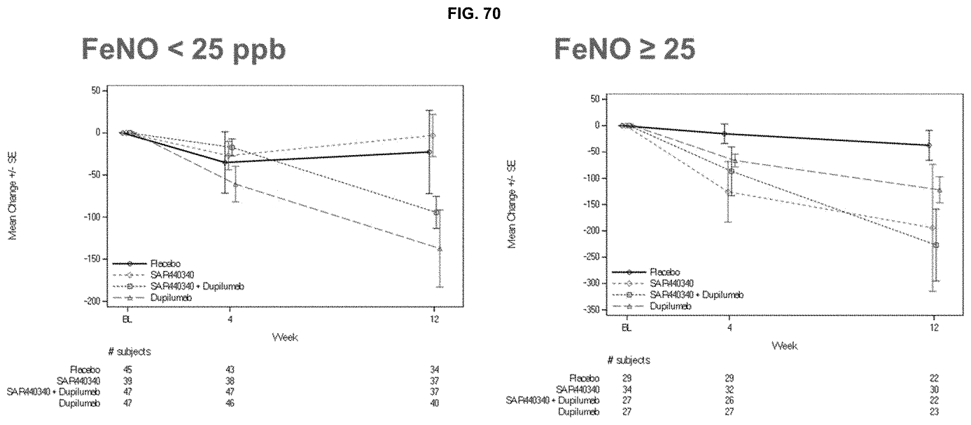

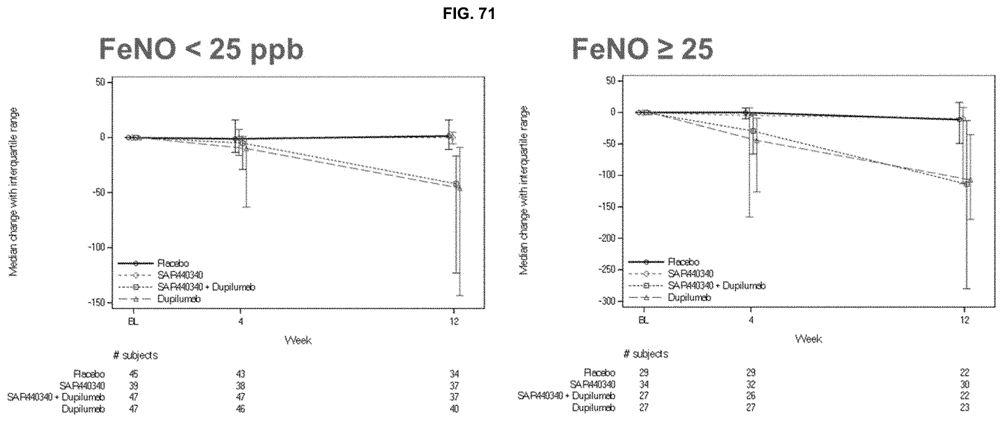

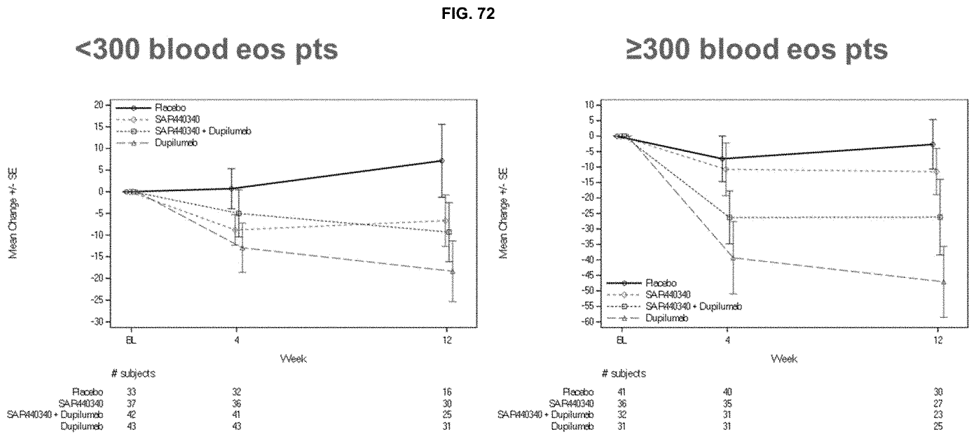

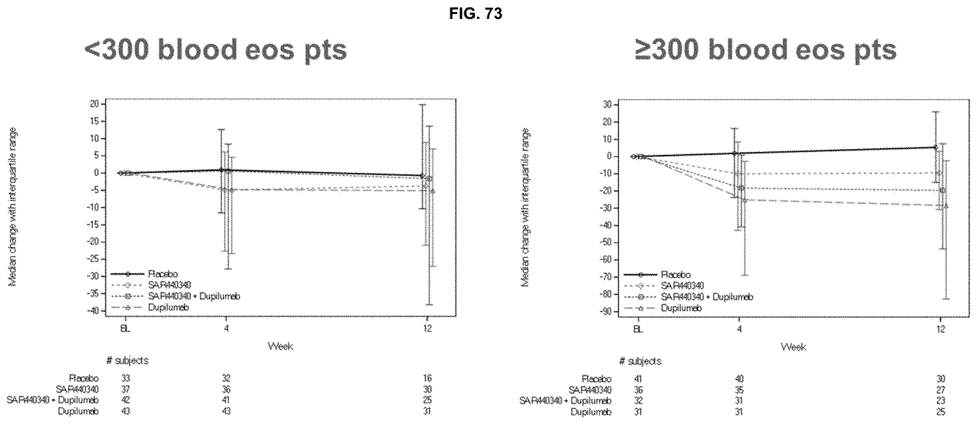

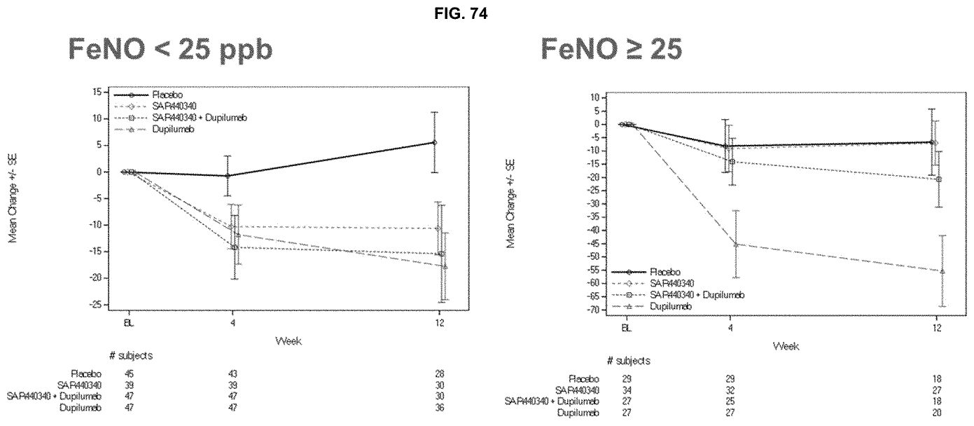

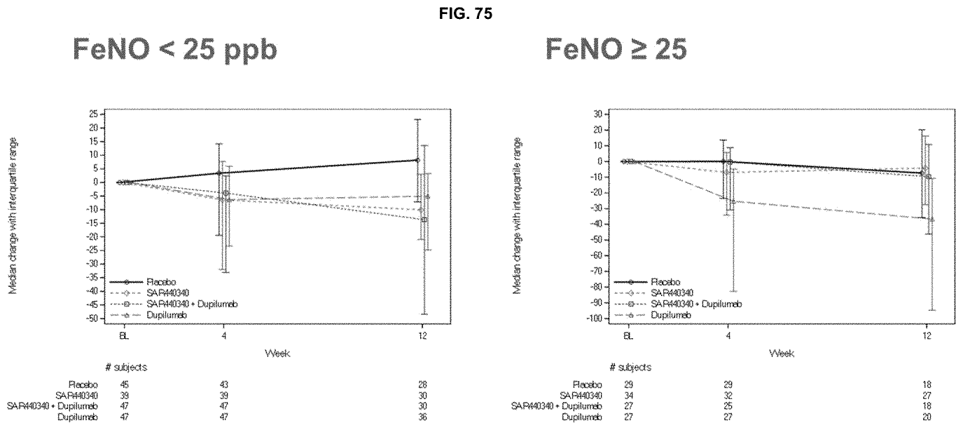

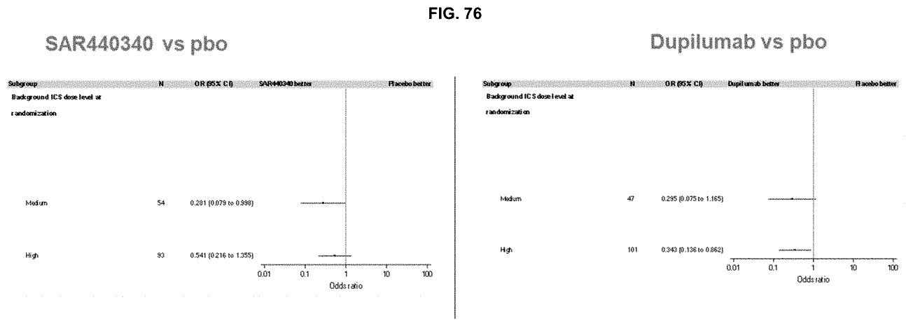

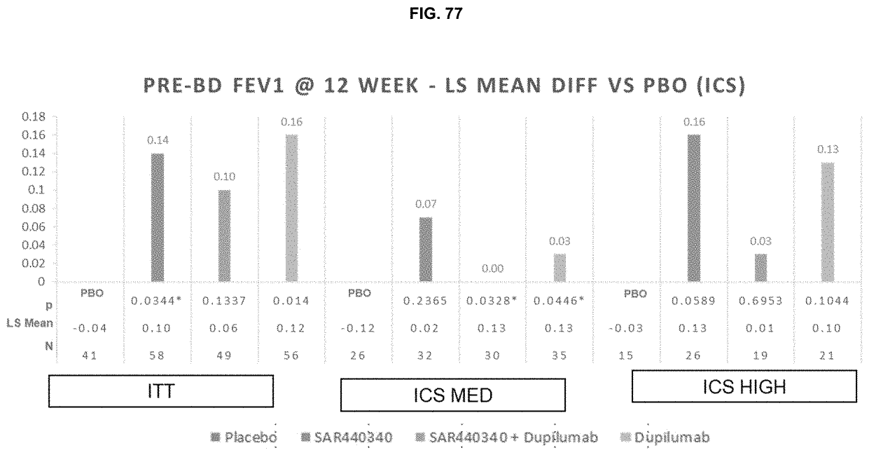

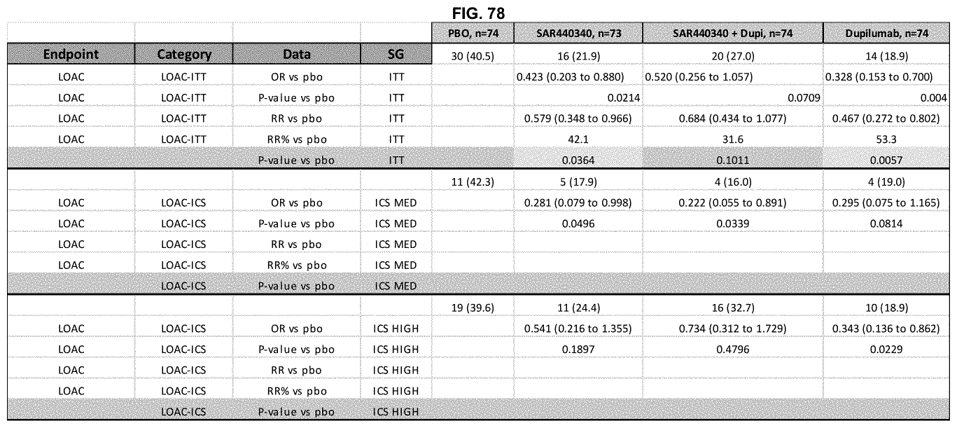

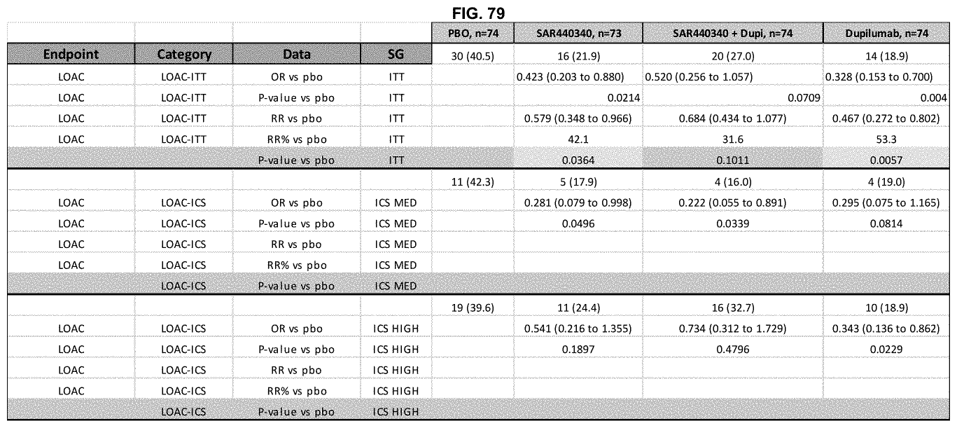

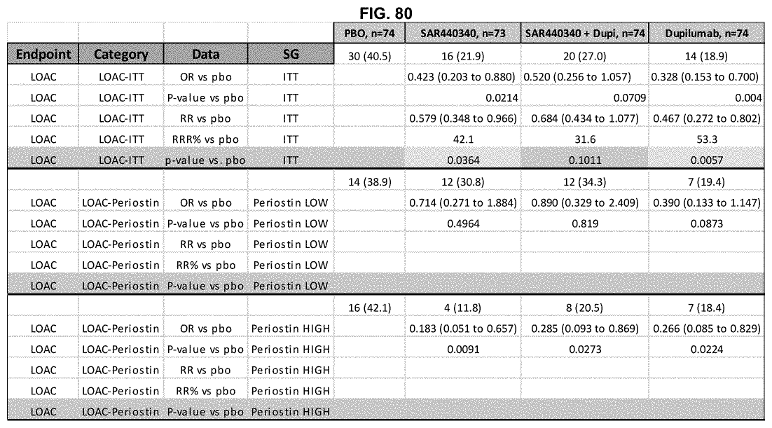

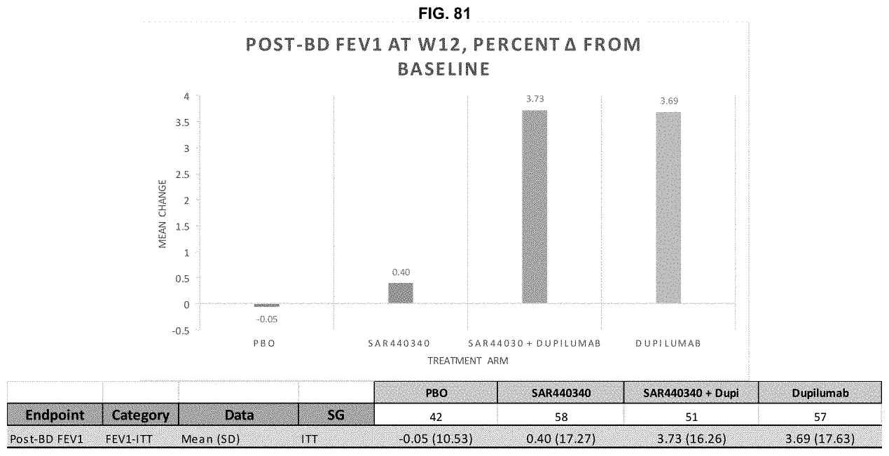

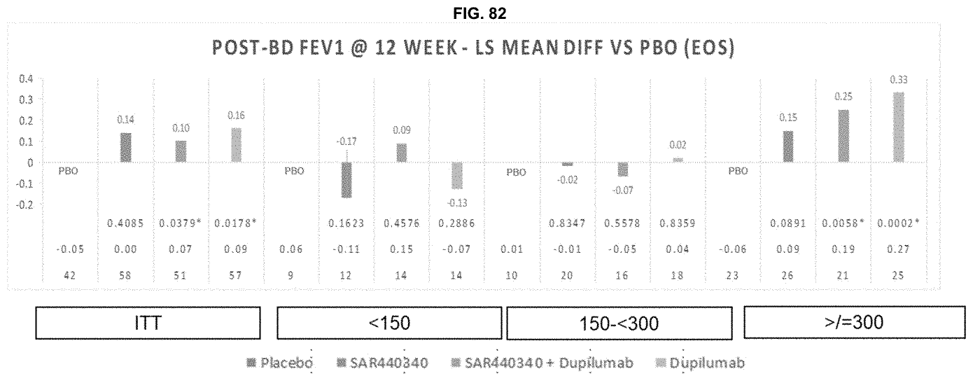

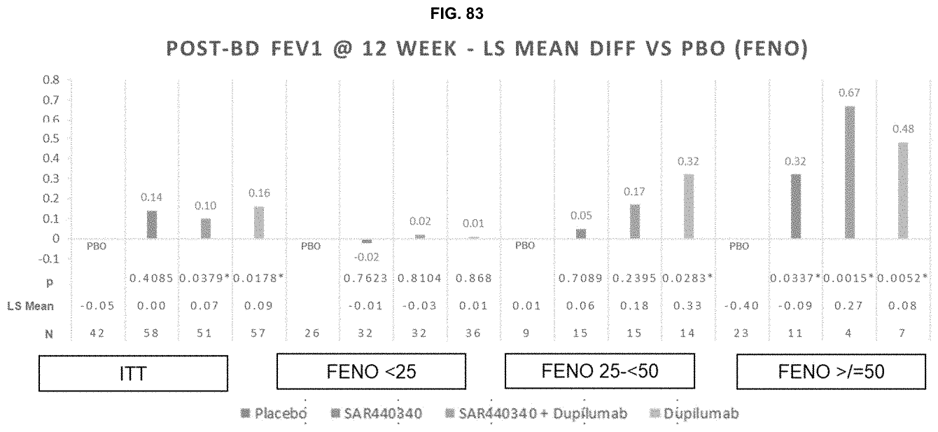

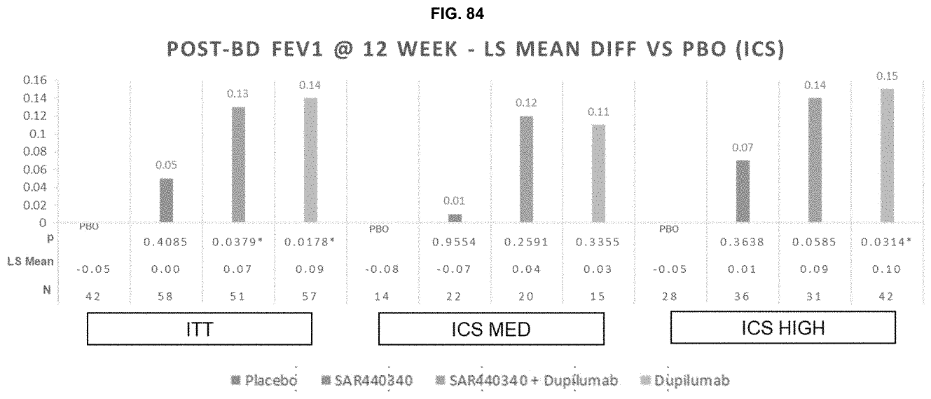

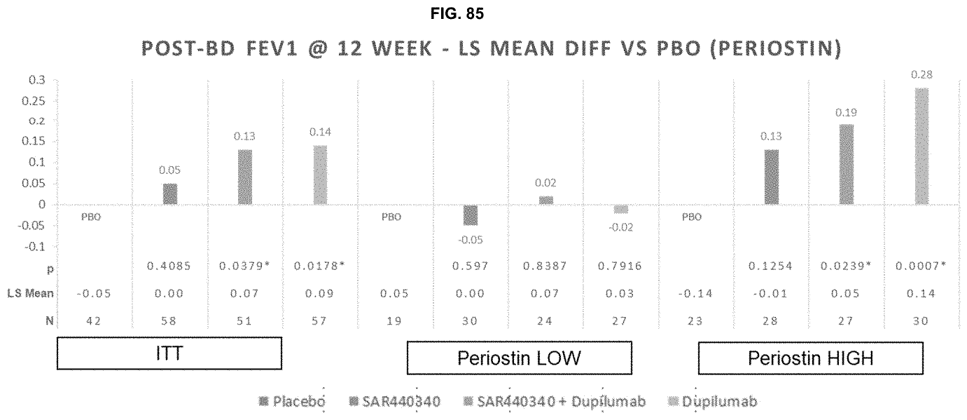

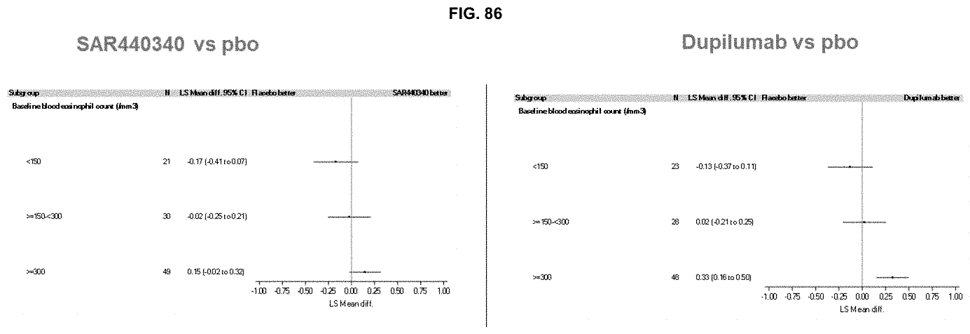

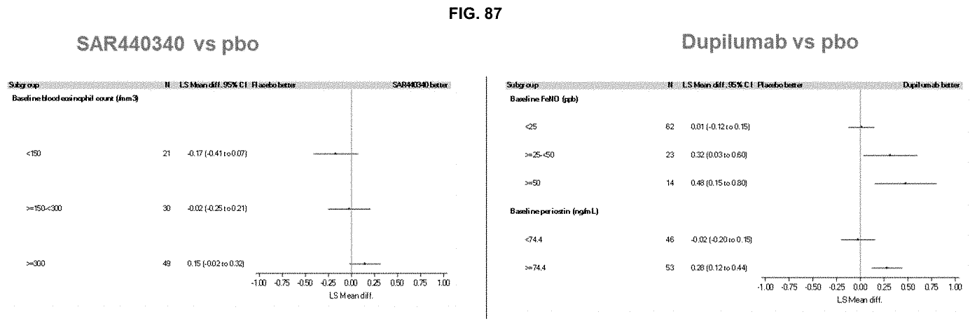

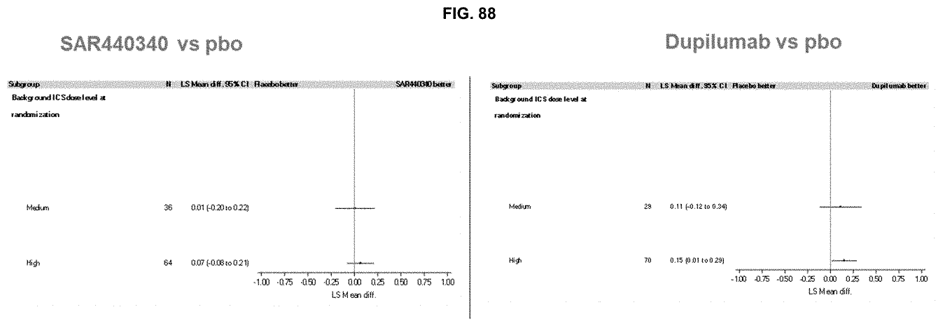

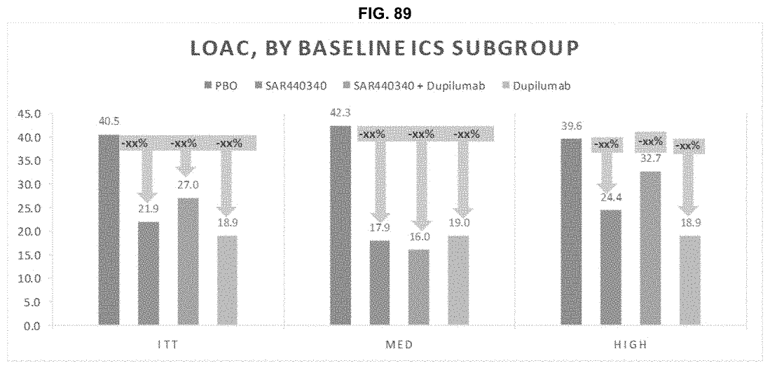

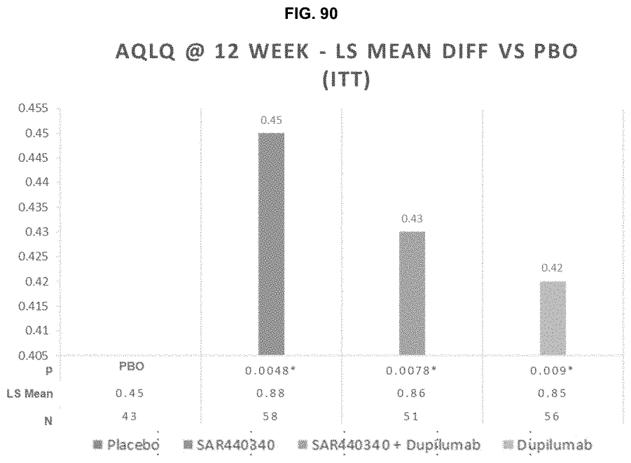

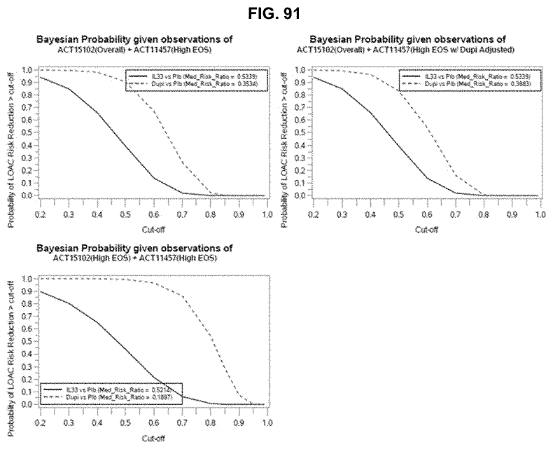

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the invention, the typical methods and materials are now described. All publications mentioned herein are incorporated herein by reference in their entirety.

### Methods for Reducing the Incidence of Asthma Exacerbations

The invention includes methods for reducing the incidence of asthma exacerbations in a subject in need thereof comprising administering a pharmaceutical composition comprising an interleukin-33 (IL-33) antagonist. The methods featured in the invention further comprise administering to a subject in need thereof a first therapeutic composition comprising an interleukin-33 (IL-33) antagonist, and a second therapeutic composition comprising an interleukin-4 receptor (IL-4R) antagonist. According to certain embodiments, the IL-33 antagonist is an antibody or antigen-binding fragment thereof that specifically binds IL-33. Exemplary anti-IL-33 antibodies that can be used in the context of the methods featured in the invention are described herein. According to certain embodiments, the IL-4R antagonist is an antibody or antigen-binding fragment thereof that specifically binds IL-4R. Exemplary anti-IL-4R antibodies that can be used in the context of the methods featured in the invention are described herein.

As used herein, the expression “asthma exacerbation” means an increase in the severity and/or frequency and/or duration of one or more symptoms or indicia of asthma. An “asthma exacerbation” also includes any deterioration in the respiratory health of a subject that requires and or is treatable by a therapeutic intervention for asthma (such as, e.g., steroid treatment, inhaled corticosteroid treatment, hospitalization, etc.). There are two types of asthma exacerbation events: a loss of asthma control (LOAC) event and a severe exacerbation event.

According to certain embodiments, a loss of asthma control (LOAC) event is defined as one or more of the following: (a) 30% or greater reduction from baseline in morning PEF on 2 consecutive days; (b) greater than or equal to 6 additional reliever puffs of salbutamol/albuterol or levosalbutamol/levalbuterol in a 24-hour period (compared to baseline) on 2 consecutive days; (c) an increase in ICS greater than or equal to 4 times the last prescribed ICS dose (or ≥50% of the prescribed ICS dose at V2 if background therapy withdrawal completed); (d) use of systemic (oral and/or parenteral) steroid treatment; or (e) hospitalization or emergency room visit because of asthma.

In certain instances, an asthma exacerbation may be categorized as a “severe asthma exacerbation event.” A severe asthma exacerbation event means an incident requiring immediate intervention in the form of treatment with either systemic corticosteroids or with inhaled corticosteroids at four or more times the dose taken prior to the incident. According to certain embodiments, a severe asthma exacerbation event is defined as a deterioration of asthma requiring: use of systemic corticosteroids for greater than or equal to 3 days; or hospitalization or emergency room visit because of asthma, requiring systemic corticosteroids. The general expression “asthma exacerbation” therefore includes and encompasses the more specific subcategory of “severe asthma exacerbations.” Accordingly, methods for reducing the incidence of severe asthma exacerbations in a patient in need thereof are included.

A “reduction in the incidence” of an asthma exacerbation means that a subject who has received a pharmaceutical composition comprising an IL-4R antagonist experiences fewer asthma exacerbations (i.e., at least one fewer exacerbation) after treatment than before treatment, or experiences no asthma exacerbations for at least 4 weeks (e.g., 4, 6, 8, 12, 14, or more weeks) following initiation of treatment with the pharmaceutical composition. A “reduction in the incidence” of an asthma exacerbation alternatively means that, following administration of the pharmaceutical composition, the likelihood that a subject experiences an asthma exacerbation is decreased by at least 10% (e.g., 10%, 15%, 20%, 25%, 30%, 35%, 40%, 45%, 50%, or more) as compared to a subject who has not received the pharmaceutical composition.

The invention includes methods for reducing the incidence of asthma exacerbations in a subject in need thereof comprising administering a pharmaceutical composition comprising an IL-4R antagonist to the subject as well as administering to the subject one or more maintenance doses of an inhaled corticosteroid (ICS) and/or one or more maintenance doses of a second controller, e.g., a long-acting beta-agonist (LABA) or a leukotriene receptor antagonist (LTA). Suitable ICSs include, but are not limited to, fluticasone (e.g., fluticasone propionate, e.g., Flovent™), budesonide, mometasone (e.g., mometasone furoate, e.g., Asmanex™), flunisolide (e.g., Aerobid™), dexamethasone acetate/phenobarbital/theophylline (e.g., Azmacort™), beclomethasone dipropionate HFA (Qvar™), and the like. Suitable LABAs include, but are not limited to, salmeterol (e.g., Serevent™), formoterol (e.g., Foradil™), and the like. Suitable LTAs include, but are not limited to, montelukast (e.g., Singulaire™), zafirlukast (e.g., Accolate™), and the like.

The invention includes methods for reducing the incidence of asthma exacerbations in a subject in need thereof comprising administering a pharmaceutical composition comprising an IL-4R antagonist to the subject as well as administering to the subject one or more reliever medications to eliminate or reduce one or more asthma-associated symptoms. Suitable reliever medications include, but are not limited to, quick-acting beta2-adrenergic receptor agonists such as, e.g., albuterol (i.e., salbutamol, e.g., Proventil™, Ventolin™, Xopenex™ and the like), pirbuterol (e.g., Maxair™), metaproterenol (e.g., Alupent™) and the like.

### Methods for Improving Asthma-Associated Parameters

The invention also includes methods for improving one or more asthma-associated parameters in a subject in need thereof, wherein the methods comprise administering a pharmaceutical composition comprising an IL-33 antagonist to the subject. The invention also includes methods for improving one or more asthma-associated parameters in a subject in need thereof, wherein the methods comprise administering a first pharmaceutical composition comprising an IL-33 antagonist and a second pharmaceutical composition comprising an IL-4R antagonist to the subject. A reduction in the incidence of an asthma exacerbation (as described above) may correlate with an improvement in one or more asthma-associated parameters; however, such a correlation is not necessarily observed in all cases.

Examples of “asthma-associated parameters” include: (1) relative percent change from baseline (e.g., at week 12) in forced expiratory volume in 1 second (FEV₁); (2) a relative percent change from baseline (e.g., at week 12) as measured by forced expiratory flow at 25-75% of the pulmonary volume (FEF25-75); (3) annualized rate of loss of asthma control events during the treatment period; (4) annualized rate of severe exacerbation events during the treatment period; (5) time to loss of asthma control events during the treatment period; (6) time to severe exacerbation events during the treatment period; (7) time to loss of asthma control events during overall study period; (8) time to severe exacerbation events during overall study period; (9) health care resource utilization; (10) change from baseline at week 12 in: i) morning and evening asthma symptom scores, ii) ACQ-5 score, iii) AQLQ score, iv) morning and evening PEF, v) number of inhalations/day of salbutamol/albuterol or levosalbutamol/levalbuterol for symptom relief, vi) nocturnal awakenings; (11) change from baseline at week 12 and week 24 in: i) 22-item Sino Nasal Outcome Test (SNOT-22), ii) Hospital Anxiety and Depression Score (HADS), iii) EuroQual questionnaire (EQ-5D-3L or EQ-5D-5L). An “improvement in an asthma-associated parameter” means an increase from baseline of one or more of FEV₁, AM PEF or PM PEF, and/or a decrease from baseline of one or more of daily albuterol/levalbuterol use, ACQ5 score, average nighttime awakenings or SNOT-22 score. As used herein, the term “baseline,” with regard to an asthma-associated parameter, means the numerical value of the asthma-associated parameter for a patient prior to or at the time of administration of a pharmaceutical composition comprising an IL-33 antagonist, or the numerical value of the asthma-associated parameter for a patient prior to or at the time of administration of a first pharmaceutical composition comprising an IL-33 antagonist and a second pharmaceutical composition comprising an IL-4R antagonist to the subject.

To determine whether an asthma-associated parameter has “improved,” the parameter is quantified at baseline and at a time point after administration of the pharmaceutical composition described herein. For example, an asthma-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the asthma associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as an asthma-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Forced Expiratory Volume in 1 Second (FEV1).

According to certain embodiments, administration of an IL-4R antagonist to a patient results in an increase from baseline of forced expiratory volume in 1 second (FEV1). Methods for measuring FEV₁ are known in the art. For example, a spirometer that meets the 2005 American Thoracic Society (ATS)/European Respiratory Society (ERS) recommendations can be used to measure FEV₁ in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FEV₁ (in liters).

The invention includes therapeutic methods that result in an increase of FEV₁ from baseline of at least 0.05 L at week 12 following initiation of treatment with a pharmaceutical composition comprising an anti-IL-33 antagonist or a first pharmaceutical composition comprising an IL-33 antagonist and a second pharmaceutical composition comprising an IL-4R antagonist. For example, administration of an IL-33 antagonist or an IL-33 antagonist and an IL-4R antagonist causes an increase of FEV₁ from baseline of about 0.05 L, 0.10 L, 0.12 L, 0.14 L, 0.16 L, 0.18 L, 0.20 L, 0.22 L, 0.24 L, 0.26 L, 0.28 L, 0.30 L, 0.32 L, 0.34 L, 0.36 L, 0.38 L, 0.40 L, 0.42 L, 0.44 L, 0.46 L, 0.48 L, 0.50 L, or more at week 12.

FEF25-75%.

According to certain embodiments, administration of an IL-33 antagonist or an IL-33 antagonist and an IL-4R antagonist to a patient results in an increase from baseline of FEF25-75%. Methods for measuring FEF are known in the art. For example, a spirometer that meets the 2005 American Thoracic Society (ATS)/European Respiratory Society (ERS) recommendations can be used to measure FEV₁ in a patient. The FEF25-75 (forced expiratory flow between 25% and 75%) is the speed (in liters per second) at which a person can empty the middle half of his or her air during a maximum expiration (i.e., Forced Vital Capacity or FVC). The parameter relates to the average flow from the point at which 25 percent of the FVC has been exhaled to the point at which 75 percent of the FVC has been exhaled. The FEF25-75% of a subject provides information regarding small airway function, such that the extent of mall airway disease and/or inflammation. A change in FEF25-75 is an early indicator of obstructive lung disease. In certain embodiments, an improvement and/or increase in the FEF25-75% parameter is an improvement of at least 10%, 25%, 50% or more as compared to baseline. In certain embodiments, the methods of the invention result in normal FEF25-75% values in a subject (e.g., values ranging from 50-60% and up to 130% of the average).

Morning and Evening Peak Expiratory Flow (AM PEF and PM PEF).

According to certain embodiments, administration of an IL-33 antagonist or an IL-33 antagonist and an IL-4R antagonist to a patient results in an increase from baseline of morning (AM) and/or evening (PM) peak expiratory flow (AM PEF and/or PM PEF). Methods for measuring PEF are known in the art. For example, according to one method for measuring PEF, patients are issued an electronic PEF meter for recording morning (AM) and evening (PM) PEF (as well as daily albuterol use, morning and evening asthma symptom scores, and number of nighttime awakenings due to asthma symptoms that require rescue medications). Patients are instructed on the use of the device, and written instructions on the use of the electronic PEF meter are provided to the patients. In addition, a medical professional may instruct the patients on how to record pertinent variables in the electronic PEF meter. AM PEF is generally performed within 15 minutes after arising (between 6 am and 10 am) prior to taking any albuterol. PM PEF is generally performed in the evening (between 6 pm and 10 pm) prior to taking any albuterol. Subjects should try to withhold albuterol for at least 6 hours prior to measuring their PEF. Three PEF efforts are performed by the patient and all 3 values are recorded by the electronic PEF meter. Usually the highest value is used for evaluation. Baseline AM PEF may be calculated as the mean AM measurement recorded for the 7 days prior to administration of the first dose of pharmaceutical composition comprising the IL-4R antagonist, and baseline PM PEF may be calculated as the mean PM measurement recorded for the 7 days prior to administration of the first dose of pharmaceutical composition comprising the IL-33 antagonist or the IL-33 antagonist and the IL-4R antagonist.

The invention includes therapeutic methods that result in an increase in AM PEF and/or PM PEF from baseline of at least 1.0 L/min at week 12 following initiation of treatment with a pharmaceutical composition comprising an anti-IL-33 antagonist or an IL-33 antagonist and an IL-4R antagonist. For example, according to the invention, administration of an IL-33 antagonist or an IL-33 antagonist and an IL-4R antagonist to a subject in need thereof causes an increase in PEF from baseline of about 0.5 L/min, 1.0 L/min, 1.5 L/min, 2.0 L/min, 2.5 L/min, 3.0 L/min, 3.5 L/min, 4.0 L/min, 4.5 L/min, 5.0 L/min, 5.5 L/min, 6.0 L/min, 6.5 L/min, 7.0 L/min, 7.5 L/min, 8.0 L/min, 8.5 L/min, 9.0 L/min, 9.5 L/min, 10.0 L/min, 10.5 L/min, 11.0 L/min, 12.0 L/min, 15 L/min, 20 L/min, or more at week 12.

Albuterol/Levalbuterol Use.

According to certain embodiments, administration of an IL-33 antagonist or an IL-33 antagonist and an IL-4R antagonist to a patient results in a decrease from baseline of daily albuterol or levalbuterol use. The number of albuterol/levalbuterol inhalations can be recorded daily by the patients in a diary, PEF meter, or other recording device. During treatment with the pharmaceutical composition described herein, use of albuterol/levalbuterol typically may be on an as-needed basis for symptoms, not on a regular basis or prophylactically. The baseline number of albuterol/levalbuterol inhalations/day may be calculated based on the mean for the 7 days prior to administration of the first dose of pharmaceutical composition comprising the IL-4R antagonist.

The invention includes therapeutic methods that result in a decrease in albuterol/levalbuterol use from baseline of at least 0.25 puffs per day at week 12 following initiation of treatment with a pharmaceutical composition comprising an anti-IL-33 antagonist or a first pharmaceutical composition comprising an IL-33 antagonist and a second pharmaceutical composition comprising an IL-4R antagonist. For example, administration of an IL-33 antagonist or an IL-33 antagonist and an IL-4R antagonist to a subject in need thereof causes a decrease in albuterol/levalbuterol use from baseline of about 0.25 puffs per day, 0.50 puffs per day, 0.75 puffs per day, 1.00 puff per day, 1.25 puffs per day, 1.5 puffs per day, 1.75 puffs per day, 2.00 puffs per day, 2.25 puffs per day, 2.5 puffs per day, 2.75 puffs per day, 3.00 puffs per day, or more at week 12.

OCS Use.

According to certain embodiments, administration of an IL-33 antagonist or an IL-33 antagonist and an IL-4R antagonist to a patient can be used in conjunction with an OCS such as oral prednisone. The number of OCS administrations can be recorded daily by the patients in a diary, PEF meter, or other recording device. During treatment with the pharmaceutical composition described herein, occasional short-term use of prednisone typically can be used to control acute asthmatic episodes, e.g., episodes in which bronchodilators and other anti-inflammatory agents fail to control symptoms. In other aspects, prednisone is used concurrent with or as a substitution for ICS. Oral prednisone may be administered in dosages of about 5 mg, 10 mg, 15 mg, 20 mg, 25 mg, 30 mg, 35 mg or 40 mg. OCS can optionally be administered once a day or multiple times a day (e.g., twice a day, three times a day, four times a day, etc.)

In certain exemplary embodiments, the invention provides methods for reducing or eliminating the dependency of the subject on OCS use. The reduction or elimination of steroid dependency is highly advantageous and desirable. In certain embodiments, a reduction of 50% or greater (e.g., 50%, 60%, 70%, 80%, 90% or more) in the OCS dose is achieved after administration of IL-4R antibody therapy at a period of time (e.g., at week 240. In certain embodiments, the OCS is substantially eliminated after 40 weeks, 45 weeks, 50 weeks, 52 weeks, or greater after the first dose following administration of the initial dose. In other embodiments, the level of OCS use is reduced to less than 5 mg per day (e.g., less than 5 mg, 4 mg, 3 mg, 2 mg or less per day). In other embodiments, the dependency on OCS use is substantially eliminated after 3 months, 6 months, 9 months or 1 year following treatment with IL-33 antagonist or an IL-33 antagonist and an IL-4R antagonist.

5-Item Asthma Control Questionnaire (ACQ) Score.

According to certain embodiments, administration of an IL-33 antagonist or an IL-33 antagonist and an IL-4R antagonist to a patient results in a decrease from baseline of five-item Asthma Control Questionnaire (ACQS) score. The ACQS is a validated questionnaire to evaluate asthma control.

The invention includes therapeutic methods that result in a decrease in ACQS score from baseline of at least 0.10 points at week 12 following initiation of treatment with a pharmaceutical composition comprising an anti-IL-33 antagonist or an IL-33 antagonist and an IL-4R antagonist. For example, administration of an IL-33 antagonist or an IL-33 antagonist and an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 12.

Night-Time Awakenings.

According to certain embodiments, administration of an IL-33 antagonist or an IL-33 antagonist and an IL-4R antagonist to a patient results in a decrease from baseline of average number of nighttime awakenings.

In certain embodiments, the methods decrease the average number of nighttime awakenings from baseline by at least about 0.10 times per night at week 12 following initiation of treatment. For example, administration of an IL-33 antagonist or an IL-33 antagonist and an IL-4R antagonist to a subject in need thereof can cause a decrease in average number of nighttime awakenings from baseline of about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 times per night, 2.0 times per night, or more at week 12.

22-Item Sinonasal Outcome Test (SNOT-22) Score.

According to certain embodiments, administration of an IL-33 antagonist or an IL-33 antagonist and an IL-4R antagonist to a patient results in a decrease from baseline of 22-item Sinonasal Outcome Test (SNOT-22). The SNOT-22 is a validated questionnaire to assess the impact of chronic rhinosinusitis on quality of life (Hopkins et al 2009, Clin. Otolaryngol. 34: 447-454).

The invention includes therapeutic methods that result in a decrease in SNOT-22 score from baseline of at least 1 point at week 12 following initiation of treatment with a pharmaceutical composition comprising an anti-IL-33 antagonist or an IL-33 antagonist and an IL-4R antagonist. For example, administration of an IL-33 antagonist or an IL-33 antagonist and an IL-4R antagonist to a subject in need thereof can cause a decrease in SNOT-22 score from baseline of about 1, 2, 3, 4, 5, 6, 7, 8, 9, 10, 11, 12, 13 points, or more at week 12.

Biomarkers.

In certain embodiments, the subject experiences an improvement in lung function as measured by a biomarker. For example, the biomarker may be fractional exhaled nitric oxide (FeNO), eotaxin-3, total IgE, periostin, or thymus and activation-regulated chemokine (TARC). In certain embodiments, an improvement in lung function is indicated by a reduction or increase (as appropriate) at week 4, week 12 or week 24 following treatment.

### Methods for Treating Asthma

In some embodiments, the invention provides methods for treating asthma, including, e.g., moderate-to-severe asthma, in a subject in need thereof, wherein the methods comprise administering a pharmaceutical composition comprising an IL-33 antagonist or an IL-33 antagonist and an IL-4R antagonist to the subject. In certain embodiments, the methods are useful for treating moderate-to-severe asthma in a subject.

As used herein, the term “asthma” can be used interchangeably with “intermittent asthma,” or “bronchial asthma.” “Asthma,” “bronchial asthma” and “intermittent asthma” refer to asthma in which one or any combination of the following are true: symptoms occur 2 or fewer days per week; symptoms do not interfere with normal activities; nighttime symptoms occur fewer than 2 days per month; or one or more lung function tests (e.g., forced expiratory volume in one second (FEV₁) and/or peak expiratory flow (PEF) of greater than 80%) are normal when the subject is not suffering from an asthma attack.

As used herein, the term “persistent asthma” or “persistent bronchial asthma” refers to asthma that is more severe than (bronchial) asthma/intermittent (bronchial) asthma. A subject suffering from persistent asthma or persistent bronchial asthma experiences one or more of the following: symptoms more than 2 days per week; symptoms that interfere with normal activities; nighttime symptoms that occur more than 2 days per month; or one or more lung function tests (e.g., forced expiratory volume in one second (FEV₁) and/or peak expiratory flow (PEF) of less than 80%) that are not normal when the subject is not suffering from an asthma attack; the subject relies on daily asthma control medication; the subject has taken a systemic steroid more than once in the last year after a severe asthma flare-up; or use of a short-acting beta-2 agonist more than two days per week for relief of asthma symptoms.

Asthma/intermittent asthma, bronchial asthma/intermittent bronchial asthma, and persistent asthma/persistent bronchial asthma can be categorized as “mild,” “moderate,” “severe” or “moderate-to-severe.” “Mild intermittent asthma” or “mild intermittent bronchial asthma” is defined as having symptoms less than once a week, and having forced expiratory volume in one second (FEV₁) or peak expiratory flow (PEF)≥80%. “Mild persistent asthma” or “mild persistent bronchial asthma” differs in that symptoms frequency is greater than once per week but less than once per day, and variability in FEV₁ or PEF is <20%-30%. “Moderate intermittent asthma” or “moderate intermittent bronchial asthma” is defined as having symptoms less than once a week, and having forced expiratory volume in one second (FEV₁) or peak expiratory flow (PEF) of 60-80%. “Moderate persistent asthma” or “moderate persistent bronchial asthma” is defined as having daily symptoms, exacerbations that may affect activity and/or sleep, nocturnal symptoms more than once a week, daily use of inhaled short-acting beta-2 agonist and having forced expiratory volume in one second (FEV₁) or peak expiratory flow (PEF) of 60-80%. “Severe intermittent asthma” or “severe intermittent bronchial asthma” is defined as having symptoms less than once a week, and having forced expiratory volume in one second (FEV₁) or peak expiratory flow (PEF) of 60%. “Severe persistent asthma” or “severe persistent bronchial asthma” is defined as having daily symptoms, frequent exacerbations that may affect activity and/or sleep, frequent nocturnal symptoms, limitation of physical activities, daily use of inhaled short-acting beta-2 agonist, and having forced expiratory volume in one second (FEV₁) or peak expiratory flow (PEF) of 60%. “Moderate-to-severe intermittent asthma” or “moderate-to-severe intermittent bronchial asthma” is defined as having symptoms between those of moderate intermittent asthma/moderate intermittent bronchial asthma and severe intermittent asthma/severe intermittent bronchial asthma. “Moderate-to-severe persistent asthma” or “moderate-to-severe persistent bronchial asthma” is defined as having symptoms between those of moderate persistent asthma/moderate persistent bronchial asthma and severe persistent asthma/severe persistent bronchial asthma.

As used herein, the term “inadequately controlled asthma” refers to patients whose asthma is either “not well controlled” or “very poorly controlled” as defined by the “Expert Panel Report 3: Guidelines for the Diagnosis and Management of Asthma,” National Heart, Blood and Lung Institute, NIH, Aug. 28, 2007. “Not well controlled asthma” is defined as having symptoms greater than two days per week, nighttime awakenings one to three times per week, some limitations on normal activity, short-acting beta2-agonist use for symptom control greater than two days per week, FEV₁ of 60-80% of predicted and/or personal best, an ATAQ score of 1-2, an ACQ score of 1.5 or greater, and an ACT score of 16-19. “Very poorly controlled asthma” is defined as having symptoms throughout the day, nighttime awakenings four times or more per week, extreme limitations on normal activity, short-acting beta2-agonist use for symptom control several times per day, FEV₁ of less than 60% of predicted and/or personal best, an ATAQ score of 3-4, an ACQ score of N/A, and an ACT score of less than or equal to 15.

In some embodiments, a subject is identified as having “moderate-to-severe uncontrolled” asthma if the subject receives such a diagnosis from a physician, based on the Global Initiative for Asthma (GINA) 2009 Guidelines, and one or more of the following criteria: i) Existing treatment with moderate-to-high dose ICS/LABA (2 fluticasone propionate 250 μg twice daily or equipotent ICS daily dosage) with a stable dose of ICS/LABA for greater than or equal to 1 month prior to administration of an initial dose of IL-33 antagonist or an initial dose of IL-33 antagonist and IL-4R antagonist; ii) FEV₁ 40 to 80% predicted normal prior to administration of an initial dose of IL-33 antagonist or an initial dose of IL-33 antagonist and IL-4R antagonist; iii) ACQ-5 score greater than or equal to 1.5 prior to administration of an initial dose of IL-33 antagonist or an initial dose of IL-33 antagonist and IL-4R antagonist; iv) reversibility of at least 12% and 200 mL in FEV₁ after 200 μg to 400 μg (2 to 4 inhalations) of salbutamol/albuterol prior to administration of an initial dose of IL-33 antagonist or an initial dose of IL-33 antagonist and IL-4R antagonist; or v) has experienced, within 1 year prior to administration of an initial dose of IL-33 antagonist or an initial dose of IL-33 antagonist and IL-4R antagonist, any of the following events: (a) treatment with greater than or equal to 1 systemic (oral or parenteral) steroid burst for worsening asthma, (b) hospitalization or an emergency/urgent medical care visit for worsening asthma.

“Severe asthma” refers to asthma in which adequate control cannot be achieved by high-dose treatment with inhaled corticosteroids and additional controllers (e.g., long-acting inhaled beta 2 agonists, montelukast, and/or theophylline) or by oral corticosteroid treatment (e.g., for at least six months per year), or is lost when the treatment is reduced. In certain embodiments, severe asthma includes asthma that is treated with high-dose ICS and at least one additional controller (e.g., LABA, montelukast, or theophylline) or oral corticosteroids>6 months/year, wherein at least one of the following occurs or would occur if treatment is reduced: ACT<20 or ACQ>1.5; at least 2 exacerbations in the last 12 months; at least 1 exacerbation treated in hospital or requiring mechanical ventilation in the last 12 months; or FEV1<80% (if FEV1/FVC below the lower limit of normal).

“Steroid-dependent asthma” refers to asthma which requires one or more of the following treatments: frequent, short term oral corticosteroid treatment bursts in the past 12 months; regular use of high dose inhaled corticosteroids in the past 12 months; regular use of injected long acting corticosteroids; daily use of oral corticosteroids; alternate-day oral corticosteroids; or prolonged use of oral corticosteroids in the past year.

“Oral corticosteroid-dependent asthma” refers to a subject having ≥3 30-day oral corticosteroid (OCS) fills over a 12-month period and a primary asthma diagnosis within 12 months of the first OCS fill. Subjects with OCS-dependent asthma may also experience one or any combination of the following: have received physician prescribed LABA and high dose ICS (total daily dose>500 μg fluticasone propionate dry powder formulation equivalent) for at least 3 months (the ICS and LABA can be parts of a combination product, or given by separate inhalers); have received additional maintenance asthma controller medications according to standard practice of care e.g., leukotriene receptor antagonists (LTRAs), theophylline, long-acting muscarinic antagonists (LAMAs), secondary ICS and cromones; received OCS for the treatment of asthma at a dose of between ≥7.5 to ≤30 mg (prednisone or prednisolone equivalent); have received an OCS dose administered every other day (or different doses every other day); morning pre-bronchodilator (BD) FEV1 of <80% predicted normal; have evidence of asthma as documented by post-BD (albuterol/salbutatomol) reversibility of FEV1≥12% and ≥200 mL (15-30 min after administration of 4 puffs of albuterol/salbutamol); or have a history of at least one asthma exacerbation event within 12 months.

In one aspect, methods for treating asthma are provided comprising: (a) selecting a patient that exhibits a blood eosinophil level of at least 300 cells per microliter; and (b) administering to the patient a pharmaceutical composition comprising an IL-33 antagonist or an IL-33 antagonist and an IL-4R antagonist.

In another aspect, methods for treating asthma are provided comprising: (a) selecting a patient that exhibits a blood eosinophil level of 150-299 cells per microliter; and (b) administering to the patient a pharmaceutical composition comprising an IL-33 antagonist or an IL-33 antagonist and an IL-4R antagonist.

In another aspect, methods for treating asthma are provided comprising: (a) selecting a patient that exhibits a blood eosinophil level of less than 150 cells per microliter; and (b) administering to the patient a pharmaceutical composition comprising an IL-33 antagonist or an IL-33 antagonist and an IL-4R antagonist.

In one aspect, methods for treating asthma are provided comprising: (a) selecting a patient that exhibits a low level of periostin level; and (b) administering to the patient a pharmaceutical composition comprising an IL-33 antagonist or an IL-33 antagonist and an IL-4R antagonist.

In another aspect, methods for treating asthma are provided comprising: (a) selecting a patient that exhibits a high level of periostin; and (b) administering to the patient a pharmaceutical composition comprising an IL-33 antagonist or an IL-33 antagonist and an IL-4R antagonist.

As used herein, a “high level of periostin” refers to a blood periostin measurement of greater than or equal to about 60 ng/mL, greater than or equal to about 65 ng/mL, greater than or equal to about 70 ng/mL, greater than or equal to about 75 ng/mL, or greater than or equal to about 80 ng/mL, greater than or equal to about 85 ng/mL, greater than or equal to about 90 ng/mL, greater than or equal to about 95 ng/mL, greater than or equal to about 100 ng/mL. In particularly exemplary embodiments, a high level of periostin is greater than or equal to about 75.0 ng/mL or greater than or equal to about 74.4 ng/mL.

As used herein, a “low level of periostin” refers to a blood periostin measurement of less than about 100 ng/mL, less than about 95 ng/mL, less than about 90 ng/mL, less than about 85 ng/mL, less than about 80 ng/mL, less than about 75 ng/mL, less than about 70 ng/mL, less than about 65 ng/mL, or less than about 60 ng/mL. In particularly exemplary embodiments, a low level of periostin is less than about 75.0 ng/mL or less than about 74.4 ng/mL.

In a related aspect, methods for treating asthma comprising an add-on therapy to background therapy are provided. In certain embodiments, an IL-33 antagonist or an IL-33 antagonist and an IL-4R antagonist is administered as an add-on therapy to an asthma patient who is on background therapy for a certain period of time (e.g., 1 week, 2 weeks, 3 weeks, 1 month, 2 months, 5 months, 12 months, 18 months, 24 months, or longer) (also called the “stable phase”). In some embodiments, the background therapy comprises a ICS and/or a LABA.

In some embodiments, the invention includes a method for reducing an asthma patient's dependence on ICS and/or LABA for the treatment of one or more asthma exacerbations comprising: (a) selecting a patient who has moderate-to-severe asthma that is not well-controlled with a background asthma therapy comprising an ICS, a LABA, or a combination thereof; and administering to the patient a pharmaceutical composition comprising an IL-33 antagonist or an IL-33 antagonist and an IL-4R antagonist.

In some embodiments, the invention encompasses methods to treat or alleviate conditions or complications associated with asthma, such as chronic rhinosinusitis, allergic rhinitis, allergic fungal rhino sinusitis, allergic broncho-pulmonary aspergillosis, unified airway disease, Churg-Strauss syndrome, vasculitis, chronic obstructive pulmonary disease (COPD), and exercise induced bronchospasm.

The invention also includes methods for treating persistent asthma. As used herein, the term “persistent asthma” means that the subject has symptoms at least once a week at day and/or at night, with the symptoms lasting a few hours to a few days. In certain alternative embodiments, the persistent asthma is “mildly persistent” (e.g., more than twice a week but less than daily with symptoms severe enough to interfere with daily activities or sleep and/or where pulmonary function is normal or reversible with inhalation of a bronchodilator), “moderately persistent” (e.g., symptoms occurring daily with sleep interrupted at least weekly and/or with pulmonary function moderately abnormal), or “severely persistent” (e.g., continuous symptoms despite the correct use of approved medications and/or where pulmonary function is severely affected).

### Interleukin-33 (IL-33) Antagonists and Interleukin-4 Receptor (IL-4R) Antagonists

The methods featured in the invention comprise administering to a subject in need thereof a therapeutic composition comprising an IL-33 antagonist. As used herein, an “IL-33 antagonist” is any agent that binds to or interacts with IL-33 and inhibits the normal biological signaling function of IL-33 when IL-33 is expressed on a cell in vitro or in vivo.

The methods featured in the invention optionally comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo.

Non-limiting examples of categories of IL-33 antagonists and IL-4R antagonists include small molecule IL-33 antagonists, small molecule IL-4R antagonists, anti-IL-33 aptamers, anti-IL-4R aptamers, peptide-based IL-33 antagonists or peptide-based IL-4R antagonists (e.g., “peptibody” molecules), and antibodies or antigen-binding fragments of antibodies that specifically bind human IL-33 or human IL-4R.

According to certain embodiments, the IL-33 antagonist comprises an anti-IL-33 antibody or antigen-binding fragment thereof that can be used in the context of the methods featured in the invention are described elsewhere herein. For example, in one embodiment, the IL-33 antagonist is an antibody or antigen-binding fragment thereof that specifically binds to an IL-33, and comprises the heavy chain and light chain (complementarity determining region) CDR sequences from the heavy chain variable region (HCVR) and light chain variable region (LCVR) of SEQ ID NOs: 2 and 10, respectively. In another embodiment, the IL-33 antagonist is an antibody or antigen-binding fragment thereof that specifically binds to an IL-33, and comprises the heavy chain and light chain CDR sequences of SEQ ID NOs: 4, 5 and 6 and SEQ ID NOs: 12, 14 and 16, respectively. In another embodiment, the IL-33 antagonist is an antibody or antigen-binding fragment thereof that specifically binds to an IL-33, and comprises an HCVR/LCVR pair of SEQ ID NOs: 2 and 10, respectively.

aggtgcagct ggtggagtct gggggaaact tggaacagcc tggggggtcc cttagactct cctgtacagc ctctggattc
acctttagca gatctgccat gaactgggtc cgccgggctc cagggaaggg gctggagtgg gtctcaggaa ttagtggtag tggtggtcga
acatactacg cagactccgt gaagggccgg ttcaccatct ccagagacaa ttccaagaat acgctatatc tgcaaatgaa cagcctgagc
gccgaggaca cggccgcata ttactgtgcg aaagattcgt atactaccag ttggtacgga ggtatggacg tctggggcca
cgggaccacg gtcaccgtct cctca (SEQ ID NO: 1), SAR440340 HCVR, DNA sequence.
VQLVESGGNLEQPGGSLRLSCTASGFTFSRSAMNWVRRAPGKGLEWVSGISGSG
GRTYYADSVKGRFTISRDNSKNTLYLQMNSLSAEDTAAYYCAKDSYTTSWYGGMDVWG
HGTTVTVSS (SEQ ID NO: 2), SAR440340 HCVR, amino acid sequence.
gattcacctt tagcagatct gcc (SEQ ID NO: 3), SAR440340 HCDR1, DNA sequence.
FTFSRSA (SEQ ID NO: 4), SAR440340 HCDR1, amino acid sequence.
ttagtggtag tggtggtcga aca (SEQ ID NO: 5), SAR440340 HCDR2, DNA sequence.
SGSGGRT (SEQ ID NO: 6), SAR440340 HCDR2, amino acid sequence.
cgaaagattc gtatactacc agttggtacg gaggtatgga cgtc (SEQ ID NO: 7), SAR440340 HCDR3,
DNA sequence.
KDSYTTSWYGGMDV (SEQ ID NO: 8), SAR440340 HCDR3, amino acid sequence.
acatccagat gacccagtct ccatcttccg tgtctgcatc tgtaggagac agagtcacca tcacttgtcg ggcgagtcag
ggtattttca gctggttagc ctggtatcag cagaaaccag gaaaagcccc taagctcctg atctatgctg cttccagttt acaaagtggg
gtcccatcaa gattcagcgg cagtggatct gggacagatt tcactctcac catcagcagc ctgcagcctg aggattttgc aatttactat
tgtcaacagg ctaacagtgt cccgatcacc ttcggccaag ggacacgact ggagattaaa cga (SEQ ID NO: 9),
SAR440340 LCVR, DNA sequence.
IQMTQSPSSVSASVGDRVTITCRASQGIFSWLAWYQQKPGKAPKLLIYAASSLQSG
VPSRFSGSGSGTDFTLTISSLQPEDFAIYYCQQANSVPITFGQGTRLEIKR (SEQ ID NO: 10)
SAR440340 LCVR, amino acid sequence.
agggtatttt cagctgg (SEQ ID NO: 11), SAR440340 LCDR1, DNA sequence.
GIFSW (SEQ ID NO: 12), SAR440340 LCDR1, amino acid sequence.
ctgcttcc (SEQ ID NO: 13), SAR440340 LCDR2, DNA sequence.
AS (SEQ ID NO: 14), SAR440340 LCDR2, amino acid sequence.
aacaggctaa cagtgtcccg atcacc (SEQ ID NO: 15), SAR440340 LCDR3, DNA sequence.
QANSVPIT (SEQ ID NO: 16), SAR440340 LCDR3, amino acid sequence.
aggtgcagct ggtggagtct gggggaaact tggaacagcc tggggggtcc cttagactct cctgtacagc ctctggattc
acctttagca gatctgccat gaactgggtc cgccgggctc cagggaaggg gctggagtgg gtctcaggaa ttagtggtag tggtggtcga
acatactacg cagactccgt gaagggccgg ttcaccatct ccagagacaa ttccaagaat acgctatatc tgcaaatgaa cagcctgagc
gccgaggaca cggccgcata ttactgtgcg aaagattcgt atactaccag ttggtacgga ggtatggacg tctggggcca
cgggaccacg gtcaccgtct cctcagcctc caccaagggc ccatcggtct tccccctggc gccctgctcc aggagcacct
ccgagagcac agccgccctg ggctgcctgg tcaaggacta cttccccgaa ccggtgacgg tgtcgtggaa ctcaggcgcc
ctgaccagcg gcgtgcacac cttcccggct gtcctacagt cctcaggact ctactccctc agcagcgtgg tgaccgtgcc ctccagcagc
ttgggcacga agacctacac ctgcaacgta gatcacaagc ccagcaacac caaggtggac aagagagttg agtccaaata
tggtccccca tgcccaccct gcccagcacc tgagttcctg gggggaccat cagtcttcct gttcccccca aaacccaagg acactctcat
gatctcccgg acccctgagg tcacgtgcgt ggtggtggac gtgagccagg aagaccccga ggtccagttc aactggtacg
tggatggcgt ggaggtgcat aatgccaaga caaagccgcg ggaggagcag ttcaacagca cgtaccgtgt ggtcagcgtc
ctcaccgtcc tgcaccagga ctggctgaac ggcaaggagt acaagtgcaa ggtctccaac aaaggcctcc cgtcctccat
cgagaaaacc atctccaaag ccaaagggca gccccgagag ccacaggtgt acaccctgcc cccatcccag gaggagatga
ccaagaacca ggtcagcctg acctgcctgg tcaaaggctt ctaccccagc gacatcgccg tggagtggga gagcaatggg
cagccggaga acaactacaa gaccacgcct cccgtgctgg actccgacgg ctccttcttc ctctacagca ggctcaccgt
ggacaagagc aggtggcagg aggggaatgt cttctcatgc tccgtgatgc atgaggctct gcacaaccac tacacacaga agtccctctc
cctgtctctg ggtaaatga (SEQ ID NO: 17), SAR440340 heavy chain DNA sequence.
VQLVESGGNLEQPGGSLRLSCTASGFTFSRSAMNWVRRAPGKGLEWVSGISGSGG
RTYYADSVKGRFTISRDNSKNTLYLQMNSLSAEDTAAYYCAKDSYTTSWYGGMDVWGH
GTTVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSRW
QEGNVFSCSVMHEALHNHYTQKSLSLSLGK (SEQ ID NO: 18), SAR440340 heavy chain
amino acid sequence.
acatccagat gacccagtct ccatcttccg tgtctgcatc tgtaggagac agagtcacca tcacttgtcg ggcgagtcag
ggtattttca gctggttagc ctggtatcag cagaaaccag gaaaagcccc taagctcctg atctatgctg cttccagttt acaaagtggg
gtcccatcaa gattcagcgg cagtggatct gggacagatt tcactctcac catcagcagc ctgcagcctg aggattttgc aatttactat
tgtcaacagg ctaacagtgt cccgatcacc ttcggccaag ggacacgact ggagattaaa cgaactgtgg ctgcaccatc tgtcttcatc
ttcccgccat ctgatgagca gttgaaatct ggaactgcct ctgttgtgtg cctgctgaat aacttctatc ccagagaggc caaagtacag
tggaaggtgg ataacgccct ccaatcgggt aactcccagg agagtgtcac agagcaggac agcaaggaca gcacctacag
cctcagcagc accctgacgc tgagcaaagc agactacgag aaacacaaag tctacgcctg cgaagtcacc catcagggcc
tgagctcgcc cgtcacaaag agcttcaaca ggggagagtg ttag (SEQ ID NO: 19), SAR440340 light chain DNA
sequence.
IQMTQSPSSVSASVGDRVTITCRASQGIFSWLAWYQQKPGKAPKLLIYAASSLQSG
VPSRFSGSGSGTDFTLTISSLQPEDFAIYYCQQANSVPITFGQGTRLEIKRTVAAPSVFIFPPSD
EQLKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 20), SAR440340 heavy chain
amino acid sequence.

According to certain embodiments, the IL-4R antagonist comprises an anti-IL-4R antibody or antigen-binding fragment thereof that can be used in the context of the methods featured in the invention are described elsewhere herein. For example, in one embodiment, the IL-4R antagonist is an antibody or antigen-binding fragment thereof that specifically binds to an IL-4R, and comprises the heavy chain and light chain (complementarity determining region) CDR sequences from the heavy chain variable region (HCVR) and light chain variable region (LCVR) of SEQ ID NOs: 27 and 28, respectively. In another embodiment, the IL-4R antagonist is an antibody or antigen-binding fragment thereof that specifically binds to an IL-4R, and comprises the heavy chain and light chain CDR sequences of SEQ ID NOs: 21, 22 and 23, and SEQ ID NOs: 24, 25 and 26, respectively. In another embodiment, the IL-4R antagonist is an antibody or antigen-binding fragment thereof that specifically binds to an IL-4R, and comprises an HCVR/LCVR pair of SEQ ID NOs: 27 and 28, respectively.

(SEQ ID NO: 21)
GFTFRDYA, dupilumab HCDR1 amino acid sequence.
(SEQ ID NO: 22)
ISGSGGNT, dupilumab HCDR2 amino acid sequence.
(SEQ ID NO: 23)
AKDRLSITIRPRYYGL, dupilumab HCDR3 amino acid
sequence.
(SEQ ID NO: 24)
QSLLYSIGYNY, dupilumab LCDR1 amino acid sequence.
(SEQ ID NO: 25)
LGS, dupilumab LCDR2 amino acid sequence.
(SEQ ID NO: 26)
MQALQTPYT, dupilumab LCDR3 amino acid sequence.
(SEQ ID NO: 27)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVS, dupilumab HCVR amino
acid sequence.
(SEQ ID NO: 28)
DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIK, dupilumab LCVR amino acid sequence.

The term “human IL-33” (hIL-33) refers to a human cytokine receptor that specifically binds to interleukin-33 (IL-33). The term “human IL-4R” (hIL-4R) refers to a human cytokine receptor that specifically binds to interleukin-4 (IL-4), such as IL-4Ra.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-33 antibody, the anti-IL-4R antibody, or an antigen-binding portion thereof may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include, but are not limited to: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies featured in the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The invention encompasses antibodies having one or more mutations in the hinge, C_{H}2, or C_{H}3 region, which may be desirable, for example, in production, to improve the yield of the desired antibody form.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-33 or IL-4R, as featured in the invention, includes antibodies that bind IL-33 or IL-4R, respectively, or portion thereof, with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-33 or human IL-4R may, however, have cross-reactivity to other antigens, such as IL-33 or IL-4R molecules from other (non-human) species.

The anti-IL-33 and anti-IL-4R antibodies useful for the methods may comprise one or more amino acid substitutions, insertions, and/or deletions (e.g. 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The invention includes methods involving the use of antibodies, and antigen-binding fragments thereof, that are derived from any of the amino acid sequences disclosed herein, wherein one or more amino acids (e.g. 1, 2, 3, 4, 5, 6, 7, 8, 9, or 10 amino acids) within one or more framework and/or one or more (e.g. 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the invention.

The invention also includes methods involving the use of anti-IL33 or anti-IL-4R antibodies comprising variants of any of the HCVR, LCVR, and/or CDR amino acid sequences disclosed herein having one or more conservative substitutions. For example, the invention includes the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

### Preparation of Human Antibodies

Methods for generating human antibodies in transgenic mice are known in the art. Any such known methods can be used to make human antibodies that specifically bind to human IL-33 or human IL-4R.

Using VELOCIMMUNE® technology (see, for example, U.S. Pat. No. 6,596,541, Regeneron Pharmaceuticals) or any other known method for generating monoclonal antibodies, high affinity chimeric antibodies to IL-33 or IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody featured in the invention, for example wild-type or modified IgG1 or IgG4. While the constant region selected may vary according to specific use, high affinity antigen-binding and target specificity characteristics reside in the variable region.

In general, the antibodies that can be used in the methods possess high affinities, as described above, when measured by binding to antigen either immobilized on solid phase or in solution phase. The mouse constant regions are replaced with desired human constant regions to generate the fully human antibodies featured in the invention. While the constant region selected may vary according to specific use, high affinity antigen-binding and target specificity characteristics reside in the variable region.

In one embodiment, human antibody or antigen-binding fragment thereof that specifically binds IL-33 that can be used in the context of the methods featured in the invention comprises the three heavy chain CDRs (HCDR1, HCDR2 and HCDR3) contained within a heavy chain variable region (HCVR) having an amino acid sequence of SEQ ID NO: 2. The antibody or antigen-binding fragment may comprise the three light chain CDRs (LCVR1, LCVR2, LCVR3) contained within a light chain variable region (LCVR) having an amino acid sequence of SEQ ID NO: 10. In another embodiment, human antibody or antigen-binding fragment thereof that specifically binds IL-4R that can be used in the context of the methods featured in the invention comprises the three heavy chain CDRs (HCDR1, HCDR2 and HCDR3) contained within a heavy chain variable region (HCVR) having an amino acid sequence of SEQ ID NO: 27. The antibody or antigen-binding fragment may comprise the three light chain CDRs (LCVR1, LCVR2, LCVR3) contained within a light chain variable region (LCVR) having an amino acid sequence of SEQ ID NO: 28.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 2 and 10.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 4/5/6/12/14/16.

In certain embodiments, the antibody or antigen-binding fragment thereof comprises HCVR/LCVR amino acid sequence pairs of SEQ ID NOs: 2 and 10.

In one embodiment, the antibody is SAR440340, which comprises the HCVR/LCVR amino acid sequence pairs of SEQ ID NOs: 2 and 10.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 27 and 28.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 21/22/23/24/25/26.

In certain embodiments, the antibody or antigen-binding fragment thereof comprises HCVR/LCVR amino acid sequence pairs of SEQ ID NOs: 27 and 28.

In one embodiment, the antibody is dupilumab, which comprises the HCVR/LCVR amino acid sequence pairs of SEQ ID NOs: 27 and 28.

### Pharmaceutical Compositions

The invention includes methods that comprise administering an IL-33 antagonist, or an IL-33 antagonist and an IL-4R antagonist, to a patient, wherein the IL-33 antagonist, or the IL-33 antagonist and the IL-4R antagonist, are contained within a pharmaceutical composition. The pharmaceutical compositions featured in the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J. Pharm. Sci. Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-33 antibodies or anti-IL-4R antibodies may be determined empirically. For example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions featured in the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition featured in the invention can be delivered subcutaneously or intravenously with a standard needle and syringe. In addition, with respect to subcutaneous delivery, a pen delivery device (e.g., an autoinjector pen) readily has applications in delivering a pharmaceutical composition featured in the invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition featured in the invention include, but are not limited to the SOLOSTAR™ pen (Sanofi-Aventis), the FLEXPEN™ (Novo Nordisk), and the KWIKPEN™ (Eli Lilly), the SURECLICK™Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions featured in the invention may be administered using, e.g., a microcatheter (e.g., an endoscope and microcatheter), an aerosolizer, a powder dispenser, a nebulizer or an inhaler. The methods include administration of an IL-33 antagonist or an IL-4R antagonist to a subject in need thereof, in an aerosolized formulation. For example, aerosolized antibodies to IL-33 or IL-4R may be administered to treat asthma in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the invention are disclosed, e.g., in US Patent Application Publication No. 2012/0097565.

### Dosage

The amount of IL-33 antagonist (e.g., an anti-IL-33 antibody or antigen-binding fragment thereof) or IL-4R antagonist (e.g., anti-IL-4R antibody or antigen-binding fragment thereof) administered to a subject according to the methods featured in the invention is, generally, a therapeutically effective amount. As used herein, the phrase “therapeutically effective amount” means an amount of IL-33 antagonist or IL-4R antagonist that results in one or more of: (a) a reduction in the incidence of asthma exacerbations; (b) an improvement in one or more asthma-associated parameters (as defined elsewhere herein); and/or (c) a detectable improvement in one or more symptoms or indicia of an upper airway inflammatory condition. A “therapeutically effective amount” also includes an amount of IL-33 antagonist or IL-4R antagonist that inhibits, prevents, lessens, or delays the progression of asthma in a subject.

In the case of an anti-IL-33 antibody or an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-33 antibody or anti-IL-4R antibody. In certain embodiments, 300 mg of an anti-IL-33 antibody is administered. In certain embodiments, 300 mg of an anti-IL-33 antibody and 300 mg of an anti-IL-4R antibody is administered.

The amount of IL-33 antagonist or IL-4R antagonist contained within the individual doses may be expressed in terms of milligrams of antibody per kilogram of patient body weight (i.e., mg/kg). For example, the IL-4R antagonist may be administered to a patient at a dose of about 0.0001 to about 10 mg/kg of patient body weight. For example, the IL-33 antagonist or the IL-4R antagonist can be administered at a dose of 1 mg/kg, 2 mg/kg, 3 mg/kg, or 4 mg/kg.

In some embodiments, the dose of IL-4R antagonist may vary according to eosinophil count. For example, the subject may have a blood eosinophil count (high blood eosinophils)≥300 cells/μL, or 300-499 cells/μL, or >500 cells/μL, (HEos); a blood eosinophil count of 200 to 299 cells/μL, (moderate blood eosinophils); or a blood eosinophil count<200 cells/μL, (low blood eosinophils).

In some embodiments, the dose of IL-4R antagonist may vary according to periostin levels. For example, the subject may have high periostin levels (e.g., ≥75.0 ng/mL or 74.4 ng/mL) or low periostin levels (e.g., <75.0 ng/mL or <74.4 ng/mL).

In certain embodiments, the methods comprise an initial dose of about 200 to about 600 mg of an IL-33 antagonist, e.g., about 300 mg of an IL-33 antagonist. In certain embodiments, the methods comprise an initial dose of about 200 to about 600 mg of an IL-4R antagonist, e.g., about 300 mg of an IL-4R antagonist.

In certain embodiments, the methods comprise one or more maintenance doses of about 200 to about 300 mg of the IL-33 antagonist. In certain embodiments, the methods comprise one or more maintenance doses of about 200 to about 300 mg of the IL-4R antagonist.

In certain embodiments, ICS and LABA are administered for the duration of administration of the IL-33 antagonist. In certain embodiments, ICS and LABA are administered for the duration of administration of the IL-4R antagonist.

In certain embodiments, the initial dose comprises 300 mg of an anti-IL-33 antibody or antigen-binding fragment thereof, and the one or more maintenance doses comprises 300 mg of the antibody or antigen-binding fragment thereof administered every other week.

In certain embodiments, the initial dose comprises 300 mg of an anti-IL-4R antibody or antigen-binding fragment thereof, and the one or more maintenance doses comprises 300 mg of the antibody or antigen-binding fragment thereof administered every other week.

In other embodiments, the initial dose comprises 300 mg of an anti-IL-33 antibody or antigen-binding fragment thereof, and the one or more maintenance doses comprises 300 mg of the antibody or antigen-binding fragment thereof administered every fourth week.

In other embodiments, the initial dose comprises 300 mg of an anti-IL-4R antibody or antigen-binding fragment thereof, and the one or more maintenance doses comprises 300 mg of the antibody or antigen-binding fragment thereof administered every fourth week.

In other embodiments, the initial dose comprises 300 mg of an anti-IL-33 antibody or antigen-binding fragment thereof, and the one or more maintenance doses comprises 300 mg of the antibody or antigen-binding fragment thereof administered once a week.

In other embodiments, the initial dose comprises 300 mg of an anti-IL-4R antibody or antigen-binding fragment thereof, and the one or more maintenance doses comprises 300 mg of the antibody or antigen-binding fragment thereof administered once a week.

In other embodiments, the initial dose comprises 300 mg of an anti-IL-33 antibody or antigen-binding fragment thereof, and the one or more maintenance doses comprises 300 mg of the antibody or antigen-binding fragment thereof administered every third week.

In other embodiments, the initial dose comprises 300 mg of an anti-IL-4R antibody or antigen-binding fragment thereof, and the one or more maintenance doses comprises 300 mg of the antibody or antigen-binding fragment thereof administered every third week.

In one embodiment, the subject is 6 to <18 years old and the IL-33 antibody or antigen-binding fragment thereof or the IL-4R antibody or antigen binding fragment thereof is administered at 2 mg/kg or 4 mg/kg.

In another embodiment, the subject is 12 to <18 years old and the IL-33 antibody or antigen-binding fragment thereof or the IL-4R antibody or antigen binding fragment thereof is administered at 2 mg/kg or 4 mg/kg.

In another embodiment, the subject is 6 to <12 years old and the IL-33 antibody or antigen-binding fragment thereof or the IL-4R antibody or antigen binding fragment thereof is administered at 2 mg/kg or 4 mg/kg.

In another embodiment, the subject is 2 to <6 years old and the IL-33 antibody or antigen-binding fragment thereof or the IL-4R antibody or antigen binding fragment thereof is administered at 2 mg/kg or 4 mg/kg.

In yet another embodiment, the subject is <2 years old and the IL-33 antibody or antigen-binding fragment thereof or the IL-4R antibody or antigen binding fragment thereof is administered at 2 mg/kg or 4 mg/kg.

### Combination Therapies

Certain embodiments of the methods featured in the invention comprise administering to the subject one or more additional therapeutic agents in combination with the IL-33 antagonist or one or more additional therapeutic agents in combination with the IL-33 antagonist and the IL-4R antagonist. As used herein, the expression “in combination with” means that the additional therapeutic agents are administered before, after, or concurrent with the pharmaceutical composition comprising the IL-4R antagonist or the IL-33 antagonist and the IL-4R antagonist. In some embodiments, the term “in combination with” includes sequential or concomitant administration of an IL-33 antagonist, or an IL-33 antagonist and an IL-4R antagonist, and an additional therapeutic agent. The invention includes methods to treat asthma or an associated condition or complication or to reduce at least one exacerbation, comprising administration of an IL-33 antagonist, or an IL-33 antagonist and an IL-4R antagonist, in combination with an additional therapeutic agent for additive or synergistic activity.

For example, when administered “before” the pharmaceutical composition comprising an IL-33 antagonist, or an IL-33 antagonist and an IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-33 antagonist, or the IL-33 antagonist and the IL-4R antagonist. When administered “after” the pharmaceutical composition comprising an IL-33 antagonist, or an IL-33 antagonist and an IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-33 antagonist, or the IL-33 antagonist and the IL-4R antagonist. Administration “concurrent” with the pharmaceutical composition comprising an IL-33 antagonist, or an IL-33 antagonist and an IL-4R antagonist, means that the additional therapeutic agent is administered to the subject in a separate dosage form within less than 5 minutes (before, after, or at the same time) of administration of the pharmaceutical composition comprising the IL-33 antagonist, or the IL-33 antagonist and the IL-4R antagonist, or administered to the subject as a single combined dosage formulation comprising both the additional therapeutic agent and the IL-33 antagonist, or the IL-33 antagonist and the IL-4R antagonist.

The additional therapeutic agent may be, e.g., another IL-33 antagonist, another IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), a TNF antagonist, an IL-8 antagonist, an IL-9 antagonist, an IL-17 antagonist, an IL-5 antagonist, an IgE antagonist, a CD48 antagonist, a leukotriene inhibitor, an anti-fungal agent, an NSAID, a long-acting beta2 agonist (e.g., salmeterol or formoterol), an inhaled corticosteroid (e.g., fluticasone or budesonide), a systemic corticosteroid (e.g., oral or intravenous), methylxanthine, nedocromil sodium, cromolyn sodium, or combinations thereof. For example, in certain embodiments, the pharmaceutical composition comprising an IL-4R antagonist, or an IL-33 antagonist and an IL-4R antagonist, is administered with a combination comprising a long-acting beta2 agonist and an inhaled corticosteroid (e.g., fluticasone+salmeterol [e.g., Advair® (GlaxoSmithKline)]; or budesonide+formoterol [e.g., SYMBICORT® (Astra Zeneca)]).

### Administration Regimens

According to certain embodiments, multiple doses of an IL-33 antagonist, or an IL-33 antagonist and an IL-4R antagonist, may be administered to a subject over a defined time course. Such methods comprise sequentially administering to a subject multiple doses of an IL-33 antagonist, or an IL-33 antagonist and an IL-4R antagonist. As used herein, “sequentially administering” means that each dose of an IL-33 antagonist, or an IL-33 antagonist and an IL-4R antagonist, is administered to the subject at a different point in time, e.g., on different days separated by a predetermined interval (e.g., hours, days, weeks, or months). Included are methods that comprise sequentially administering to the patient a single initial dose of an IL-33 antagonist, or an IL-33 antagonist and an IL-4R antagonist, followed by one or more secondary doses of the IL-33 antagonist, or the IL-33 antagonist and the IL-4R antagonist, and optionally followed by one or more tertiary doses of the IL-33 antagonist, or the IL-33 antagonist and the IL-4R antagonist.

The invention includes methods comprising administering to a subject a pharmaceutical composition comprising an IL-33 antagonist or an IL-33 antagonist and an IL-4R antagonist at a dosing frequency of about four times a week, twice a week, once a week (q1w), once every two weeks (bi-weekly or q2w), once every three weeks (tri-weekly or q3w), once every four weeks (monthly or q4w), once every five weeks (q5w), once every six weeks (q6w), once every eight weeks (q8w), once every twelve weeks (q12w), or less frequently so long as a therapeutic response is achieved. In certain embodiments involving the administration of a pharmaceutical composition comprising an anti-IL-33 antibody or an anti-IL-4R antibody, once a week dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every two weeks dosing (bi-weekly dosing) of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every three weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every four weeks dosing (monthly dosing) of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every five weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every six weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every eight weeks dosing of an amount of about 75 mg, 100 mg, 150 mg, 200 mg, or 300 mg, can be employed. In other embodiments involving the administration of a pharmaceutical composition comprising an anti-IL-33 antibody or an anti-IL-4R antibody, once every twelve weeks dosing of an amount of about 75 mg, 100 mg, 150 mg, 200 mg, or 300 mg, can be employed. In one embodiment, the route of administration is subcutaneous.

The term “week” or “weeks” refers to a period of (n×7 days)±2 days, e.g. (n×7 days)±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose”); the “secondary doses” are the doses that are administered after the initial dose; and the “tertiary doses” are the doses that are administered after the secondary doses. The initial, secondary, and tertiary doses may all contain the same amount of IL-33 antagonist or IL-4R antagonist, but generally may differ from one another in terms of frequency of administration. In certain embodiments, however, the amount of IL-33 antagonist or IL-4R antagonist contained in the initial, secondary and/or tertiary doses varies from one another (e.g., adjusted up or down as appropriate) during the course of treatment. In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “maintenance doses”). In one embodiment, the maintenance dose may be lower than the loading or initial dose. For example, one or more loading doses of 600 mg of IL-4R antagonist may be administered followed by maintenance doses of about 75 mg to about 300 mg.

In certain embodiments, the initial dose is about 400 to about 600 mg of the IL-33 antagonist or the IL-4R antagonist. In one embodiment, the initial dose is 400 mg of the IL-33 antagonist or the IL-4R antagonist. In another embodiment, the initial dose is 600 mg of the IL-33 antagonist or the IL-4R antagonist.

In certain embodiments, the maintenance dose is about 200 to about 300 mg of the IL-33 antagonist or the IL-4R antagonist. In one embodiment, the maintenance dose is 200 mg of the IL-33 antagonist or the IL-4R antagonist. In another embodiment, the maintenance dose is 300 mg of the IL-33 antagonist or the IL-4R antagonist.

In certain embodiments, the loading dose is two times the maintenance dose. In certain embodiments, the initial dose is the same amount as the maintenance dose.

In some embodiments, the initial dose comprises 300 mg of the antibody or antigen-binding fragment thereof, and the one or more maintenance doses comprises 300 mg of the antibody or antigen-binding fragment thereof administered every other week.

In some embodiments, a subject has moderate-to-severe asthma, and the initial dose comprises 300 mg of the antibody or antigen-binding fragment thereof, and the one or more maintenance doses comprises 300 mg of the antibody or antigen-binding fragment thereof administered every other week.

In some embodiments, the initial dose comprises 300 mg of the antibody or antigen-binding fragment thereof, and the one or more maintenance doses comprises 300 mg of the antibody or antigen-binding fragment thereof administered every fourth week.

In some embodiments, a subject has moderate-to-severe asthma, and the initial dose comprises 300 mg of the antibody or antigen-binding fragment thereof, and the one or more maintenance doses comprises 300 mg of the antibody or antigen-binding fragment thereof administered every fourth week.

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-33 antagonist or IL-4R antagonist that is administered to a patient prior to the administration of the very next dose in the sequence with no intervening doses.

The methods may include administering to a patient any number of secondary and/or tertiary doses of an IL-33 antagonist or an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The invention includes methods comprising sequential administration of an IL-33 antagonist or an IL-33 antagonist and an IL-4R antagonist and an additional therapeutic agent, to a patient to treat asthma or an associated condition. In some embodiments, the methods comprise administering one or more doses of an IL-33 antagonist or one or more doses of both an IL-33 antagonist and an IL-4R antagonist followed by one or more doses (e.g., 2, 3, 4, 5, 6, 7, 8, or more) of an additional therapeutic agent. For example, one or more doses of about 75 mg to about 300 mg of an IL-33 antagonist or one or more doses of both an IL-33 antagonist and an IL-4R antagonist may be administered after which one or more doses (e.g., 2, 3, 4, 5, 6, 7, 8, or more) of an additional therapeutic agent (e.g., an inhaled corticosteroid or a beta2-agonist or any other therapeutic agent, as described elsewhere herein) may be administered to treat, alleviate, reduce or ameliorate one or more symptoms of asthma. In some embodiments, an IL-33 antagonist or an IL-33 antagonist and an IL-4R antagonist are administered at one or more doses (e.g., 2, 3, 4, 5, 6, 7, 8, or more) resulting in an improvement in one or more asthma-associated parameters followed by the administration of a second therapeutic agent to prevent recurrence of at least one symptom of asthma. Alternative embodiments pertain to concomitant administration of an IL-33 antagonist or both an IL-33 antagonist and an IL-4R antagonist, and an additional therapeutic agent. For example, one or more doses (e.g., 2, 3, 4, 5, 6, 7, 8, or more) of an IL-33 antagonist or both an IL-33 antagonist and an IL-4R antagonist are administered and an additional therapeutic agent is administered at a separate dosage at a similar or different frequency relative to an IL-33 antagonist or both an IL-33 antagonist and an IL-4R antagonist. In some embodiments, the additional therapeutic agent is administered before, after or concurrently with the IL-33 antagonist, or the IL-33 antagonist and the IL-4R antagonist.

In certain embodiments, an IL-33 antagonist, or both an IL-33 antagonist and an IL-4R antagonist, are administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, an IL-33 antagonist, or both an IL-33 antagonist and an IL-4R antagonist, are administered every four weeks for 12 weeks, 16 weeks, 20 weeks, 24 weeks, 28 weeks, 32 weeks, 36 weeks, 40 weeks, 44 weeks, 48 weeks or more. In specific embodiments, an IL-33 antagonist, or both an IL-33 antagonist and an IL-4R antagonist, are administered for at least 24 weeks.

The invention includes methods for treating a subject having moderate-to-severe asthma comprising administering to the subject a loading dose of an antibody or an antigen-binding fragment thereof that specifically binds to IL-4R, or both an antibody or an antigen-binding fragment thereof that specifically binds to IL-33 and an antibody or an antigen-binding fragment thereof that specifically binds to IL-4R. In certain embodiments, the methods comprise administering to the subject a plurality of maintenance doses of the antibody(ies) or the antigen-binding fragment(s) thereof, wherein the plurality of maintenance doses are administered during a treatment phase. The treatment phase comprises an induction phase, an OCS reduction phase, and an OCS maintenance phase.

In certain exemplary embodiments, the induction phase comprises a period during which subjects continuously receive their OCS dose(s). In certain exemplary embodiments, the reduction phase comprises a period during which subjects receive a lower OCS dose relative to the dose received during the induction phase. In certain exemplary embodiments, the maintenance phase comprises a period during which a subject receives a certain stable amount or dose(s) of OCS. Alternatively, the maintenance phase comprises a period in which OCS therapy/administration is reduced or eliminated. In certain embodiments, OCS use by the patient is completely eliminated and the patient is steroid free within less than 1 year of treatment with the IL4R antibody or fragment thereof (e.g., within 1 year, 6 months, 3 months or 1 month of initial treatment).

In another aspect, a method for treating a subject having moderate-to-severe asthma comprises administering to the subject an initial dose of about 300 mg of an antibody or an antigen-binding fragment thereof that specifically binds to interleukin-4 receptor (IL-33), and administering to the subject a plurality of maintenance doses of the antibody or the antigen-binding fragment thereof. Each maintenance dose is about 300 mg of the antibody or antigen-binding fragment thereof, wherein the plurality of maintenance doses are administered during a treatment phase comprising an induction phase, an oral corticosteroid (OCS) reduction phase, and a maintenance phase, and wherein the antibody or antigen-binding fragment thereof comprises heavy and light chain CDR sequences comprise SEQ ID NOs: 4, 5, 6, 12, 14 and 16.

### Treatment Populations

The methods featured in the invention include administering to a subject in need thereof a therapeutic composition comprising an IL-4R antagonist or both an IL-33 antagonist and an IL-4R antagonist. The expression “a subject in need thereof” means a human or non-human animal that exhibits one or more symptoms or indicia of asthma (e.g., moderate-to-severe asthma), or who has been diagnosed with asthma. For example, “a subject in need thereof” may include, e.g., subjects who, prior to treatment, exhibit (or have exhibited) one or more asthma-associated parameter, such as, e.g., impaired FEV₁ (e.g., less than 2.0 L), impaired FEF25-75%; impaired AM PEF (e.g., less than 400 L/min), impaired PM PEF (e.g., less than 400 L/min), an ACQS score of at least 2.5, at least 1 nighttime awakenings per night, and/or a SNOT-22 score of at least 20. In various embodiments, the methods may be used to treat mild, moderate-to-severe, and severe asthma in patients in need thereof.

In a related embodiment, a “subject in need thereof” may be a subject who, prior to receiving an IL-4R antagonist or both an IL-33 antagonist and an IL-4R antagonist, has been prescribed or is currently taking a combination of ICS/LABA. Examples of ICS include mometasone furoate, budesonide, and fluticasone propionate. Examples of LABA include formoterol and salmeterol. Examples of ICS/LABA therapies include fluticasone/salmeterol combination therapy and budesonide/formoterol combination therapy. For example, the invention includes methods that comprise administering an IL-4R antagonist or both an IL-33 antagonist and an IL-4R antagonist to a patient who has been taking a regular course of ICS/LABA for two or more weeks immediately preceding the administration of the IL-4R antagonist or both the IL-33 antagonist and the IL-4R antagonist (such prior treatments are referred to herein as “background treatments”). The invention includes therapeutic methods in which background treatments are continued in combination with administration of the IL-4R antagonist or both the IL-33 antagonist and the IL-4R antagonist. In yet other embodiments, the amount of the ICS component, the LABA component, or both, is gradually decreased prior to or after the start of IL-4R antagonist or both IL-33 antagonist and IL-4R antagonist administration. In some embodiments, the invention includes methods to treat patients with persistent asthma for at least ≥12 months. In one embodiment, a patient with moderate-to-severe persistent asthma may be resistant to treatment by a therapeutic agent, such as a corticosteroid, and may be administered an IL-4R antagonist or both an IL-33 antagonist and an IL-4R antagonist according to the present methods.

In some embodiments, a “subject in need thereof” may be a subject with elevated levels of an asthma-associated biomarker. Examples of asthma-associated biomarkers include, but are not limited to, IgE, thymus and activation regulated chemokine (TARC), eotaxin-3, CEA, YKL-40, and periostin. In some embodiments, a “subject in need thereof” may be a subject with blood eosinophils≥300 cells/μL, 150-299 cells/μL, or <150 cells/μL. In one embodiment, a “subject in need thereof” may be a subject with elevated level of bronchial or airway inflammation as measured by the fraction of exhaled nitric oxide (FeNO).

In some embodiments, a “subject in need thereof” is selected from the group consisting of: a subject age 18 years old or older, a subject 12 years or older, a subject age 12 to 17 years old (12 to <18 years old), a subject age 6 to 11 years old (6 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months.

In some embodiments, a “subject in need thereof” is a subject who is a current smoker. In some embodiments, the subject is a current smoker who smokes, e.g., cigarettes, cigars, pipes, water pipes, and/or vaporizers (i.e., “vapes”). In some embodiments, the subject is a current smoker who has a smoking history of smoking greater than or equal to 10 packs of cigarettes per year. In some embodiments, the subject is a current smoker and has a smoking history of smoking fewer than 10 packs of cigarettes per year. In some embodiments, the subject is a current smoker and has a smoking history of smoking more than 1, 5, 10, 15, 20, 25, 30, 35, 40, 45, 50 or more packs of cigarettes per year. In some embodiments, the subject is a current smoker who has a smoking history of smoking for 6 months, 1 year, 2 years, 3 years, 5 years, 10 years or longer.

In some embodiments, a “subject in need thereof” is a subject who is a former smoker. In some embodiments, the subject is a former smoker who has a history of smoking cigarettes, cigars, pipes, water pipes and/or vapes. In some embodiments, the subject is a former smoker who has a smoking history of smoking greater than or equal to 10 packs of cigarettes per year. In some embodiments, the subject is a former smoker who has a smoking history of smoking fewer than 10 packs per year. In some embodiments, the subject is a former smoker who has a smoking history of smoking more than 1, 5, 10, 15, 20, 25, 30, 35, 40, 45, 50 or more packs of cigarettes per year. In some embodiments, the subject is a former smoker who has a smoking history of smoking for 6 months, 1 year, 2 years, 3 years, 5 years, 10 years or longer. In some embodiments, the subject is a former smoker who has ceased smoking for at least 1, 2, 3, 4, 5, 6, 7, 8, 9, 10, 11, or 12 months. In some embodiments, the subject is a former smoker who has ceased smoking for at least 6 months. In some embodiments, the subject is a former smoker that intends to quit permanently.

In some embodiments, a “subject in need thereof” is a subject who is a non-smoker. In some embodiments, a subject is a non-smoker that does not have a history of smoking cigarettes, cigars, pipes, water pipes and/or vapes. In some embodiments, a subject is a non-smoker that does not have a history of smoking tobacco.

A normal IgE level in healthy subjects is less than about 100 kU/L (e.g., as measured using the IMMUNOCAP® assay [Phadia, Inc. Portage, Mich.]). Thus, the invention includes methods comprising selecting a subject who exhibits an elevated serum IgE level, which is a serum IgE level greater than about 100 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L, and administering to the subject a pharmaceutical composition comprising a therapeutically effective amount of an IL-33 antagonist, or an IL-33 antagonist and an IL-4R antagonist.

TARC levels in healthy subjects are in the range of 106 ng/L to 431 ng/L, with a mean of about 239 ng/L. (An exemplary assay system for measuring TARC level is the TARC quantitative ELISA kit offered as Cat. No. DDN00 by R&D Systems, Minneapolis, Minn.) Thus, the invention involves methods comprising selecting a subject who exhibits an elevated TARC level, which is a serum TARC level greater than about 431 ng/L, greater than about 500 ng/L, greater than about 1000 ng/L, greater than about 1500 ng/L, greater than about 2000 ng/L, greater than about 2500 ng/L, greater than about 3000 ng/L, greater than about 3500 ng/L, greater than about 4000 ng/L, greater than about 4500 ng/L, or greater than about 5000 ng/L, and administering to the subject a pharmaceutical composition comprising a therapeutically effective amount of an IL-33 antagonist, or an IL-33 antagonist and an IL-4R antagonist.

Eotaxin-3 belongs to a group of chemokines released by airway epithelial cells, which is up-regulated by the Th2 cytokines IL-4 and IL-13 (Lilly et al 1999, J. Allergy Clin. Immunol. 104: 786-790). The invention includes methods comprising administering an IL-4R antagonist to treat patients with elevated levels of eotaxin-3, such as more than about 100 pg/ml, more than about 150 pg/ml, more than about 200 pg/ml, more than about 300 pg/ml, or more than about 350 pg/ml. Serum eotaxin-3 levels may be measured, for example, by ELISA.

Fractional exhaled NO (FeNO) is a biomarker of bronchial or airway inflammation. FeNO is produced by airway epithelial cells in response to inflammatory cytokines including IL-4 and IL-13 (Alwing et al 1993, Eur. Respir. J. 6: 1368-1370). FeNO levels in healthy adults range from 2 to 30 parts per billion (ppb). An exemplary assay for measuring FeNO is by using a NIOX instrument by Aerocrine AB, Solna, Sweden. The assessment may be conducted prior to spirometry and following a fast of at least an hour. Included here are methods comprising administering an IL-33 antagonist, or an IL-33 antagonist and an IL-4R antagonist, to patients with elevated levels of exhaled NO (FeNO), such as more than about 30 ppb, more than about 31 ppb, more than about 32 ppb, more than about 33 ppb, more than about 34 ppb, or more than about 35 ppb.

Carcinoembryogenic antigen (CEA) (also known as CEA cell adhesion molecule 5 [CEACAMS]) is a tumor marker that is found correlated to non-neoplastic diseases of the lung (Marechal et al 1988, Anticancer Res. 8: 677-680). CEA levels in serum may be measured by ELISA. The invention includes methods comprising administering an IL-33 antagonist, or an IL-33 antagonist and an IL-4R antagonist, to patients with elevated levels of CEA, such as more than about 1.0 ng/ml, more than about 1.5 ng/ml, more than about 2.0 ng/ml, more than about 2.5 ng/ml, more than about 3.0 ng/ml, more than about 4.0 ng/ml, or more than about 5.0 ng/ml.

YKL-40 (named for its N-terminal amino acids tyrosine (Y), lysine (K) and leucine (L) and its molecular mass of 40 kD) is a chitinase-like protein found to be up regulated and correlated to asthma exacerbation, IgE, and eosinophils (Tang et al 2010 Eur. Respir. J. 35: 757-760). Serum YKL-40 levels are measured by, for example, ELISA. The invention includes methods comprising administering an IL-33 antagonist, or an IL-33 antagonist and an IL-4R antagonist, to patients with elevated levels of YKL-40, such as more than about 40 ng/ml, more than about 50 ng/ml, more than about 100 ng/ml, more than about 150 ng/ml, more than about 200 ng/ml, or more than about 250 ng/ml.

Periostin is a secreted matricellular protein associated with fibrosis, and its expression is upregulated by recombinant IL-4 and IL-13 in cultured bronchial epithelial cells and bronchial fibroblasts (Jia et al. (2012) J. Allergy Clin. Immuno1.130:647). In human asthmatic patients periostin expression levels correlate with reticular basement membrane thickness, an indicator of subepithelial fibrosis. Id. Included here are methods comprising administering an IL-33 antagonist, or an IL-33 antagonist and an IL-4R antagonist, to patients with elevated levels of periostin (e.g., 74.4 ng/mL).

Induced sputum eosinophils and neutrophils are well-established direct markers of airway inflammation (Djukanovic et al 2002, Eur. Respire. J. 37: 1S-2S). Sputum is induced with inhalation of hypertonic saline solution and processed for cell counts according to methods known in the art, for example, the guidelines of European Respiratory Society.

In some embodiments, the subjects are stratified into the following groups: a blood eosinophil count (high blood eosinophils)≥300 cells/μL (HEos) or 300-499 cells/μL or ≥500 cells/μL, a blood eosinophil count of 200 to 299 cells/μL (moderate blood eosinophils), or a blood eosinophil count<200 cells/μL (low blood eosinophils), and are administered an IL-33 antagonist, or an IL-33 antagonist and an IL-4R antagonist, at a dose or dosing regimen based upon the eosinophil level.

In some embodiments, the subjects are stratified into the following groups: a blood eosinophil count of ≥300 cells/μL, of 300-499 cells/μL, or of ≥500 cells/μL (high blood eosinophils); a blood eosinophil count of ≥150 cells/μL (moderate blood eosinophils); or a blood eosinophil count of <150 cells/μL (low blood eosinophils), and are administered an IL-33 antagonist, or an IL-33 antagonist and an IL-4R antagonist, at a dose or dosing regimen based upon the eosinophil level.

In some embodiments, a subject has “eosinophilic phenotype” asthma defined by a blood eosinophil count of ≥150 cells/μL, a blood eosinophil count of ≥300 cells/μL, a blood eosinophil count of 300-499 cells/μL, or a blood eosinophil count of ≥500 cells/μL, and are administered an IL-33 antagonist, or an IL-33 antagonist and an IL-4R antagonist.

In some embodiments, a subject has “periostin phenotype” asthma defined by a high blood periostin level as defined herein, and are administered an IL-33 antagonist, or an IL-33 antagonist and an IL-4R antagonist.

### Methods for Assessing Pharmacodynamic Asthma-Associated Parameters

The invention also includes methods for assessing one or more pharmacodynamic asthma-associated parameters a subject in need thereof, caused by administration of a pharmaceutical composition comprising an IL-33 antagonist, or an IL-33 antagonist and an IL-4R antagonist. A reduction in the incidence of an asthma exacerbation (as described above) or an improvement in one or more asthma-associated parameters (as described above) may correlate with an improvement in one or more pharmacodynamic asthma-associated parameters; however, such a correlation is not necessarily observed in all cases.

Examples of “pharmacodynamic asthma-associated parameters” include, for example, the following: (a) biomarker expression levels; (b) serum protein and RNA analysis; (c) induced sputum eosinophils and neutrophil levels; (d) exhaled nitric oxide (FeNO); and (e) blood eosinophil count. An “improvement in a pharmacodynamic asthma-associated parameter” means, for example, a decrease from baseline of one or more biomarkers, such as periostin, TARC, eotaxin-3 or IgE, a decrease in sputum eosinophils or neutrophils, FeNO, periostin or blood eosinophil count. As used herein, the term “baseline,” with regard to a pharmacodynamic asthma-associated parameter, means the numerical value of the pharmacodynamic asthma-associated parameter for a patient prior to or at the time of administration of a pharmaceutical composition described herein.

To assess a pharmacodynamic asthma-associated parameter, the parameter is quantified at baseline and at a time point after administration of the pharmaceutical composition. For example, a pharmacodynamic asthma-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic asthma-associated parameter (e.g., an increase or decrease, as the case may be, depending on the specific parameter being measured).

In certain embodiments, administration of an IL-33 antagonist, or an IL-33 antagonist and an IL-4R antagonist, to a patient causes a change, such as a decrease or increase, in expression of a particular biomarker. Asthma-associated biomarkers include, but are not limited to, the following: (a) total IgE; (b) thymus and activation-regulated chemokine (TARC); (c) YKL-40; (d) carcinoembryonic antigen in serum; (e) eotaxin-3 in plasma; and (f) periostin in serum. For example, administration of an IL-33 antagonist, or an IL-33 antagonist and an IL-4R antagonist, to an asthma patient can cause one or more of a decrease in TARC or eotaxin-3 levels, or a decrease in total serum IgE levels. The decrease can be detected at week 1, week 2, week 3, week 4, week 5, or longer following administration of the IL-33 antagonist, or the IL-33 antagonist and the IL-4R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-33 antagonist, or an IL-33 antagonist and an IL-4R antagonist, IL-4/IL-13 signaling, asthma, atopy or eosinophilic diseases (e.g., by measuring soluble IL-4Rα, IL-4, IL-13, periostin and the like). In some embodiments, RNA samples are used to determine RNA levels (non-genetic analysis), e.g., RNA levels of biomarkers, and in other embodiments, RNA samples are used for transcriptome sequencing (e.g., genetic analysis).

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-33 antagonist used in the following Examples is the human anti-IL-33 antibody named SAR440340. The exemplary IL-4R antagonist used in the following Examples is the human anti-IL-4R antibody named dupilumab.

### Example 1. A Randomized, Double-Blind, Placebo-Controlled, Multiple Ascending Dose Study of the Safety, Tolerability, Pharmacokinetics and Pharmacodynamic Effects of Subcutaneously Administered SAR440340 in Adult Patients with Moderate Asthma

The primary objective of the study was to evaluate the safety and tolerability of multiple ascending SC doses of SAR440340 administered to moderate asthmatics.

The secondary objectives of the study were: to characterize the pharmacokinetics following multiple SC administrations of SAR440340 to moderate asthmatics; to assess the immunogenicity of SAR440340 after multiple SC doses in moderate asthmatics; to assess the in-clinic airway response (forced expiratory volume at 1 second [FEV1]) of multiple SC doses of SAR440340 in moderate asthmatics; and to assess changes in biomarkers (fractional expired nitric oxide [FeNO] in exhaled breath and calcitonin, a putative marker of interleukin-33 (IL-33) activity in circulation) following multiple SC doses of SAR440340 administered to moderate asthmatics.

The exploratory objectives of the study were: to assess the effect of SAR440340 on potential circulating PD markers of IL-33 pathway activation including but not limited to circulating concentrations of soluble IL-33 receptor (sST2); to assess the effect of SAR440340 on measures of daily FEV1 as measured by an ambulatory at-home spirometry monitoring device; to assess the effect of SAR440340 on measures of asthma symptom scores as measured by paper asthma control questionnaire (ACQ-6); and to assess total circulating IL-33 pre- and post-treatment with SAR440340.

### Trial Design

This was a first-in-patient study of SAR440340 intended to elucidate the safety and pharmacokinetic/pharmacodynamic (PK/PD) profile of repeated subcutaneous (SC) dosing of this monoclonal antibody (mAb) in asthmatic patients.

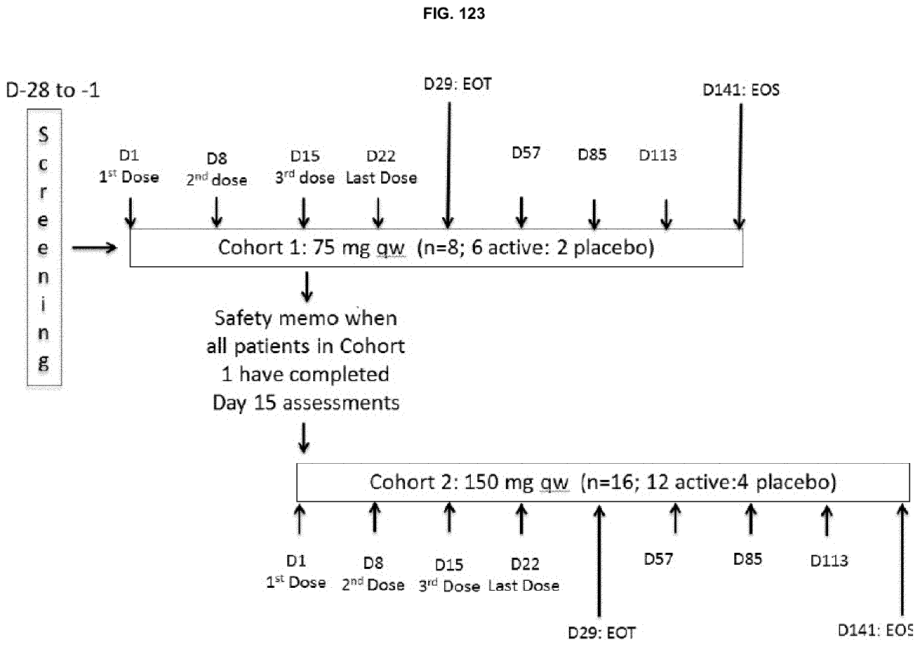

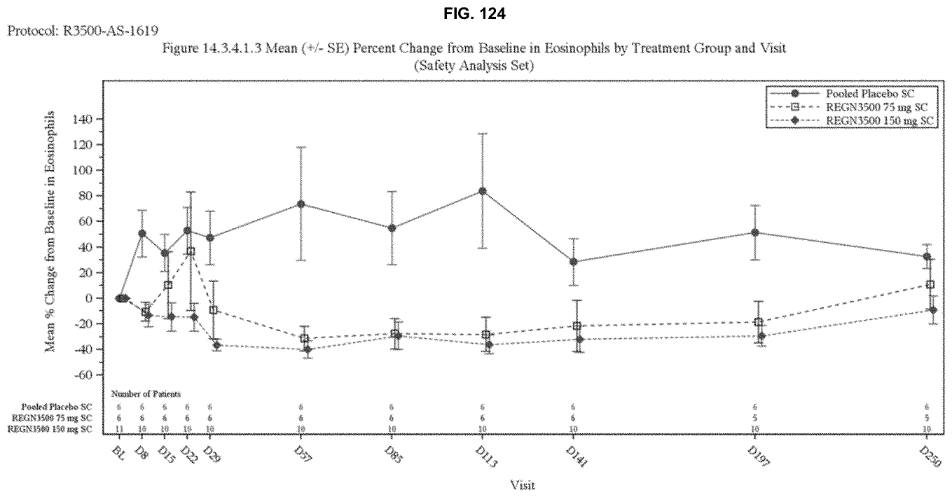

Twenty-three patients enrolled in the study, and were randomized to receive either SAR440340 or placebo in one of the following 2 sequential ascending SC dose cohorts: cohort 1 of SAR440340 75 mg SC QW (6 patients) or placebo SC QW (2 patients), and cohort 2 of SAR440340 150 mg SC QW (11 patients) or placebo SC QW (4 patients) (FIG. 123).

For each cohort, the study consisted of a screening period (day −28 to day −1, with an in-clinic visit to be performed between day −28 and day −14), a baseline visit (day 1), a treatment period (day 8 to day 22), and a follow-up period (day 29 to day 250), with an end of study visit at day 250. The total planned duration of a patient's participation in the study is approximately 40 weeks (including the screening period of up to 4 weeks).

### Summary of Results

A total of 23 moderate asthmatics were enrolled: 6 on PBO, 6 on SAR440340 at 75 mg SC QW×4 W, 11 on 150 mg SC QW×4W. SAR440340 was well-tolerated.

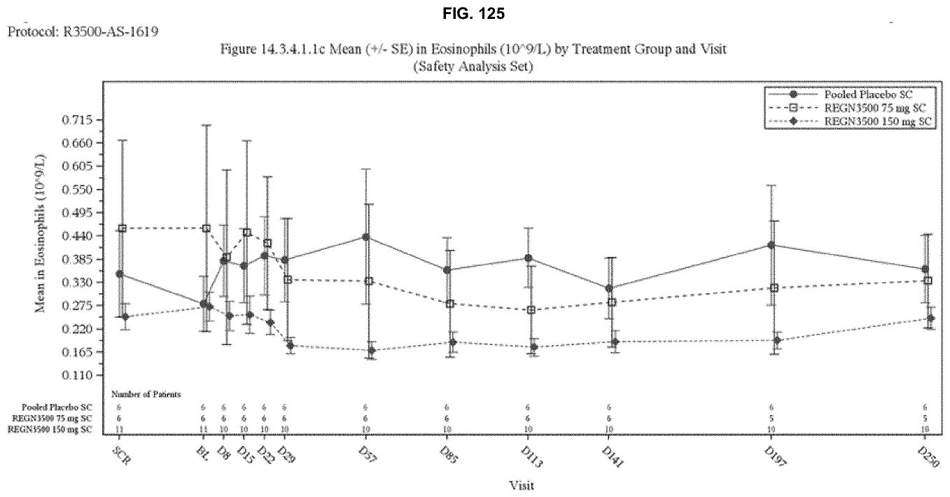

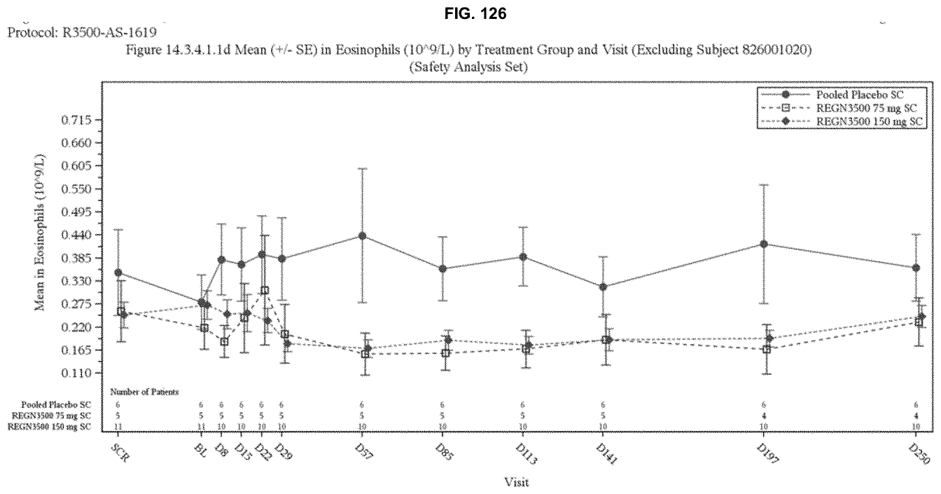

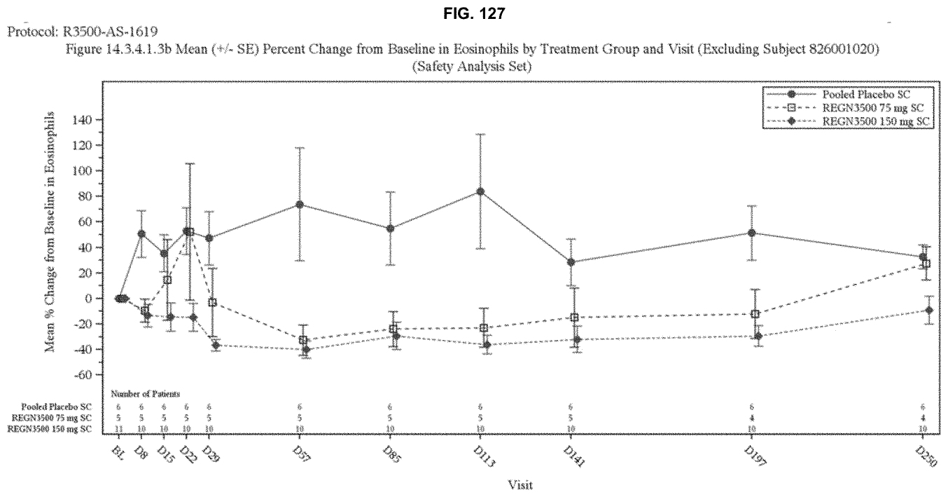

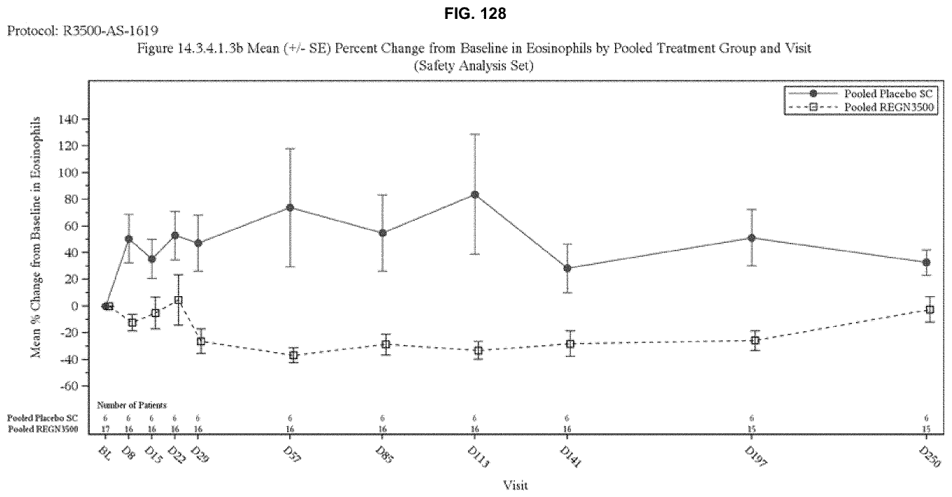

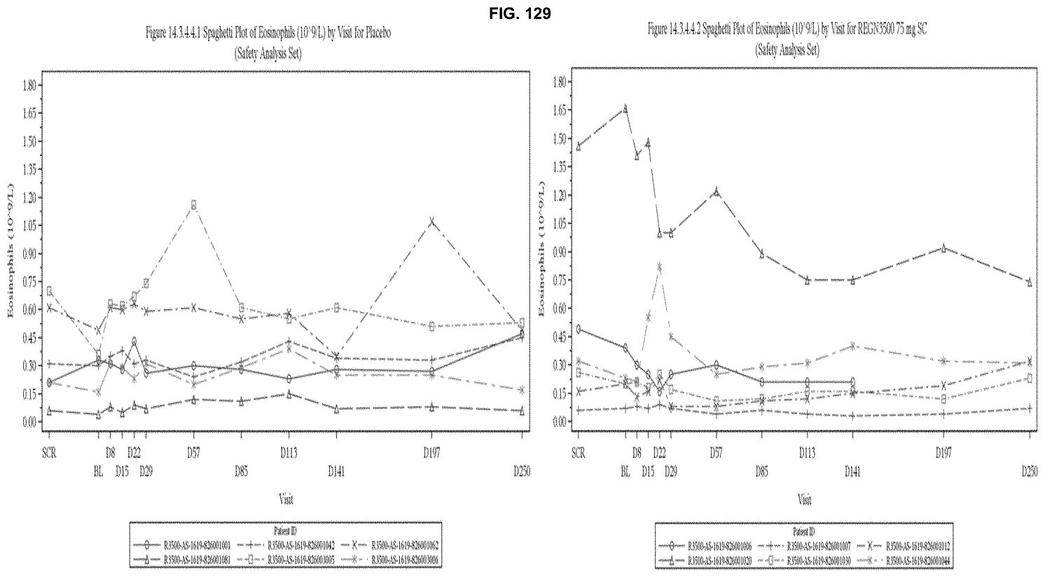

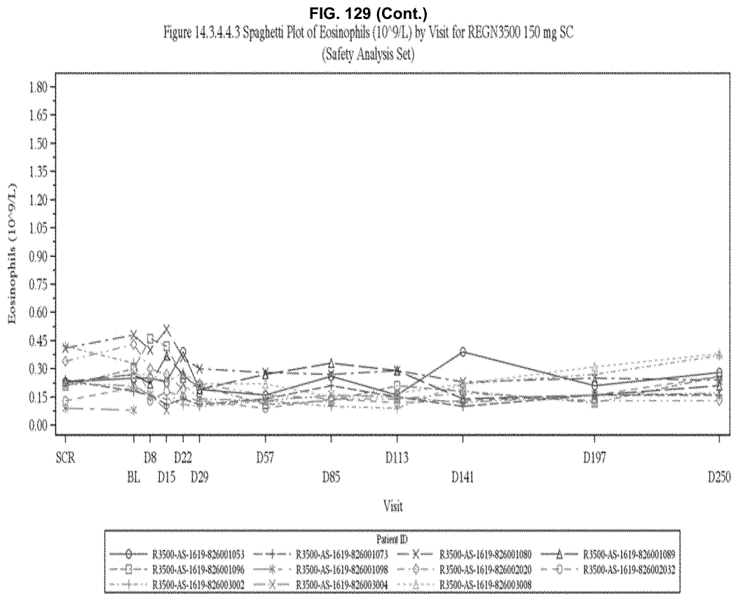

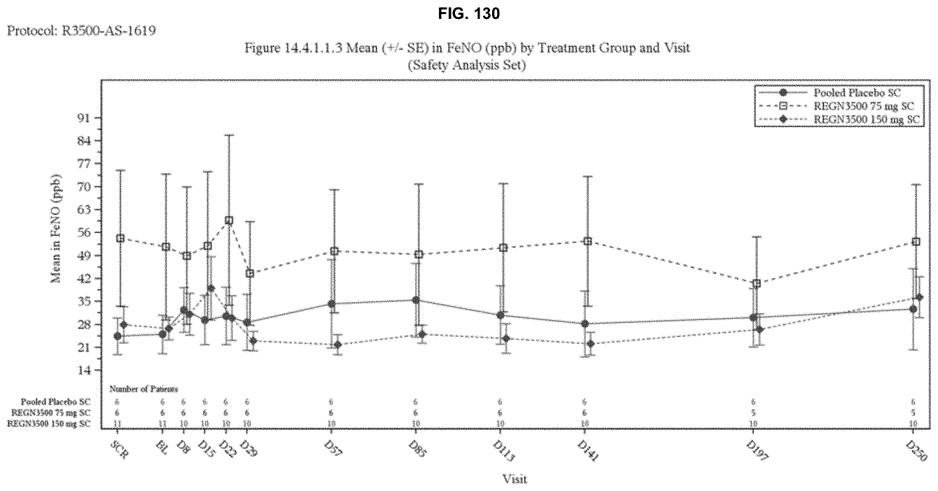

Blood EOS decreased with treatment of SAR440340 (FIG. 124-FIG. 129). Active treatment with SAR440340 (75 mg and 150 mg) resulted in an approximately 35% decrease from baseline observed at day 29 and sustained until day 197. Data are consistent with preclinical data demonstrating that treatment with SAR440340 lowers IL-5 levels.

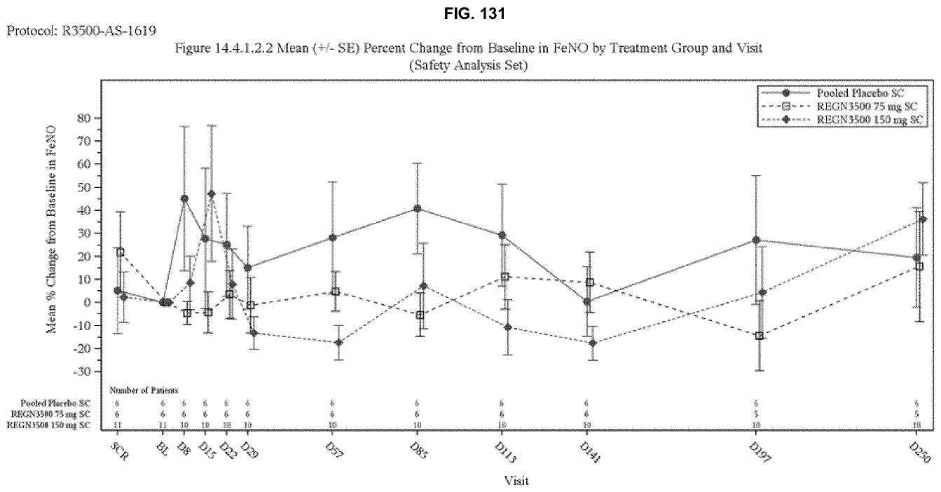

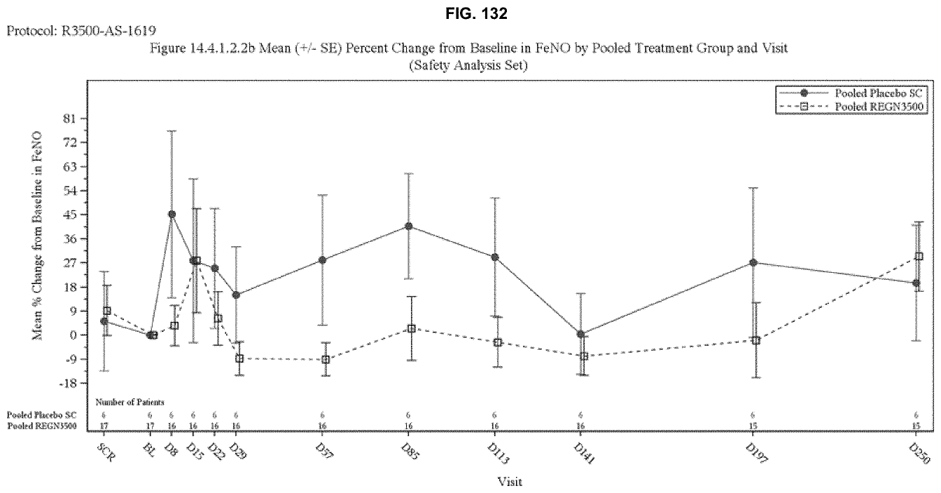

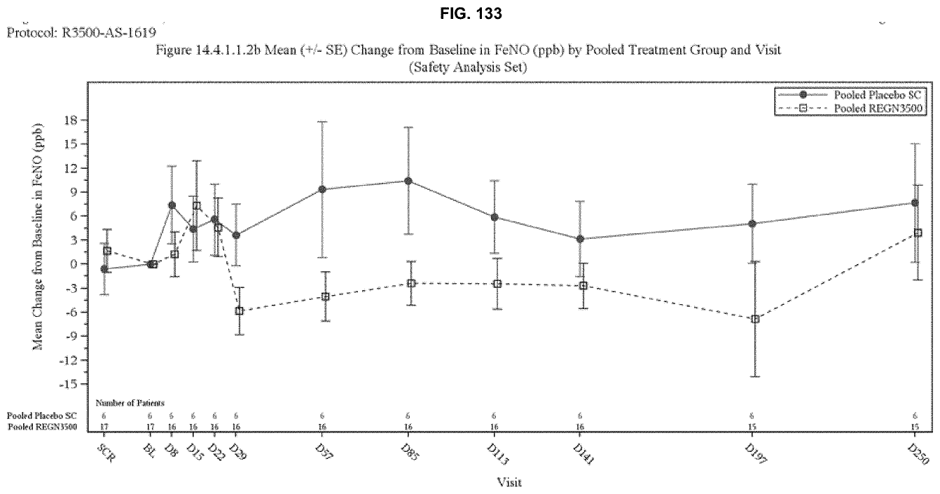

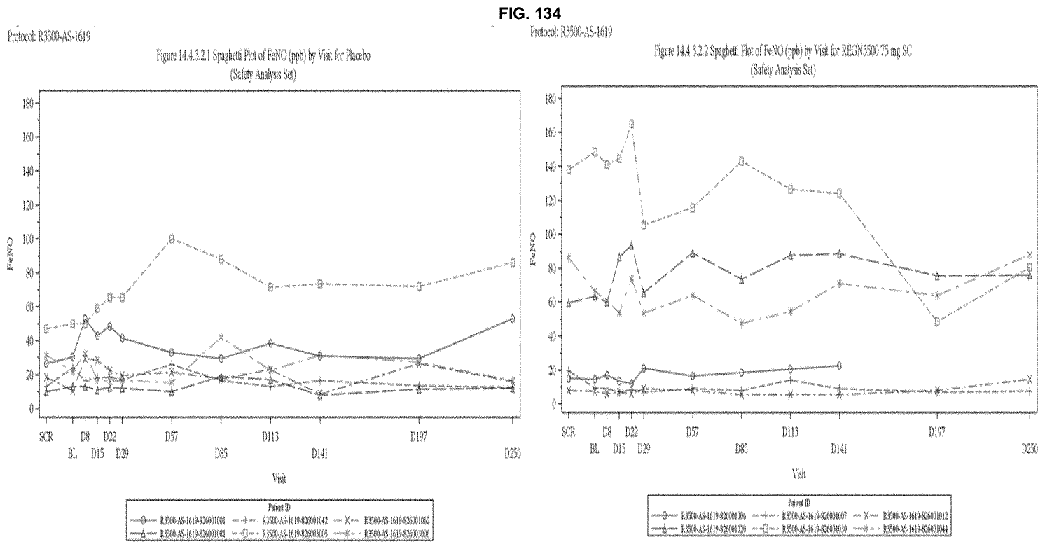

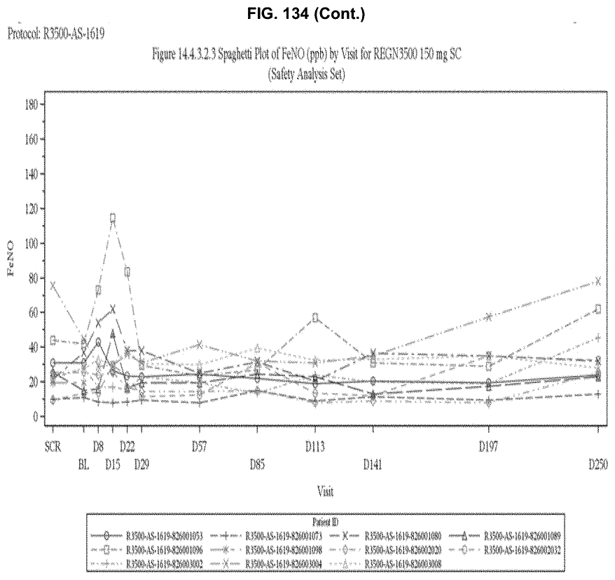

The biomarker FeNO demonstrated high variability in a small number of patients, but there was a potential modest effect by active treatment (FIG. 130-FIG. 134).

SAR440340 PK was similar in asthmatics as compared with healthy volunteers. It exhibited linear clearance kinetics with dose proportional AUC, t_{1/2}=30 days. No evident dose response was observed from 75 mg to 150 mg SC dose on Total IL-33 concentrations over time post-dose.

### Treatment Emergent Adverse Events

There were no deaths. There was no treatment discontinuation due to adverse events (AEs). There were no serious treatment-emergent adverse effects (TEAEs). There were no severe TEAEs.

The frequency of the number of patients with at least one TEAE was similar between SAR440340 and placebo.

The most frequent TEAEs were headache (1 [16.7%] in placebo and 4 [23.5%] in SAR440340), upper respiratory tract infection (1 [16.7%] in placebo and 2 [11.8%] in SAR440340), gastroenteritis (2 [33.3%] in placebo and 1 [5.9%] in SAR440340) and nasopharyngitis (2 [33.3%] in placebo and 1 [5.9%] in SAR440340).

The frequency of the number of patients with at least one drug-related TEAE was similar between SAR440340 and placebo. The only drug-related TEAE that was reported in the study was headache (1 [16.7%] in placebo and 1 [5.9%] in SAR440340).

### Laboratory Parameters, Vital Signs and ECGs

The treatment-emergent potential clinical significant values (PCSVs) for labs, vital signs and ECGs were similar between placebo and SAR440340-treated groups except that there was a higher percentage of patients with at least one treatment-emergent PCSV hematology in the placebo group (83.3%) than in the SAR440340 group (29.4%).

### Example 2. A Randomized, Double-Blind, Placebo-Controlled, Parallel-Group, 12-Week Proof-of-Concept (PoC) Study to Assess the Efficacy, Safety, and Tolerability of SAR440340, and the Co-Administration of SAR440340 and Dupilumab in Patients with Moderate-to-Severe Asthma Who are not Well Controlled on Inhaled Corticosteroid (ICS) Plus Long-Acting β2 Adrenergic Agonist (LABA) Therapy (NCT03387852)

### Primary Objective:

To evaluate the effects of SAR440340 with or without dupilumab, compared to placebo, on reducing the incidence of “loss of asthma control” (LOAC) events.

The protocol defined criteria for LOAC included the occurrence of at least one of the following: 1) A 30% or greater reduction from baseline in morning PEF on 2 consecutive days; ≥6 additional reliever puffs of salbutamol/albuterol or levosalbutamol/levalbuterol in a 24 hour period (compared to baseline) on 2 consecutive days; Increase in ICS≥4 times the last prescribed ICS dose (or ≥50% of the prescribed ICS dose at V2 if background therapy withdrawal completed); Requiring use of systemic (oral and/or parenteral) steroid treatment; and Requiring hospitalization or emergency room visit.

### Secondary Objectives:

To evaluate the effects of SAR440340 and co-administration of SAR440340 and dupilumab, compared with placebo, on FEV₁. To estimate the effects of co-administration of SAR440340 and dupilumab, compared with SAR440340 and compared with dupilumab, on FEV₁. To determine the safety and tolerability of SAR440340 alone and in co-administration with dupilumab.

Methodology:

Randomized, double-blind, placebo-controlled, parallel-group (4 groups), 12-week proof-of-concept (PoC) study assessing the efficacy, safety, and tolerability of SAR440340 300 mg q2w, and the co-administration of SAR440340 300 mg q2w and dupilumab 300 mg q2w in patients with moderate-to-severe asthma who are not well controlled on inhaled corticosteroid (ICS) plus long-acting β2 adrenergic agonist (LABA) therapy. Background therapy (ICS and LABA) was gradually withdrawn and patients were without any background therapy for a period of 3-4 weeks at the end of treatment phase. SAR440340 was administered as 2 subcutaneous injections. Dupilumab was administered as 1 subcutaneous (SC) injection.

### Diagnosis and Criteria for Inclusion:

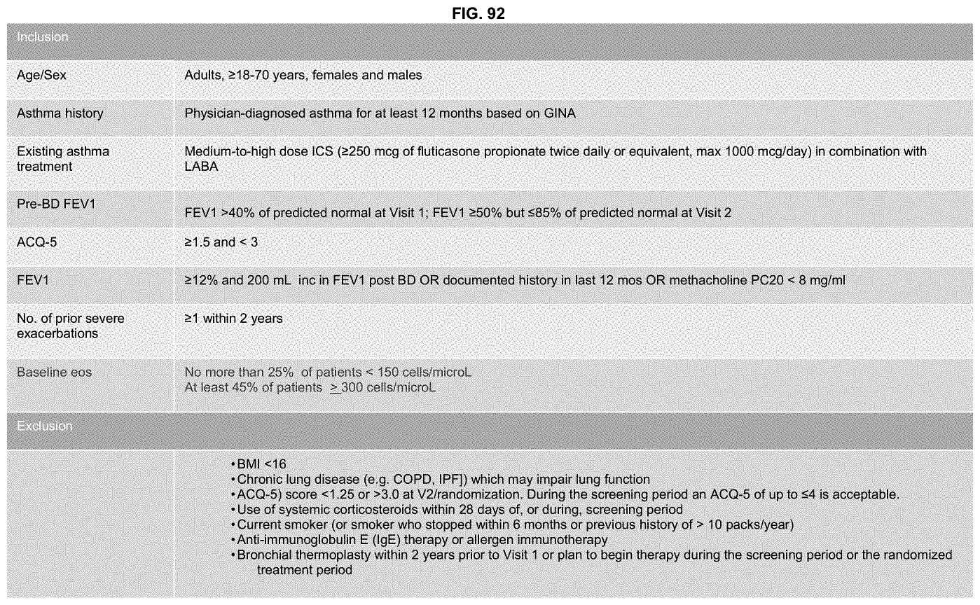

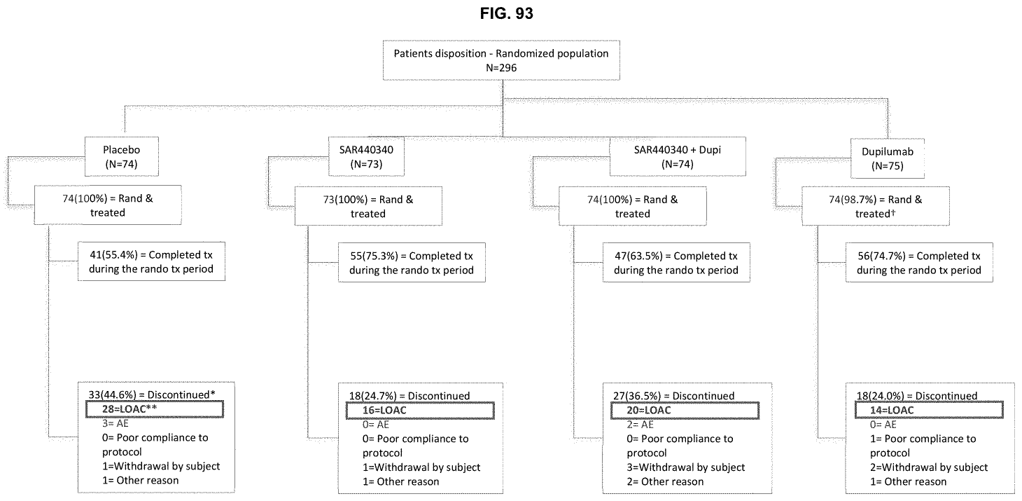

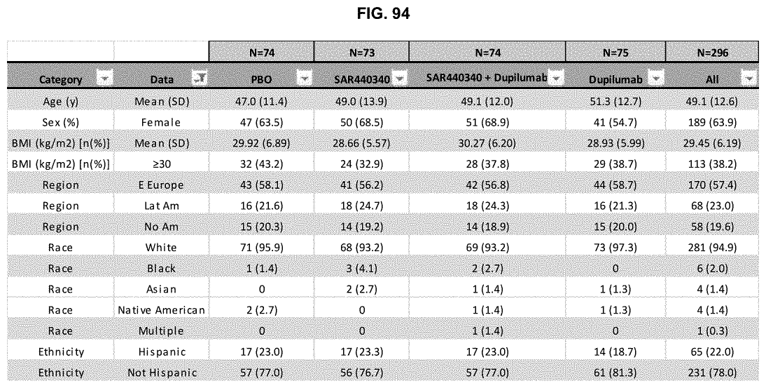

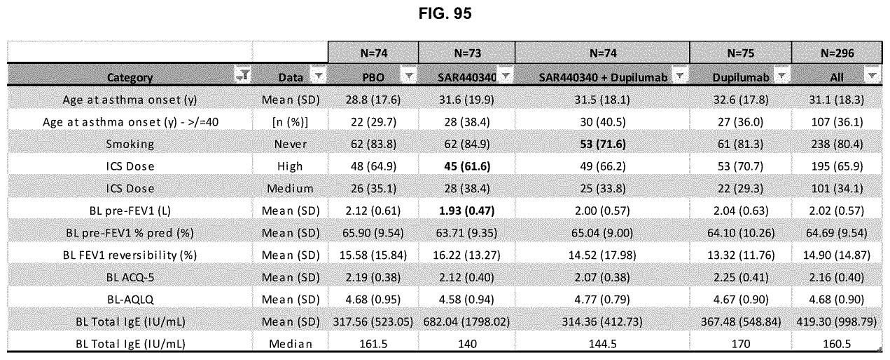

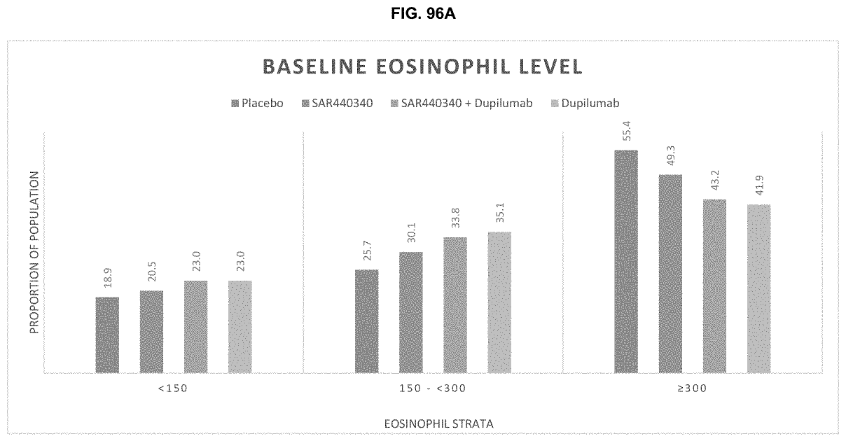

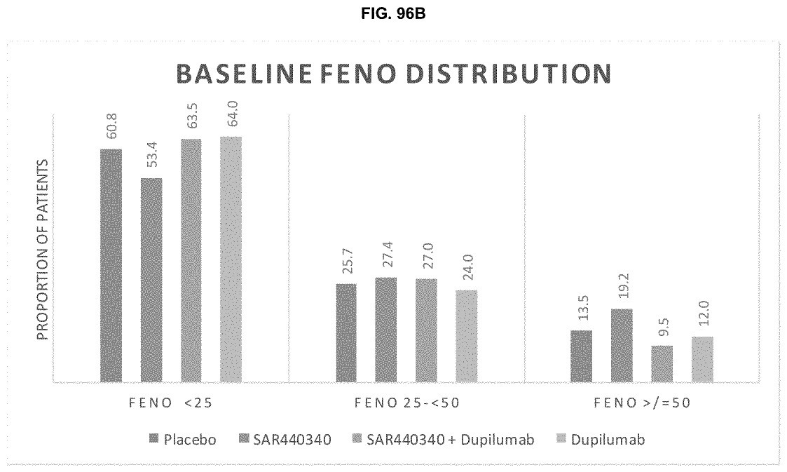

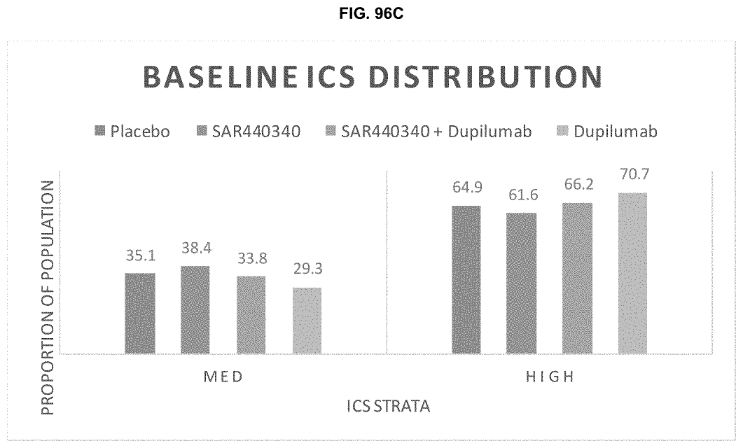

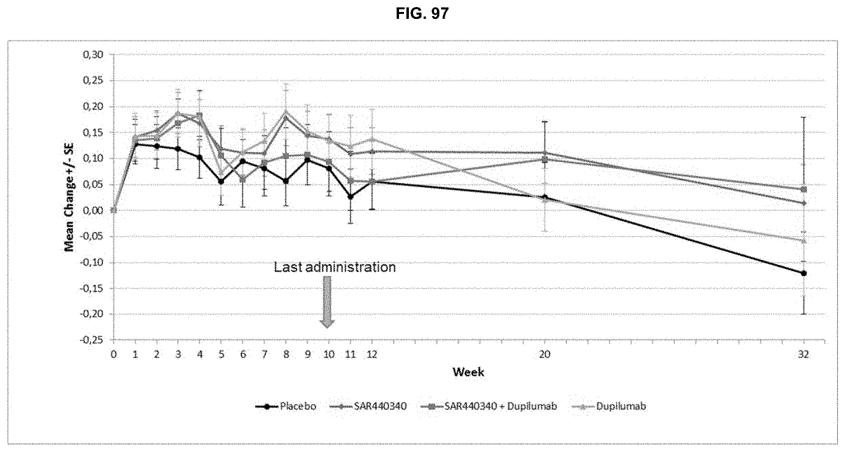

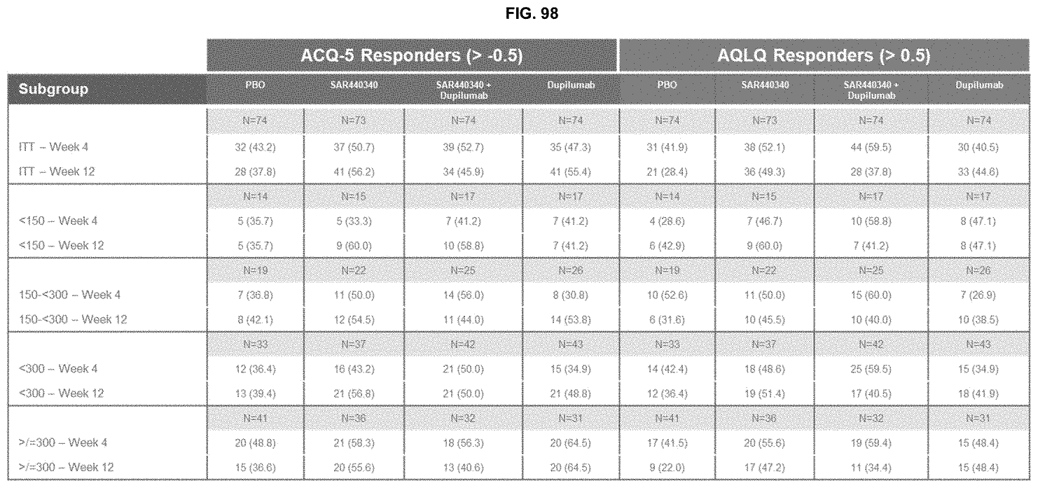

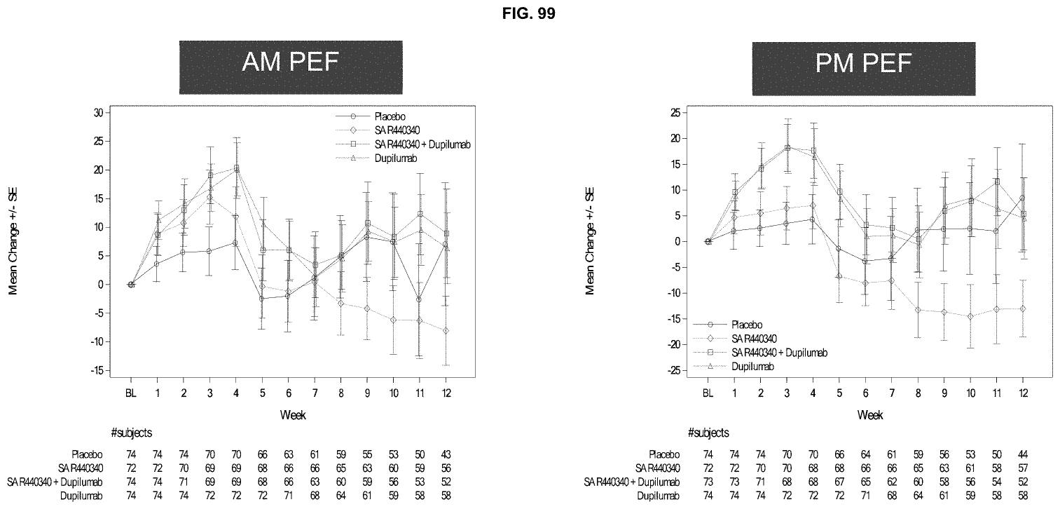

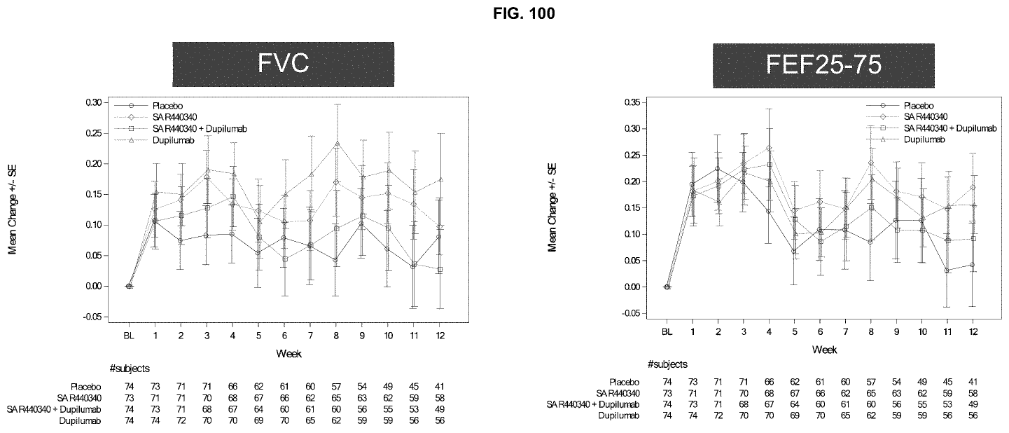

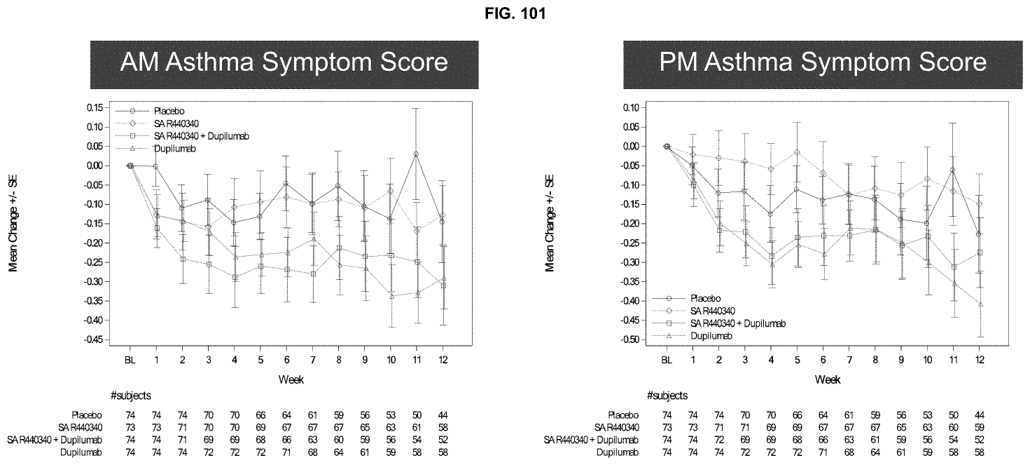

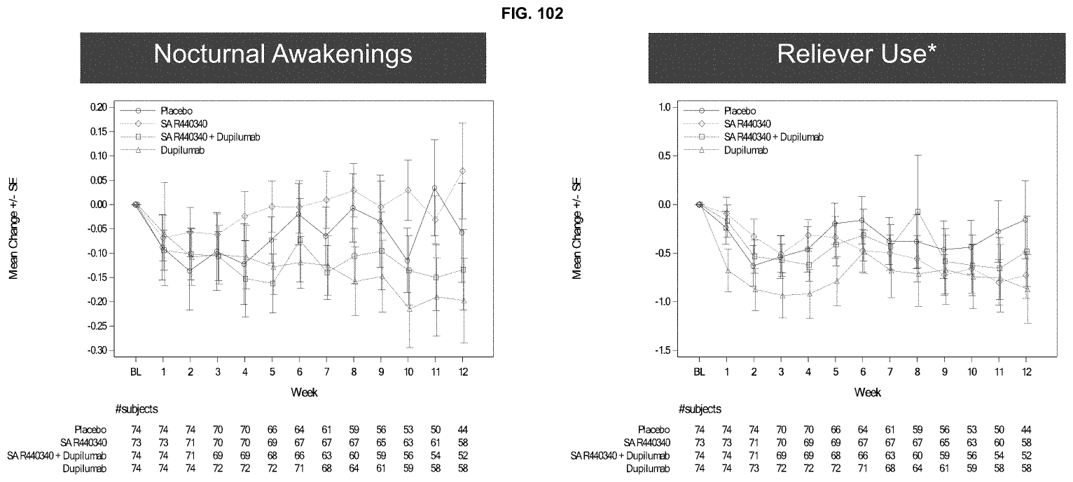

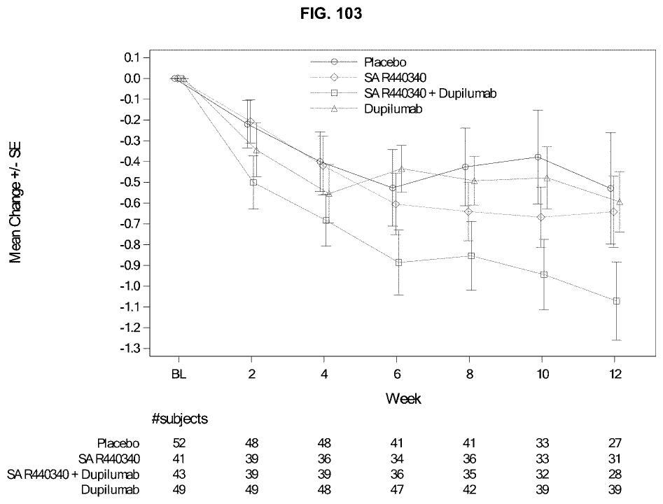

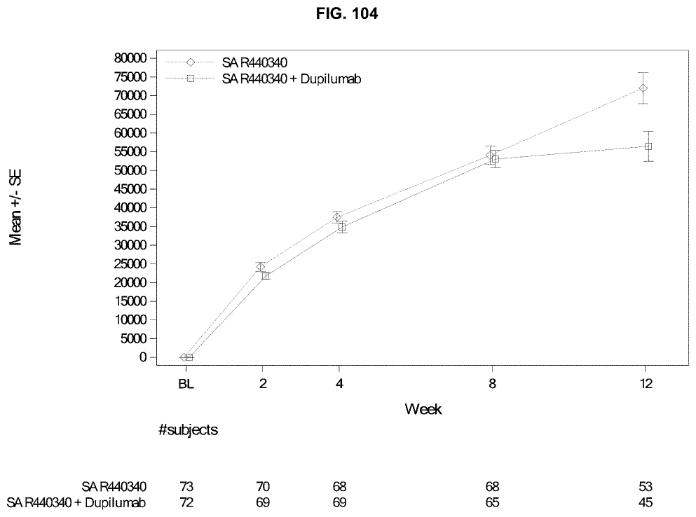

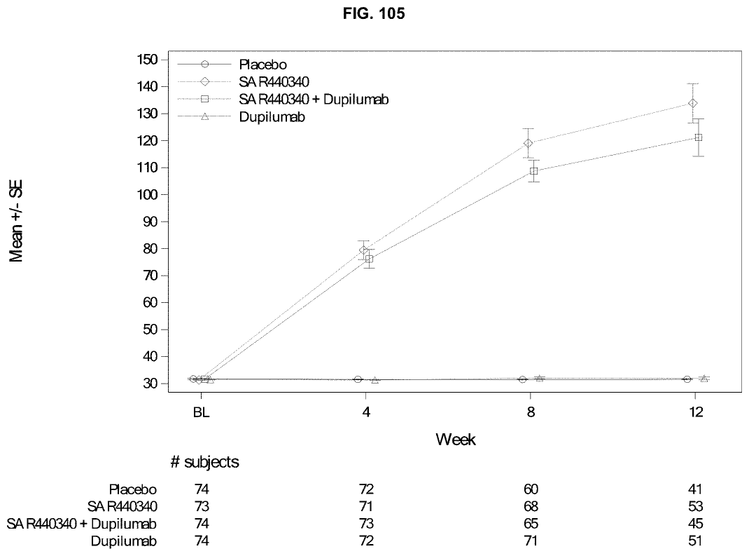

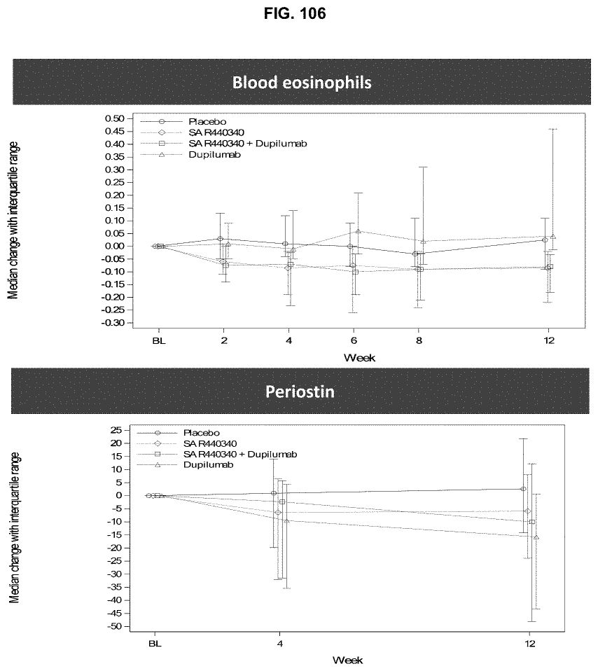

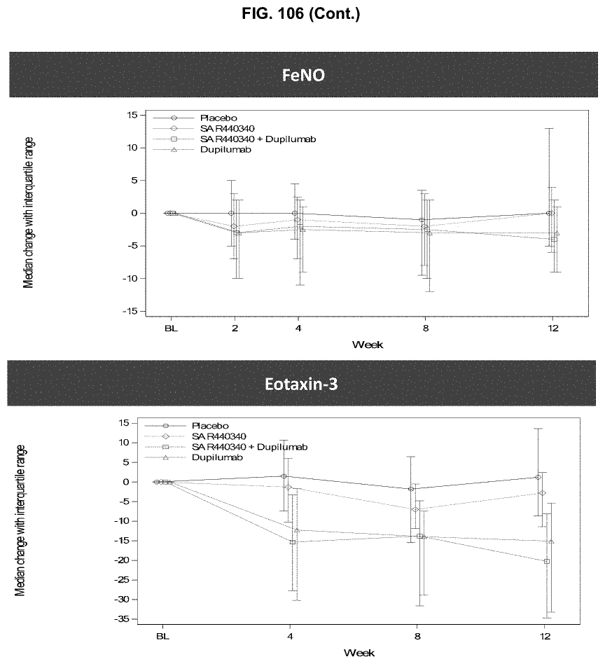

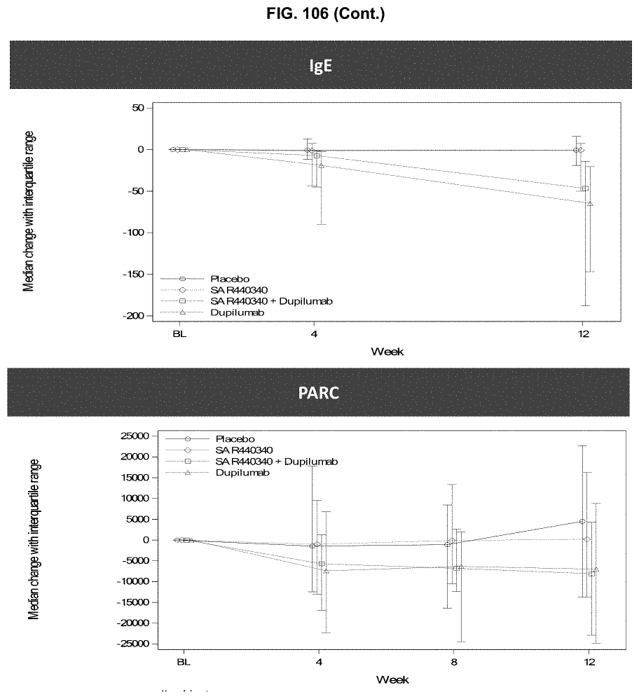

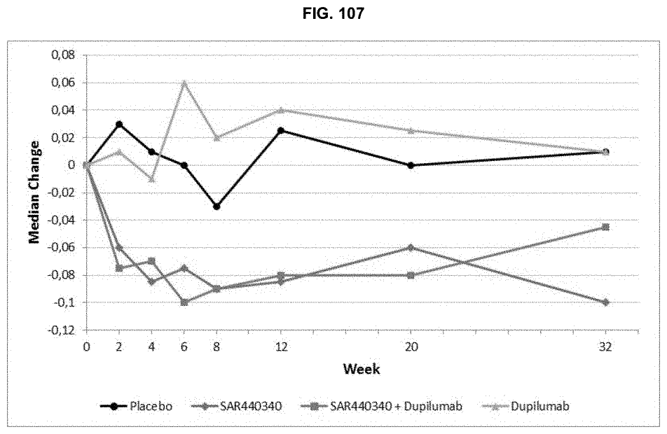

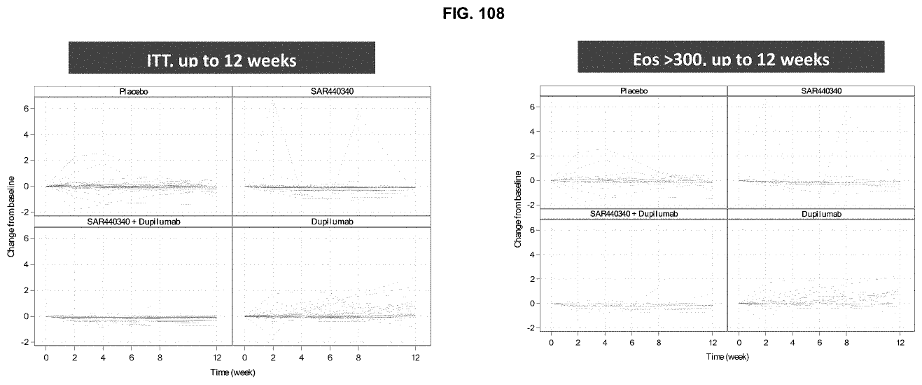

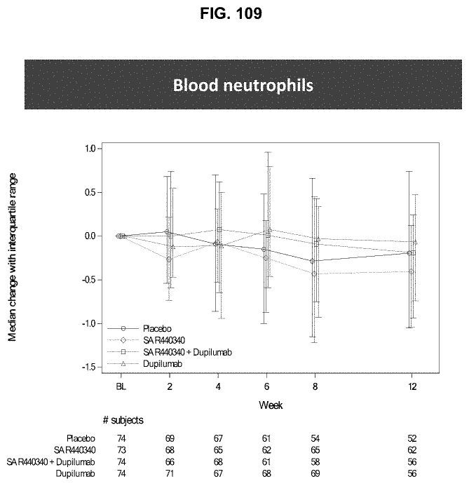

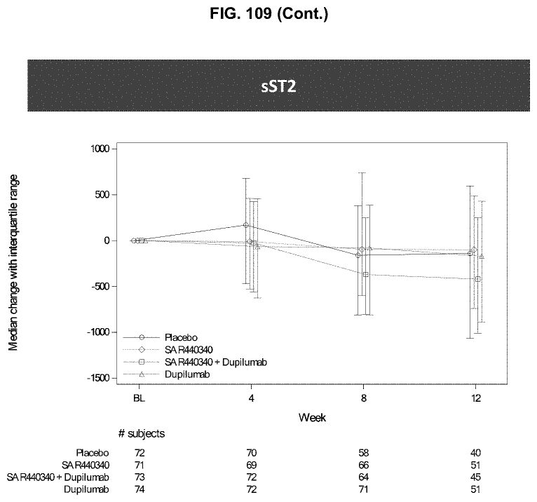

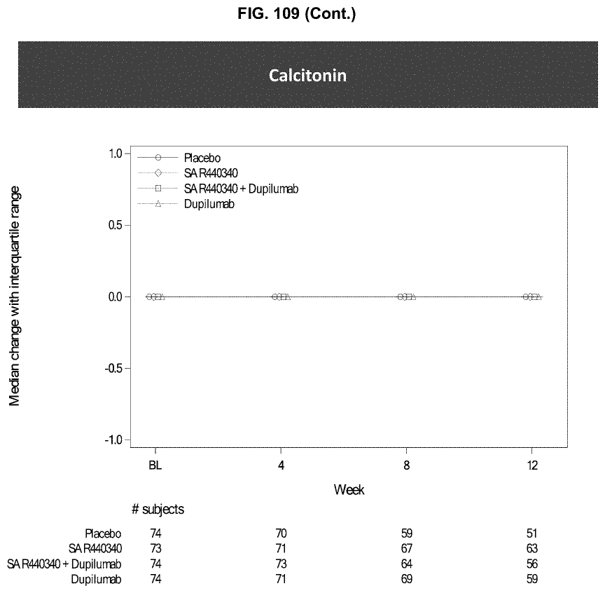

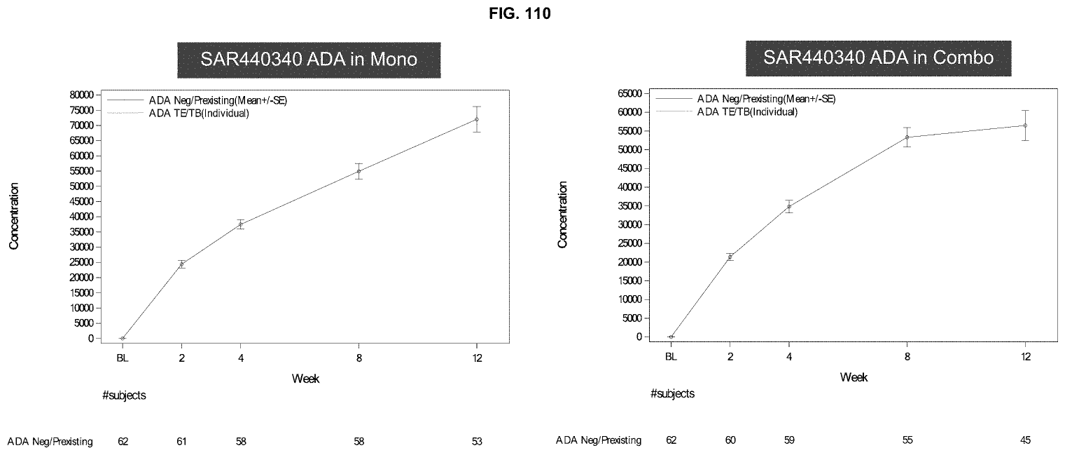

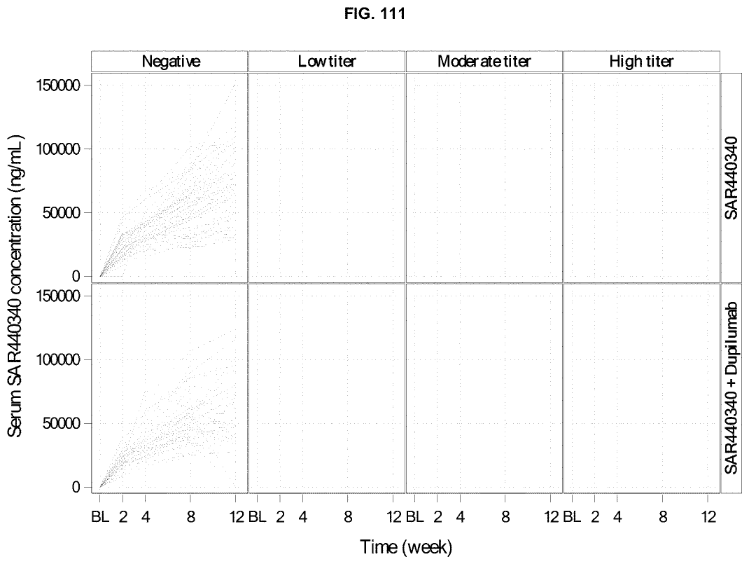

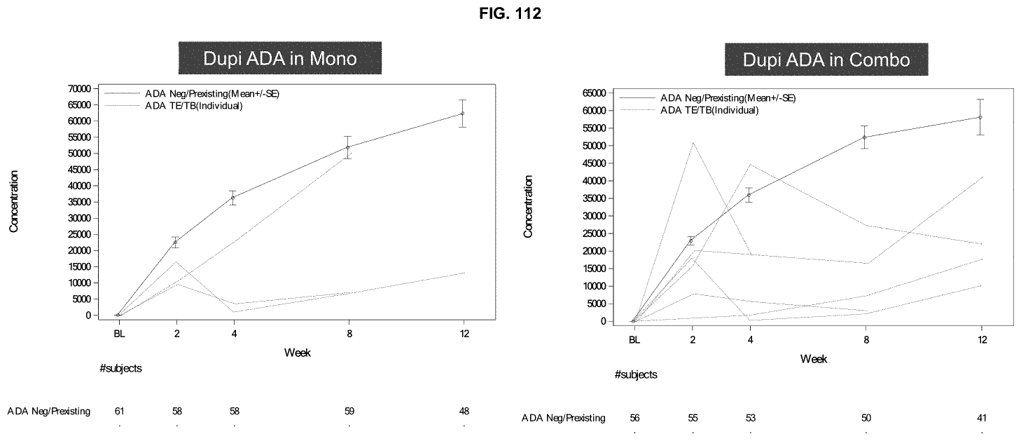

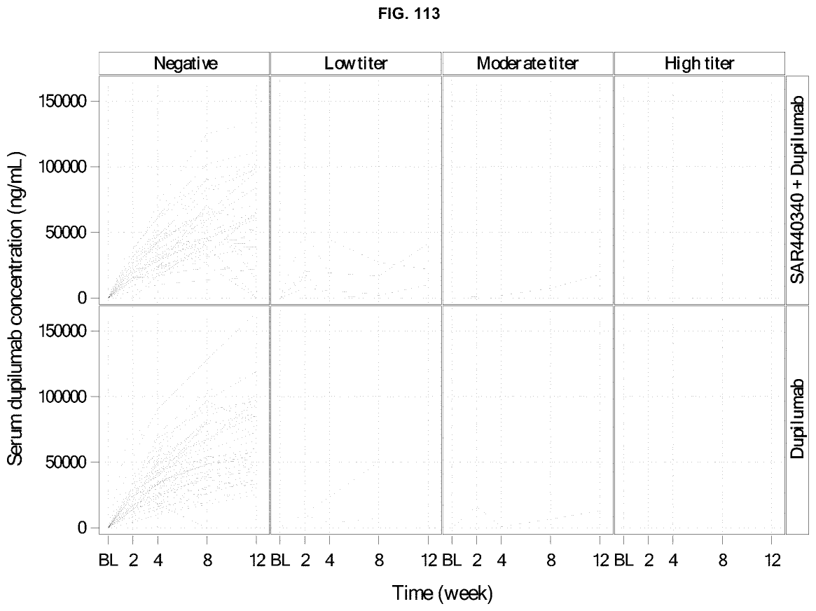

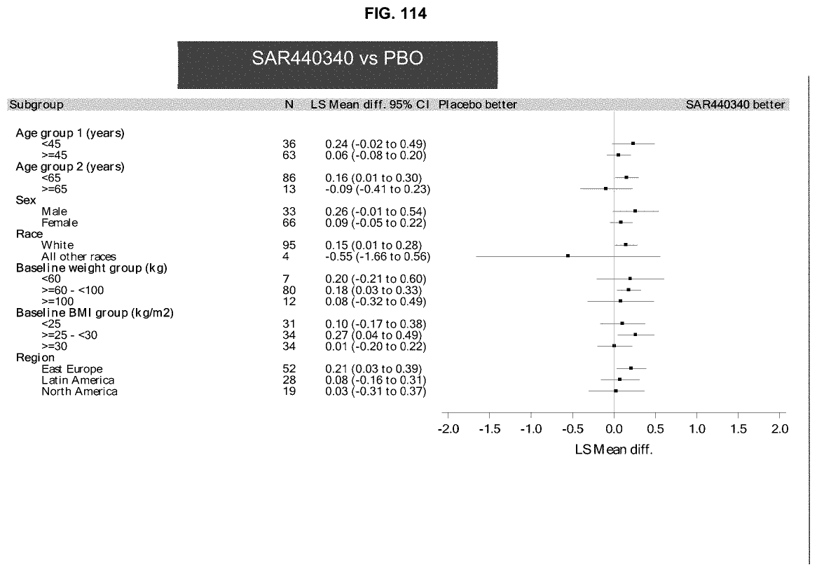

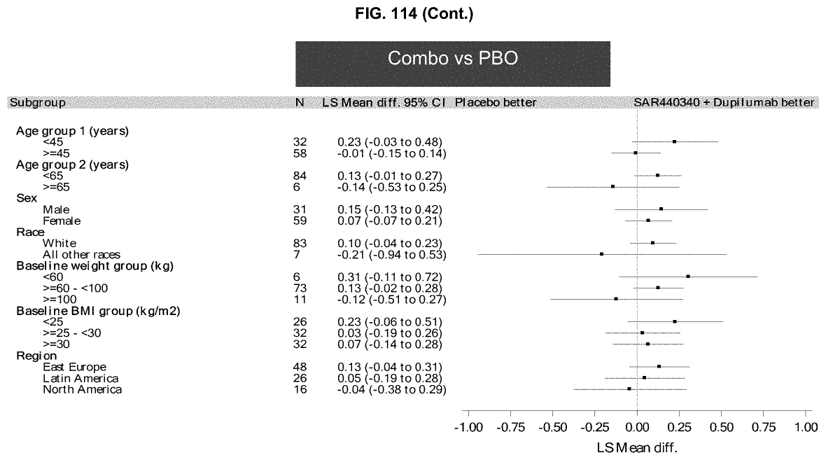

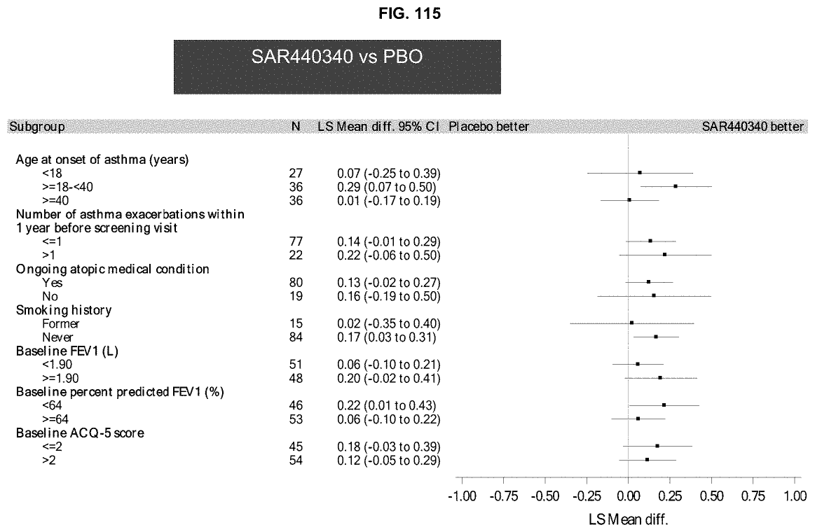

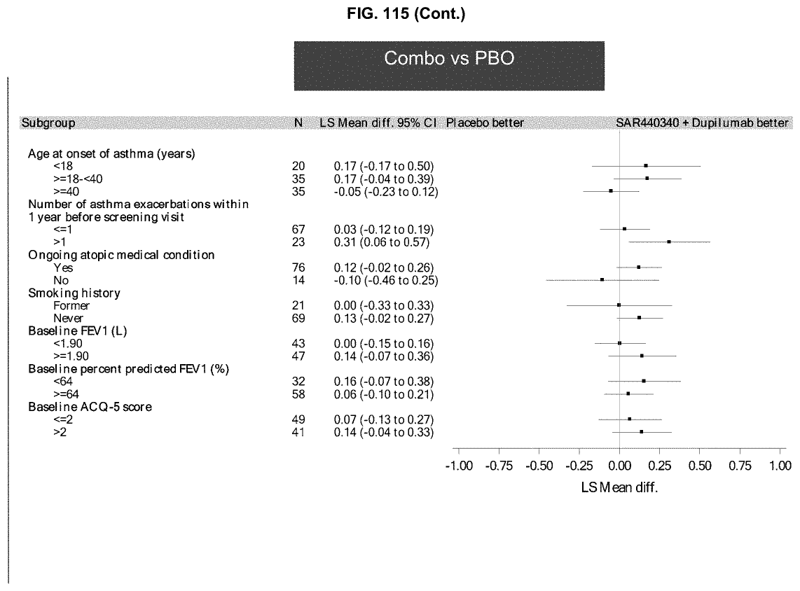

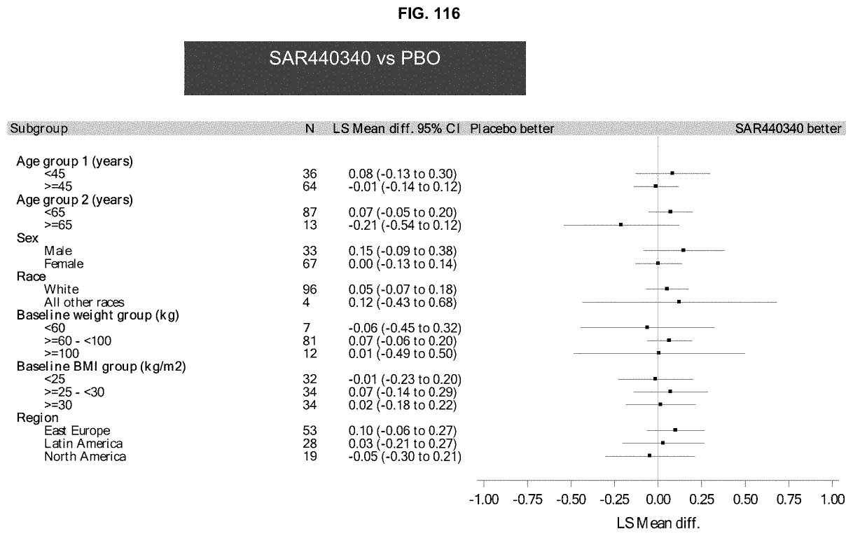

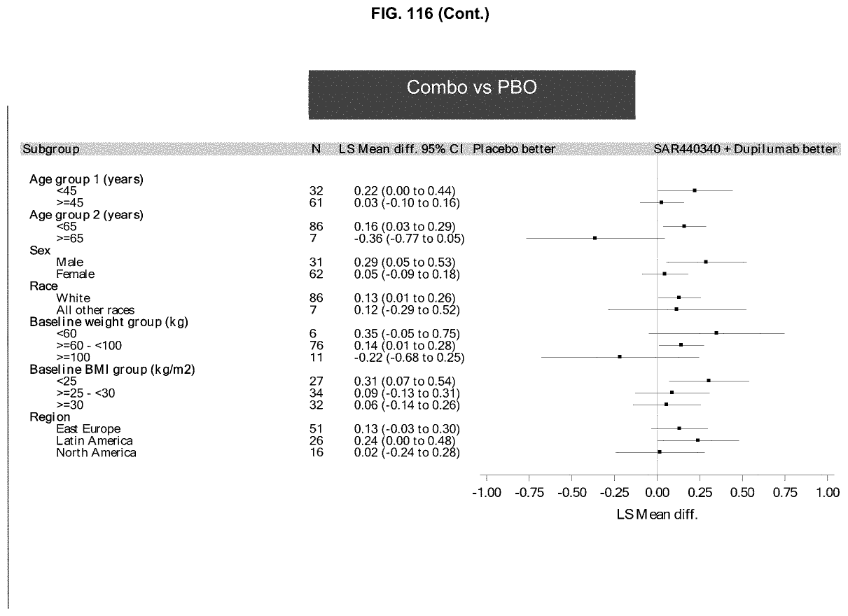

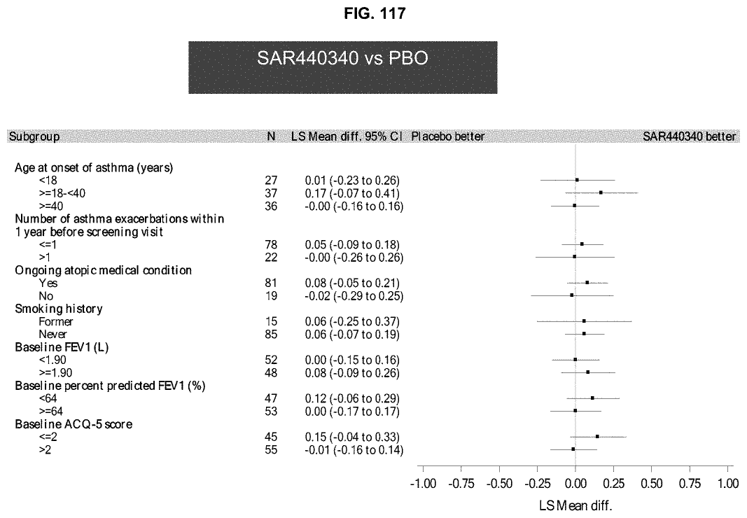

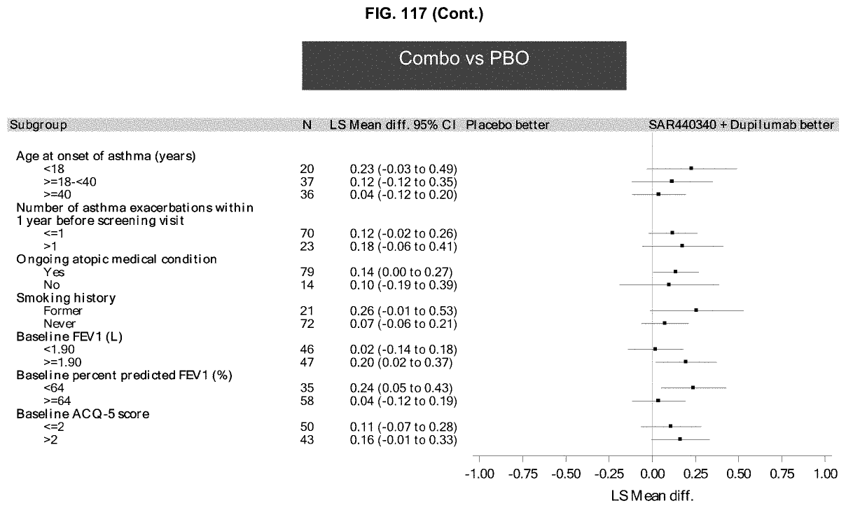

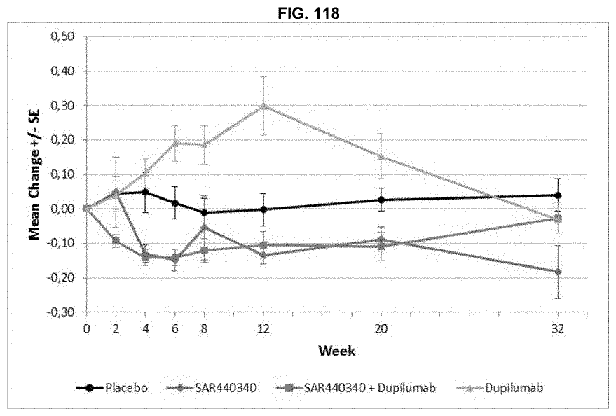

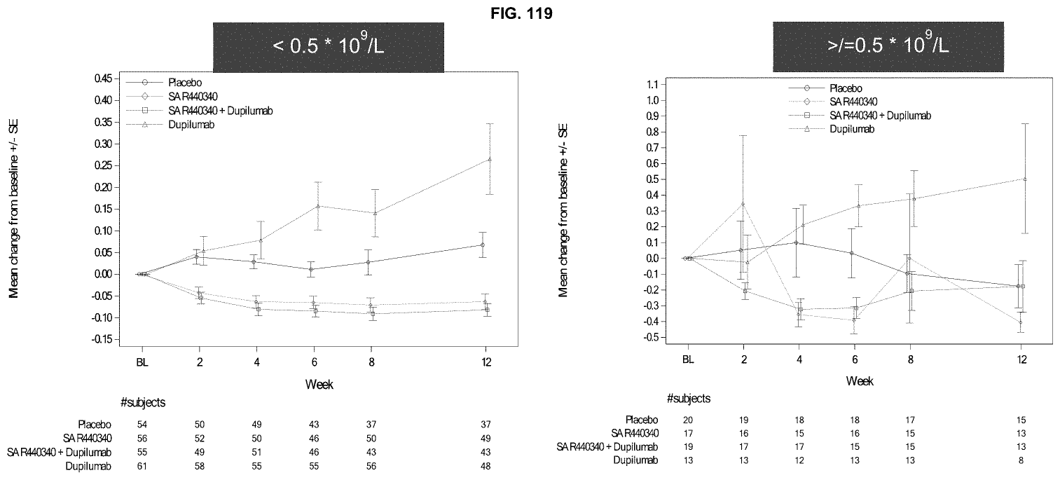

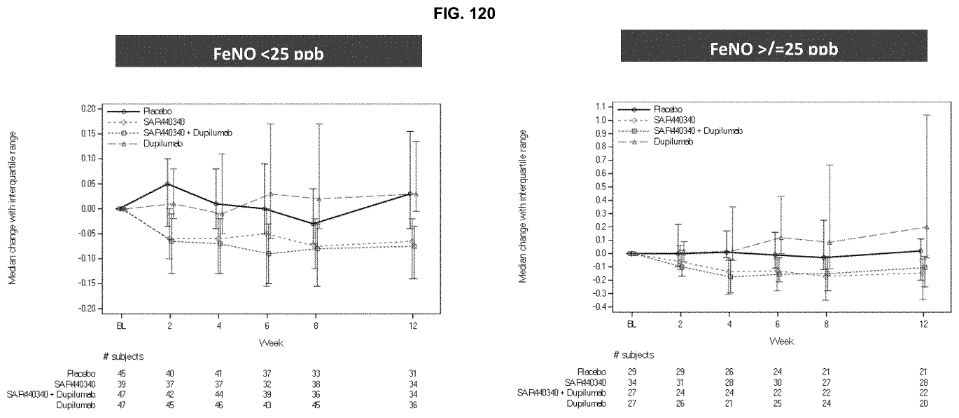

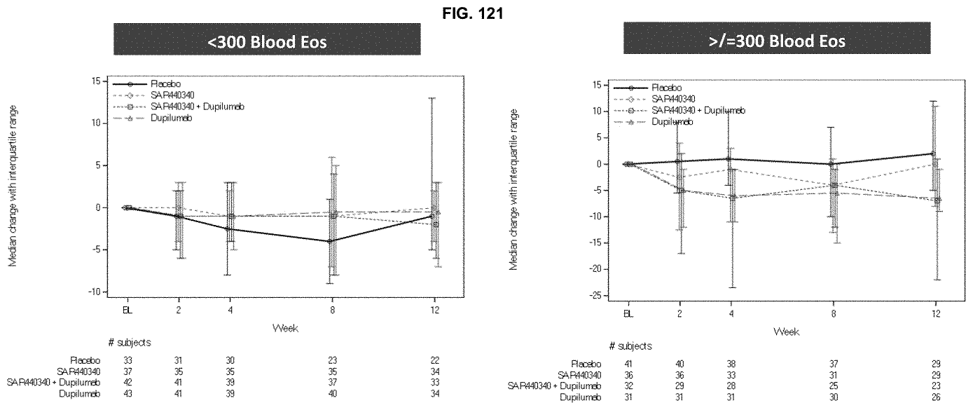

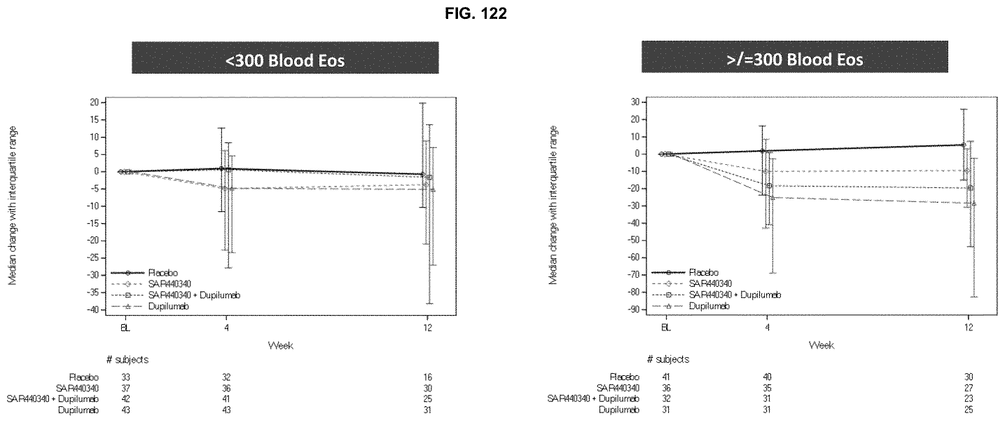

1. Adult patients with a physician diagnosis of asthma for at least 12 months based on the Global Initiative for Asthma (GINA) 2017 Guidelines. 2. Existing treatment with medium-to-high dose ICS (≥250 mcg of fluticasone propionate twice daily (BID) or equipotent ICS daily dosage to a maximum of 2000 mcg/day of fluticasone propionate or clinically comparable) in combination with a LABA as second controller for at least 3 months with a stable dose≥1 month prior to visit 1. 3. Pre-bronchodilator forced expiratory volume (FEV1)>40% of predicted normal at visit 1/screening. 4. Pre-bronchodilator FEV1≥50% but ≤85% of predicted normal at visit 2/baseline. 5. Reversibility of at least 12% and 200 mL in FEV1 after administration of 2 to 4 puffs (200-400 mcg) of albuterol/salbutamol or levalbuterol/levosalbutamol during screening or documented history of a reversibility test that meets this criteria within 12 months prior to visit 1 or documented positive response to methacholine challenge (a decrease in FEV by 20% [PC20] of <8 mg/mL) within 12 months prior to visit 1/screening. 6. Experienced at least once, within 1 year prior to visit 1, either hospitalization or emergency medical care visit for worsening asthma or treatment with a systemic steroid (oral or parenteral) for worsening asthma. (See also FIG. 92-FIG. 96.)

### Primary and Main Secondary Key Endpoints:

Efficacy:

The primary endpoint was the proportion of patients with LOAC. The secondary endpoint was FEV1 change from baseline at week 12 (pre- and post-bronchodilator).

Safety: Adverse events (AE), standard hematology and blood chemistry, vital signs, physical examination, and electrocardiogram (ECG).

Statistical Methods:

The efficacy analysis population was the modified intent-to-treat (mITT) population, defined as all randomized patients who received at least one dose of investigational product. Patients were analyzed according to the treatment group allocated by randomization. Randomization was stratified by blood eosinophil count at screening visit (<0.15 Giga/L, 0.15 −<0.3 Giga/L, ≥0.3 Giga/L) and by country.

The primary endpoint of incidence of LOAC was analyzed by a logistic regression model. Covariates included in the model were treatment, baseline eosinophil strata, region (pooled countries), background ICS dose level at randomization and number of exacerbation events within 1 year prior to screening.

The secondary endpoints, change from baseline in pre- and post-BD FEV1 at week 12, were analyzed using a mixed effect model with repeated measures (MIVIRM) approach. The model included change from baseline values up to week 12 as response variable and treatment, gender, baseline height, baseline eosinophil strata, region, background ICS dose level at randomization, visit, treatment-by-visit interaction, baseline value and baseline-by-visit interaction as covariates. For patients who experienced LOAC with rescue medications, FEV1 collected at and after start of the event were set to missing for the primary analysis. Missing data were not imputed.

All pairwise treatment comparisons in efficacy analyses were tested at a two-sided 5% significance level. The safety population included all patients who were exposed to IMP, regardless of the amount of exposure. Patients were analyzed according to the treatment actually received. Safety summaries were descriptive and no hypothesis testing was conducted.

Summary

Population Characteristics:

For this core database lock, out of 296 patients randomized, 228 (77.0%) patients were ongoing in the post-treatment follow-up period, while 58 (19.6%) patients completed the study as planned and 10 (3.4%) patients discontinued from the follow-up. Out of the 295 patients randomized and treated, 199 (67.5%) patients completed treatment as planned. Ninety-six (32.5%) patients discontinued treatment prematurely.

Seventy-eight patients permanently discontinued treatment due to loss of asthma control (protocol requirement), 5 patients due to adverse event, 1 patient due to low compliance, and 5 patients due to other reason not related to safety. Seven patients withdrew treatment due to their request. More patients in the placebo arm discontinued the treatment due to LOAC. Early treatment discontinuation due to adverse event was low, with two patients discontinued in SAR440340+dupilumab group, and three patients discontinued in the placebo group.

Patients' demographics, other characteristics, and disease characteristics at baseline were generally similar across the four treatment arms, with an overall mean number of asthma exacerbations the previous year of 1.3, mean pre-bronchodilator FEV1 of 2.02 L, mean pre-bronchodilator FEV1 percent predicted of 64.69%, mean FEV1 reversibility 14.9%, mean ACQ-5 score (Asthma Control Questionnaire 5-question version) of 2.16, mean eosinophils 0.37 10\hat{ }9/L and mean fractional exhaled nitric oxide (FeNO) of 29.2 ppb. In the SAR440340+dupilumab group, a higher proportion of patients experienced two or more exacerbations or exacerbations leading to hospitalization than in other treatment groups. There were also more patients who were former smokers in this treatment group.

Efficacy Results:

The proportion of patients with LOAC was 21.9% in SAR440340, 27% in SAR440340+dupilumab, 18.9% in dupilumab, and 40.5% in the placebo group. SAR440340 monotherapy reduced the odds of experiencing LOAC compared to placebo (p=0.02). The odds ratio reduction in SAR440340+dupilumab group was not significant (p=0.07). The odds ratios (95% CIs) were 0.423 (0.203 to 0.880) for SAR440340 versus placebo and 0.520 (0.256 to 1.057) for the combination versus placebo.

Although there was no evidence of a differential treatment effect across subgroups defined by baseline eosinophil count (p=0.5365 for the treatment by subgroup interaction), the number of patients within each subgroup was small. Generally, the largest effect versus placebo was observed in the subgroup of patients with baseline eosinophil count≥300/mm3 for both SAR440340 and the combination of SAR440340 with dupilumab.

The LS mean change from baseline in pre-bronchodilator FEV1 at week 12 was 0.10 L for SAR440340, 0.06 L for SAR440340+dupilumab, 0.12 L for dupilumab, and −0.04 L for the placebo group. The LS mean difference in change from baseline to week 12 in pre-bronchodilator FEV1 compared to placebo was significant for the SAR440340 group (0.14 [0.01 to 0.27], p=0.03), but not for the SAR440340+dupilumab group (0.10 [−0.03 to 0.23], p=0.13).

Although there was no evidence of a differential treatment effect across subgroups defined by baseline eosinophil count (p=0.1111 for the treatment by subgroup interaction), the number of patients within each subgroup was small. Generally, the largest effect versus placebo was observed in the subgroup of patients with baseline eosinophil count≥300/mm3 for both SAR440340 and the combination of SAR440340 with dupilumab.

The LS mean change from baseline in post-bronchodilator FEV1 at week 12 was −0.00 L for SAR440340, 0.07 L for SAR440340+dupilumab, 0.09 L for dupilumab, and −0.05 L for the placebo group. The LS mean difference in change from baseline to week 12 in post-bronchodilator FEV1 compared to placebo was significant for the SAR440340+dupilumab group (0.13 [0.01 to 0.25], p=0.04) but not for the SAR440340 group (0.05 [−0.07 to 0.17], p=0.41).

Safety Results (Incidence Reported as SAR440340, SAR440340+Dupilumab, Dupilumab, Placebo in all Cases):

Cumulative exposure to SAR440340 alone or in combination with dupilumab was 30.2 patient-years. The incidence of treatment-emergent adverse events (TEAEs) was balanced across treatment groups (60.3%, 66.2%, 55.4% and 64.9%). Most TEAEs were mild or moderate intensity. The most frequent TEAEs were in the system organ class (SOC) of infections and infestations (mainly due to nasopharyngitis and viral upper respiratory tract infections).

The incidence of treatment-emergent serious adverse events (SAEs) was low. In each of the SAR440340, the SAR440340+dupilumab, and the dupilumab treatment groups, one patient (1.4%) had treatment emergent SAE compared to three patients (4.1%) in the placebo group. One patient died due to ethyl alcohol poisoning in the post-treatment follow-up period (information received after database lock).

The overall treatment discontinuation rate due to TEAEs was low (2 patients (2.7%) discontinued in SAR440340+dupilumab group and 3 patients (4.1%) discontinued in placebo group).

Preliminary Conclusions:

In patients with moderate-to-severe persistent asthma, SAR440340 monotherapy was efficacious. Compared to placebo, SAR440340 monotherapy showed significant reduction in proportion of patients with LOAC and improvement in pre-bronchodilator FEV1 at week 12 in the overall population. No treatment effect has been observed on improvement of post-bronchodilator FEV1 at week 12. Subgroup analyses based on the blood eosinophil count at baseline showed trend to reduction of LOAC incidence across eosinophil subgroups. In contrast, a trend to improvement in pre-bronchodilator FEV1 at week 12 was primarily observed in the ≥300/mm3 subgroup compared to placebo.

SAR440340 in combination with dupilumab did not show efficacy compared to placebo on reduction of patients with LOAC and on improvement in pre-bronchodilator FEV1 at week 12 in the overall population. However, efficacy was observed on improvement of post-bronchodilator FEV1 at week 12. Subgroup analyses based on the blood eosinophil count at baseline showed in general the largest treatment effect, for both LOAC and pre-bronchodilator FEV1, in the subgroup of patients with baseline EOS count≥300/mm3, compared to placebo.

A dupilumab arm was included in this study as a calibrator. For both LOAC and FEV1, dupilumab performed as expected.

SAR440340 alone and in combination with dupilumab was generally well tolerated with an acceptable safety profile.

Results

Patient Disposition

Out of 498 patients screened, 296 patients were randomized (screen failure rate of 41%). The main reasons for screen failure were positive tuberculosis screening and prebronchodilator FEV1 being out of the pre-specified range for inclusion. Patient disposition by randomized treatment group is shown in Table 1. Assignment to treatment groups matched the planned randomization scheme of 1:1:1:1.

The final distribution according to the baseline eosinophil count stratum (used for statistical analysis) was: 63 patients (21.4%) with eosinophil count<0.15×10⁹/L, 92 patients (31.2%)≥0.15×10⁹/L and <0.3×10⁹/L, 140 patients (47.5%)≥0.3×10⁹/L (Table 12). This slightly differed from IVRS stratification that was based on screening eosinophil count (Table 3).

Out of the 295 patients randomized and treated, 199 (67.5%) patients completed treatment as planned. Ninety-six (32.5%) patients discontinued treatment prematurely. For this core database lock, out of 296 patients randomized, 228 (77.0%) patients were ongoing in the post-treatment follow-up period, while 58 (19.6%) patients completed the study as planned and 10 patients (3.4%) discontinued from the follow-up.

Seventy-eight patients permanently discontinued treatment due to loss of asthma control (protocol requirement), five patients due to adverse event, one patient due to low compliance, and five patients due to other reasons not related to safety. Seven patients withdrew treatment due to their request. More patients in the placebo group discontinued treatment due to LOAC. Early treatment discontinuation due to adverse event was low, with two patients discontinued in the SAR440340+placebo group and three patients discontinued in the placebo group.

There was 1 patient reported lost to follow-up in the dupilumab treatment group. After database lock, new information has been received that the patient died in the study post-treatment follow-up due to SAE of ethyl alcohol poisoning. The event was assessed as not related to IMP.

TABLE 1
Patient disposition, randomized population

| | | SAR440340 | SAR440340 q2w + | Dupilumab |
|---|---|---|---|---|
| | Placebo | 300 mg q2w | Dupilumab q2w | 300 mg q2w |
| n(%) | (N = 74) | (N = 73) | (N = 74) | (N = 75) |
| Randomized and not treated | 0 | 0 | 0 | 1 (1.3) |
| Randomized and treated | 74 (100) | 73 (100) | 74 (100) | 74 (98.7) |
| Completed the study treatment | 41 (55.4) | 55 (75.3) | 47 (63.5) | 56 (74.7) |
| Did not complete the study | 33 (44.6) | 18 (24.7) | 27 (36.5) | 18 (24.0) |
| treatment | | | | |

Reason for treatment discontinuation

| | | | | |
|---|---|---|---|---|
| Adverse event | 3 (4.1) | 0 | 2 (2.7) | 0 |
| Lack of efficacy | 0 | 0 | 0 | 0 |
| Loss of asthma control | 28 (37.8) | 16 (21.9) | 20 (27.0) | 14 (18.7) |
| Poor compliance to protocol | 0 | 0 | 0 | 1 (1.3) |
| Withdrawal by subject | 1 (1.4) | 1 (1.4) | 3 (4.1) | 2 (2.7) |
| Other | 1 (1.4) | 1 (1.4) | 2 (2.7) | 1 (1.3) |

Reason for treatment withdrawal by subject

| | | | | |
|---|---|---|---|---|
| Adverse event | 0 | 0 | 0 | 0 |
| Other | 1 (1.4) | 1 (1.4) | 3 (4.1) | 2 (2.7) |
| Completed the study period | 18 (24.3) | 15 (20.5) | 14 (18.9) | 11 (14.7) |
| Discontinued from the study | 2 (2.7) | 1 (1.4) | 3 (4.1) | 4 (5.3) |

Reason for study discontinuation

| | | | | |
|---|---|---|---|---|
| Adverse event | 0 | 0 | 0 | 0 |
| Lost to follow-up | 0 | 0 | 0 | 1 (1.3) |
| Other | 2 (2.7) | 1 (1.4) | 3 (4.1) | 3 (4.0) |

Status at last study contact

| | | | | |
|---|---|---|---|---|
| Ongoing in the follow-up | 54 (73.0) | 57 (78.1) | 57 (77.0) | 60 (80.0) |
| period | | | | |
| Alive | 20 (27.0) | 16 (21.9) | 17 (23.0) | 15 (20.0) |
| Dead | 0 | 0 | 0 | 0 |

Note:
Percentages are calculated using the number of patients randomized as denominator.

### Demographics and Baseline Characteristics

Patient demographics and baseline characteristics were well-balanced across treatment groups (Table 2). The mean age of the overall population was 49 years with a range of 18 to 70 years. Approximately two-thirds of the patients were females (63.9%). 94.9% of the patients were white, 2.0% black, 1.4% Asian, 1.4% American Indian or Alaska native and 0.3% were of multiple races.

TABLE 2
Demographics and patient characteristics at baseline, randomized population

| | | | | |
|---|---|---|---|---|
| | SAR440340 | SAR440340 q2w + | Dupilumab | |
| Placebo | 300 mg q2w | Dupilumab q2w | 300 mg q2w | All |
| (N = 74) | (N = 73) | (N = 74) | (N = 75) | (N = 296) |

Age (years)

| | | | | | |
|---|---|---|---|---|---|
| Number | 74 | 73 | 74 | 75 | 296 |
| Mean (SD) | 47.0 (11.4) | 49.0 (13.9) | 49.1 (12.0) | 51.3 (12.7) | 49.1 (12.6) |
| Median | 48.0 | 52.0 | 49.0 | 52.0 | 50.0 |
| Q1; Q3 | 37.0; 55.0 | 40.0; 60.0 | 39.0; 60.0 | 42.0; 61.0 | 39.0; 59.0 |
| Min; Max | 23; 70 | 21; 69 | 24; 69 | 18; 70 | 18; 70 |

Age group 1 (years) [n (%)]

| | | | | | |
|---|---|---|---|---|---|
| Number | 74 | 73 | 74 | 75 | 296 |
| <45 | 29 (39.2) | 28 (38.4) | 28 (37.8) | 22 (29.3) | 107 (36.1) |
| ≥45 | 45 (60.8) | 45 (61.6) | 46 (62.2) | 53 (70.7) | 189 (63.9) |

Age group 2 (years) [n (%)]

| | | | | | |
|---|---|---|---|---|---|
| Number | 74 | 73 | 74 | 75 | 296 |
| <65 | 69 (93.2) | 62 (84.9) | 69 (93.2) | 62 (82.7) | 262 (88.5) |
| ≥65 | 5 (6.8) | 11 (15.1) | 5 (6.8) | 13 (17.3) | 34 (11.5) |

Sex [n (%)]

| | | | | | |
|---|---|---|---|---|---|
| Number | 74 | 73 | 74 | 75 | 296 |
| Male | 27 (36.5) | 23 (31.5) | 23 (31.1) | 34 (45.3) | 107 (36.1) |
| Female | 47 (63.5) | 50 (68.5) | 51 (68.9) | 41 (54.7) | 189 (63.9) |

Race [n (%)]

| | | | | | |
|---|---|---|---|---|---|
| Number | 74 | 73 | 74 | 75 | 296 |
| White | 71 (95.9) | 68 (93.2) | 69 (93.2) | 73 (97.3) | 281 (94.9) |
| Black or African American | 1 (1.4) | 3 (4.1) | 2 (2.7) | 0 | 6 (2.0) |
| Asian | 0 | 2 (2.7) | 1 (1.4) | 1 (1.3) | 4 (1.4) |
| Native Hawaiian or Other | 0 | 0 | 0 | 0 | 0 |
| Pacific Islander | | | | | |
| American Indian or Alaska | 2 (2.7) | 0 | 1 (1.4) | 1 (1.3) | 4 (1.4) |
| Native | | | | | |
| Multiple | 0 | 0 | 1 (1.4) | 0 | 1 (0.3) |
| Unknown | 0 | 0 | 0 | 0 | 0 |
| Not Reported | 0 | 0 | 0 | 0 | 0 |

Ethnicity [n (%)]

| | | | | | |
|---|---|---|---|---|---|
| Number | 74 | 73 | 74 | 75 | 296 |
| Hispanic or Latino | 17 (23.0) | 17 (23.3) | 17 (23.0) | 14 (18.7) | 65 (22.0) |
| Not Hispanic or Latino | 57 (77.0) | 56 (76.7) | 57 (77.0) | 61 (81.3) | 231 (78.0) |
| Unknown | 0 | 0 | 0 | 0 | 0 |
| Not Reported | 0 | 0 | 0 | 0 | 0 |

Weight (kg)

| | | | | | |
|---|---|---|---|---|---|
| Number | 74 | 73 | 74 | 75 | 296 |
| Mean (SD) | 83.11 (19.74) | 78.20 (15.31) | 83.35 (19.17) | 81.31 (18.42) | 81.50 (18.27) |
| Median | 80.20 | 76.50 | 81.00 | 78.30 | 79.60 |
| Q1; Q3 | 69.00; 90.50 | 67.00; 90.00 | 68.00; 94.00 | 68.00; 89.50 | 67.70; 91.35 |
| Min; Max | 54.0; 176.3 | 46.0; 116.8 | 55.0; 147.8 | 46.3; 139.8 | 46.0; 176.3 |

Weight group (kg) [n (%)]

| | | | | | |
|---|---|---|---|---|---|
| Number | 74 | 73 | 74 | 75 | 296 |
| <60 | 3 (4.1) | 5 (6.8) | 4 (5.4) | 5 (6.7) | 17 (5.7) |
| ≥60-<100 | 59 (79.7) | 62 (84.9) | 60 (81.1) | 57 (76.0) | 238 (80.4) |
| ≥100 | 12 (16.2) | 6 (8.2) | 10 (13.5) | 13 (17.3) | 41 (13.9) |

BMI (kg/m²)

| | | | | | |
|---|---|---|---|---|---|
| Number | 74 | 73 | 74 | 75 | 296 |
| Mean (SD) | 29.92 (6.89) | 28.66 (5.57) | 30.27 (6.20) | 28.93 (5.99) | 29.45 (6.19) |
| Median | 29.31 | 27.67 | 28.53 | 27.50 | 28.41 |
| Q1; Q3 | 24.24; 33.40 | 25.00; 31.43 | 25.59; 34.13 | 24.52; 32.39 | 24.79; 32.86 |
| Min; Max | 19.6; 57.6 | 17.3; 47.1 | 20.4; 49.1 | 17.4; 43.2 | 17.3; 57.6 |

BMI group (kg/m²) [n (%)]

| | | | | | |
|---|---|---|---|---|---|
| Number | 74 | 73 | 74 | 75 | 296 |
| <25 | 22 (29.7) | 18 (24.7) | 16 (21.6) | 21 (28.0) | 77 (26.0) |
| ≥25-<30 | 20 (27.0) | 31 (42.5) | 30 (40.5) | 25 (33.3) | 106 (35.8) |
| ≥30 | 32 (43.2) | 24 (32.9) | 28 (37.8) | 29 (38.7) | 113 (38.2) |

Region^{a} [n (%)]

| | | | | | |
|---|---|---|---|---|---|
| Number | 74 | 73 | 74 | 75 | 296 |
| East Europe | 43 (58.1) | 41 (56.2) | 42 (56.8) | 44 (58.7) | 170 (57.4) |
| Latin America | 16 (21.6) | 18 (24.7) | 18 (24.3) | 16 (21.3) | 68 (23.0) |
| North America | 15 (20.3) | 14 (19.2) | 14 (18.9) | 15 (20.0) | 58 (19.6) |

Frequency of alcohol drinking in the past 12 months [n (%)]

| | | | | | |
|---|---|---|---|---|---|
| Number | 74 | 73 | 74 | 75 | 296 |
| Never | 46 (62.2) | 50 (68.5) | 42 (56.8) | 53 (70.7) | 191 (64.5) |
| At least monthly | 21 (28.4) | 18 (24.7) | 27 (36.5) | 15 (20.0) | 81 (27.4) |
| At least weekly | 7 (9.5) | 5 (6.8) | 4 (5.4) | 6 (8.0) | 22 (7.4) |
| At least daily | 0 | 0 | 1 (1.4) | 1 (1.3) | 2 (0.7) |

Number of standard alcohol drinks on a typical day when drinking [n (%)]

| | | | | | |
|---|---|---|---|---|---|
| Number | 28 | 23 | 32 | 22 | 105 |
| 1 or 2 | 26 (92.9) | 22 (95.7) | 32 (100) | 21 (95.5) | 101 (96.2) |
| >2 | 2 (7.1) | 1 (4.3) | 0 | 1 (4.5) | 4 (3.8) |

BMI: Body mass index
Note:
Number = Number of patients assessed. Percentages are calculated using number of patients assessed as denominator.
^{a}East Europe: Poland, Russia, Turkey and Ukraine; Latin America: Argentina, Chile and Mexico; North America: USA

The patients' disease characteristics at baseline were generally similar across treatment groups in the overall population (Table 3). In the SAR440340+dupilumab group, a higher proportion of patients experienced two or more exacerbations or exacerbations leading to hospitalization in the prior year than patients in other treatment groups. There were also more patients who were former smokers in this treatment group.

Overall, 27.4% of patients had onset of asthma at age<18 years, 36.5% of patients at the age of 18 to 40 years, and 36.1% at the age of ≥40 years. In all, 65.9% of patients were on high dose ICS at baseline and most (85.5%) had an ongoing atopic medical condition. The mean number of asthma exacerbations in the prior year was similar among treatment groups (1.3-1.4), with 26.4% of all randomized patients (78) experiencing more than one asthma exacerbation in the prior year.

Asthma-specific baseline characteristics as well as biomarker baseline characteristics (Table 12) were similar among treatment groups, with an overall mean pre-bronchodilator FEV1 of 2.02 L, mean pre-bronchodilator FEV1 percent predicted 64.69%, mean FEV1 reversibility 14.9%, mean ACQ-5 score (asthma control questionnaire, 5-question version) of 2.16, mean AQLQ global score (asthma quality of life questionnaire with standardized activities) of 4.68, mean eosinophil count of 0.37×10⁹/L, and mean fractional exhaled nitric oxide (FeNO) of 29.2 ppb.

There were differences observed among treatment groups in mean baseline total IgE levels, likely caused by outliers, since the median baseline values were comparable.

TABLE 3
Disease characteristics at baseline, randomized population

| | | | | |
|---|---|---|---|---|
| | SAR440340 | SAR440340 q2w + | Dupilumab | |
| Placebo | 300 mg q2w | Dupilumab q2w | 300 mg q2w | All |
| (N = 74) | (N = 73) | (N = 74) | (N = 75) | (N = 296) |

Background ICS dose level at randomization [n (%)]

| | | | | | |
|---|---|---|---|---|---|
| Number | 74 | 73 | 74 | 75 | 296 |
| High | 48 (64.9) | 45 (61.6) | 49 (66.2) | 53 (70.7) | 195 (65.9) |
| Medium | 26 (35.1) | 28 (38.4) | 25 (33.8) | 22 (29.3) | 101 (34.1) |

Age at onset of asthma (years)

| | | | | | |
|---|---|---|---|---|---|
| Number | 74 | 73 | 74 | 75 | 296 |
| Mean (SD) | 28.8 (17.6) | 31.6 (19.9) | 31.5 (18.1) | 32.6 (17.8) | 31.1 (18.3) |
| Median | 30.5 | 34.0 | 34.5 | 34.0 | 33.0 |
| Q1; Q3 | 12.0; 42.0 | 14.0; 49.0 | 16.0; 44.0 | 19.0; 48.0 | 15.0; 45.5 |
| Min; Max | 0; 63 | 0; 65 | 2; 62 | 1; 63 | 0; 65 |
| <12 | 18 (24.3) | 18 (24.7) | 16 (21.6) | 13 (17.3) | 65 (22.0) |
| ≥12-<18 | 3 (4.1) | 5 (6.8) | 4 (5.4) | 4 (5.3) | 16 (5.4) |
| ≥18-<40 | 31 (41.9) | 22 (30.1) | 24 (32.4) | 31 (41.3) | 108 (36.5) |
| ≥40 | 22 (29.7) | 28 (38.4) | 30 (40.5) | 27 (36.0) | 107 (36.1) |

Time since first diagnosis of asthma at randomization (years)

| | | | | | |
|---|---|---|---|---|---|
| Number | 74 | 73 | 74 | 75 | 296 |
| Mean (SD) | 19.13 (15.13) | 18.14 (13.21) | 18.42 (10.49) | 19.50 (14.68) | 18.80 (13.45) |
| Median | 16.38 | 15.83 | 18.13 | 16.83 | 16.75 |
| Q1; Q3 | 4.75; 29.67 | 7.25; 25.92 | 10.00; 24.92 | 6.83; 30.50 | 6.92; 28.50 |
| Min; Max | 1.3; 60.6 | 1.3; 59.8 | 1.9; 43.0 | 1.3; 54.6 | 1.3; 60.6 |

Time since last asthma exacerbation^{a} at randomization (months)

| | | | | | |
|---|---|---|---|---|---|
| Number | 74 | 73 | 74 | 75 | 296 |
| Mean (SD) | 6.3 (2.9) | 6.5 (2.5) | 6.4 (2.8) | 6.2 (2.9) | 6.3 (2.8) |
| Median | 6.0 | 6.0 | 6.0 | 6.0 | 6.0 |
| Q1; Q3 | 4.0; 8.0 | 5.0; 8.0 | 4.0; 8.0 | 4.0; 8.0 | 4.0; 8.0 |
| Min; Max | 2; 13 | 2; 13 | 2; 13 | 2; 13 | 2; 13 |

Number of asthma exacerbations^{a} experienced within 1 year before screening visit

| | | | | | |
|---|---|---|---|---|---|
| Number | 74 | 73 | 74 | 75 | 296 |
| Mean (SD) | 1.3 (0.8) | 1.3 (0.5) | 1.4 (0.7) | 1.3 (0.6) | 1.3 (0.6) |
| Median | 1.0 | 1.0 | 1.0 | 1.0 | 1.0 |
| Q1; Q3 | 1.0; 1.0 | 1.0; 1.0 | 1.0; 2.0 | 1.0; 1.0 | 1.0; 2.0 |
| Min; Max | 1; 6 | 1; 3 | 1; 4 | 1; 3 | 1; 6 |
| ≤1 | 56 (75.7) | 55 (75.3) | 50 (67.6) | 57 (76.0) | 218 (73.6) |
| 2 | 14 (18.9) | 15 (20.5) | 20 (27.0) | 13 (17.3) | 62 (20.9) |
| 3 | 3 (4.1) | 3 (4.1) | 2 (2.7) | 5 (6.7) | 13 (4.4) |
| ≥4 | 1 (1.4) | 0 | 2 (2.7) | 0 | 3 (1.0) |

Number of asthma exacerbations^{a} requiring hospitalization or urgent medical care visit experienced within 1 year before screening visit

| | | | | | |
|---|---|---|---|---|---|
| Number | 74 | 73 | 74 | 75 | 296 |
| Mean (SD) | 0.4 (0.6) | 0.3 (0.7) | 0.5 (0.8) | 0.3 (0.6) | 0.4 (0.7) |
| Median | 0.0 | 0.0 | 0.0 | 0.0 | 0.0 |
| Q1; Q3 | 0.0; 1.0 | 0.0; 1.0 | 0.0; 1.0 | 0.0; 1.0 | 0.0; 1.0 |
| Min; Max | 0; 2 | 0; 3 | 0; 4 | 0; 3 | 0; 4 |
| 0 | 49 (66.2) | 53 (72.6) | 43 (58.1) | 55 (73.3) | 200 (67.6) |
| 1 | 22 (29.7) | 17 (23.3) | 24 (32.4) | 17 (22.7) | 80 (27.0) |
| 2 | 3 (4.1) | 1 (1.4) | 6 (8.1) | 2 (2.7) | 12 (4.1) |
| 3 | 0 | 2 (2.7) | 0 | 1 (1.3) | 3 (1.0) |
| ≥4 | 0 | 0 | 1 (1.4) | 0 | 1 (0.3) |

With ongoing atopic medical condition^{b} [n (%)]

| | | | | | |
|---|---|---|---|---|---|
| Number | 74 | 73 | 74 | 75 | 296 |
| Yes | 67 (90.5) | 58 (79.5) | 62 (83.8) | 66 (88.0) | 253 (85.5) |

Smoking history [n (%)]

| | | | | | |
|---|---|---|---|---|---|
| Number | 74 | 73 | 74 | 75 | 296 |
| Former | 12 (16.2) | 11 (15.1) | 21 (28.4) | 14 (18.7) | 58 (19.6) |
| Never | 62 (83.8) | 62 (84.9) | 53 (71.6) | 61 (81.3) | 238 (80.4) |

Time since cessation (years)

| | | | | | |
|---|---|---|---|---|---|
| Number | 12 | 11 | 21 | 14 | 58 |
| Mean (SD) | 15.15 (14.14) | 12.84 (7.84) | 14.62 (12.17) | 17.44 (10.72) | 15.07 (11.40) |
| Median | 12.17 | 11.75 | 11.92 | 15.54 | 12.33 |
| Q1; Q3 | 3.96; 23.21 | 5.33; 18.92 | 4.75; 20.75 | 9.50; 27.83 | 5.33; 23.67 |
| Min; Max | 0.7; 46.6 | 3.3; 23.9 | 0.8; 40.9 | 2.9; 33.5 | 0.7; 46.6 |

Pack-year

| | | | | | |
|---|---|---|---|---|---|
| Number | 12 | 11 | 21 | 14 | 58 |
| Mean (SD) | 3.48 (2.92) | 4.33 (2.62) | 3.54 (2.96) | 4.62 (3.08) | 3.94 (2.89) |
| Median | 3.00 | 5.00 | 2.50 | 5.00 | 4.00 |
| Q1; Q3 | 0.95; 5.00 | 2.50; 6.00 | 0.80; 6.00 | 2.20; 7.50 | 1.00; 6.00 |
| Min; Max | 0.1; 8.5 | 0.2; 9.0 | 0.1; 9.0 | 0.2; 9.0 | 0.1; 9.0 |

Blood eosinophil randomization strata^{c} (/mm³) [n (%)]

| | | | | | |
|---|---|---|---|---|---|
| Number | 74 | 73 | 74 | 75 | 296 |
| <150 | 15 (20.3) | 14 (19.2) | 16 (21.6) | 16 (21.3) | 61 (20.6) |
| 150-<300 | 23 (31.1) | 22 (30.1) | 24 (32.4) | 25 (33.3) | 94 (31.8) |
| ≥300 | 36 (48.6) | 37 (50.7) | 34 (45.9) | 34 (45.3) | 141 (47.6) |

Baseline pre-bronchodilator FEV1 (L)

| | | | | | |
|---|---|---|---|---|---|
| Number | 74 | 73 | 74 | 75 | 296 |
| Mean (SD) | 2.12 (0.61) | 1.93 (0.47) | 2.00 (0.57) | 2.04 (0.63) | 2.02 (0.57) |
| Median | 1.93 | 1.85 | 1.88 | 2.01 | 1.90 |
| Q1; Q3 | 1.72; 2.44 | 1.62; 2.23 | 1.59; 2.31 | 1.52; 2.43 | 1.62; 2.33 |
| Min; Max | 1.2; 4.0 | 1.0; 3.2 | 1.0; 3.5 | 1.1; 3.6 | 1.0; 4.0 |

Baseline pre-bronchodilator FEV1 percent predicted (%)

| | | | | | |
|---|---|---|---|---|---|
| Number | 74 | 73 | 74 | 75 | 296 |
| Mean (SD) | 65.90 (9.54) | 63.71 (9.35) | 65.04 (9.00) | 64.10 (10.26) | 64.69 (9.54) |
| Median | 64.64 | 61.45 | 65.27 | 62.86 | 63.82 |
| Q1; Q3 | 58.61; 74.20 | 56.71; 69.96 | 58.97; 70.35 | 55.68; 71.72 | 56.94; 71.83 |
| Min; Max | 47.4; 84.8 | 50.9; 96.7 | 44.4; 83.7 | 49.6; 85.3 | 44.4; 96.7 |

Baseline post-bronchodilator FEV1 (L)

| | | | | | |
|---|---|---|---|---|---|
| Number | 74 | 73 | 74 | 75 | 296 |
| Mean (SD) | 2.44 (0.71) | 2.24 (0.62) | 2.29 (0.71) | 2.30 (0.70) | 2.32 (0.69) |
| Median | 2.30 | 2.19 | 2.18 | 2.26 | 2.22 |
| Q1; Q3 | 1.94; 2.92 | 1.83; 2.54 | 1.80; 2.73 | 1.73; 2.74 | 1.82; 2.72 |
| Min; Max | 1.2; 4.5 | 1.1; 4.0 | 0.9; 4.9 | 1.2; 3.9 | 0.9; 4.9 |

Baseline FEV1 reversibility (%)

| | | | | | |
|---|---|---|---|---|---|
| Number | 74 | 73 | 74 | 75 | 296 |
| Mean (SD) | 15.58 (15.84) | 16.22 (13.27) | 14.52 (17.98) | 13.32 (11.76) | 14.90 (14.87) |
| Median | 12.17 | 13.87 | 11.42 | 10.47 | 12.02 |
| Q1; Q3 | 7.21; 19.58 | 5.31; 24.03 | 3.64; 20.00 | 4.18; 19.23 | 5.13; 20.22 |
| Min; Max | −6.7; 88.1 | −4.3; 51.7 | −11.9; 111.9 | −4.8; 51.5 | −11.9; 111.9 |

Baseline AM PEF (L/min)

| | | | | | |
|---|---|---|---|---|---|
| Number | 74 | 72 | 74 | 75 | 295 |
| Mean (SD) | 335.5 (123.0) | 311.7 (111.0) | 308.6 (100.5) | 302.2 (109.2) | 314.5 (111.3) |
| Median | 317.0 | 297.0 | 281.5 | 292.0 | 299.0 |
| Q1; Q3 | 245.0; 405.0 | 234.5; 391.5 | 222.0; 391.0 | 222.0; 385.0 | 228.0; 392.0 |
| Min; Max | 117; 645 | 121; 635 | 140; 549 | 98; 534 | 98; 645 |

Baseline PM PEF (L/min)

| | | | | | |
|---|---|---|---|---|---|
| Number | 74 | 72 | 73 | 75 | 294 |
| Mean (SD) | 343.8 (120.2) | 323.3 (113.5) | 316.6 (102.5) | 311.4 (109.2) | 323.8 (111.7) |
| Median | 326.5 | 305.5 | 306.0 | 300.0 | 310.0 |
| Q1; Q3 | 247.0; 411.0 | 240.5; 402.5 | 233.0; 389.0 | 231.0; 376.0 | 238.0; 399.0 |
| Min; Max | 76; 631 | 128; 668 | 123; 556 | 96; 565 | 76; 668 |

Baseline AM asthma symptom score

| | | | | | |
|---|---|---|---|---|---|
| Number | 74 | 73 | 74 | 75 | 296 |
| Mean (SD) | 0.85 (0.78) | 0.77 (0.71) | 0.93 (0.68) | 1.00 (0.83) | 0.89 (0.76) |
| Median | 0.92 | 0.86 | 1.00 | 1.00 | 1.00 |
| Q1; Q3 | 0.14; 1.00 | 0.14; 1.00 | 0.43; 1.29 | 0.29; 1.57 | 0.18; 1.24 |
| Min; Max | 0.0; 3.0 | 0.0; 3.6 | 0.0; 2.6 | 0.0; 3.9 | 0.0; 3.9 |

Baseline PM asthma symptom score

| | | | | | |
|---|---|---|---|---|---|
| Number | 74 | 73 | 74 | 75 | 296 |
| Mean (SD) | 1.09 (0.80) | 0.96 (0.75) | 1.02 (0.72) | 1.14 (0.84) | 1.05 (0.78) |
| Median | 1.00 | 0.86 | 1.00 | 1.17 | 1.00 |
| Q1; Q3 | 0.43; 1.71 | 0.33; 1.43 | 0.29; 1.50 | 0.33; 1.71 | 0.37; 1.67 |
| Min; Max | 0.0; 3.0 | 0.0; 2.9 | 0.0; 2.4 | 0.0; 4.0 | 0.0; 4.0 |

Baseline number of nocturnal awakenings

| | | | | | |
|---|---|---|---|---|---|
| Number | 74 | 73 | 74 | 75 | 296 |
| Mean (SD) | 0.35 (0.86) | 0.20 (0.51) | 0.32 (0.60) | 0.41 (0.79) | 0.32 (0.71) |
| Median | 0.00 | 0.00 | 0.00 | 0.00 | 0.00 |
| Q1; Q3 | 0.00; 0.14 | 0.00; 0.14 | 0.00; 0.33 | 0.00; 0.57 | 0.00; 0.31 |
| Min; Max | 0.0; 5.4 | 0.0; 3.2 | 0.0; 3.3 | 0.0; 5.0 | 0.0; 5.4 |

Baseline ACQ-5 score

| | | | | | |
|---|---|---|---|---|---|
| Number | 74 | 73 | 74 | 75 | 296 |
| Mean (SD) | 2.19 (0.38) | 2.12 (0.40) | 2.07 (0.38) | 2.25 (0.41) | 2.16 (0.40) |
| Median | 2.20 | 2.20 | 2.00 | 2.20 | 2.20 |
| Q1; Q3 | 2.00; 2.40 | 1.80; 2.40 | 1.80; 2.40 | 2.00; 2.60 | 1.80; 2.40 |
| Min; Max | 1.4; 3.0 | 1.4; 2.8 | 1.4; 3.0 | 1.2; 3.0 | 1.2; 3.0 |

Baseline global AQLQ(S) score

| | | | | | |
|---|---|---|---|---|---|
| Number | 74 | 73 | 74 | 74 | 295 |
| Mean (SD) | 4.68 (0.95) | 4.58 (0.94) | 4.77 (0.79) | 4.67 (0.90) | 4.68 (0.90) |
| Median | 4.75 | 4.63 | 4.77 | 4.67 | 4.66 |
| Q1; Q3 | 3.97; 5.31 | 3.97; 5.28 | 4.25; 5.38 | 4.03; 5.31 | 4.03; 5.31 |
| Min; Max | 2.2; 6.9 | 1.7; 6.6 | 2.6; 6.6 | 2.1; 6.7 | 1.7; 6.9 |

Baseline global RQLQ(S) score^{d}

| | | | | | |
|---|---|---|---|---|---|
| Number | 52 | 41 | 43 | 49 | 185 |
| Mean (SD) | 2.19 (1.35) | 1.92 (1.28) | 1.88 (1.05) | 2.02 (0.90) | 2.01 (1.16) |
| Median | 2.07 | 1.57 | 1.71 | 1.89 | 1.86 |
| Q1; Q3 | 1.11; 3.07 | 1.07; 2.46 | 1.14; 2.36 | 1.29; 2.61 | 1.21; 2.64 |
| Min; Max | 0.2; 5.6 | 0.0; 6.0 | 0.0; 5.0 | 0.3; 3.9 | 0.0; 6.0 |

Baseline number of inhalations of salbutamol/albuterol and levosalbutamol/levabuterol/24 hrs

| | | | | | |
|---|---|---|---|---|---|
| Number | 74 | 73 | 74 | 75 | 296 |
| Mean (SD) | 2.2 (3.0) | 1.8 (2.0) | 2.1 (2.2) | 2.7 (3.2) | 2.2 (2.7) |
| Median | 1.0 | 1.0 | 2.0 | 2.0 | 1.0 |
| Q1; Q3 | 0.0; 4.0 | 0.0; 3.0 | 0.0; 4.0 | 0.0; 4.0 | 0.0; 4.0 |
| Min; Max | 0; 13 | 0; 9 | 0; 8 | 0; 14 | 0; 14 |

With hypersensitivity to aspirin [n (%)]

| | | | | | |
|---|---|---|---|---|---|
| Number | 74 | 73 | 74 | 75 | 296 |
| Yes | 5 (6.8) | 6 (8.2) | 4 (5.4) | 6 (8.0) | 21 (7.1) |
| Ongoing condition | 5 (6.8) | 6 (8.2) | 4 (5.4) | 6 (8.0) | 21 (7.1) |

With hypersensitivity to NSAID [n (%)]

| | | | | | |
|---|---|---|---|---|---|
| Number | 74 | 73 | 74 | 75 | 296 |
| Yes | 5 (6.8) | 5 (6.8) | 3 (4.1) | 5 (6.7) | 18 (6.1) |
| Ongoing condition | 5 (6.8) | 5 (6.8) | 3 (4.1) | 5 (6.7) | 18 (6.1) |

ICS: Inhaled corticosteroid, FEV1: Forced expiratory volume in one second, PEF: Peak expiratory flow, ACQ-5: Asthma control questionnaire, 5 question version, AQLQ(S): Asthma quality of life questionnaire with standardized activities, RQLQ(S): Rhinoconjunctivitis quality of life questionnaire with standardized activities, NSAID: Nonsteroidal anti-inflammatory drug, IRT: Interactive response technology.
^{a}Asthma exacerbation prior to the study is defined as any treatment with 1 systemic (oral or parenteral) steroid burst or more for worsening asthma or hospitalization or an emergency medical care visit for worsening asthma.
^{b}A patient is considered to have atopic medical condition if he/she has any of the following ongoing conditions: atopic dermatitis, allergic conjunctivitis, allergic rhinitis, eosinophilic esophagitis, food allergy, hives; or has baseline total IgE >=100 IU/mL.
^{c}Randomization strata from IRT system.
^{d}Assessed only in patients with comorbid allergic rhinitis.

Dosage and Duration

The extent of exposure to the investigational medicinal product (IMP) in the safety population is summarized in Table 13. The mean duration of treatment exposure was 77.3 days for the SAR440340 group, 72.7 days for the SAR440340+dupilumab group, 76.2 days for the dupilumab group, and 70.6 days for the placebo group. The lower exposure observed in the placebo arm was caused by the higher discontinuation rate due to LOAC. Cumulative exposure to SAR440340 alone and in combination with dupilumab was 30.2 patient-years.

Efficacy

Primary Efficacy Endpoint

The primary endpoint of the study was the proportion of patients with loss of asthma control (LOAC). The incidence of LOAC was lower for patients in the SAR440340 and SAR440340+dupilumab groups in comparison with the placebo group: 21.9% and 27% versus 40.5%. SAR440340 monotherapy significantly reduced the odds of experiencing LOAC compared to placebo; p=0.02. Odds ratio reduction in the SAR440340+dupilumab arm compared to placebo was not significant (p=0.07). Odds ratios (95% CIs) were 0.423 (0.203 to 0.880) for SAR440340 versus placebo and 0.520 (0.256 to 1.057) for the combination versus placebo (Table 4).

SAR440340 in combination with dupilumab did not reduce the odds of experiencing LOAC compared to SAR440340 (p=0.60) or compared to dupilumab (p=0.25). Odds ratios (95% CIs) were 1.231 (0.571 to 2.653) for comparison with SAR440340 and 1.589 (0.723 to 3.492) for comparison with dupilumab (Table 14). Sensitivity analyses confirmed conclusions of the primary analysis.

TABLE 4
Primary approach: Incidence of LOAC (primary comparisons), mITT population

| | | SAR440340 | SAR440340 q2w + | Dupilumab |
|---|---|---|---|---|
| | Placebo | 300 mg q2w | Dupilumab q2w | 300 mg q2w |
| Number of patients with LOAC | (N = 74) | (N = 73) | (N = 74) | (N = 74) |
| Number | 74 | 73 | 74 | 74 |
| No | 44 (59.5) | 57 (78.1) | 54 (73.0) | 60 (81.1) |
| Yes | 30 (40.5) | 16 (21.9) | 20 (27.0) | 14 (18.9) |
| OR vs placebo (95% CI)^{a} | | 0.423 (0.203 to 0.880) | 0.520 (0.256 to 1.057) | |
| P-value vs placebo^{a} | | 0.0214 | 0.0709 | |

LOAC: Loss of asthma control, OR: Odds ratio, ICS: Inhaled corticosteroid.
^{a}Derived from logistic regression with treatment, baseline eosinophil strata, region, background ICS dose level at randomization and number of exacerbation events within 1 year prior to screening.

Overall, 80 LOAC events were reported across the treatment groups during the study. Seventy-eight discontinued due to LOAC as per protocol. Two patients had concurrent occurrence of LOAC and adverse event and investigators considered the primary reason for treatment withdrawal the adverse event. The majority of the LOAC were due to reductions from baseline in morning PEF, followed by increase in asthma reliever puffs and use of systemic steroids (Table 15).

Although there was no evidence of a differential treatment effect across subgroups defined by baseline eosinophil count (p=0.5365 for the treatment by subgroup interaction), the number of patients within each subgroup was small. SAR440340 monotherapy showed reduction of LOAC across all subgroups based on the baseline eosinophil count, with odds ratios 0.401 (0.072 to 2.239) for <150/mm³, 0.473 (0.099 to 2.270) for ≥150-<300/mm³, and 0.388 (0.143 to 1.054) for 300/mm³.

SAR440340 in combination with dupilumab showed a numerical reduction of LOAC in the subgroup of patients with baseline eosinophil count<150/mm³ and 300/mm³, with odds ratios 0.623 (0.122 to 3.182) and 0.298 (0.102 to 0.868), respectively. For the subgroup of patients with baseline eosinophil count 150-<300/mm³, there was no reduction observed, odds ratio 1.086 (0.270 to 4.372). (Table 16).

Main Secondary Key Efficacy Endpoints

Pre-Bronchodilator FEV1 Change from Baseline at Week 12

The LS mean difference in change from baseline to week 12 in pre-bronchodilator FEV1 (95% CIs, p-value) was significant for the SAR440340 group (0.14 [0.01 to 0.27], p=0.03), but not for the SAR440340+dupilumab group (0.10 [−0.03 to 0.23], p=0.13) (Table 5).

The LS mean difference in change from baseline to week 12 in pre-bronchodilator FEV1 in the SAR440340+dupilumab group versus the SAR440340 group was −0.04 ([−0.17 to 0.09], p=0.53). The LS mean difference in change from baseline to week 12 in pre-bronchodilator FEV1 in the SAR440340+dupilumab group versus the dupilumab group was −0.06 ([−0.19 to 0.06], p=0.33).

Change from baseline in pre-bronchodilator FEV1 at week 12 for all comparisons is presented at Table 17.

TABLE 5
Primary approach: Change from baseline in pre-bronchodilator
FEV1 (L) at week 12 (primary comparisons) - mITT population

| | | | | |
|---|---|---|---|---|
| | | SAR440340 | SAR440340 q2w + | Dupilumab |
| | Placebo | 300 mg q2w | Dupilumab q2w | 300 mg q2w |
| Pre-bronchodilator FEV1 (L) | (N = 74) | (N = 73) | (N = 74) | (N = 74) |

Baseline

| | | | | |
|---|---|---|---|---|
| Number | 74 | 73 | 74 | 74 |
| Mean (SD) | 2.12 (0.61) | 1.93 (0.47) | 2.00 (0.57) | 2.03 (0.62) |
| Median | 1.93 | 1.85 | 1.88 | 1.99 |
| Q1; Q3 | 1.72; 2.44 | 1.62; 2.23 | 1.59; 2.31 | 1.52; 2.27 |
| Min; Max | 1.2; 4.0 | 1.0; 3.2 | 1.0; 3.5 | 1.1; 3.6 |

Week 12

| | | | | |
|---|---|---|---|---|
| Number | 41 | 58 | 49 | 56 |
| Mean (SD) | 2.32 (0.74) | 2.03 (0.60) | 2.01 (0.59) | 2.20 (0.81) |
| Median | 2.17 | 1.92 | 1.86 | 2.11 |
| Q1; Q3 | 1.73; 2.85 | 1.68; 2.19 | 1.55; 2.59 | 1.58; 2.70 |
| Min; Max | 1.2; 4.3 | 1.0; 3.8 | 0.9; 3.2 | 0.8; 4.5 |

Change from baseline

| | | | | |
|---|---|---|---|---|
| Number | 41 | 58 | 49 | 56 |
| Mean (SD) | 0.06 (0.35) | 0.11 (0.34) | 0.06 (0.37) | 0.14 (0.43) |
| Median | 0.01 | 0.06 | −0.03 | 0.06 |
| Q1; Q3 | −0.18; 0.21 | −0.11; 0.34 | −0.13; 0.29 | −0.18; 0.39 |
| Min; Max | −0.6; 1.2 | −0.7; 1.0 | −1.0; 1.0 | −0.9; 1.4 |
| LS Mean (SE)^{a} | −0.04 (0.05) | 0.10 (0.05) | 0.06 (0.05) | 0.12 (0.05) |
| LS Mean diff vs. placebo | | 0.14 (0.01 to 0.27) | 0.10 (−0.03 to 0.23) | |
| (95% CI)^{a} | | | | |
| P-value vs. placebo^{a} | | 0.0344 | 0.1337 | |
| LS Mean diff vs. | | | −0.04 (−0.17 to 0.09) | |
| SAR440340 (95% CI)^{a} | | | | |
| P-value vs. SAR440340^{a} | | | 0.5289 | |
| LS Mean diff vs. | | | −0.06 (−0.19 to 0.06) | |
| dupilumab (95% CI)^{a} | | | | |
| P-value vs. dupilumab^{a} | | | 0.3293 | |

FEV1: Forced expiratory volume in one second, LOAC: Loss of asthma control, ICS: Inhaled corticosteroid.
Data collected up to end of treatment visit were included. For patients who experienced LOAC with rescue medications, FEV1 collected at and after start of the event were set to missing. Missing data were not imputed.
^{a}Derived from MMRM model with change from baseline values up to week 12 as the response variable, and treatment, sex, baseline height, baseline eosinophil strata, region, background ICS dose level at randomization, visit, treatment by-visit interaction, baseline value and baseline-by-visit interaction as covariates.

Sensitivity and per protocol analyses confirmed conclusions of the primary analysis. Although there was no evidence of a differential treatment effect across subgroups defined by baseline eosinophil count (p=0.1111 for the treatment by subgroup interaction), the number of patients within each subgroup was small.

For the subgroup of patients with baseline eosinophil count≥300/mm³, the LS mean difference in change from baseline to week 12 in pre-bronchodilator FEV1 (0.22 [0.02 to 0.41]) was significant for the comparison of SAR440340 alone vs placebo. SAR440340 in combination with dupilumab showed a trend towards improvement, with LS mean 0.19 (−0.01 to 0.40).

For the subgroup of patients with baseline eosinophil count≥150-<300/mm³, the LS mean difference in change from baseline to week 12 in pre-bronchodilator FEV1 was not significant for SAR440340 alone or in combination with dupilumab. The high FEV1 in the placebo group may have resulted in the negative trajectory of the FEV1 across all treatment groups.

In the subgroup of patients with baseline eosinophil count<150/mm³, the LS mean difference in change from baseline to week 12 in pre-bronchodilator FEV1 showed a trend towards improvement: 0.17 (−0.12 to 0.45) for the SAR440340 group and 0.11 (−0.17 to 0.39) for the SAR440340+dupilumab group (Table 18).

Post-Bronchodilator FEV1 Change from Baseline at Week 12

The LS mean difference in change from baseline to week 12 in post-bronchodilator FEV1 was significant for the SAR440340+dupilumab group (0.13 [0.01 to 0.25], p=0.04) but not for the SAR440340 group (0.05 [−0.07 to 0.17], p=0.41) (Table 6).

The LS mean difference in change from baseline to week 12 in post-bronchodilator FEV1 in the SAR440340+dupilumab group versus the SAR440340 group was (0.08 [−0.04 to 0.19], p=0.19). The LS mean difference in absolute change from baseline to week 12 in post-bronchodilator FEV1 in the SAR440340+dupilumab group versus the dupilumab group was (−0.02 [−0.13 to 0.10], p=0.78). Sensitivity and per protocol analyses confirmed the conclusions of the primary analysis.

TABLE 6
Primary approach: Change from baseline in post-bronchodilator
FEV1 (L) at week 12 (primary comparisons), mITT population

| | | | | |
|---|---|---|---|---|
| | | SAR440340 | SAR440340 q2w + | Dupilumab |
| | Placebo | 300 mg q2w | Dupilumab q2w | 300 mg q2w |
| Post-bronchodilator FEV1 (L) | (N = 74) | (N = 73) | (N = 74) | (N = 74) |

Baseline

| | | | | |
|---|---|---|---|---|
| Number | 74 | 73 | 74 | 74 |
| Mean (SD) | 2.44 (0.71) | 2.24 (0.62) | 2.29 (0.71) | 2.30 (0.70) |
| Median | 2.30 | 2.19 | 2.18 | 2.24 |
| Q1; Q3 | 1.94; 2.92 | 1.83; 2.54 | 1.80; 2.73 | 1.73; 2.74 |
| Min; Max | 1.2; 4.5 | 1.1; 4.0 | 0.9; 4.9 | 1.2; 3.9 |

Week 12

| | | | | |
|---|---|---|---|---|
| Number | 42 | 58 | 51 | 57 |
| Mean (SD) | 2.54 (0.75) | 2.23 (0.64) | 2.24 (0.64) | 2.43 (0.86) |
| Median | 2.32 | 2.14 | 2.14 | 2.39 |
| Q1; Q3 | 2.04; 2.92 | 1.83; 2.48 | 1.73; 2.82 | 1.82; 2.98 |
| Min; Max | 1.1; 4.6 | 1.1; 3.9 | 1.0; 3.4 | 1.0; 4.7 |

Change from baseline

| | | | | |
|---|---|---|---|---|
| Number | 42 | 58 | 51 | 57 |
| Mean (SD) | −0.02 (0.27) | −0.00 (0.33) | 0.06 (0.41) | 0.09 (0.42) |
| Median | −0.02 | −0.05 | 0.03 | 0.03 |
| Q1; Q3 | −0.09; 0.08 | −0.17; 0.11 | −0.09; 0.26 | −0.15; 0.28 |
| Min; Max | −1.0; 0.5 | −0.7; 1.4 | −2.0; 0.8 | −0.7; 1.6 |
| LS Mean (SE)^{a} | −0.05 (0.05) | −0.00 (0.04) | 0.07 (0.04) | 0.09 (0.04) |
| LS Mean diff vs. placebo | | 0.05 (−0.07 to 0.17) | 0.13 (0.01 to 0.25) | |
| (95% CI)^{a} | | | | |
| P-value vs. placebo^{a} | | 0.4085 | 0.0379 | |
| LS Mean diff vs. | | | 0.08 (−0.04 to 0.19) | |
| SAR440340 (95% CI)^{a} | | | | |
| P-value vs. SAR440340^{a} | | | 0.1935 | |
| LS Mean diff vs. | | | −0.02 (−0.13 to 0.10) | |
| dupilumab (95% CI)^{a} | | | | |
| P-value vs. dupilumab^{a} | | | 0.7800 | |

FEV1: Forced expiratory volume in one second,
LOAC: Loss of asthma control,
ICS: Inhaled corticosteroid
Data collected up to end of treatment visit were included. For patients who experienced LOAC with rescue medications, FEV1 collected at and after start of the event were set to missing. Missing data were not imputed.
^{a}Derived from MMRM model with change from baseline values up to Week 12 as the response variable, and treatment, sex, baseline height, baseline eosinophil strata, region, background ICS dose level at randomization, visit, treatment by-visit interaction, baseline value and baseline-by-visit interaction as covariates.

The study was well-performed, the four treatment arms were balanced on baseline characteristics, and the dupilumab arm, used as a calibrator, showed the expected outcomes in term of safety and efficacy (ITT: LOAC: 53% RR; FEV1: 160 mL; better efficacy in Eos≥300/μl: LOAC: 72% RR; FEV1: 340 mL).

SAR440340 showed a favorable safety profile, and demonstrated significant efficacy on all endpoints (ITT: LOAC 42% RR; FEV1: 140 mL; ACQS and AQLQ, mean change vs placebo −0.42 and 0.45, resp.).

Although LOAC data point to a more uniform efficacy profile across low/high blood Eos* (SAR: 42%/45%; dupi: 30%/72%) and FeNO* strata (SAR: 48%/39%; dupi: 43%/67%), FEV1 data profile resembled that of dupilumab, with superior efficacy in Type 2 high (220 mL in high Eos* vs 20 mL in low Eos; 200 mL in high FeNO* vs 70 mL in low FeNO).

Beyond the expected decrease of blood Eos, there was no significant impact on all other Type 2 biomarkers.

Safety

Safety findings indicated that SAR440340 could be safely used in patients with moderate-to-severe asthma. SAR440340, alone and in combination with dupilumab, was safe and generally well-tolerated. Overall treatment discontinuation rate due to AEs was low. The overall rate of AESIs or other selected AE grouping events was low. The most frequent events were hypersensitivity and injection site reactions. ISRs were less frequent in the SAR440340 monotherapy arm.

Treatment-Emergent Adverse Events (TEAEs)

The incidence of TEAEs was similar across the treatment groups. The number of treatment emergent SAEs and TEAEs leading to discontinuation was low (Table 7). One patient died in dupilumab group due to ethyl alcohol poisoning in post-treatment follow-up (information received after database lock).

TABLE 7
Overview of adverse event profile: Treatment-emergent adverse events, safety population

| | | SAR440340 | SAR440340 q2w + | Dupilumab |
|---|---|---|---|---|
| | Placebo | 300 mg q2w | Dupilumab q2w | 300 mg q2w |
| n (%) | (N = 74) | (N = 73) | (N = 74) | (N = 74) |
| Patients with any TEAE | 48 (64.9) | 44 (60.3) | 49 (66.2) | 41 (55.4) |
| Patients with any severe TEAE | 2 (2.7) | 0 | 1 (1.4) | 1 (1.4) |
| Patients with any treatment-emergent | 3 (4.1) | 1 (1.4) | 1 (1.4) | 1 (1.4) |
| SAE | | | | |
| Patients with any TEAE leading to | 0 | 0 | 0 | 0 |
| death | | | | |
| Patients with any TEAE leading to | 3 (4.1) | 0 | 2 (2.7) | 0 |
| permanent treatment discontinuation | | | | |
| Patients with any TEAE related to | 5 (6.8) | 1 (1.4) | 8 (10.8) | 3 (4.1) |
| SAR440340 or matching placebo^{a} | | | | |
| Patients with any TEAE related to | 7 (9.5) | 1 (1.4) | 7 (9.5) | 8 (10.8) |
| dupilumab or matching placebo^{a} | | | | |

TEAE: Treatment-emergent adverse event, SAE: Serious adverse event, IMP: investigational medicinal product.
n (%) = number and percentage of patients with at least one TEAE.
^{a}As reported by the investigator.

The majority of the TEAEs were mild or moderate intensity. The number of severe TEAEs was low. Out of 4 events, 3 events were treatment-emergent SAEs and 1 event was AESI of severe injection site reaction.

Table 8 presents the number (%) of patients with TEAEs that occurred with a frequency of ≥3% in any treatment group by primary SOC and preferred term (PT). The most frequent TEAEs were in the SOCs of infections and infestations, mainly due to nasopharyngitis and viral upper respiratory tract infection, and nervous system disorders, mainly due to headache. Slightly more patients in the SAR440340 group experienced nasopharyngitis compare to other treatment groups, but there was lower frequency of other upper respiratory tract infections in the arm when compared to other arms. The incidence of injection site reactions (PT injection site erythema and injection site rash) was less frequent in SAR440340 group when compared to other treatment groups.

There were three patients with PT rash pruritic reported in the SAR440340+dupilumab group compare to none patient in other treatment arms. Two of the events were assessed as AESI of hypersensitivity.

As per e-CRF reporting rules, the relationship to SAR440340/matching placebo and dupilumab/matching placebo should be assessed separately for each adverse event. For most of the events, with the exception of injection site reactions, the assessment can be difficult and the investigators may tend to relate adverse events to both study medications. Due to this database set-up, there were two patients in dupilumab group with TEAEs assessed related to both dupilumab/matching placebo and SAR440340/matching placebo. One patient experienced nausea and tachycardia the day of the IMP administration, and a second patient experienced neutropenia. Additionally, in the SAR440340 group, there was one patient with injection site reaction reported related to dupilumab/matching placebo in error, and in the dupilumab group there was one patient who experienced an injection site reaction in the SAR440340/matching placebo site in addition to injection site reaction in the dupilumab injection site.

TABLE 8
Number (%) of patients with TEAE(s) that occurred with a frequency >=3%
in any treatment group by primary SOC and PT, safety population

| PRIMARY SYSTEM ORGAN | | SAR440340 | SAR440340 q2w + | Dupilumab |
|---|---|---|---|---|
| CLASS | Placebo | 300 mg q2w | Dupilumab q2w | 300 mg q2w |
| Preferred Term n(%) | (N = 74) | (N = 73) | (N = 74) | (N = 74) |
| Any Class | 48 (64.9) | 44 (60.3) | 49 (66.2) | 41 (55.4) |
| INFECTIONS AND INFESTATIONS | 27 (36.5) | 27 (37.0) | 25 (33.8) | 23 (31.1) |
| Nasopharyngitis | 9 (12.2) | 12 (16.4) | 8 (10.8) | 9 (12.2) |
| Viral upper respiratory tract | 5 (6.8) | 3 (4.1) | 5 (6.8) | 2 (2.7) |
| infection | | | | |
| Upper respiratory tract infection | 2 (2.7) | 1 (1.4) | 4 (5.4) | 2 (2.7) |
| bacterial | | | | |
| Urinary tract infection | 1 (1.4) | 1 (1.4) | 3 (4.1) | 2 (2.7) |
| NERVOUS SYSTEM DISORDERS | 8 (10.8) | 7 (9.6) | 5 (6.8) | 10 (13.5) |
| Headache | 7 (9.5) | 6 (8.2) | 5 (6.8) | 10 (13.5) |
| RESPIRATORY, THORACIC AND | 11 (14.9) | 8 (11.0) | 4 (5.4) | 4 (5.4) |
| MEDIASTINAL DISORDERS | | | | |
| Cough | 5 (6.8) | 1 (1.4) | 0 | 1 (1.4) |
| Dyspnoea | 3 (4.1) | 0 | 0 | 0 |
| GASTROINTESTINAL DISORDERS | 7 (9.5) | 8 (11.0) | 8 (10.8) | 5 (6.8) |
| Nausea | 2 (2.7) | 4 (5.5) | 2 (2.7) | 1 (1.4) |
| SKIN AND SUBCUTANEOUS | 1 (1.4) | 3 (4.1) | 4 (5.4) | 4 (5.4) |
| TISSUE DISORDERS | | | | |
| Rash pruritic | 0 | 0 | 3 (4.1) | 0 |
| MUSCULOSKELETAL AND | 5 (6.8) | 7 (9.6) | 2 (2.7) | 9 (12.2) |
| CONNECTIVE TISSUE | | | | |
| DISORDERS | | | | |
| Arthralgia | 1 (1.4) | 1 (1.4) | 1 (1.4) | 4 (5.4) |
| Back pain | 1 (1.4) | 3 (4.1) | 1 (1.4) | 1 (1.4) |
| GENERAL DISORDERS AND | 7 (9.5) | 3 (4.1) | 8 (10.8) | 7 (9.5) |
| ADMINISTRATION SITE | | | | |
| CONDITIONS | | | | |
| Injection site reaction | 4 (5.4) | 1 (1.4) | 4 (5.4) | 4 (5.4) |
| Injection site erythema | 2 (2.7) | 1 (1.4) | 3 (4.1) | 1 (1.4) |

TEAE: Treatment-emergent adverse event,
SOC: System organ class,
PT: Preferred term
MedDRA 21.1.
n (%) = number and percentage of patients with at least one TEAE.
Note:
Table sorted by SOC internationally agreed order and decreasing frequency of PT in the coadministration group.
Only SOC with at least one PT >=3% in at least one group are presented.

Deaths, Serious Treatment-Emergent Adverse Events

There was one patient in dupilumab group who died during post-treatment follow-up. The patient was determined as the patient lost to follow-up at the time of database lock. After database lock new information has been received that the patient died due to ethyl alcohol poisoning, reported not related to IMP. The patient was 62-year old male from Russia with medical history of arterial hypertension. At screening, he reported that he drinks more than 2 drinks of alcohol at least weekly. The patient discontinued treatment due to LOAC (having 30% or greater reduction in morning PEF on 2 consecutive days) after 4th IMP administration. He died about 40 days after last IMP dose. During the study the patient reported no adverse event, his ECG and laboratory results were unremarkable.

The incidence of treatment-emergent SAEs was low. Treatment-emergent SAEs by PT were reported by single patients only (Table 9).

TABLE 9
Number (%) of patients with treatment-emergent
SAE(s) by Primary SOC and PT, safety population

| PRIMARY SYSTEM ORGAN | | SAR440340 | SAR440340 q2w + | Dupilumab |
|---|---|---|---|---|
| CLASS | Placebo | 300 mg q2w | Dupilumab q2w | 300 mg q2w |
| Preferred Term n(%) | (N = 74) | (N = 73) | (N = 74) | (N = 74) |
| Any Class | 3 (4.1) | 1 (1.4) | 1 (1.4) | 1 (1.4) |
| INFECTIONS AND INFESTATIONS | 0 | 0 | 0 | 1 (1.4) |
| Abscess jaw | 0 | 0 | 0 | 1 (1.4) |
| NERVOUS SYSTEM DISORDERS | 1 (1.4) | 0 | 0 | 0 |
| Vascular encephalopathy | 1 (1.4) | 0 | 0 | 0 |
| RESPIRATORY, THORACIC AND | 1 (1.4) | 1 (1.4) | 0 | 0 |
| MEDIASTINAL DISORDERS | | | | |
| Asthma | 0 | 1 (1.4) | 0 | 0 |
| Nasal polyps | 1 (1.4) | 0 | 0 | 0 |
| HEPATOBILIARY DISORDERS | 1 (1.4) | 0 | 0 | 0 |
| Cholecystitis acute | 1 (1.4) | 0 | 0 | 0 |
| INVESTIGATIONS | 0 | 0 | 1 (1.4) | 0 |
| Alanine aminotransferase | 0 | 0 | 1 (1.4) | 0 |
| increased | | | | |

SAE: Serious adverse event,
SOC: System organ class,
PT: Preferred term
MedDRA 21.1.
n (%) = number and percentage of patients with at least one treatment-emergent SAE.
Note:
Table sorted by SOC internationally agreed order and decreasing frequency of PT in the coadministration group.

Adverse Events Leading to Withdrawal

The overall treatment discontinuation rate due to AEs was low (Table 10). Only 5 patients discontinued treatment due to a TEAE(s) (2 in the SAR440340+dupilumab group and 3 in the placebo group). As per protocol, 2 patients discontinued IMP due to systemic hypersensitivity considered related to IMP and requiring treatment (PT rash pruritic and urticaria).

TABLE 10
Number (%) of patients with TEAE(s) leading to permanent treatment
discontinuation by primary SOC and PT, safety population

| PRIMARY SYSTEM ORGAN | | SAR440340 | SAR440340 q2w + | Dupilumab |
|---|---|---|---|---|
| CLASS | Placebo | 300 mg q2w | Dupilumab q2w | 300 mg q2w |
| Preferred Term n(%) | (N = 74) | (N = 73) | (N = 74) | (N = 74) |
| Any Class | 3 (4.1) | 0 | 2 (2.7) | 0 |
| CARDIAC DISORDERS | 1 (1.4) | 0 | 0 | 0 |
| Palpitations | 1 (1.4) | 0 | 0 | 0 |
| HEPATOBILIARY DISORDERS | 1 (1.4) | 0 | 0 | 0 |
| Cholecystitis acute | 1 (1.4) | 0 | 0 | 0 |
| SKIN AND SUBCUTANEOUS | 1 (1.4) | 0 | 1 (1.4) | 0 |
| TISSUE DISORDERS | | | | |
| Rash pruritic | 0 | 0 | 1 (1.4) | 0 |
| Urticaria | 1 (1.4) | 0 | 0 | 0 |
| GENERAL DISORDERS AND | 0 | 0 | 1 (1.4) | 0 |
| ADMINISTRATION SITE | | | | |
| CONDITIONS | | | | |
| Injection site reaction | 0 | 0 | 1 (1.4) | 0 |

TEAE: Treatment-emergent adverse event,
SOC: System organ class,
PT: Preferred term
Medora 21.1
n (%) = number and percentage of patients with at least one TEAE leading to permanent treatment discontinuation
Note:
Table sorted by SOC internationally agreed order and decreasing frequency of PT in the coadministration group.
PGM = DEVOPS/SAR440340/ACT15102/CSR/REPORT/PGM/ae_socpt_s_t.sas
OUT = REPORT/OUTPUT/ae_socpt_disc_s_t_i.rtf (22 APR. 2019 5:16)

Other Significant Adverse Events (Including AESI, Labs)

Table 11 provides an overview of the number (%) of patients with a treatment-emergent AESI or other selected AE grouping event. The overall rate of AESIs or other selected AE grouping events was low. The most frequent events were hypersensitivity and injection site reactions.

TABLE 11
Number (%) of patients with treatment-emergent AESIs and other
selected AE grouping events by category, safety population

| | | SAR440340 | SAR440340 q2w + | Dupilumab |
|---|---|---|---|---|
| | Placebo | 300 mg q2w | Dupilumab q2w | 300 mg q2w |
| Category n(%) | (N = 74) | (N = 73) | (N = 74) | (N = 74) |
| Any treatment-emergent AESI | 3 (4.1) | 4 (5.5) | 5 (6.8) | 2 (2.7) |
| Anaphylactic reaction (medically | 0 | 0 | 0 | 0 |
| reviewed) | | | | |
| Hypersensitivity (medically | 1 (1.4) | 3 (4.1) | 3 (4.1) | 1 (1.4) |
| reviewed) | | | | |
| Injection site reaction (serious or | 0 | 0 | 1 (1.4) | 0 |
| severe and lasting 24 hours or | | | | |
| longer) | | | | |
| Infection | 2 (2.7) | 0 | 0 | 1 (1.4) |
| Parasitic infection | 0 | 0 | 0 | 0 |
| Opportunistic infection | 0 | 0 | 0 | 0 |
| Potential drug-related hepatic | 0 | 0 | 1 (1.4) | 0 |
| disorder | | | | |
| Malignancy | 0 | 0 | 0 | 0 |
| Pregnancy | 0 | 1 (1.4) | 0 | 0 |
| Symptomatic overdose with IMP | 0 | 0 | 0 | 0 |
| Symptomatic overdose with NIMP | 0 | 0 | 0 | 0 |
| Any treatment-emergent other AE | 6 (8.1) | 4 (5.5) | 7 (9.5) | 6 (8.1) |
| grouping event | | | | |
| Injection site reaction | 5 (6.8) | 3 (4.1) | 7 (9.5) | 6 (8.1) |
| Eosinophilia | 1 (1.4) | 1 (1.4) | 0 | 0 |

AESI: Adverse event of special interest,
IMP: Investigational medicinal product,
NIMP: Noninvestigational medicinal product
MedDRA 21.1
n (%) = number and percentage of patients with at least one treatment-emergent AESI/other AE grouping event
Note:
This table is based on MedDRA SMQ or company defined search criteria.

Adverse Events of Special Interest (AESI)

Anaphylactic Reaction/Systemic Hypersensitivity

The incidence of hypersensitivity was slightly higher in the SAR440340 group and the SAR440340+dupilumab group compared to the placebo and the dupilumab groups. Hypersensitivity was reported for 3 (4.1%) patients each in the SAR440340 group and the SAR440340+dupilumab group, and for 1 (1.4%) patient each in the dupilumab and the placebo groups. Hypersensitivity events included PTs of lip swelling, pruritus, and pruritus generalized in the SAR440340 group; rash pruritic and hypersensitivity in the SAR440340+dupilumab group; conjunctivitis in the dupilumab group; and urticaria in the placebo group. All TEAEs of hypersensitivity were mild or moderate by intensity and none was SAE.

Injection Site Reaction

Injection site reactions were primarily injection site erythema and were of mild intensity. There was one patient with severe, non-serious injection site reaction (AESI) in the SAR440340+dupilumab group that led to permanent treatment discontinuation. Incidence of injection site reactions was slightly lower in SAR440340 group compare to other treatment groups.

Severe and Serious Infections

One patient had a severe treatment emergent SAE of abscess jaw in dupilumab group. The event was reported as recovered after the corrective treatment. There were two non-serious infections of moderate intensity (two pneumonias) in the placebo group and were reported as recovered after the corrective treatment.

Potentially Drug-Related Hepatic Disorders

One treatment emergent SAE of alanine aminotransferase increased was reported in a patient in the SAR440340+dupilumab group and assessed as of moderate intensity. The patient's ALT increased to 431 U/L (13.33×ULN); baseline ALT was 11 U/L (normal range: 10-33 U/L). It was reported that the ALT increase was due to alcohol intake (2 glasses of beer, 1 glass of liquor). No corrective treatment was received. IMP treatment was temporarily withdrawn. The event recovered.

Pregnancy

One patient in the SAR440340 treatment group reported to be pregnant. Her last menses occurred 8 days after last IMP dose. At 6-week gestation period, the patient had elective termination of pregnancy.

Other Selected Adverse Event Groupings

Eosinophilia

Two patients reported eosinophilia. One patient in the SAR440340 group experienced worsening of eosinophilic colitis of moderate intensity. The patient had eosinophilic colitis ongoing since 2017. Her eosinophil count levels were constantly low during the study (<0.3 GI/L). The patient recovered on corrective treatment. One patient in the placebo group experienced an asymptomatic eosinophil count increase of mild intensity with eosinophil count>3 GI/L, with no recurrence (per protocol any eosinophil count increase>3 GI/L should be reported as AE). The patient recovered with no corrective treatment.

Laboratory Data, ECG and Vital Signs

There were no clinically meaningful significant differences between groups with regards to potentially clinically significant abnormality (PCSA) laboratory results.

There were no significant differences in all vital sign parameters, body weight, and ECG among treatment groups. One 62-years-old male patient had QTCF interval increase from baseline>60 msec in the SAR440340 treatment group. The patient had past medical history of hypertension. The ECG showed left ventricular hypertrophy. No relevant TEAE with regards to QTCF prolongation has been reported.

TABLE 12
Summary of Baseline Biomarkers, Randomized Population.

| | | | | |
|---|---|---|---|---|
| | SAR440340 | SAR440340 q2w + | Dupilumab | |
| Placebo | 300 mg q2w | Dupilumab q2w | 300 mg q2w | All |
| (N = 74) | (N = 73) | (N = 74) | (N = 75) | (N = 296) |

Baseline blood eosinophil (10⁹/L)

| | | | | | |
|---|---|---|---|---|---|
| Number | 74 | 73 | 74 | 74 | 295 |
| Mean (SD) | 0.41 (0.35) | 0.42 (0.57) | 0.34 (0.25) | 0.33 (0.26) | 0.37 (0.38) |
| Median | 0.33 | 0.29 | 0.25 | 0.26 | 0.28 |
| Q1; Q3 | 0.19; 0.51 | 0.17; 0.47 | 0.15; 0.50 | 0.16; 0.44 | 0.16; 0.48 |
| Min; Max | 0.0; 1.9 | 0.0; 4.5 | 0.0; 1.0 | 0.0; 1.8 | 0.0; 4.5 |
| <0.15 | 14 (18.9) | 15 (20.5) | 17 (23.0) | 17 (23.0) | 63 (21.4) |
| ≥0.15-<0.3 | 19 (25.7) | 22 (30.1) | 25 (33.8) | 26 (35.1) | 92 (31.2) |
| ≥0.3 | 41 (55.4) | 36 (49.3) | 32 (43.2) | 31 (41.9) | 140 (47.5) |

Baseline blood neutrophil (10⁹/L)

| | | | | | |
|---|---|---|---|---|---|
| Number | 74 | 73 | 74 | 74 | 295 |
| Mean (SD) | 4.20 (1.69) | 4.27 (1.77) | 4.20 (1.62) | 4.19 (1.82) | 4.22 (1.72) |
| Median | 3.88 | 3.84 | 3.78 | 3.67 | 3.80 |
| Q1; Q3 | 3.19; 5.05 | 3.31; 4.89 | 3.16; 5.15 | 2.98; 4.95 | 3.16; 4.97 |
| Min; Max | 1.9; 12.1 | 2.0; 11.7 | 2.1; 11.2 | 1.5; 11.3 | 1.5; 12.1 |

Baseline FeNO (ppb)

| | | | | | |
|---|---|---|---|---|---|
| Number | 74 | 73 | 74 | 75 | 296 |
| Mean (SD) | 34.1 (42.9) | 30.2 (24.6) | 24.1 (18.0) | 28.5 (27.4) | 29.2 (29.7) |
| Median | 19.5 | 23.0 | 18.5 | 19.0 | 20.0 |
| Q1; Q3 | 14.0; 33.0 | 12.0; 41.0 | 13.0; 29.0 | 12.0; 32.0 | 13.0; 32.0 |
| Min; Max | 2; 238 | 4; 111 | 5; 87 | 4; 151 | 2; 238 |
| <25 | 45 (60.8) | 39 (53.4) | 47 (63.5) | 48 (64.0) | 179 (60.5) |
| ≥25-<50 | 19 (25.7) | 20 (27.4) | 20 (27.0) | 18 (24.0) | 77 (26.0) |
| ≥50 | 10 (13.5) | 14 (19.2) | 7 (9.5) | 9 (12.0) | 40 (13.5) |

Baseline Total IL33 (pg/mL)

| | | | | | |
|---|---|---|---|---|---|
| Number | 74 | 73 | 74 | 74 | 295 |
| Mean (SD) | 17.1 (5.7) | 16.1 (2.7) | 16.4 (5.0) | 16.4 (3.9) | 16.5 (4.5) |
| Median | 15.7 | 15.7 | 15.7 | 15.7 | 15.7 |
| Q1; Q3 | 15.7; 15.7 | 15.7; 15.7 | 15.7; 15.7 | 15.7; 15.7 | 15.7; 15.7 |
| Min; Max | 16; 49 | 16; 32 | 16; 55 | 16; 36 | 16; 55 |

Baseline sST2 (pg/mL)

| | | | | | |
|---|---|---|---|---|---|
| Number | 72 | 71 | 73 | 74 | 290 |
| Mean (SD) | 6423.9 (3914.4) | 5708.6 (2549.2) | 5571.5 (2234.2) | 6408.4 (5095.3) | 6030.2 (3646.0) |
| Median | 5720.0 | 5280.0 | 5050.0 | 5350.0 | 5270.0 |
| Q1; Q3 | 3935.0; 7405.0 | 3950.0; 6890.0 | 4310.0; 6630.0 | 4090.0; 7120.0 | 4040.0; 6960.0 |
| Min; Max | 1300; 22800 | 1690; 16700 | 1960; 14900 | 1010; 35700 | 1010; 35700 |

Baseline calcitonin (pg/mL)

| | | | | | |
|---|---|---|---|---|---|
| Number | 74 | 73 | 74 | 75 | 296 |
| Mean (SD) | 2.20 (2.34) | 2.99 (5.04) | 2.07 (2.65) | 2.29 (2.76) | 2.39 (3.36) |
| Median | 1.00 | 1.00 | 1.00 | 1.00 | 1.00 |
| Q1; Q3 | 1.00; 2.80 | 1.00; 2.80 | 1.00; 1.00 | 1.00; 2.70 | 1.00; 2.60 |
| Min; Max | 1.0; 13.4 | 1.0; 33.0 | 1.0; 14.9 | 1.0; 13.8 | 1.0; 33.0 |

Baseline PARC (pg/mL)

| | | | | | |
|---|---|---|---|---|---|
| Number | 74 | 73 | 74 | 74 | 295 |
| Mean (SD) | 70144.6 (35045.7) | 75116.4 (67004.6) | 66778.9 (41623.6) | 76910.5 (54318.6) | 72227.9 (50827.3) |
| Median | 64550.0 | 59500.0 | 53900.0 | 62150.0 | 61800.0 |
| Q1; Q3 | 47600.0; 84100.0 | 44900.0; 78300.0 | 39700.0; 85700.0 | 43600.0; 90000.0 | 43500.0; 85700.0 |
| Min; Max | 17500; 201000 | 21500; 466000 | 940; 231000 | 2080; 326000 | 940; 466000 |

Baseline eotaxin-3 (pg/mL)

| | | | | | |
|---|---|---|---|---|---|
| Number | 74 | 73 | 74 | 74 | 295 |
| Mean (SD) | 47.14 (26.50) | 50.83 (67.80) | 77.85 (206.33) | 99.44 (327.66) | 68.88 (197.44) |
| Median | 46.65 | 34.20 | 41.95 | 41.60 | 41.40 |
| Q1; Q3 | 25.80; 64.60 | 28.10; 51.50 | 23.60; 63.40 | 31.10; 66.60 | 26.80; 61.00 |
| Min; Max | 4.6; 134.0 | 2.0; 559.0 | 2.0; 1760.0 | 2.0; 2820.0 | 2.0; 2820.0 |

Baseline Total IgE (kU/L)

| | | | | | |
|---|---|---|---|---|---|
| Number | 74 | 73 | 74 | 75 | 296 |
| Mean (SD) | 317.56 (523.05) | 682.04 (1798.02) | 314.36 (412.73) | 367.48 (548.84) | 419.30 (998.79) |
| Median | 161.50 | 140.00 | 144.50 | 170.00 | 160.50 |
| Q1; Q3 | 41.90; 400.00 | 47.30; 510.00 | 63.10; 396.00 | 57.30; 462.00 | 55.00; 416.00 |
| Min; Max | 9.2; 3339.0 | 1.0; 12975.0 | 3.7; 2556.0 | 1.0; 3452.0 | 1.0; 12975.0 |

Baseline periostin (ng/mL)

| | | | | | |
|---|---|---|---|---|---|
| Number | 74 | 73 | 74 | 74 | 295 |
| Mean (SD) | 84.72 (45.93) | 86.12 (48.17) | 83.45 (47.18) | 95.13 (58.45) | 87.36 (50.14) |
| Median | 74.40 | 73.30 | 77.90 | 76.20 | 74.40 |
| Q1; Q3 | 52.20; 94.80 | 58.80; 98.00 | 47.80; 105.00 | 56.10; 107.00 | 55.40; 101.00 |
| Min; Max | 38.4; 290.0 | 7.3; 267.0 | 3.1; 278.0 | 30.9; 326.0 | 3.1; 326.0 |

TABLE 3
Extent of Exposure to Investigational Product, Safety Population.

| | | SAR440340 | SAR440340 q2w + | Dupilumab |
|---|---|---|---|---|
| | Placebo | 300 mg q2w | Dupilumab q2w | 300 mg q2w |
| | (N = 39) | (N = 71) | (N = 104) | (N = 81) |
| Cumulative exposure to treatment | 6.5 | 12.3 | 16.5 | 14.6 |
| (patient years) | | | | |

Duration of study treatment (days)

| | | | | |
|---|---|---|---|---|
| Number | 39 | 71 | 104 | 81 |
| Mean (SD) | 61.0 (24.7) | 63.2 (21.2) | 58.1 (23.5) | 65.7 (21.3) |
| Median | 68.0 | 69.0 | 56.0 | 70.0 |
| Q1; Q3 | 42.0; 84.0 | 43.0; 84.0 | 42.0; 84.0 | 46.0; 84.0 |
| Min; Max | 14; 86 | 14; 89 | 14; 92 | 14; 92 |

Duration of study treatment by category [n(%)]

| | | | | |
|---|---|---|---|---|
| >0 and ≤2 weeks | 4 (10.3) | 2 (2.8) | 6 (5.8) | 3 (3.7) |
| >2 and ≤4 weeks | 2 (5.1) | 3 (4.2) | 7 (6.7) | 3 (3.7) |
| >4 and ≤8 weeks | 11 (28.2) | 23 (32.4) | 44 (42.3) | 24 (29.6) |
| >8 and <12 weeks − 3 days | 5 (12.8) | 14 (19.7) | 9 (8.7) | 14 (17.3) |
| ≥12 weeks − 3 days | 17 (43.6) | 29 (40.8) | 38 (36.5) | 37 (45.7) |

Number of patients with duration of study treatment by category [n(%)]

| | | | | |
|---|---|---|---|---|
| >0 week | 39 (100) | 71 (100) | 104 (100) | 81 (100) |
| >2 weeks | 35 (89.7) | 69 (97.2) | 98 (94.2) | 78 (96.3) |
| >4 weeks | 33 (84.6) | 66 (93.0) | 91 (87.5) | 75 (92.6) |
| >8 weeks | 22 (56.4) | 43 (60.6) | 47 (45.2) | 51 (63.0) |
| ≥12 weeks − 3 days | 17 (43.6) | 29 (40.8) | 38 (36.5) | 37 (45.7) |

Note:
Patients are considered in the treatment group they actually received.
Duration is defined as last dose date − first dose date + 14 days, regardless of unplanned intermittent discontinuations.

TABLE 4
Primary approach: Incidence of LOAC (all comparisons), mITT population.

| | | SAR440340 | SAR440340 q2w + | Dupilumab |
|---|---|---|---|---|
| | Placebo | 300 mg q2w | Dupilumab q2w | 300 mg q2w |
| Number of patients with LOAC | (N = 74) | (N = 73) | (N = 74) | (N = 74) |
| Number | 74 | 73 | 74 | 74 |
| No | 44 (59.5) | 57 (78.1) | 54 (73.0) | 60 (81.1) |
| Yes | 30 (40.5) | 16 (21.9) | 20 (27.0) | 14 (18.9) |
| OR vs placebo (95% CI)^{a} | | 0.423 (0.203 to 0.880) | 0.520 (0.256 to 1.057) | 0.328 (0.153 to 0.700) |
| P-value vs placebo^{a} | | 0.0214 | 0.0709 | 0.0040 |
| OR vs SAR440340 (95% CI)^{a} | | | 1.231 (0.571 to 2.653) | |
| P-value vs SAR440340^{a} | | | 0.5956 | |
| OR vs dupilumab (95% CI)^{a} | | | 1.589 (0.723 to 3.492) | |
| P-value vs dupilumab^{a} | | | 0.2494 | |

LOAC: Loss of asthma control,
OR: Odds ratio,
ICS: Inhaled corticosteroid
^{a}Derived from logistic regression with treatment, baseline eosinophil strata, region, background ICS dose level at randomization and number of exacerbation events within 1 year prior to screening.

TABLE 5
Summary of LOAC Events, mITT Population.

| | | SAR440340 | SAR440340 q2w + | Dupilumab |
|---|---|---|---|---|
| | Placebo | 300 mg q2w | Dupilumab q2w | 300 mg q2w |
| Number of patients with LOAC | (N = 74) | (N = 73) | (N = 74) | (N = 74) |
| Number | 74 | 73 | 74 | 74 |
| No | 44 (59.5) | 57 (78.1) | 54 (73.0) | 60 (81.1) |
| Yes | 30 (40.5) | 16 (21.9) | 20 (27.0) | 14 (18.9) |
| With rescue medication^{a} | 13 (17.6) | 6 (8.2) | 11 (14.9) | 5 (6.8) |
| Without rescue medication^{b} | 17 (23.0) | 10 (13.7) | 9 (12.2) | 9 (12.2) |
| Number of patients with ≥30% | 22 (29.7) | 12 (16.4) | 14 (18.9) | 11 (14.9) |
| reduction from baseline in AM | | | | |
| PEF on 2 consecutive days | | | | |
| Number of patients with ≥6 | 7 (9.5) | 3 (4.1) | 9 (12.2) | 3 (4.1) |
| additional reliever puffs of | | | | |
| salbutamol/albuterol or | | | | |
| levosalbutamol/levalbuterol in a 24 | | | | |
| hour period (compared with | | | | |
| baseline) on 2 consecutive days | | | | |
| Number of patients with increase | 1 (1.4) | 0 | 0 | 0 |
| in ICS ≥4 times the last prescribed | | | | |
| ICS dose (or ≥50% of the | | | | |
| prescribed ICS dose at baseline if | | | | |
| background therapy withdrawal | | | | |
| completed) | | | | |
| Number of patients requiring use | 7 (9.5) | 3 (4.1) | 3 (4.1) | 3 (4.1) |
| of systemic (oral and/or parenteral) | | | | |
| steroid treatment | | | | |
| Number of patients requiring | 0 | 1 (1.4) | 0 | 0 |
| hospitalization or emergency room | | | | |
| visit | | | | |

LOAC: Loss of asthma control, PEF: Peak expiratory flow, ICS: Inhaled corticosteroid.
^{a}If the patient met at least one of the following three criteria: (1) >=6 additional reliever puffs of salbutamol/albuterol or levosalbutamol/levalbuterol in a 24 hour period (compared to baseline) on 2 consecutive days, (2) Increase in ICS >=4 times the last prescribed ICS dose (or >=50% of the prescribed ICS dose at V2 if background therapy withdrawal completed), (3) Requiring use of systemic (oral and/or parenteral) steroid treatment.
^{b}If the patient met none of the three criteria listed above and met one or both of the following: (1) A 30% or greater reduction from baseline in AM PEF on 2 consecutive days, (2) Requiring hospitalization or emergency room visit.

TABLE 6
Subgroup Analysis: Incidence of LOAC by Baseline Blood Eosinophil
Count (Primary Comparisons), mITT Population.

| | | | | |
|---|---|---|---|---|
| | SAR440340 | SAR440340 q2w + | Dupilumab | |
| Placebo | 300 mg q2w | Dupilumab q2w | 300 mg q2w | p-value for |
| (N = 74) | (N = 73) | (N = 74) | (N = 74) | interaction^{a} |

| | |
|---|---|
| Baseline blood eosinophil count | 0.5365 |
| (/mm³) | |

<150

| | | | | |
|---|---|---|---|---|
| Number | 14 | 15 | 17 | 17 |
| No | 9 (64.3) | 12 (80.0) | 13 (76.5) | 13 (76.5) |
| Yes | 5 (35.7) | 3 (20.0) | 4 (23.5) | 4 (23.5) |
| OR vs placebo (95% CI)^{b} | | 0.401 (0.072 to 2.239) | 0.623 (0.122 to 3.182) | |
| P-value vs placebo^{b} | | 0.2975 | 0.5694 | |

≥150-<300

| | | | | |
|---|---|---|---|---|
| Number | 19 | 22 | 25 | 26 |
| No | 13 (68.4) | 18 (81.8) | 16 (64.0) | 20 (76.9) |
| Yes | 6 (31.6) | 4 (18.2) | 9 (36.0) | 6 (23.1) |
| OR vs placebo (95% CI)^{b} | | 0.473 (0.099 to 2.270) | 1.086 (0.270 to 4.372) | |
| P-value vs placebo^{b} | | 0.3495 | 0.9079 | |

≥300

| | | | | |
|---|---|---|---|---|
| Number | 41 | 36 | 32 | 31 |
| No | 22 (53.7) | 27 (75.0) | 25 (78.1) | 27 (87.1) |
| Yes | 19 (46.3) | 9 (25.0) | 7 (21.9) | 4 (12.9) |
| OR vs placebo (95% CI)^{b} | | 0.388 (0.143 to 1.054) | 0.298 (0.102 to 0.868) | |
| P-value vs placebo^{b} | | 0.0633 | 0.0265 | |

LOAC: Loss of asthma control, OR: Odds ratio, ICS: Inhaled corticosteroid.
^{a}Derived from logistic regression with treatment, baseline eosinophil strata, region, background ICS dose level at randomization, number of exacerbation events within 1 year prior to screening, subgroup (if different from the aforementioned covariates), and subgroup-by-treatment interaction.
^{b}Derived from logistic regression with treatment, baseline eosinophil strata, region, background. ICS dose level at randomization and number of exacerbation events within 1 year prior to screening.

TABLE 7
Primary Approach: Change From Baseline in Pre-Bronchodilator
FEV1 (L) at Week 12 (All Comparisons), mITT Population.

| | | | | |
|---|---|---|---|---|
| | | SAR440340 | SAR440340 q2w + | Dupilumab |
| | Placebo | 300 mg q2w | Dupilumab q2w | 300 mg q2w |
| Pre-bronchodilator FEV1 (L) | (N = 74) | (N = 73) | (N = 74) | (N = 74) |

Baseline

| | | | | |
|---|---|---|---|---|
| Number | 74 | 73 | 74 | 74 |
| Mean (SD) | 2.12 (0.61) | 1.93 (0.47) | 2.00 (0.57) | 2.03 (0.62) |
| Median | 1.93 | 1.85 | 1.88 | 1.99 |
| Q1; Q3 | 1.72; 2.44 | 1.62; 2.23 | 1.59; 2.31 | 1.52; 2.27 |
| Min; Max | 1.2; 4.0 | 1.0; 3.2 | 1.0; 3.5 | 1.1; 3.6 |

Week 12

| | | | | |
|---|---|---|---|---|
| Number | 41 | 58 | 49 | 56 |
| Mean (SD) | 2.32 (0.74) | 2.03 (0.60) | 2.01 (0.59) | 2.20 (0.81) |
| Median | 2.17 | 1.92 | 1.86 | 2.11 |
| Q1; Q3 | 1.73; 2.85 | 1.68; 2.19 | 1.55; 2.59 | 1.58; 2.70 |
| Min; Max | 1.2; 4.3 | 1.0; 3.8 | 0.9; 3.2 | 0.8; 4.5 |

Change from baseline

| | | | | |
|---|---|---|---|---|
| Number | 41 | 58 | 49 | 56 |
| Mean (SD) | 0.06 (0.35) | 0.11 (0.34) | 0.06 (0.37) | 0.14 (0.43) |
| Median | 0.01 | 0.06 | −0.03 | 0.06 |
| Q1; Q3 | −0.18; 0.21 | −0.11; 0.34 | −0.13; 0.29 | −0.18; 0.39 |
| Min; Max | −0.6; 1.2 | −0.7; 1.0 | −1.0; 1.0 | −0.9; 1.4 |
| LS Mean (SE)^{a} | −0.04 (0.05) | 0.10 (0.05) | 0.06 (0.05) | 0.12 (0.05) |
| LS Mean diff vs. placebo | | 0.14 (0.01 to 0.27) | 0.10 (−0.03 to 0.23) | 0.16 (0.03 to 0.29) |
| (95% CI)^{a} | | | | |
| P-value vs. placebo^{a} | | 0.0344 | 0.1337 | 0.0140 |
| LS Mean diff vs. | | | −0.04 (−0.17 to 0.09) | |
| SAR440340 (95% CI)^{a} | | | | |
| P-value vs. SAR440340^{a} | | | 0.5289 | |
| LS Mean diff vs. | | | −0.06 (−0.19 to 0.06) | |
| dupilumab (95% CI)^{a} | | | | |
| P-value vs. dupilumab^{a} | | | 0.3293 | |

FEV1: Forced expiratory volume in one second, LOAC: Loss of asthma control, ICS: Inhaled corticosteroid.
Data collected up to end of treatment visit were included. For patients who experienced LOAC with rescue medications, FEV1 collected at and after start of the event were set to missing. Missing data were not imputed.
^{a}Derived from MMRM model with change from baseline values up to week 12 as the response variable, and treatment, sex, baseline height, baseline eosinophil strata, region, background ICS dose level at randomization, visit, treatment by-visit interaction, baseline value and baseline-by-visit interaction as covariates.

TABLE 8
Subgroup Analysis: Change from Baseline in Pre-Bronchodilator FEV1 (L) at Week
12 by Baseline Blood Eosinophil Count (primary comparisons) - mITT Population.

| | | | | | |
|---|---|---|---|---|---|
| | | SAR440340 | SAR440340 q2w + | Dupilumab | |
| | Placebo | 300 mg q2w | Dupilumab q2w | 300 mg q2w | p-value for |
| Pre-bronchodilator FEV1 (L) | (N = 74) | (N = 73) | (N = 74) | (N = 74) | interaction^{a} |

| | |
|---|---|
| Baseline blood eosinophil count | 0.1111 |
| (/mm³) | |

<150

| | | | | |
|---|---|---|---|---|
| Number | 9 | 12 | 14 | 13 |
| Baseline mean (SD) | 2.50 (0.93) | 2.07 (0.49) | 1.97 (0.49) | 2.19 (0.77) |
| Week 12 mean (SD) | 2.51 (1.00) | 2.16 (0.70) | 1.98 (0.66) | 2.19 (1.08) |
| Change mean (SD) | 0.02 (0.24) | 0.09 (0.32) | 0.01 (0.28) | −0.00 (0.53) |
| LS Mean (SE)^{a} | −0.06 (0.11) | 0.10 (0.10) | 0.05 (0.10) | −0.05 (0.09) |
| LS Mean diff vs. placebo | | 0.17 (−0.12 to 0.45) | 0.11 (−0.17 to 0.39) | 0.01 (−0.27 to 0.29) |
| (95% CI)^{b} | | | | |
| P-value vs. placebo^{b} | | 0.2407 | 0.4304 | 0.9457 |
| LS Mean diff vs. | | | −0.06 (−0.33 to 0.21) | |
| SAR440340 (95% CI)^{b} | | | | |
| P-value vs. SAR440340^{b} | | | 0.6627 | |
| LS Mean diff vs. | | | 0.10 (−0.16 to 0.36) | |
| dupilumab (95% CI)^{b} | | | | |
| P-value vs. dupilumab^{b} | | | 0.4494 | |

≥150-<300

| | | | | |
|---|---|---|---|---|
| Number | 10 | 20 | 16 | 18 |
| Baseline mean (SD) | 2.06 (0.47) | 1.78 (0.45) | 1.87 (0.46) | 1.92 (0.67) |
| Week 12 mean (SD) | 2.28 (0.48) | 1.81 (0.42) | 1.83 (0.50) | 1.92 (0.73) |
| Change mean (SD) | 0.22 (0.41) | 0.03 (0.29) | −0.04 (0.26) | −0.00 (0.29) |
| LS Mean (SE)^{a} | 0.13 (0.09) | 0.00 (0.07) | −0.07 (0.07) | 0.06 (0.07) |
| LS Mean diff vs. placebo | | −0.13 (−0.34 to 0.07) | −0.21 (−0.41 to 0.00) | −0.07 (−0.28 to 0.14) |
| (95% CI)^{b} | | | | |
| P-value vs. placebo^{b} | | 0.2063 | 0.0506 | 0.5087 |
| LS Mean diff vs. | | | −0.08 (−0.26 to 0.11) | |
| SAR440340 (95% CI)^{b} | | | | |
| P-value vs. SAR440340^{b} | | | 0.4108 | |
| LS Mean diff vs. | | | −0.14 (−0.32 to 0.05) | |
| dupilumab (95% CI)^{b} | | | | |
| P-value vs. dupilumab^{b} | | | 0.1488 | |

≥300

| | | | | |
|---|---|---|---|---|
| Number | 22 | 26 | 19 | 25 |
| Baseline mean (SD) | 2.26 (0.64) | 1.95 (0.43) | 2.00 (0.39) | 2.11 (0.54) |
| Week 12 mean (SD) | 2.26 (0.74) | 2.14 (0.65) | 2.18 (0.60) | 2.41 (0.67) |
| Change mean (SD) | 0.00 (0.35) | 0.19 (0.38) | 0.17 (0.49) | 0.31 (0.40) |
| LS Mean (SE)^{a} | −0.04 (0.07) | 0.18 (0.07) | 0.15 (0.08) | 0.30 (0.08) |
| LS Mean diff vs. placebo | | 0.22 (0.02 to 0.41) | 0.19 (−0.01 to 0.40) | 0.34 (0.14 to 0.54) |
| (95% CI)^{b} | | | | |
| P-value vs. placebo^{b} | | 0.0312 | 0.0624 | 0.0010 |
| LS Mean diff vs. | | | −0.02 (−0.23 to 0.19) | |
| SAR440340 (95% CI)^{b} | | | | |
| P-value vs. SAR440340^{b} | | | 0.8218 | |
| LS Mean diff vs. | | | −0.14 (−0.35 to 0.07) | |
| dupilumab (95% CI)^{b} | | | | |
| P-value vs. dupilumab^{b} | | | 0.1780 | |

FEV1: Forced expiratory volume in one second, ICS: Inhaled corticosteroid, LOAC: Loss of asthma control.
Data collected up to end of treatment visit were included. For patients who experienced LOAC with rescue medications, FEV1 collected at and after start of the event were set to missing. Missing data were not imputed.
^{a}Derived from MMRM model with change from baseline values up to Week 12 as the response variable, and treatment, sex, baseline height, baseline eosinophil strata, region, background ICS dose level at randomization, visit, treatment by-visit interaction, baseline value, baseline-by-visit interaction, subgroup (if different from the aforementioned covariates), subgroup-by-treatment interaction and subgroup-by-treatment-by-visit interaction as covariates.
^{b}Derived from MMRM model with change from baseline values up to Week 12 as the response variable, and treatment, sex, baseline height, baseline eosinophil strata, region, background ICS dose level at randomization, visit, treatment by-visit interaction, baseline value and baseline-by-visit interaction as covariates.

TABLE 9
Summary of Hypersensitivity Reaction (Medically Reviewed), Safety Population

| | | SAR440340 | SAR440340 q2w + | Dupilumab |
|---|---|---|---|---|
| | Placebo | 300 mg q2w | Dupilumab q2w | 300 mg q2w |
| | (N = 74) | (N = 73) | (N = 74) | (N = 74) |
| Patients with any TEAE | 1 (1.4) | 3 (4.1) | 3 (4.1) | 1 (1.4) |
| Patients with any SAE | 0 | 0 | 0 | 0 |
| Patients with any treatment-emergent | 0 | 0 | 0 | 0 |
| SAE | | | | |
| Patients with any AE leading to death | 0 | 0 | 0 | 0 |
| Patients with any TEAE leading to | 0 | 0 | 0 | 0 |
| death | | | | |
| Patients with any TEAE leading to | 1 (1.4) | 0 | 1 (1.4) | 0 |
| permanent treatment discontinuation | | | | |
| Patients with any TEAE related to | 1 (1.4) | 0 | 1 (1.4) | 0 |
| SAR440340 or matching placebo as | | | | |
| reported by investigator | | | | |
| Patients with any TEAE related to | 1 (1.4) | 0 | 0 | 0 |
| Dupilumab or matching placebo as | | | | |
| reported by investigator | | | | |
| Number of TEAE (Number of TEAE | 1 (3.5) | 3 (10.1) | 3 (10.3) | 1 (3.4) |
| per 100 patient-years) | | | | |
| Number of patients with at least one | 1 (3.5) | 3 (10.1) | 3 (10.3) | 1 (3.4) |
| TEAE (Number of patients with at | | | | |
| least one TEAE per 100 patient-years) | | | | |

Maximal intensity

| | | | | |
|---|---|---|---|---|
| Mild | 0 | 2 (2.7) | 0 | 0 |
| Moderate | 1 (1.4) | 1 (1.4) | 3 (4.1) | 1 (1.4) |
| Severe | 0 | 0 | 0 | 0 |

Corrective treatment

| | | | | |
|---|---|---|---|---|
| No | 0 | 0 | 0 | 0 |
| Yes | 1 (1.4) | 3 (4.1) | 3 (4.1) | 1 (1.4) |

Outcome

| | | | | |
|---|---|---|---|---|
| Fatal | 0 | 0 | 0 | 0 |
| Not Recovered/Not Resolved | 0 | 0 | 0 | 0 |
| Recovered or Resolved | 1 (1.4) | 3 (4.1) | 3 (4.1) | 1 (1.4) |
| Recovered or Resolved with | 0 | 0 | 0 | 0 |
| Sequelae | | | | |
| Recovering or Resolving | 0 | 0 | 0 | 0 |
| Unknown | 0 | 0 | 0 | 0 |

Primary System Organ Class/Preferred Term n(%)

| | | | | |
|---|---|---|---|---|
| Immune system disorders | 0 | 0 | 1 (1.4) | 0 |
| Hypersensitivity | 0 | 0 | 1 (1.4) | 0 |
| Eye disorders | 0 | 0 | 0 | 1 (1.4) |
| Conjunctivitis allergic | 0 | 0 | 0 | 1 (1.4) |
| Gastrointestinal disorders | 0 | 1 (1.4) | 0 | 0 |
| Lip swelling | 0 | 1 (1.4) | 0 | 0 |
| Skin and subcutaneous tissue disorders | 1 (1.4) | 2 (2.7) | 2 (2.7) | 0 |
| Rash pruritic | 0 | 0 | 2 (2.7) | 0 |
| Pruritus | 0 | 1 (1.4) | 0 | 0 |
| Pruritus generalized | 0 | 1 (1.4) | 0 | 0 |
| Urticaria | 1 (1.4) | 0 | 0 | 0 |

TEAE: Treatment-emergent adverse event, SAE: Serious adverse event, SOC: System organ class, PT: Preferred term.
MedDRA 21.1.
Note:
Table sorted by SOC internationally agreed order and decreasing frequency of PT in the coadministration group.

1. A method for treating asthma in a subject in need thereof comprising administering to the subject:
an initial dose of about 300 mg of a first antibody or antigen-binding fragment thereof that specifically binds interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16; and
one or more maintenance doses of about 300 mg of the antibody or antigen-binding fragment thereof, and optionally administering to the subject:
an initial dose of about 300 mg of a second antibody or antigen-binding fragment thereof that specifically binds interleukin-4 receptor (IL-4R) and comprises three heavy chain complementary determining region (HCDR) sequences comprising SEQ ID NOs: 21, 22 and 23, and three light chain complementary determining region (LCDR) sequences comprising SEQ ID NOs: 24, 25 and 26; and
one or more maintenance doses of about 300 mg of the second antibody or antigen-binding fragment thereof.

2. The method of claim 1, wherein:
(a) loss of asthma control (LOAC) is reduced in the subject;
(b) one or more asthma-associated parameter(s) are improved in the subject,
optionally selected from the group consisting of forced expiratory volume in 1 second (FEV1), peak expiratory flow (PEF), forced vital capacity (FVC), forced expiratory flow (FEF) 25%-75%, frequency or dosage of a long-acting β2 adrenergic agonist (LABA), frequency or dosage of an inhaled corticosteroid, and frequency or dosage of a systemic steroid,
optionally wherein pre-bronchodilator FEV1 is improved and/or the frequency or the dosage of the long-acting β2 adrenergic agonist (LABA) is reduced, the frequency or the dosage of the inhaled corticosteroid is reduced, or the frequency or the dosage of the systemic steroid is reduced;
(c) one or both of asthma control questionnaire 5-question version (ACQ-5) score and asthma quality of life questionnaire with standardized activities (AQLQ) score are improved, optionally wherein the emotional function score of the AQLQ is improved;
(d) the asthma is moderate-to-severe asthma that is not well-controlled on a background therapy, optionally wherein the background therapy comprises an inhaled corticosteroid (ICS) and a long-acting β2 adrenergic agonist (LABA), and optionally moderate-to-high dose ICS/LABA;
(e) the subject has a blood eosinophil count of: greater than or equal to about 300 cells per μl; of about 150 to 299 cells/μL; or of about <150 cells/μL;
(f) blood eosinophil levels are reduced; and/or
(g) the subject has high blood periostin levels or low blood periostin levels, optionally wherein the subject has high blood periostin levels of about ≥74.4 ng/mL.

3-16. (canceled)

17. The method of claim 1, wherein the first antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10, optionally wherein the second antibody comprises SAR440340 and
wherein the second antibody or antigen-binding fragment thereof comprises a heavy chain variable region (HCVR) comprising the amino acid sequence of SEQ ID NO: 27 and a light chain variable region (LCVR) comprising the amino acid sequence of SEQ ID NO: 28, optionally wherein the second antibody comprises dupilumab.

18. (canceled)

19. The method of claim 1, wherein the first antibody or antigen-binding fragment thereof and the second antibody or antigen-binding fragment thereof are each administered:
(a) every other week;
(b) subcutaneously, optionally using an autoinjector, a needle and syringe, or a pen delivery device; and/or
(c) as two injections.

20-39. (canceled)

40. The method of claim 1, wherein the second antibody or antigen-binding fragment thereof is administered to the subject before, after, or concurrent with the first antibody or antigen-binding fragment thereof.

41. The method of claim 1, wherein the first antibody or antigen-binding fragment thereof is administered as two injections and the second antibody or antigen-binding fragment thereof is administered as one injection.

42. (canceled)

43. The method of claim 1, wherein at least one additional therapeutic agent is administered to the subject.

44. The method of claim 43, wherein the at least one additional therapeutic agent comprises one or both of an ICS, optionally fluticasone or budesonide, and a LABA, optionally salmeterol or formoterol.

45-48. (canceled)

49. A method for treating moderate-to-severe asthma in a subject in need thereof comprising administering to the subject:
an initial dose of about 300 mg of SAR440340; and
one or more maintenance doses of about 300 mg of SAR440340; and optionally:
an initial dose of about 300 mg of dupilumab; and
one or more maintenance doses of about 300 mg of dupilumab;
wherein SAR440340 and dupilumab are administered subcutaneously every other week.

50. A method for reducing an asthma patient's dependence or one or both of an inhaled corticosteroid (ICS) and a long-acting β2 adrenergic agonist (LABA) for the treatment of one or more asthma exacerbations comprising:
administering to a subject who has moderate-to-severe asthma that is partially controlled or uncontrolled with a background asthma therapy comprising an ICS, a LABA, or a combination thereof, a defined dose of an antibody or antigen-binding fragment thereof that specifically binds to interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16, at a defined frequency for an initial treatment period while maintaining the subject's background asthma therapy for the initial treatment period; and
gradually reducing or eliminating the dosage of the ICS, the LABA or the combination thereof administered to the subject over the course of a subsequent treatment period while continuing to administer the antibody or antigen-binding fragment thereof to the subject at the defined frequency and dose used during the initial treatment period.

51. The method of claim 50,
(a) wherein the ICS is fluticasone, budesonide, or mometasone, and the LABA is salmeterol or formoterol;
(b) comprising an ICS/LABA combination selected from the group consisting of fluticasone/salmeterol, budesonide/formoterol, and mometasone/formoterol;
(c) wherein the dosage of one or both of the LABA and the ICS are eliminated at the end of the initial treatment period; and/or
(d) wherein the dosage of one or both of the LABA and the ICS are gradually reduced or eliminated over the course of 2 to 8 weeks.

52-54. (canceled)

55. The method of claim 50, further comprising administering to the subject a second antibody or antigen-binding fragment thereof that specifically binds interleukin-4 receptor (IL-4R) and comprises three HCDR sequences comprising SEQ ID NOs: 21, 22 and 23, and three LCDR sequences comprising SEQ ID NOs: 24, 25 and 26.

56. (canceled)

57. A method for treating asthma in a subject in need thereof comprising administering to the subject:
a dose of about 300 mg of a first antibody or antigen-binding fragment thereof that specifically binds interleukin-33 (IL-33) and comprises three heavy chain complementary determining region (HCDR) sequences comprising SEQ ID NOs: 4, 5 and 6, and three light chain complementary determining region (LCDR) sequences comprising SEQ ID NOs: 12, 14 and 16; and optionally:
a dose of about 300 mg of a second antibody or antigen-binding fragment thereof that specifically binds interleukin-4 receptor (IL-4R) and comprises three heavy chain complementary determining region (HCDR) sequences comprising SEQ ID NOs: 21, 22 and 23, and three light chain complementary determining region (LCDR) sequences comprising SEQ ID NOs: 24, 25 and 26.

58. The method of claim 57, wherein:
(a) loss of asthma control (LOAC) is reduced in the subject;
(b) one or more asthma-associated parameter(s) are improved in the subject,
optionally selected from the group consisting of forced expiratory volume in 1 second (FEV1), peak expiratory flow (PEF), forced vital capacity (FVC), forced expiratory flow (FEF) 25%-75%, frequency or dosage of a long-acting β2 adrenergic agonist (LABA), frequency or dosage of an inhaled corticosteroid, and frequency or dosage of a systemic steroid,
optionally wherein pre-bronchodilator FEV1 is improved and/or the frequency or the dosage of the long-acting β2 adrenergic agonist (LABA) is reduced, the frequency or the dosage of the inhaled corticosteroid is reduced, or the frequency or the dosage of the systemic steroid is reduced;
(c) one or both of asthma control questionnaire 5-question version (ACQ-5) score and asthma quality of life questionnaire with standardized activities (AQLQ) score are improved, optionally wherein the emotional function score of the AQLQ is improved;
(d) the asthma is moderate-to-severe asthma that is not well-controlled on a background therapy, optionally wherein the background therapy comprises an inhaled corticosteroid (ICS) and a long-acting β2 adrenergic agonist (LABA), and optionally moderate-to-high dose ICS/LABA;
(e) the subject has a blood eosinophil count of: greater than or equal to about 300 cells per μl; of about 150 to 299 cells/μL; or of about <150 cells/μL;
(f) blood eosinophil levels are reduced;
(g) the subject has high blood periostin levels or low blood periostin levels, optionally wherein the subject has high blood periostin levels of about ≥74.4 ng/mL, and/or
(h) the asthma is moderate-to-severe asthma that is not well-controlled on a background therapy, optionally wherein the background therapy comprises an inhaled corticosteroid (ICS) and a long-acting β2 adrenergic agonist (LABA), and optionally wherein the background therapy comprises moderate-to-high dose ICS/LABA.

59-74. (canceled)

75. The method of claim 57, wherein the first antibody or antigen-binding fragment thereof comprises a heavy chain variable region (HCVR) comprising the amino acid sequence of SEQ ID NO: 2 and a light chain variable region (LCVR) comprising the amino acid sequence of SEQ ID NO: 10 and
wherein the second antibody or antigen-binding fragment thereof comprises a heavy chain variable region (HCVR) comprising the amino acid sequence of SEQ ID NO: 27 and a light chain variable region (LCVR) comprising the amino acid sequence of SEQ ID NO: 28.

76. The method of claim 75, wherein the first antibody comprises SAR440340.

77. (canceled)

78. The method of claim 75, wherein the second antibody comprises dupilumab.

79. The method of claim 57, wherein the antibody or antigen-binding fragment thereof is administered subcutaneously using an autoinjector, a needle and syringe, or a pen delivery device.
